SEQUENCE LISTING (6 sequences)

SEQ ID NO: 1 - protein, 19 aa, synthetic construct
  MOD_RES 19: note = C-terminal amidation
DLDLEMLAPYIPMDDDFQL

SEQ ID NO: 2 - protein, 12 aa, synthetic construct
RSKKRKKSKSRS

SEQ ID NO: 3 - protein, 14 aa, synthetic construct
  MOD_RES 9: note = lysine tri-methylation
ARTAQTARKSTGGI

SEQ ID NO: 4 - protein, 21 aa, synthetic construct
  MOD_RES 4: note = lysine tri-methylation
ARTKQTARKSTGGKAPRKQLA

SEQ ID NO: 5 - protein, 17 aa, synthetic construct
  MOD_RES 8: note = lysine tri-methylation
LATKAARKSAPATGGVK

SEQ ID NO: 6 - protein, 30 aa, synthetic construct
  MOD_RES 30: note = amidated C-terminus
AKGIVLEKVGVEAKQPNSAIRKAVRVQLIK

(19) United States
(12) Patent Application Publication
(10) Pub. No.: US 2026/0226055 A1
(43) Pub. Date: Aug. 6, 2026

(54) PYRAZOLE DERIVATIVES AS PHD INHIBITORS

(71) Applicant: Oxford University Innovation Limited, Oxford (GB)

(72) Inventors: Christopher Joseph Schofield, Oxford (GB); James Preston Holt-Martyn, Oxford (GB)

(21) Appl. No.: 19/149,981

(22) PCT Filed: Jan. 25, 2024
(86) PCT No.: PCT/GB2024/050198

§ 371 Date: Jul. 22, 2025

(30) Foreign Application Priority Data
Jan. 25, 2023 (GB) 2301050.7

(51) Int. Cl. C07D 471/14; A61K 31/4439; A61K 31/444; A61K 31/501; A61K 31/506; A61K 31/519; A61K 31/5377; A61K 45/06; A61P 7/00; A61P 7/06; C07D 401/04; C07D 401/14; C07D 403/04; C07D 417/14

(52) U.S. Cl. CPC C07D 471/14; A61K 31/4439; A61K 31/444; A61K 31/501; A61K 31/506; A61K 31/519; A61K 31/5377; A61K 45/06; A61P 7/00; A61P 7/06; C07D 401/04; C07D 401/14; C07D 403/04; C07D 417/14

(57) ABSTRACT

A compound which is a substituted azine of formula (I), a substituted pyrimidine of formula (IV), or a pharmaceutically acceptable salt thereof

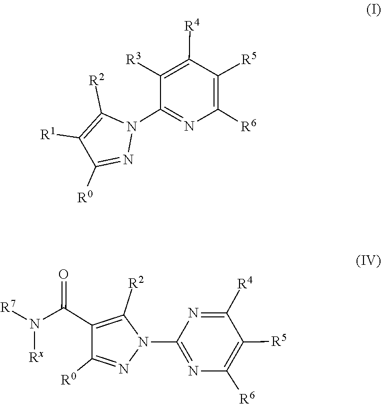

### FIELD OF THE INVENTION

The invention relates to a series of novel compounds and their use as hypoxia inducible factor prolyl hydroxylase domain inhibitors (PHD inhibitors).

### BACKGROUND TO THE INVENTION

The hypoxia inducible factor (HIF) prolyl hydroxylases (PHDs) are therapeutic targets for applications, including for treatment of anaemia and other ischemia-related diseases, including cancer and inflammation. The PHDs are Fe(II) and 2-oxoglutarate (2OG) dependent oxygenases that catalyse the hydroxylation of specific prolyl residues within the oxygen degradation domains (ODDs) of the HIF-α subunits. As oxygen levels decrease HIF-α levels rise; HIF-α dimerises with HIF-β, and the α,β-HIF complex promotes transcription of HIF target genes. However, it is likely that none of the current ‘clinical’ PHD inhibitors are completely selective for the PHDs over other human 2OG oxygenases. This lack of selectivity can lead to unpredictable off-target effects which mean that treatment may be unsafe. There is therefore a need for more selective small molecule PHD inhibitors.

Anaemia, a decrease in the amount of red blood cells (erythrocytes) in circulation, is a major contributor to human global mortality and morbidity. A recent study suggested one third of the global population may be affected. Anaemia can occur in various ways, including infection, nutrition, chronic kidney disease and iron deficiency.

Deficiencies in erythropoietin (EPO) are broadly linked to anaemia associated with multiple chronic diseases, especially those linked to systemic inflammation. EPO production mainly occurs in the liver and kidneys, although many other organs express the EPO gene. EPO production has been shown to be substantially sensitive to changes in oxygen concentrations in the atmosphere blood.

Recombinant human EPO (rhEPO) is currently used to treat anaemia in patients, however, EPO cannot be delivered orally. Many clinical studies have indicated that higher doses of rhEPO correlate with an increase in cardiovascular events. This has been linked to the high doses of rhEPO rather than the increased levels of haemoglobin. High plasma concentrations of EPO have been linked to vascular toxicity.

There is a need therefore, to find alternative treatments for anaemia which can be administered more easily and do not present the above described risks.

Previous clinical studies using PHD inhibitors have shown therapeutically useful levels of haemoglobin can be achieved corresponding to normal physiological plasma concentrations of EPO. HIF target genes are reported to be involved in iron metabolism, transport and absorption, thereby potentially enhancing iron availability for erythropoiesis. However, the effects of HIF beyond EPO regulation are difficult to predict due to the broad tissue distribution and the complexity of the HIF system.

Hypoxia occurs in ischaemic environments. Poor blood flow (organ ischemia) is a major clinical problems of the modern age, occurring in circulatory and cardiovascular diseases and potentially during surgical operations and in impaired wound healing. Poor blood flow commonly affects the kidneys, limbs, heart and the brain and can be either chronic or acute. It has been envisaged that PHD inhibitors will stabilise HIF and be protective and/or reparative will respond to ischaemic diseases.

When designing PHD inhibitors, as well as target selectivity, it is desirable to find candidates which have a high efficacy, and which display good properties suited for use as pharmaceuticals.

Currently available PHD inhibitors, including roxadustat, daprodustat, molidustat, desidustat and vadadustat, display only limited target selectivity for the PHDs, with, for example, inhibition by one or more of them being observed with collagen prolyl hydroxylases (CPHs); 2-oxoglutarate and iron dependent oxygenase domain containing 1 (OFGOD1); and jumonji domain containing 6 (JMJD6). It is well known that a lack of selectivity of an enzyme inhibitor can lead to unpredictable and undesirable off-target effects.

Therefore, there is a need for more specific PHD inhibitors with physical properties suitable for use as drugs.

### SUMMARY OF THE INVENTION

The present invention provides a series of novel compounds that have been shown to have high efficacy and selectivity as human hypoxia inducible factor (HIF) prolyl hydroxylase (PHD) inhibitors. Some compounds of the invention have been shown to have an IC₅₀ for PHD2 of less than 200 nM, which is a substantial improvement compared to known clinically applied inhibitors (e.g. roxadustat has an IC₅₀ of 2.7 μM in a liquid chromatography based PHD2 hydroxylation assay).

As well as their potency, compounds of the invention have been found to be highly selective for the PHDs, with greater than 100-fold selectivity compared to other tested 2OG oxygenases.

As well as these desirable biochemical properties, compounds of the invention have been shown to have desirable physiochemical properties including good solubility and permeability in cells.

These physiochemical properties mean that compounds of the invention of have been found to stabilise cellular HIF-α at concentrations in the nM potency range as measured by enzyme assays. Additionally, low doses of the compounds have been shown to induce erythropoiesis in animal models.

The compounds therefore have potential utility in treating conditions for which HIF-PHD is a therapeutic target, including for instance anaemia and other ischemia-related diseases, inflammation, and further conditions as mentioned below.

Accordingly, the present invention provides a compound which is a substituted azine of formula (I) or a pharmaceutically acceptable salt thereof

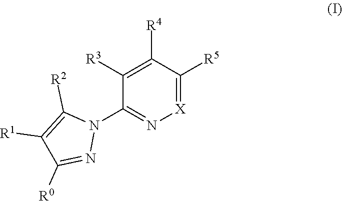

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w} or —C(O)N(R^{x})R⁷;
R² is H, —OR^{q} or unsubstituted or substituted C₁₋₆ alkyl; and R³ is H, —OR⁸ or unsubstituted or substituted C₁₋₆ alkyl; or R² is —N═ and R³ is ═C(R^{y})— and R² and R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN, —N(R^{t})C(O)N(R^{u})R⁰, —C(O)OR^{w} or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸, R⁹ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl;
   R^{q} is H, unsubstituted or substituted C₁₋₆ alkyl, or unsubstituted or substituted phenyl;
provided that one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —CN, —N(R^{t})C(O)N(R^{u})R⁰ or —C(O)OR^{w}.

Preferred embodiments of the compound which is a substituted azine of formula (I) or a pharmaceutically acceptable salt thereof are described hereinbelow, including the substituted azines of formulae (Ia), (Ib), (Ic) and (Id) as defined hereinbelow and pharmaceutically acceptable salts thereof.

In another aspect the invention provides a compound which is a substituted pyrimidine of formula (IV) or a pharmaceutically acceptable salt thereof

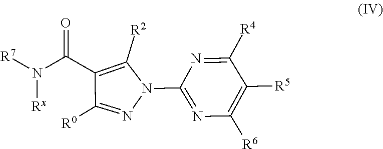

wherein
R⁰ is H or unsubstituted or substituted C₁₋₆ alkyl;
R² is H, —OR^{q} or unsubstituted or substituted C₁₋₆ alkyl;
R⁴ is —OR⁹;
R⁵ is H, unsubstituted or substituted C₁₋₆ alkyl, unsubstituted or substituted aryl, unsubstituted or substituted heteroaryl, —CN or —C(O)OR¹;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is selected from H and unsubstituted or substituted C₁₋₆ alkyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

The invention also provides a pharmaceutical composition comprising a compound of the invention as defined above and a pharmaceutically acceptable carrier or diluent. The pharmaceutical composition may further comprise one or more additional active agents, for instance as mentioned below.

In another aspect the invention provides a compound of the invention as defined above, or the pharmaceutical composition of the invention, for use in the treatment of the human or animal body by therapy.

The invention also provides a compound of the invention as defined above, or a pharmaceutical composition of the invention, for use as a modulator of hypoxia inducible factor prolyl hydroxylase activity. Typically, the compound or composition is for use as an inhibitor of hypoxia inducible factor prolyl hydroxylase activity. The invention additionally provides a compound of the invention as defined above, or the pharmaceutical composition of the invention, for use in treating a PHD related disorder, i.e. a disorder that can be treated by modulating (for instance inhibiting) hypoxia inducible factor prolyl hydroxylase activity. As mentioned below such conditions include, but are not limited to, anaemia, ischemia-related diseases, inflammation, non-fatty liver disease, irritable bowel disease, cardiovascular disease, cardiac insufficiency, chronic kidney disease, renal insufficiency, Parkinson's disease, Alzheimer's disease, sickle cell anaemia and cancer. The disorder may for instance be a HIF-related disorder, an EPO-related disorder, or a VHL-related disorder, for instance Von Hippel-Lindau syndrome.

The invention also provides a compound of the invention as defined above, or the pharmaceutical composition of the invention, for use in treating anaemia, ischemia, inflammation, Parkinson's disease, Alzheimer's disease, non-fatty liver disease, irritable bowel disease, cardiovascular disease, cardiac insufficiency, chronic kidney disease, sickle cell anaemia, cancer, or renal insufficiency; or for use in skeletal muscle injury repair, in increasing red blood cell count (RBC), increasing haemoglobin (HGB) production, increasing haematocrit (HCT) production, increasing erythropoietin (EPO) production, wound healing, angiogenesis, revascularisation, stem cell activation, or cardioprotection after myocardial infarction. The anaemia may be renal anaemia, for instance anaemia associated with chronic kidney disease, or anaemia in a dialysis patient, anaemia induced by chemotherapy, sickle cell anaemia (including via upregulation of fetal haemoglobin F), age-related anaemia, or anaemia occurring as a result of cancer such as leukaemia, multiple myeloma and smouldering myeloma. The ischemia may be ischemia in a circulatory or cardiovascular disease, myocardial infarction, ischemia during a surgical operation, organ ischemia, an ischaemic disease or diabetic limb ischemia or sickle cell anaemia.
The invention also provides a method for treating a subject suffering from or susceptible to a PHD-related disorder, which method comprises administering to said subject an effective amount of a compound of the invention as defined above, or the pharmaceutical composition of the invention.
The invention additionally provides a method for treating a subject suffering from or susceptible to anaemia, ischemia, inflammation, Parkinson's disease, Alzheimer's disease, non-fatty liver disease, irritable bowel disease, cardiovascular disease, cardiac insufficiency, chronic kidney disease, renal insufficiency, or skeletal muscle injury repair, which method comprises administering to said subject an effective amount of a compound of the invention as defined above, or the pharmaceutical composition of the invention.
The invention additionally provides a method of: increasing red blood cell count (RBC), increasing haemoglobin (HGB) production, increasing haematocrit (HCT) production, increasing Erythropoietin (EPO) production, wound healing, angiogenesis, revascularisation, stem cell activation, or cardioprotection after myocardial infarction, in a subject, which method comprises administering to said subject an effective amount of a compound of the invention as defined above, or the pharmaceutical composition of the invention.

### BRIEF DESCRIPTION OF THE FIGURES

FIG. 1 shows immunoblots of Hep3B cells treated with compounds 117, 119, 122 and 123 (Examples 93, 95, 98 and 99) at 10 μM (A) and 2 μM (B) PHD inhibitors for 3 hours. The blots show protein levels of HIF1-α and β-actin at the 3^{rd} hour after treatment.

FIG. 2 shows immunoblots of HEK293 T cells treated with compound 68 (Example 46) at 0.5, 1, 5, 10, 20, 50 and 10 μM for 18 hours. The blots show protein levels of HIF1-α and GAPDH at the 18th hour after treatment.

FIG. 3 shows (A) the red blood cell count (10⁶/μL) (y-axis) for groups of seven C57BL/6 mice treated with either vehicle (1% methylcellulose) or control (daprodustat) compared to compound 68 of the invention (Example 46) (x-axis); (B) mouse haemoglobin levels (g/dL) (y-axis) for mice treated with either vehicle (1% methylcellulose) or a control (daprodustat) compared to compound 68 of the invention (Example 46) (x-axis); and (C) percentage mouse haemocrit (y-axis) for mice treated with either vehicle (1% methylcellulose) or a control (daprodustat) compared to compound 68 of the invention (Example 46) (x-axis). Values are shown pre-treatment, and 4 and 8 days after treatment.

FIG. 4 shows IC₅₀ of compound 68 (Example 46) of the invention when tested with off-target sites commonly inhibited by existing PHD inhibitors.

### DETAILED DESCRIPTION OF THE INVENTION

### Definitions

The term “alkyl”, as used herein, refers to a linear or branched chain saturated hydrocarbon radical. A “Cₙ₋ₘ alkyl” refers to an alkyl having from n to m carbon atoms. Thus, an alkyl group may be a C₁₋₂₀ alkyl group, a C₁₋₁₈ alkyl group, a C₁₋₁₄ alkyl group, a C₁₋₁₀ alkyl group, a C₁₋₆ alkyl group or a C₁₋₄ alkyl group. Examples of a C₁₋₁₀ alkyl group are methyl, ethyl, propyl, butyl, pentyl, hexyl, heptyl, octyl, nonyl or decyl. Examples of C₁₋₆ alkyl groups are methyl, ethyl, propyl, butyl, pentyl or hexyl. Examples of C₁₋₄ alkyl groups are methyl, ethyl, i-propyl, n-propyl, t-butyl, s-butyl or n-butyl. If the term “alkyl” is used without a prefix specifying the number of carbons anywhere herein, it has from 1 to 6 carbons. For the avoidance of doubt, where two alkyl moieties are present in a group, the alkyl moieties may be the same or different.

The term “cycloalkyl”, as used herein, refers to a saturated or partially unsaturated cyclic hydrocarbon radical. A “Cₙ₋ₘ cycloalkyl” refers to a cycloalkyl having from n to m carbon atoms. Thus, a cycloalkyl group may be a C₃₋₂₀ cycloalkyl group, a C₃₋₁₀ cycloalkyl group, a C₃₋₈ cycloalkyl group or a C₃₋₆ cycloalkyl group. Examples of a C₃₋₈ cycloalkyl group include cyclopropyl, cyclobutyl, cyclopentyl, cyclohexyl, cyclohexenyl, cyclohex-1,3-dienyl, cycloheptyl and cyclooctyl. Examples of a C₃₋₆ cycloalkyl group include cyclopropyl, cyclobutyl, cyclopentyl, and cyclohexyl.

The term “alkenyl”, as used herein, refers to a linear or branched chain hydrocarbon radical containing one or more double bonds. A “Cₙ₋ₘ alkenyl” refers to an alkenyl having from n to m carbon atoms. Thus, an alkenyl group may be a C₂₋₁₈ alkenyl group, a C₂₋₁₄ alkenyl group, a C₂₋₁₀ alkenyl group, a C₂₋₆ alkenyl group or a C₂₋₄ alkenyl group. Examples of a C₂₋₁₀ alkenyl group are ethenyl (vinyl), propenyl, butenyl, pentenyl, hexenyl, heptenyl, octenyl, nonenyl or decenyl. Examples of C₂₋₆ alkenyl groups are ethenyl, propenyl, butenyl, pentenyl or hexenyl. Examples of C₂₋₄ alkenyl groups are ethenyl, i-propenyl, n-propenyl, s-butenyl or n-butenyl. Alkenyl groups typically comprise one or two double bonds.

The term “alkynyl”, as used herein, refers to a linear or branched chain hydrocarbon radical containing one or more triple bonds. A “Cₙ₋ₘ alkynyl” refers to an alkynyl having from n to m carbon atoms. Thus, an alkynyl group may be a C₂₋₁₈ alkynyl group, a C₂₋₁₄ alkynyl group, a C₂₋₁₀ alkynyl group, a C₂₋₆ alkynyl group or a C₂-4 alkynyl group. Examples of a C₂₋₁₀ alkynyl group are ethynyl, propynyl, butynyl, pentynyl, hexynyl, heptynyl, octynyl, nonynyl or decynyl. Examples of C₁₋₆ alkynyl groups are ethynyl, propynyl, butynyl, pentynyl or hexynyl. Alkynyl groups typically comprise one or two triple bonds.

A C₃₋₂₀ heterocyclyl group is a monovalent moiety obtained by removing a hydrogen atom from a ring atom of a heterocyclic compound, which moiety has from 3 to 20 ring atoms (unless otherwise specified), of which from 1 to 10 are ring heteroatoms. A “Cₙ₋ₘ heterocyclyl” refers to a heterocyclyl having from n to m ring atoms. Preferably, the ring has from 3 to 7 ring atoms (i.e. it is a C₃₋₇ heterocyclyl), of which from 1 to 4 are ring heteroatoms.

Examples of 5- and 6-membered saturated heterocyclyl groups include piperazine, piperidine, morpholine, 1,3-oxazinane, pyrrolidine, imidazolidine, and oxazolidine, including quaternised derivatives thereof, as defined herein. Examples of 5- and 6-membered partially saturated heterocyclyl groups include tetrahydropyrazine, tetrahydropyridine, dihydro-1,4-oxazine, tetrahydropyrimidine, dihydro-1,3-oxazine, dihydropyrrole, dihydroimidazole and dihydrooxazole, including quaternised derivatives thereof, as defined herein. Thus, heterocyclyl groups include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolyl and pyrazolinyl groups and moieties. Pyrazolidinyl, piperidyl, piperazinyl, pyrazolidinyl morpholinyl and imidazolidinyl groups and moieties are typical examples.

Examples of 9- and 10-membered fused heterobicyclyl groups include 9-membered fused heterobicyclic groups such as indoline, 2,3-dihydrobenzofuran, 2,3-dihydrobenzo[b]thiophene, 2,3-dihydro-1H-benzo[d]imidazole, 2,3-dihydrobenzo[d]oxazole, 2,3-dihydrobenzo[d]thiazole, benzo[d][1,3]dioxole, 4,5,6,7-tetrahydrothiazolo[5,4-c]pyridine and 4,5,6,7-tetrahydrothiazolo[4,5-c]pyridine, including quaternised derivatives thereof, as defined herein; and 10-membered heterobicyclyl groups such as 1,2,3,4-tetrahydroquinoline, 1,2,3,4-tetrahydroisoquinoline, chromane, isochromane, thiochromane, isothiochromane, 1,2,3,4-tetrahydroquinoxaline, 1,2,3,4-tetrahydroquinazoline, 1,4-dihydro-2H-benzo[d][1,3]oxazine, 3,4-dihydro-2H-benzo[b][1,4]oxazine, 3,4-dihydro-2H-benzo[b][1,4]thiazine, 1,4-dihydro-2H-benzo[d][1,3]thiazine, 4H-benzo[d][1,3]dioxine and 2,3-dihydrobenzo[b][1,4]dioxine, including quaternised derivatives thereof. Preferably, the fused heterobicyclyl group comprises 1, 2 or 3, preferably 1 or 2 nitrogen atoms.

For the avoidance of doubt, references to a heterocyclyl group also include fused polycyclic ring systems, including for instance fused bicyclic systems in which a heterocyclic group is fused to an aryl group. When the heterocyclyl group is such a fused heterocyclyl group, preferred examples are fused ring systems wherein a 5-to 6-membered heterocyclyl group is fused to a phenyl group. References to a heterocyclyl group also include spiro ring systems, for example 7-membered heterocyclic groups e.g. 2,6-diazaspiro[3.3]heptane.

The term “aryl”, as used herein, refers to a monocyclic, bicyclic or polycyclic aromatic ring which contains up to 14 carbon atoms, typically from 6 to 10 carbon atoms, in the ring portion. Examples include phenyl, naphthyl, indenyl and indanyl groups. Phenyl is preferred.

The term “heteroaryl”, as used herein, refers to monocyclic or bicyclic heteroaromatic rings which typically contains from five to ten, for instance from six to ten, atoms in the ring portion including one or more heteroatoms. A heteroaryl group is generally a 5- or 6-membered ring, containing at least one heteroatom selected from O, S, N, P, Se and Si, more typically selected from O, S and N. It may contain, for example, one, two or three heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, pyridazolyl, quinolyl and isoquinolyl. Furanyl, thienyl, pyridazolyl, pyrazolyl, pyrimidinyl and thiazolyl groups are typical examples.

The terms “alkylene”, “cycloalkylene”, “heterocyclylene”, “alkenylene”, “alkynylene”, “arylene” and “heteroarylene”, as used herein, refer to bivalent groups obtained by removing a hydrogen atom from an alkyl, cycloalkyl, heterocyclyl, alkenyl, alkynyl, aryl or heteroaryl group, respectively. Such bidentate groups may be substituted or unsubstituted. An alkylene group may be a C₁₋₂₀ alkylene group, a C₁₋₁₈ alkylene group, a C₁₋₁₄ alkylene group, a C₁₋₁₀ alkylene group, a C₁₋₆ alkylene group or a C₁₋₄ alkylene group. Examples of C₁₋₆ alkylene groups are methylene, ethylene, propylene, butylene, pentylene and hexylene. A cycloalkylene group may be a C₃₋₁₀ cycloalkylene group, a C₃₋₈ cycloalkylene group or a C₃₋₆ cycloalkylene group. Examples of C_{3.6} cycloalkylene groups include cyclopentylene and cyclohexylene. An alkenylene group may be a C₂₋₁₈ alkenylene group, a C₂₋₁₄ alkenylene group, a C₂₋₁₀ alkenylene group, a C₂₋₆ alkenylene group or a C₂₋₄ alkenylene group. Examples of a C₂₋₄ alkenylene group include ethenylene (vinylene), propenylene and butenylene. An alkynylene group may be a C₂₋₁₈ alkynylene group, a C₂₋₁₄ alkynylene group, a C₂₋₁₀ alkynylene group, a C₂₋₆ alkynylene group or a C₂₋₄ alkynylene group. Examples of a C₂₋₄ alkynylene group include ethynylene and propynylene. Examples of arylene groups include phenylene, and examples of heteroarylene groups include, for instance, a diradical derived from pyridine, a diradical derived from thiophene, a diradical derived from chromane, and a diradical derived from chromanol. For alkylene, cycloalkylene, alkenylene, alkynylene, arylene and heteroarylene, these groups may be bonded to other groups at any two positions on the group (which positions are typically carbon atoms in the case of heteroarylene and heterocyclylene). Thus, propylene includes —CH₂CH₂CH₂— and —CH₂CH(CH₃)—, and phenylene includes ortho-, meta- and para-phenylene.

The term “substituted”, as used herein, in the context of substituted organic compounds and groups, refers to an organic compound or group (e.g. an alkyl group, an alkylene group, a cycloalkyl group, a heterocyclyl group, an aryl group, an arylene group, a heteroaryl group, or a heteroarylene group) which bears one or more substituents selected from C₁₋₁₀ alkyl, C₃₋₁₀ cycloalkyl, C₃₋₇ heterocyclyl, aryl, heteroaryl, cyano, amino, nitro, C₂₋₁₀ alkenyl, C₂₋₁₀ alkynyl, C₁₋₁₀ alkylamino, di(C₁₋₁₀)alkylamino, arylamino, diarylamino, aryl(C₁₋₁₀)alkylamino, amido, acylamido, hydroxy, oxo, halo, carboxy, ester, acyl, acyloxy, C₁₋₁₀ alkoxy, aryloxy, halo(C₁₋₁₀)alkyl, sulfonic acid, thiol, C₁₋₁₀ alkylthio, arylthio, sulfonyl, phosphoric acid, phosphate ester, phosphonic acid, phosphonate ester and SO₃—. Typically, the one or more substituents are selected from cyano, amino, nitro, amido, acylamido, hydroxy, oxo, halo, carboxy, ester, acyl, acyloxy, sulfonic acid, thiol, sulfonyl, phosphoric acid, phosphate ester, phosphonic acid, phosphonate ester and SO₃—. When a compound or group is substituted, it typically bears 1, 2, 3 or 4 substituents. For instance, a substituted compound or group may have 1, 2 or 3 substituents, or for example 1 or 2 substituents.

However, when a group is halo-substituted, for instance fluoro-substituted, the group may bear 1, 2, 3 or 4 halo substituents, or it may bear more than four halo substituents. In fact, the group may be perhalo-substituted, i.e. all hydrogen atoms of the group may be replaced by halogen atoms. The group may for instance be perfluoro-substituted, i.e. perfluorinated, i.e. all hydrogen atoms of the group may be replaced by fluorine atoms. Accordingly, the term “substituted”, as used herein, in the context of substituted organic groups, for instance in the context of substituted hydrocarbyl groups, substituted alkyl groups, substituted cycloalkyl groups, substituted alkenyl groups, substituted alkynyl groups, substituted aryl groups, substituted hydrocarbylene groups, substituted alkylene groups, substituted cycloalkylene groups, substituted alkenylene groups, substituted alkynylene groups, and substituted arylene (including substituted heteroarylene) groups, encompasses the perhalo-substituted groups, in particular the perfluoro-substituted groups. Thus, for example, the term “substituted Cₙ₋ₘ alkyl” as used herein encompasses Cₙ₋ₘ perfluoroalkyl, the term “substituted Cₙ₋ₘ alkylene” as used herein encompasses Cₙ₋ₘ perfluoroalkylene, the term “substituted Cₙ₋ₘ hydrocarbyl” as used herein encompasses Cₙ₋ₘ perfluorohydrocarbyl and the term “substituted Cₙ₋ₘ hydrocarbylene” as used herein encompasses Cₙ₋ₘ perfluorohydrocarbylene, the term “substituted Cₙ₋ₘ alkoxy” as used herein encompasses Cₙ₋ₘ perfluoroalkoxy, and so-on.

As used herein the term oxo represents a group of formula: ═O

As used herein the term acyl represents a group of formula: —C(═O)R, wherein R is an acyl substituent, for example, a substituted or unsubstituted C₁₋₂₀ alkyl group, a substituted or unsubstituted C₃₋₂₀ heterocyclyl group, or a substituted or unsubstituted aryl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term ester (or carboxylate, carboxylic acid ester or oxycarbonyl) represents a group of formula: —C(═O)OR, wherein R is an ester substituent, for example, a substituted or unsubstituted C₁₋₂₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term acyloxy (or reverse ester) represents a group of formula: —OC(═O)R, wherein R is an acyloxy substituent, for example, substituted or unsubstituted C₁₋₂₀ alkyl group, a substituted or unsubstituted C₃₋₂₀ heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term phosphonic acid represents a group of the formula: —P(═O)(OH)₂. As would be understood by the skilled person, a phosphonic acid group can exist in protonated and deprotonated forms (i.e. —P(═O)(OH)₂, —P(═O)(O⁻)₂ and —P(═O)(OH)(O⁻)) all of which are within the scope of the term “phosphonic acid”.

As used herein the term phosphonic acid salt represents a group which is a salt of a phosphonic acid group. For example a phosphonic acid salt may be a group of the formula —P(═O)(OH)(O⁻X⁺) wherein X is a monovalent cation. X⁺ may be an alkali metal cation. X⁺ may be Na⁺ or K⁺, for example.

As used herein the term phosphonate ester represents a group of one of the formulae: —P(═O)(OR)₂ and —P(═O)(OR)O— wherein each R is independently a phosphonate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphonate ester groups include, but are not limited to, —P(═O)(OCH₃)₂, —P(═O)(OCH₂CH₃)₂, —P(═O)(O⁻ t-Bu)₂, and —P(═O)(OPh)₂,

As used herein the term phosphoric acid represents a group of the formula: —OP(═O)(OH)₂.

As used herein the term phosphate ester represents a group of one of the formulae: —OP(═O)(OR)₂ and —OP(═O)(OR)O— wherein each R is independently a phosphate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphate ester groups include, but are not limited to, —OP(═O)(OCH₃)₂, —OP(═O)(OCH₂CH₃)₂, —OP(═O)(O⁻ t-Bu)₂, and —OP(═O)(OPh)₂.

As used herein the term amino represents a group of formula —NH₂. The term C₁-C₁₀ alkylamino represents a group of formula -NHR′ wherein R′ is a C₁₋₁₀ alkyl group, preferably a C₁₋₆ alkyl group, as defined previously. The term di(C₁₋₁₀)alkylamino represents a group of formula -NR′R″ wherein R′ and R″ are the same or different and represent C₁₋₁₀ alkyl groups, preferably C₁₋₆ alkyl groups, as defined previously. The term arylamino represents a group of formula -NHR′ wherein R′ is an aryl group, preferably a phenyl group, as defined previously. The term diarylamino represents a group of formula -NR′R″ wherein R′ and R″ are the same or different and represent aryl groups, preferably phenyl groups, as defined previously. The term arylalkylamino represents a group of formula -NR′R″ wherein R′ is a C₁₋₁₀ alkyl group, preferably a C₁₋₆ alkyl group, and R″ is an aryl group, preferably a phenyl group.

As used herein the term amido represents a group of formula: —C(═O)NR′R″, wherein R′ and R″ are independently amino substituents, as defined for di(C₁₋₁₀. )alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: -NR¹C(═O)R², wherein R¹ is an amide substituent, for example, hydrogen, a C₁₋₂₀alkyl group, a C₃₋₂₀ heterocyclyl group, an aryl group, preferably hydrogen or a C₁₋₂₀ alkyl group, and R² is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

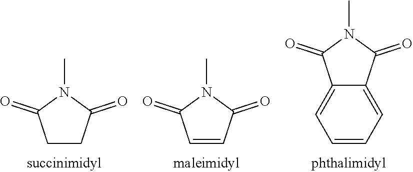

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. A substituted C₁₋₂₀ alkoxy group includes a C₁₋₂₀ perfluoroalkoxy group. A C₁₋₂₀ perfluoroalkoxy group is a C₁₋₂₀ perfluoroalkyl group attached to an oxygen atom. An example of a C₁₋₂₀ perfluoroalkoxy group is a tert-nonafluorobutyloxy group, —OC(CF₃)₃.

An aryloxy group is a substituted or unsubstituted aryl group, as defined herein, attached to an oxygen atom. It may for instance be unsubstituted or substituted phenoxy. An example of an aryloxy group is —OPh (phenoxy).

The term “amino acid”, as used herein, in connection with any of the compounds described herein, means an amino acid residue. The amino acid residue is typically bonded via its C— terminus or via its N-terminus to the atom in the compound described herein to which it is said to be bonded. For example, as would be understood by the skilled person, when an amino acid is said to be bonded to a carbon atom of a carbonyl group in a compound described herein, the nitrogen atom at the N-terminus of the amino acid is typically bonded to that carbon atom. Similarly, where an amino acid is said to be bonded to a nitrogen atom of an amine group in a compound described herein, the carbon atom of the C-terminus of the amino acid would generally be bonded to that nitrogen atom. The carbon atom of the C-terminus of an amino acid may alternatively be bonded to an oxygen atom in a compound as described herein. An amino acid in any of the compounds described herein may for instance be an amino acid residue selected from arginine (Arg), histidine (His), lysine (Lys), aspartic acid (Asp), glutamic acid (Glu), serine (Ser), threonine (Thr), asparagine (Asn), glutamine (Gln), cysteine (Cys), selenocysteine (Sec), glycine (Gly), proline (Pro), alanine (Ala), valine (Val), isoleucine (Ile), leucine (Leu), methionine (Met), phenylalanine (Phe), tyrosine (Tyr) and tryptophan (Trp).

The term azine, as used herein, means a heterocyclic compound containing a 6-membered aromatic ring, in which one or more of the ring carbon atoms has been replaced by a nitrogen atom. For example, pyridine is an azine, as is pyridazine.

Unless otherwise specified, included in the above are the well known ionic, salt, solvate, and protected forms of these substituents. For example, a reference to carboxylic acid or carboxyl group (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

The compounds of the invention can exist in various tautomeric forms and it is to be understood that the invention encompasses all such tautomeric forms.

In certain of the compounds of the invention, dependent on the nature of the substituent, there may be chiral carbon atoms and therefore the compounds may exist as stereoisomers. The invention extends to all optical isomers such as stereoisomeric forms of the compounds of the invention, including enantiomers, diastereomers and mixtures thereof, such as racemates. The different stereoisomeric forms may be separated or resolved one from the other by conventional methods or any given isomer may be obtained by conventional stereoselective or sterospecific syntheses.

It is also to be understood that any atom present in a compound of the invention may be present in any available naturally-occuring isotopic form. For instance, a carbon atom may be¹²C or ¹³C. A hydrogen atom may be ¹H or ²H (deuterium).

As used herein, the terms “treat”, “treating” and “treatment” refer to both therapeutic treatment and prophylactic or preventative measures, wherein the object is to prevent or slow down (lessen) an undesired physiological change or disorder, such as the development or spread of disease. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “pharmaceutically acceptable” indicates that the substance or composition must be compatible chemically and/or toxicologically with the other ingredients comprising a formulation, and/or the patient being treated therewith.

### Compounds of the Invention

The invention relates to a series of novel compounds and their use as hypoxia inducible factor prolyl hydroxylase domain inhibitors (PHD inhibitors). The compounds therefore have potential utility in treating conditions for which HIF-PHD is a therapeutic target, including for instance anaemia and other ischemia-related diseases, and further conditions as mentioned below.

Accordingly the present invention provides a compound which is a substituted azine of formula (I) or a pharmaceutically acceptable salt thereof

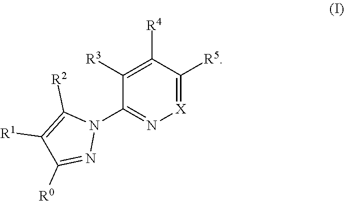

In the above formula (I), X is C(R⁶) or N. Preferably X is C(R⁶), i.e. preferably X is a ring-carbon atom bonded to R⁶ (in which case, the substituted azine is a substituted pyridine). Often, however, X is N (in which case, the substituted azine is a substituted pyridazine).

R⁰ is H or unsubstituted or substituted C₁₋₆ alkyl. Typically, R⁰ is H or unsubstituted C₁₋₆ alkyl. Usually, R⁰ is selected from H, methyl and ethyl. Often, R⁰ is H or methyl.

R¹ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w} or —C(O)N(R^{x})R⁷.

Typically R¹ is —C(O)N(R^{x})R⁷. This is especially typical when R⁵ is other than —C(O)N(R^{x})R⁷. Thus, R¹ being —C(O)N(R^{x})R⁷ is especially typical when R⁵ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}.

R¹ may also typically be H, unsubstituted or substituted C₁₋₆ alkyl, unsubstituted or substituted aryl, unsubstituted or substituted heteroaryl, —CN, -N(R^{t})C(O)N(R^{u})R^{v}, or —C(O)OR^{w}. This is especially typical when R⁵ is —C(O)N(R^{x})R⁷. For instance, R¹ may be H, unsubstituted or substituted C₁₋₆ alkyl, —CN or —C(O)OR^{w}, preferably H, —CN, or —C(O)OR^{w}. These are often the case when R⁵ is —C(O)N(R^{x})R⁷.

Thus, R¹ may be selected from H, unsubstituted or substituted C₁₋₆ alkyl, —CN, —C(O)OR^{w} and —C(O)N(R^{x})R⁷. R¹ may for instance be selected from H, —CN, —C(O)OR^{w} and —C(O)N(R^{x})R⁷. R¹ may for instance be selected from H, unsubstituted or substituted C₁₋₆ alkyl, —CN and —C(O)OR^{w}, or, for instance, from H, —CN and —C(O)OR^{w}.

R² is H, —OR^{q} or unsubstituted or substituted C₁₋₆ alkyl. Alternatively, R² is —N═, in which case R³ is ═C(R^{y})— and R² and R³ together form a group of formula —N═C(R^{y})—.

R² may for instance be H, —OR^{q} or unsubstituted or substituted C₁₋₆ alkyl. Typically, in that case, R² is H or unsubstituted or substituted C₁₋₆ alkyl. For instance, R² may be H or unsubstituted C₁₋₆ alkyl, for instance R² may be H, ethyl or methyl. R² may for example be H or methyl.

R³ is H, —OR⁸ or unsubstituted or substituted C₁₋₆ alkyl. Alternatively, R³ is ═C(R^{y})—, in which case R² is —N═ and and R² and R³ together form a group of formula —N═C(R^{y})—.

R³ may for instance be H, —OR⁸ or unsubstituted or substituted C₁₋₆ alkyl. Typically, in that case, R³ is H or —OR⁸. R³ may also usually be selected from H or unsubstituted or substituted C₁₋₆ alkyl. R³ is often, however, —OR⁸.

In some embodiments, R² is —N═ and R³ is ═C(R^{y})— and R² and R³ together form a group of formula —N═C(R^{y})—.

R⁴ is H, unsubstituted or substituted C₁₋₆ alkyl, —OR⁹ or —C(O)OR¹⁰. R⁴ may for instance be H, unsubstituted C₁₋₆ alkyl, —OR⁹ or —C(O)OR¹⁰. Often, however, R⁴ is H, —OR⁹ or —C(O)OR¹⁰. Typically R⁴ is selected from —OR⁹ and —C(O)OR¹⁰, or R⁴ is —OR⁹. Alternatively, R⁴ may be selected from H or unsubstituted or substituted C₁₋₆ alkyl, for instance R⁴ may be H. Often, though, R⁴ is C(O)OH or OH. R⁴ is often, for instance, OH.

R⁵ is H, unsubstituted or substituted C₁₋₆ alkyl, unsubstituted or substituted aryl, unsubstituted or substituted heteroaryl, —CN, —N(R^{t})C(O)N(R^{u})R, —C(O)OR^{w}, or —C(O)N(R^{x})R⁷.

R⁵ is typically H, unsubstituted or substituted C₁₋₆ alkyl, unsubstituted or substituted aryl, unsubstituted or substituted heteroaryl, —CN, -N(R^{t})C(O)N(R^{u})R^{v} or —C(O)OR^{w}. This is especially typical when R¹ is —C(O)N(R^{x})R⁷. For instance, R⁵ may be H, unsubstituted or substituted C₁₋₆ alkyl, —CN or —C(O)OR^{w}, preferably H, —CN, or —C(O)OR^{w}. These are often the case when R¹ is —C(O)N(R^{x})R⁷.

R⁵ may also typically be —C(O)N(R^{x})R⁷. This is especially typical when R¹ is other than —C(O)N(R^{x})R⁷. Thus, R⁵ being —C(O)N(R^{x})R⁷ is especially typical when R¹ is H, unsubstituted or substituted C₁₋₆ alkyl, unsubstituted or substituted aryl, unsubstituted or substituted heteroaryl, —CN, —N(R^{t})C(O)N(R^{u})R or —C(O)OR^{w}.

Typically, R⁵ is H, unsubstituted or substituted C₁₋₆ alkyl, unsubstituted or substituted aryl, unsubstituted or substituted heteroaryl, —CN, —C(O)OR^{w}, or —C(O)N(R^{x})R⁷. Often, R⁵ is selected from H, unsubstituted or substituted C₁₋₆ alkyl, —CN, —C(O)OR^{w} and —C(O)N(R^{x})R⁷. R⁵ may for instance be selected from H, —CN, —C(O)OR^{w} and —C(O)N(R^{x})R⁷. R⁵ may for instance be selected from H, unsubstituted or substituted C₁₋₆ alkyl, —CN and —C(O)OR^{w}, or, for instance, from H, —CN and —C(O)OR^{w}. R⁵ may for instance be —C(O)OR^{w}. Often R⁵ is —C(O)OH.

R⁶ is H or unsubstituted or substituted C₁₋₆ alkyl. Usually R⁶ is H or unsubstituted C₁₋₆ alkyl. Typically R⁶ is H.

R⁷ is —CH(R¹¹)—Ar, —CH(R¹¹)-Ary-Ar, -Ary-Ar, —CH(R¹¹)-Cyc, —CH₂C≡CCH₃, -Cyc or —Ar. R⁷ may for instance be —CH(R¹¹)—Ar, —CH(R¹¹)-Ary-Ar, -Ary-Ar or —CH(R¹¹)-Cyc.

Ar is unsubstituted or substituted aryl or unsubstituted or substituted heteroaryl. For instance, Ar may be unsubstituted or substituted aryl or unsubstituted heteroaryl. For instance, Ar may be unsubstituted or substituted phenyl, or unsubstituted heteroaryl. Ar may for instance be selected from unsubstituted phenyl, unsubstituted pyrimidyl, unsubstituted benzothiazole, and phenyl substituted with —C(O)OH, —C(O)OMe, —C(O)OEt, —C(O)NH₂, —C(O)N(H)Me, —OMe or N-morpholino.

Ary is unsubstituted or substituted arylene, or unsubstituted or substituted heteroarylene. Ary may for instance be unsubstituted arylene, or unsubstituted heteroarylene. Typically Ary is unsubstituted phenylene or unsubstituted pyridylene.

Cyc is unsubstituted or substituted C₃₋₁₀ cycloalkyl. Typically, Cyc is unsubstituted or substituted cyclohexyl. For instance Cyc may be unsubstituted cyclohexyl or cyclohexyl substituted with —CF₃ or —OCF₃.

R¹¹ is H, —C(O)OR^{z} or unsubstituted or substituted C₁₋₄ alkyl. R″ may for instance be H, —C(O)OR^{z} or unsubstituted C₁₋₄ alkyl. Usually, R¹¹ is H, —C(O)OR^{z} or methyl.

R⁸, R⁹ and R¹⁰ are each independently selected from H and unsubstituted or substituted C₁₋₆ alkyl.

Therefore R⁸ may be H or unsubstituted or substituted C₁₋₆ alkyl. Typically R⁸ is H.

R⁹ may also be selected from H or unsubstituted or substituted C₁₋₆ alkyl. Typically R⁹ is H.

R¹⁰ may also be selected from H or unsubstituted or substituted C₁₋₆ alkyl. Typically R¹⁰ is H.

Usually R⁸, R⁹ and R¹⁰, which may be the same or different, are each independently selected from H, unsubstituted C₁₋₆ alkyl, and C₁₋₆ alkyl which is substituted with phenyl or —OC(O)R⁹⁹. R⁹⁹ is phenyl, unsubstituted C₁₋₆ alkyl, -N(R^{a})(R^{b}), —C(O)R⁰, —OR^{d} or an amino acid. Ra, R^{b}, R^{c} and R^{d} are each independently selected from H, unsubstituted or substituted C₁₋₆ alkyl and an amino acid.

Thus, often, R⁸ is selected from H, unsubstituted C₁₋₆ alkyl, and C₁₋₆ alkyl which is substituted with phenyl or —OC(O)R⁹⁹, wherein R⁹⁹ is as defined above.

Typically, R⁹ is selected from H, unsubstituted C₁₋₆ alkyl, and C₁₋₆ alkyl which is substituted with phenyl or —OC(O)R⁹⁹, wherein R⁹⁹ is as defined above.

Often, R¹⁰ is selected from H, unsubstituted C₁₋₆ alkyl, and C₁₋₆ alkyl which is substituted with phenyl or —OC(O)R⁹⁹, wherein R⁹⁹ is as defined above.

Structures of formula (I) in which R⁸, R⁹ or R¹⁰ is other than H include prodrug compounds. In particular, the substituted azines of formula (I) in which R⁸, R⁹ or R¹⁰ is unsubstituted or substituted C₁₋₆ alkyl, and particularly substituted C₁₋₆ alkyl, include prodrug compounds. For instance, substituted azines of formula (I) in which R⁸, R⁹ or R¹⁰ is C₁₋₆ alkyl which is substituted with phenyl or —OC(O)R⁹⁹, wherein R⁹⁹ is as defined above, include prodrug compounds. R⁸, R⁹ or R¹⁰ may for instance be substituted C₁₋₆ alkyl, wherein the, or one of the, substituents on the C₁₋₆ alkyl is a group of formula —OC(O)R⁹⁹, wherein R⁹⁹ is phenyl, unsubstituted C₁₋₆ alkyl, —N(R^{a})(R), —C(O)R^{c}, —OR^{d} or an amino acid, and wherein Ra, R^{b}, R^{c} and R^{d} are each independently selected from H, unsubstituted or substituted C₁₋₆ alkyl and an amino acid. Such compounds include prodrugs. Thus, often, R⁸ is C₁₋₆ alkyl which is substituted with —OC(O)R⁹⁹, wherein R⁹⁹ is as defined above. Similarly, R⁹ may be C₁₋₆ alkyl which is substituted with —OC(O)R⁹⁹, wherein R⁹⁹ is as defined above. R¹⁰ may be C₁₋₆ alkyl which is substituted with —OC(O)R⁹⁹, wherein R⁹⁹ is as defined above.

Typically, Ra, R^{b}, R^{c} and R^{d} are each independently selected from H, or unsubstituted or substituted C₁₋₆ alkyl. Usually, Ra, R^{b}, R^{c} and R^{d} are each independently selected from H, methyl or ethyl.

R^{t}, R^{u}, R^{v}, R^{w}, R^{x}, R^{y}, and R^{z} are each independently selected from H, unsubstituted or substituted C₁₋₆ alkyl, and unsubstituted or substituted phenyl.

Usually R^{t} is unsubstituted or substituted C₁₋₄ alkyl, or H. Typically, R^{t} is unsubstituted C₁₋₄ alkyl or H. Usually, R^{t} is H.

Usually R^{u} is unsubstituted or substituted C₁₋₄ alkyl, or H. Typically, R^{u} is unsubstituted C₁₋₄ alkyl or H. Usually, R^{u} is H.

Usually R is unsubstituted or substituted C₁₋₄ alkyl, or H. Typically, R is unsubstituted C₁₋₄ alkyl or H. Usually, R is H.

Thus, often, R^{t}, R^{u} and R are all H.

Usually Rx is H.

Typically R is H or unsubstituted C₁₋₆ alkyl, for example R^{y} may be H or methyl.

Typically Rz is H.

R^{w} is H, unsubstituted or substituted C₁₋₆ alkyl, or unsubstituted or substituted phenyl. R^{w} may be H, or unsubstituted or substituted C₁₋₆ alkyl. Typically, for instance, R^{w} is H, unsubstituted C₁₋₆ alkyl, or C₁₋₆ alkyl which is substituted with phenyl or —OC(O)R^{ww}, wherein R^{ww} is phenyl, unsubstituted C₁₋₆ alkyl, —N(R^{a})(R), —C(O)R⁰, —OR^{d} or an amino acid, wherein Ra, R^{b}, R^{c} and R^{d} are each independently selected from H, unsubstituted or substituted C₁₋₆ alkyl and an amino acid.

Substituted azines of formula (I) in which R^{w} is other than H include prodrug compounds. In particular, compounds of formula (I) in which R^{w} is unsubstituted or substituted C₁₋₆ alkyl, or unsubstituted or substituted phenyl, include prodrug compounds. For instance, compounds of formula (I) in which R^{w} is C₁₋₆ alkyl which is substituted with phenyl or —OC(O)R^{ww}, wherein R^{ww} is is as defined above, include prodrug compounds. R^{w} may for instance be substituted C₁₋₆ alkyl, wherein the, or one of the, substituents on the C₁₋₆ alkyl is a group of formula —OC(O)R^{ww}, wherein R″ is phenyl, unsubstituted C₁₋₆ alkyl, —N(R^{a})(R^{b}), —C(O)R⁰, —OR^{d} or an amino acid, wherein Ra, R^{b}, R^{c} and R^{d} are each independently selected from H, unsubstituted or substituted C₁₋₆ alkyl and an amino acid. Such compounds include prodrugs. Thus, often, R^{w} is C₁₋₆ alkyl which is substituted with —OC(O)R^{ww}, wherein R″ is as defined above. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R^{w} may be H, unsubstituted C₁₋₆ alkyl, or C₁₋₆ alkyl which is substituted with phenyl or —OC(O)R^{ww} wherein R″ is phenyl or unsubstituted C₁₋₆ alkyl.

R^{w} may be H.

R^{q} is H, unsubstituted or substituted C₁₋₆ alkyl, or unsubstituted or substituted phenyl. R^{q} is typically H.

Typically, in formula (I), one of R¹ and R⁵ is —C(O)N(R^{x})R⁷ and the other of R¹ and R⁵ is other than —C(O)N(R^{x})R⁷. When one of R¹ and R⁵ is other than —C(O)N(R^{x})R⁷, it may be any of the other definitions for R¹ or R⁵ specified herein. Thus, it may be any of H, unsubstituted or substituted C₁₋₆ alkyl, unsubstituted or substituted aryl, unsubstituted or substituted heteroaryl, —CN, -N(R^{t})C(O)N(R^{u})R^{v} or —C(O)OR^{w}.

Often, in formula (I), one of R¹ and R⁵ is —C(O)N(R^{x})R⁷ and the other of R¹ and R⁵ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}. Typically, one of R¹ and R⁵ is —C(O)N(R^{x})R⁷ and the other of R¹ and R⁵ is H, unsubstituted or substituted C₁₋₆ alkyl, —CN or —C(O)OR^{w}. For instance, one of R¹ and R⁵ may be —C(O)N(R^{x})R⁷ and the other of R¹ and R⁵ may be H, —CN or —C(O)OR^{w}.

In some embodiments of the substituted azine of formula (I):
R⁰ is H or unsubstituted C₁₋₆ alkyl;
R¹ is H, —CN, —C(O)OR^{w} or —C(O)N(R^{x})R⁷;
R² is H or unsubstituted C₁₋₆ alkyl; and R³ is H or —OR⁸; or R² is —N═ and R³ is ═C(R^{y})— and R² and R³ together form a group of formula —N═C(R^{y})—. In such embodiments, R⁴ may be H, —OR⁹ or —C(O)OR¹⁰; R⁵ may be H, —CN, —C(O)OR^{w}, or —C(O)N(R^{x})R⁷; and R⁶ is H. Furthermore, R⁷ is typically —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR^{z} or unsubstituted C₁₋₄ alkyl.

Usually in such embodiments R⁸, R⁹ and R¹⁰ are each independently selected from H and unsubstituted or substituted C₁₋₆ alkyl. Typically, R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R^{ww} wherein R″ is phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl.

Typically such compounds are provided wherein one of R¹ and R⁵ is —C(O)N(R)R⁷ and the other of R¹ and R⁵ is H, —CN or —C(O)OR^{w}.

Thus, typically, R⁰ is H or unsubstituted C₁₋₆ alkyl; R¹ is H, —CN, —C(O)OR^{w} or —C(O)N(R^{x})R⁷; R² is H or unsubstituted C₁₋₆ alkyl; and R³ is H or —OR⁸; or R² is —N═ and R³ is ═C(R^{y})— and R² and R³ together form a group of formula —N═C(R^{y})—; R⁴ is H, —OR⁹ or —C(O)OR¹⁰; R⁵ is H, —CN, —C(O)OR^{w}, or —C(O)N(R^{x})R⁷; R⁶ is H; R⁷ is —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR^{z} or unsubstituted C₁₋₄ alkyl; R⁸, R⁹ and R¹⁰ are each independently selected from H and unsubstituted or substituted C₁₋₆ alkyl; and R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R″ wherein R^{ww} is phenyl, unsubstituted C₁₋₆ alkyl, —N(R^{a})(R^{b}), —C(O)R⁰, —OR^{d} or an amino acid, wherein R^{a}, R^{b}, R⁰ and R^{d} are each independently selected from H, unsubstituted or substituted C₁₋₆ alkyl and an amino acid; provided that one of R¹ and R⁵ is —C(O)N(R^{x})R⁷ and the other of R¹ and R⁵ is H, —CN or —C(O)OR^{w}.

In some embodiments of the substituted azine of formula (I), R⁰ is H or methyl; R¹ is H, —CN, —C(O)OR^{w}, or —C(O)N(R′)R⁷; R² is H or methyl; and R³ is H or —OR⁸; or R² is —N═ and R³ is =C(R^{y})— and R² and R³ together form a group of formula —N=C(R^{y})—.

Typically, in such compounds R⁴ is H, —OR⁹ or —C(O)OR¹⁰; R⁵ is H, —CN, —C(O)OR^{w}, or —C(O)N(R^{x})R⁷; R⁶ is H; and R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, -OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl.

Usually, R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, and unsubstituted or substituted C₁₋₆ alkyl. Usually, R^{a}, R^{b}, R^{c} and R^{d} are each independently selected from H, methyl or ethyl.

Usually, R^{x} is H; R^{z} is H; R^{w} is H, unsubstituted C₁₋₆ alkyl, or C₁₋₆ alkyl which is substituted with phenyl or —OC(O)R^{ww} wherein R″ is phenyl or unsubstituted C₁₋₆ alkyl; and R^{y} is H or methyl.

For such compounds of the invention, it is usually the case that one of R¹ and R⁵ is —C(O)N(R^{x})R⁷ and the other of R¹ and R⁵ is H, —CN or —C(O)OR^{w}.

Thus, typically, R⁰ is H or methyl; R¹ is H, —CN, —C(O)OR^{w}, or —C(O)N(R^{x})R⁷; R² is H or methyl; and R³ is H or —OR⁸; or R² is —N═ and R³ is =C(R^{y})— and R² and R³ together form a group of formula —N═C(R^{y})—; R⁴ is H, —OR⁹ or —C(O)OR¹⁰; R⁵ is H, —CN, —C(O)OR^{w}, or —C(O)N(R^{x})R⁷; R⁶ is H; R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, -OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl; R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, -N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid; and R^{x} is H; R^{z} is H; R^{w} is H, unsubstituted C₁₋₆ alkyl, or C₁₋₆ alkyl which is substituted with phenyl or —OC(O)R^{ww} wherein R″ is phenyl or unsubstituted C₁₋₆ alkyl; and R^{y} is H or methyl; provided that one of R¹ and R⁵ is —C(O)N(R)R⁷ and the other of R¹ and R⁵ is H, —CN or —C(O)OR^{w}.

Often, in the substituted azine of formula (I), it is the case that (a) R⁵ is —C(O)N(R)R⁷ and (b) R³ is —OR⁸ or R⁴ is —OR⁹. RX, R⁷, R¹ and R⁹, in these embodiments, may be as defined anywhere herein for the compounds of the invention.

In another typical case, in the substituted azine of formula (I), (a) R¹ is —C(O)N(R^{x})R⁷, and (b) R⁴ is —OR⁹ or —C(O)OR¹⁰, or R⁵ is —C(O)OR^{w}. RX, R⁷, R¹ and R⁹, in these embodiments, may be as defined anywhere herein for the compounds of the invention.

Thus, typically, in the substituted azine of formula (I), either:
(1) (a) R⁵ is —C(O)N(R^{x})R⁷ and (b) R³ is —OR⁸ or R⁴ is —OR⁹; or
(2) (a) R¹ is —C(O)N(R^{x})R⁷ and (b) R⁴ is —OR⁹ or —C(O)OR¹⁰, or R⁵ is —C(O)OR^{w}.

R^{x}, R⁷, R⁸ and R⁹ may be as further defined herein. In some embodiments, for instance, R^{x} is H, R⁸ is H, R⁹ is H, and R⁷ is as defined anywhere herein. For instance R⁷ may be —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR^{z} or unsubstituted C₁₋₄ alkyl. R^{z} may be as defined anywhere herein but is often H. R⁷ may for instance be —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, -OMe or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl. R^{z} may be as defined anywhere herein but is often H.

In the compounds of the invention, the substituted azine may have the formula (Ia) shown below. Accordingly, in some embodiments the invention relates to a compound which is a substituted azine of formula (Ia) or a pharmaceutically acceptable salt thereof.

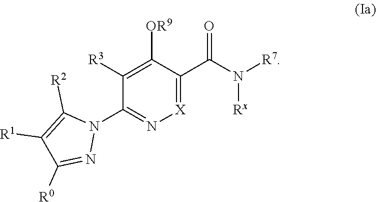

Each of R⁰, R⁹, X, R^{x} and R⁷ in formula (Ia) may be as defined anywhere herein for formula (I). R¹ in formula (Ia) may be H, unsubstituted or substituted C₁₋₆ alkyl, unsubstituted or substituted aryl, unsubstituted or substituted heteroaryl, —CN, -N(R^{t})C(O)N(R^{u})R or —C(O)OR^{w}, wherein R^{t}, R^{u}, R and R^{w} may be as defined anywhere herein for formula (I). R² in formula (Ia) is H, —OR^{q} or unsubstituted or substituted C₁₋₆ alkyl, wherein R^{q} is H, unsubstituted or substituted C₁₋₆ alkyl, or unsubstituted or substituted phenyl. R³ in formula (Ia) is H or unsubstituted or substituted C₁₋₆ alkyl.

Thus, when the substituted azine of the compound of the invention has formula (Ia), typically X is C(R⁶) or N. Preferably X is C(R⁶). Alternatively, however, X may be N.

Typically R⁰ is H or unsubstituted or substituted C₁₋₆ alkyl. However, R⁰ in formula (Ia) may be as further defined anywhere herein for formula (I).

R¹ is usually H, unsubstituted or substituted C₁₋₆ alkyl, unsubstituted or substituted aryl, unsubstituted or substituted heteroaryl, —CN or —C(O)OR^{w}. However, R¹ in formula (Ia) may be as further defined anywhere herein for formula (I).

R² may be H, —OR^{q} or unsubstituted or substituted C₁₋₆ alkyl, and R³ may be H or unsubstituted or substituted C₁₋₆ alkyl. However, R² and R³ in formula (Ia) may be as further defined anywhere herein for formula (I).

Typically R⁶ is H or unsubstituted or substituted C₁₋₆ alkyl. However, R⁶ in formula (Ia) may be as further defined anywhere herein for formula (I).

R⁷ is usually —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R″ is typically H, —C(O)OR^{z} or unsubstituted or substituted C₁₋₄ alkyl. However, R⁷ and R″ in formula (Ia) may be as further defined anywhere herein for formula (I).

In embodiments wherein the substituted azine has the formula (Ia), R⁹ is typically H or unsubstituted or substituted C₁₋₆ alkyl. R^{w}, R^{x} and R^{z} may be each independently selected from H, unsubstituted or substituted C₁₋₄ alkyl, and unsubstituted or substituted phenyl. R^{q} is typically H, unsubstituted or substituted C₁₋₆ alkyl, or unsubstituted or substituted phenyl. However, R⁹, R^{w}, R^{x}, R^{z} and R^{q} in formula (Ia) may be as further defined anywhere herein for formula (I).

In some preferred embodiments wherein the substituted azine has the formula (Ia), R⁹ is H.

The compound of the invention may be a substituted azine of formula (Ia) selected from any one of the following structures, or a pharmaceutically acceptable salt thereof:

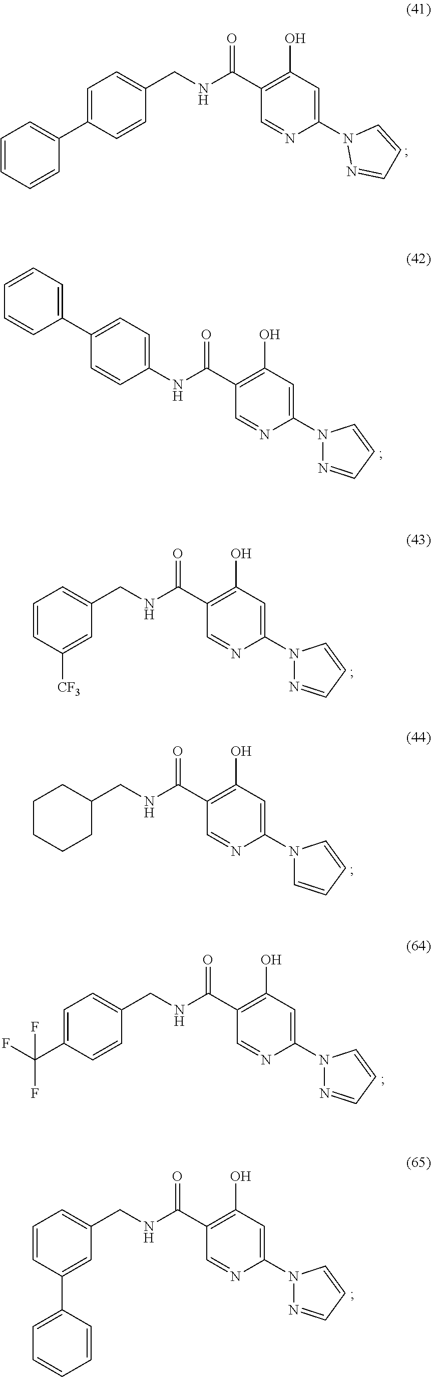 [SMILES: O=C(NCc1ccc(-c2ccccc2)cc1)c1cnc(-n2cccn2)cc1O ; O=C(Nc1ccc(-c2ccccc2)cc1)c1cnc(-n2cccn2)cc1O ; O=C(NCc1cccc(C(F)(F)F)c1)c1cnc(-n2cccn2)cc1O ; O=C(NCC1CCCCC1)c1cnc(-n2cccc2)cc1O ; O=C(NCc1ccc(C(F)(F)F)cc1)c1cnc(-n2cccn2)cc1O ; O=C(NCc1cccc(-c2ccccc2)c1)c1cnc(-n2cccn2)cc1O]

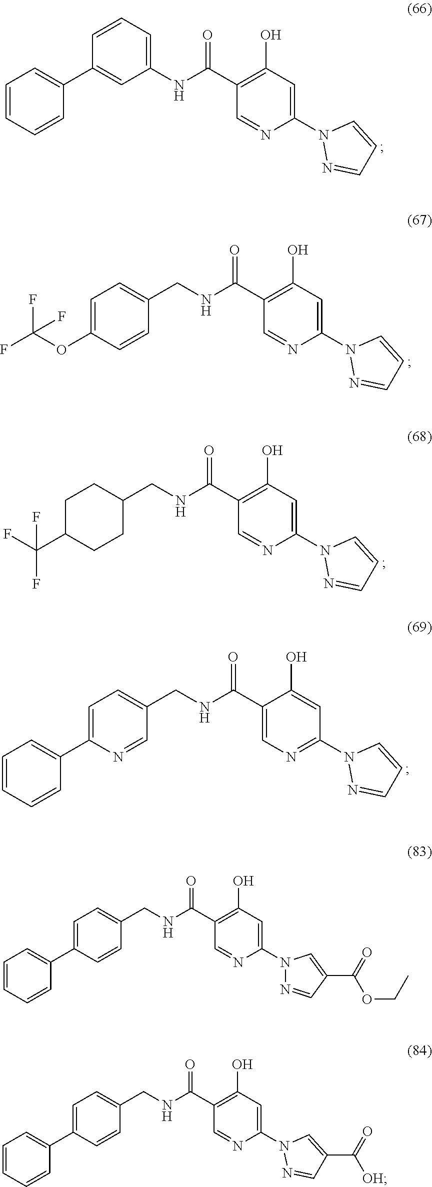 [SMILES: O=C(Nc1cccc(-c2ccccc2)c1)c1cnc(-n2cccn2)cc1O ; O=C(NCc1ccc(OC(F)(F)F)cc1)c1cnc(-n2cccn2)cc1O ; O=C(NCC1CCC(C(F)(F)F)CC1)c1cnc(-n2cccn2)cc1O ; O=C(NCc1ccc(-c2ccccc2)nc1)c1cnc(-n2cccn2)cc1O ; CCOC(=O)c1cnn(-c2cc(O)c(C(=O)NCc3ccc(-c4ccccc4)cc3)cn2)c1 ; O=C(O)c1cnn(-c2cc(O)c(C(=O)NCc3ccc(-c4ccccc4)cc3)cn2)c1]

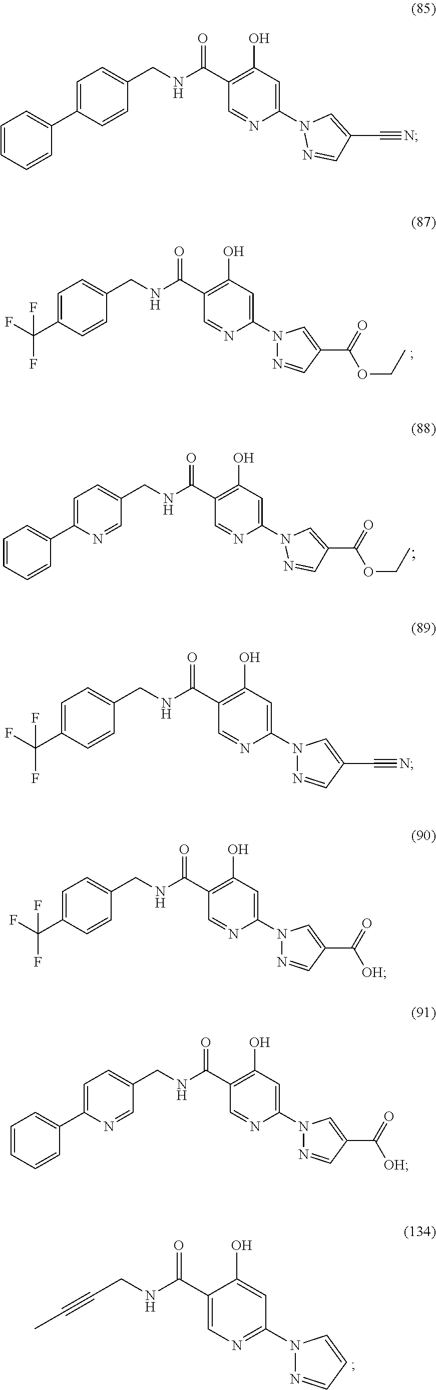

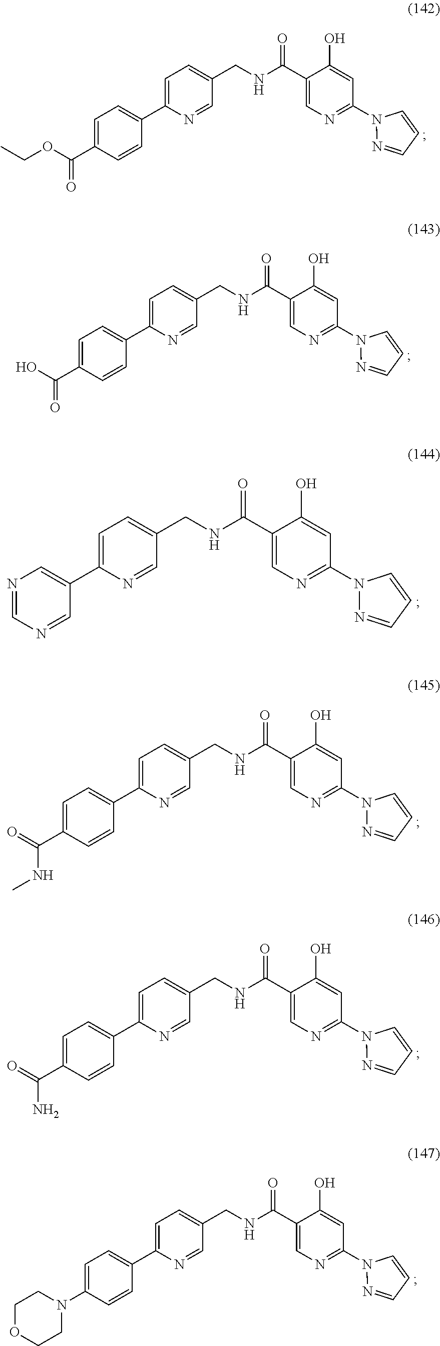 [SMILES: CCOC(=O)c1ccc(-c2ccc(CNC(=O)c3cnc(-n4cccn4)cc3O)cn2)cc1 ; O=C(O)c1ccc(-c2ccc(CNC(=O)c3cnc(-n4cccn4)cc3O)cn2)cc1 ; O=C(NCc1ccc(-c2cncnc2)nc1)c1cnc(-n2cccn2)cc1O ; CNC(=O)c1ccc(-c2ccc(CNC(=O)c3cnc(-n4cccn4)cc3O)cn2)cc1 ; NC(=O)c1ccc(-c2ccc(CNC(=O)c3cnc(-n4cccn4)cc3O)cn2)cc1 ; O=C(NCc1ccc(-c2ccc(N3CCOCC3)cc2)nc1)c1cnc(-n2cccn2)cc1O]

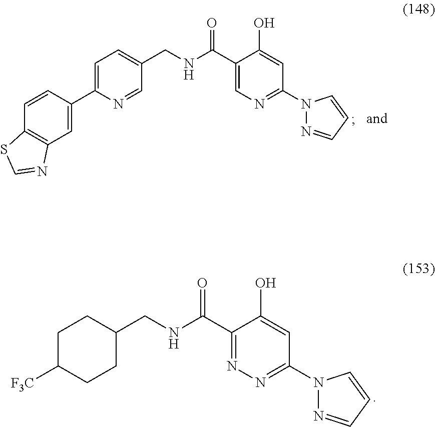 [SMILES: O=C(NCc1ccc(-c2ccc3scnc3c2)nc1)c1cnc(-n2cccn2)cc1O ; O=C(NCC1CCC(C(F)(F)F)CC1)c1nnc(-n2cccn2)cc1O]

The numbers in parentheses next to the structures above match the compound numbers given in the Examples section hereinbelow.

In the compounds of the invention, the substituted azine may have the formula (Ib) shown below. Accordingly, in some embodiments the invention relates to a compound which is a substituted azine of formula (Ib) or a pharmaceutically acceptable salt thereof:

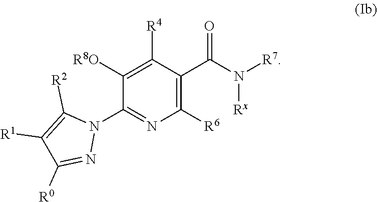

Each of R⁰, R⁴, R⁶, R⁷, R⁸ and R^{x} in formula (Ib) may be as defined anywhere herein for formula (I). R¹ in formula (Ib) may be H, unsubstituted or substituted C₁₋₆ alkyl, unsubstituted or substituted aryl, unsubstituted or substituted heteroaryl, —CN, -N(R^{t})C(O)N(R^{u})R or —C(O)OR^{w}, wherein R^{t}, R^{u}, R and R^{w} may be as defined anywhere herein for formula (I). R² in formula (Ib) is H, —OR^{q} or unsubstituted or substituted C₁₋₆ alkyl, wherein R^{q} is H, unsubstituted or substituted C₁₋₆ alkyl, or unsubstituted or substituted phenyl.

Thus, when the substituted azine of the compound of the invention has formula (Ib), typically R⁰ is H or unsubstituted or substituted C₁₋₆ alkyl. However, R⁰ in formula (Ib) may be as further defined anywhere herein for formula (I).

R¹ may be H, unsubstituted or substituted C₁₋₆ alkyl, unsubstituted or substituted aryl, unsubstituted or substituted heteroaryl, —CN or —C(O)OR^{w}. However, R¹ in formula (Ib) may be as further defined anywhere herein for formula (I).

Usually, R² is H, —OR^{q} or unsubstituted or substituted C₁₋₆ alkyl. However, R² in formula (Ib) may be as further defined anywhere herein for formula (I).

Typically, R⁴ in formula (Ib) is H or unsubstituted or substituted C₁₋₆ alkyl. R⁶ may be H or unsubstituted or substituted C₁₋₆ alkyl. For instance R⁴ may be H, or unsubstituted C₁₋₆ alkyl, and is often H. R⁴ in formula (Ib) may be as further defined anywhere herein for formula (I).

Typically R⁶ is H or unsubstituted or substituted C₁₋₆ alkyl. For instance R⁶ may be H, or unsubstituted C₁₋₆ alkyl, and is often H. R⁶ in formula (Ib) may be as further defined anywhere herein for formula (I).

Typically, R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R″ is H, —C(O)OR^{z} or unsubstituted or substituted C₁₋₄ alkyl. However, R⁷ and R″ in formula (Ib) may be as further defined anywhere herein for formula (I).

Typically, for compounds of formula (Ib), or pharmaceutically acceptable salts thereof, R⁸ is H or unsubstituted or substituted C₁₋₆ alkyl. However, R⁸ in formula (Ib) may be as further defined anywhere herein for formula (I).

Usually, R^{w}, R^{x} and R^{z} are each independently selected from H, unsubstituted or substituted C₁₋₄ alkyl, and unsubstituted or substituted phenyl. R^{q} is typically H, unsubstituted or substituted C₁₋₆ alkyl, or unsubstituted or substituted phenyl. R^{q} is often for instance H. However, R^{w}, R^{x}, R^{z} and R^{q} in formula (Ib) may be as further defined anywhere herein for formula (I).

In some preferred substituted azines of formula (Ib), R⁸ is H.

The compound of the invention may be a substituted azine of formula (Ib) selected from any one of the following structures, or a pharmaceutically acceptable salt thereof:

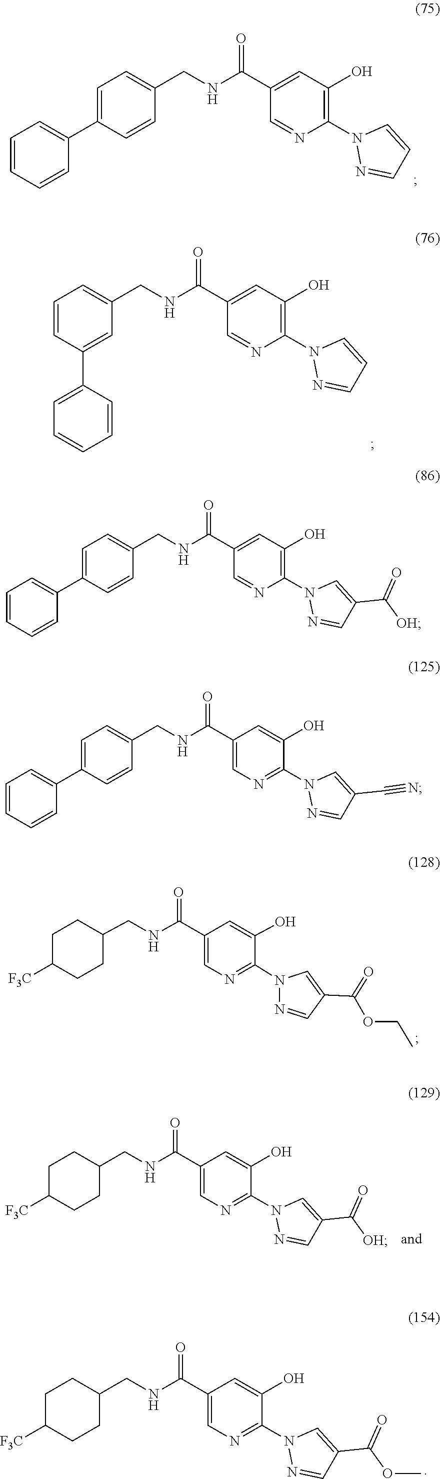

The numbers in parentheses next to the structures above match the compound numbers given in the Examples section hereinbelow.

In the compounds of the invention, the substituted azine may have the formula (Ic) shown below. Accordingly, in some embodiments the invention relates to a compound which is a substituted azine of formula (Ic) or a pharmaceutically acceptable salt thereof:

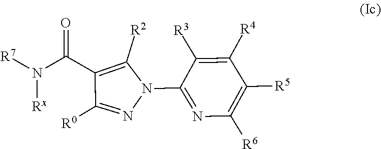

Each of R⁰, R⁴, R⁶, R^{x} and R⁷ in formula (Ic) may be as defined anywhere herein for formula (I). R⁵ in formula (Ic) may be H, unsubstituted or substituted C₁₋₆ alkyl, unsubstituted or substituted aryl, unsubstituted or substituted heteroaryl, —CN, -N(R^{t})C(O)N(R^{u})R or —C(O)OR^{w}, wherein R^{t}, R^{u}, R and R^{w} may be as defined anywhere herein for formula (I). R² in formula (Ic) is H, —OR^{q} or unsubstituted or substituted C₁₋₆ alkyl, wherein R^{q} is H, unsubstituted or substituted C₁₋₆ alkyl, or unsubstituted or substituted phenyl. R³ in formula (Ic) is H, —OR⁸ or unsubstituted or substituted C₁₋₆ alkyl, wherein R′ is selected from H and unsubstituted or substituted C₁. 6 alkyl.

Thus, when the substituted azine of the compound of the invention has formula (Ic), typically R⁰ is H or unsubstituted or substituted C₁₋₆ alkyl. However, R⁰ in formula (Ic) may be as further defined anywhere herein for formula (I). Usually, R² is H, —OR^{q} or unsubstituted or substituted C₁₋₆ alkyl. However, R² in formula (Ic) may be as further defined anywhere herein for formula (I). R³ may be H, —OR⁸ or unsubstituted or substituted C₁₋₆ alkyl. However, R³ in formula (Ic) may be as further defined anywhere herein for formula (I), as may R⁸. R³ in formula (Ic) is often H, or —OR⁸. R⁸ may for instance be unsubstituted C₁₋₆ alkyl.

For the substituted azine of formula (Ic), usually R⁴ is H, unsubstituted or substituted C₁₋₆ alkyl, —OR⁹ or —C(O)OR¹⁰, wherein R⁹ and R¹⁰ are as defined anywhere herein for formula (I). However, R⁴ in formula (Ic) may be as further defined anywhere herein for formula (I). R⁴ may for instance be selected from H, —OR⁹ or —C(O)OR¹⁰. R⁴ may for example be selected from H, —OH and —C(O)OH.

R⁵ in formula (Ic) may be H, unsubstituted or substituted C₁₋₆ alkyl, unsubstituted or substituted aryl, unsubstituted or substituted heteroaryl, —CN or —C(O)OR^{w}. However, R⁵ in formula (Ic) may be as further defined anywhere herein for formula (I), as may R^{w}.

Typically, R⁶ is H or unsubstituted or substituted C₁₋₆ alkyl. However, R⁶ in formula (Ic) may be as further defined anywhere herein for formula (I).

R⁷ may be —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl. However, R⁷ and R″ in formula (Ic) may be as further defined anywhere herein for formula (I).

In embodiments wherein the substituted azine has the formula (Ic), R⁸, R⁹ and R¹⁰ are each independently selected from H and unsubstituted or substituted C₁₋₆ alkyl. However, each of R⁸, R⁹ and R¹⁰ in formula (Ic) may be as further defined anywhere herein for formula (I).

R^{w}, R^{x} and R^{z} are usually each independently selected from H, unsubstituted or substituted C₁₋₄ alkyl, and unsubstituted or substituted phenyl. However, each of R^{w}, R^{x} and R^{z} in formula (Ic) may be as further defined anywhere herein for formula (I).

R^{q} may be H, unsubstituted or substituted C₁₋₆ alkyl, or unsubstituted or substituted phenyl. However, R^{q} in formula (Ic) may be as further defined anywhere herein for formula (I).

Often, in the substituted azine of formula (Ic), R⁴ is OH or C(O)OH. For instance, in the substituted azine of formula (Ic), it is often the case that R⁴ is OH or C(O)OH and R⁵ is H. For instance R⁴ may be OH and R⁵ may be H. Also, typically, R⁴ is C(O)OH and R⁵ is H. Also, typically, R⁴ is OH or C(O)OH and R⁵ is CN. For instance R⁴ may be OH and R⁵ may be CN.

It is also often the case, in the substituted azine of formula (Ic), that R⁵ is C(O)OH. For instance, in the substituted azine of formula (Ic), it is often the case that R⁵ is C(O)OH and R⁴ is H. Also, typically, R⁵ is C(O)OH and R⁴ is OH.

Thus, in some preferred embodiments of the substituted azine of formula (Ic): (i) R⁴ is OH or C(O)OH, and/or (ii) R⁵ is C(O)OH.

Indeed, preferably, in the substituted azine of formula (Ic): (a) R⁴ is OH; or (b) R⁴ is C(O)OH; or (c) R⁵ is C(O)OH; or (d) R⁴ is OH and R⁵ is C(O)OH.

The compound of the invention may be a substituted azine of formula (Ic) selected from any one of the following structures, or a pharmaceutically acceptable salt thereof:

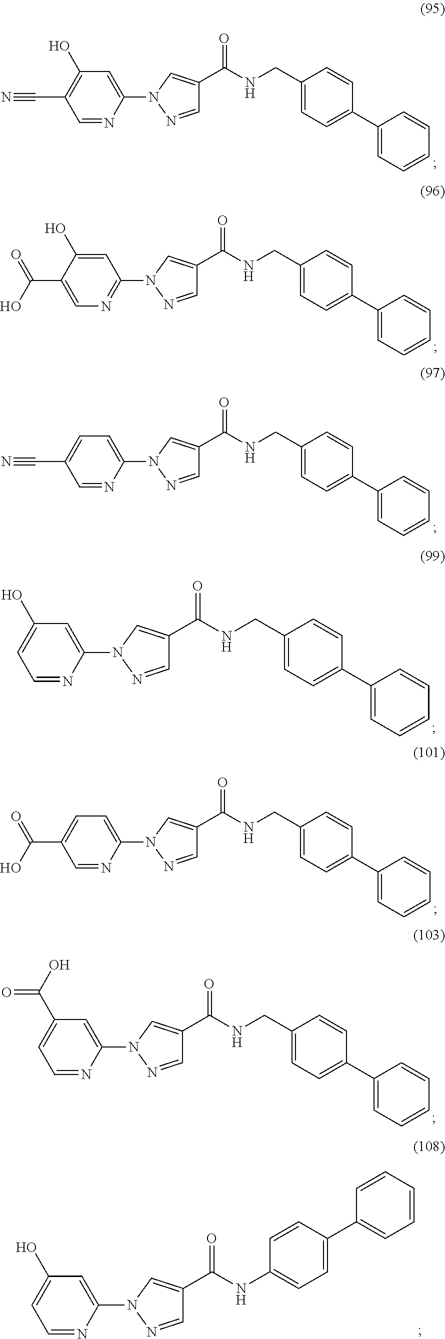

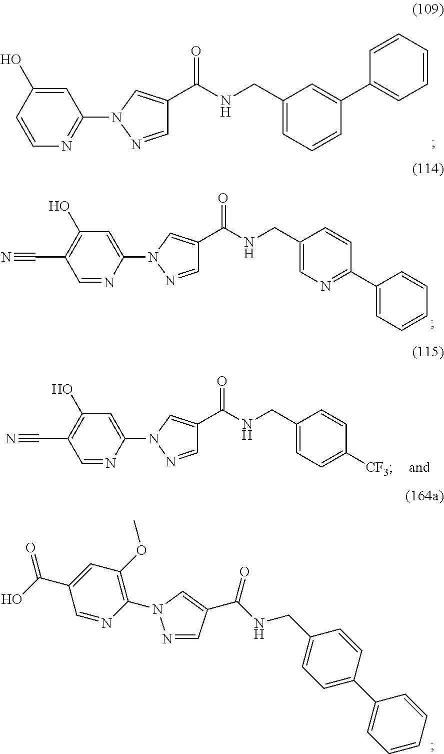 [SMILES: O=C(NCc1cccc(-c2ccccc2)c1)c1cnn(-c2cc(O)ccn2)c1 ; N#Cc1cnc(-n2cc(C(=O)NCc3ccc(-c4ccccc4)nc3)cn2)cc1O ; N#Cc1cnc(-n2cc(C(=O)NCc3ccc(C(F)(F)F)cc3)cn2)cc1O ; COc1cc(C(=O)O)cnc1-n1cc(C(=O)NCc2ccc(-c3ccccc3)cc2)cn1]

The numbers in parentheses next to the structures above match the compound numbers given in the Examples section hereinbelow.

In the compounds of the invention, the substituted azine may have the formula (Id) shown below. Accordingly, in some embodiments the invention relates to a compound which is a substituted azine of formula (Id) or a pharmaceutically acceptable salt thereof:

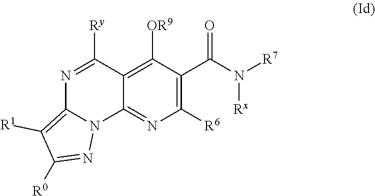

Each of R⁰, R¹, R⁶, R⁹, R^{y}, R^{x} and R in formula (Id) may be as defined anywhere herein for formula (I). However, R¹ in formula (Id) is usually H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}, wherein R^{t}, R^{u}, R and R^{w} may be as defined anywhere herein for formula (I).

Thus, when the substituted azine of the compound of the invention has formula (Id), typically R⁰ is H or unsubstituted or substituted C₁₋₆ alkyl. However, R⁰ in formula (Id) may be as further defined anywhere herein for formula (I). R⁰ in formula (Id) is often H or methyl. Typically, it is methyl.

R¹ in formula (Id) is typically H, unsubstituted or substituted C₁₋₆ alkyl, unsubstituted or substituted aryl, unsubstituted or substituted heteroaryl, —CN or —C(O)OR^{w}. However, R¹ in formula (Id) may be as further defined anywhere herein for formula (I). R¹ in formula (Id) is preferably H.

R^{w} in formula (Id) is selected from H, unsubstituted or substituted C₁₋₄ alkyl, and unsubstituted or substituted phenyl. However, R^{w} in formula (Id) may be as further defined anywhere herein for formula (I).

R^{y} is typically selected from H, unsubstituted or substituted C₁₋₄ alkyl, and unsubstituted or substituted phenyl. R^{y} in formula (Id) may be as further defined anywhere herein for formula (I). Often, however, R^{y} in formula (Id) is H or methyl.

Often, in formula (Id), R^{y} and R⁰ are both methyl. R^{y} and R⁰ may both be H.

Typically R⁶ is H or unsubstituted or substituted C₁₋₆ alkyl. R⁶ in formula (Id) may be as further defined anywhere herein for formula (I). Often, however, R⁶ in formula (Id) is H.

For compounds of formula (Id) or pharmaceutically acceptable salts thereof, R⁷ is usually —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R″ is typically H, —C(O)OR^{z} or unsubstituted or substituted C₁₋₄ alkyl. However, R⁷ and R″ in formula (Id) may be as further defined anywhere herein for formula (I). R^{z} is selected from H, unsubstituted or substituted C₁₋₄ alkyl, and unsubstituted or substituted phenyl. However, R^{z} in formula (Id) may also be as further defined anywhere herein for formula (I).

R⁹ is typically H or unsubstituted or substituted C₁₋₆ alkyl.

R^{x} in formula (Id) is selected from H, unsubstituted or substituted C₁₋₄ alkyl, and unsubstituted or substituted phenyl. However, R^{x} in formula (Id) may be as further defined anywhere herein for formula (I). Usually, R^{x} in formula (Id) is H.

In some preferred embodiments wherein the substituted azine has the formula (Id), R⁹ is H.

The compound of the invention may be a substituted azine of formula (Id) selected from any one of the following structures, or a pharmaceutically acceptable salt thereof:

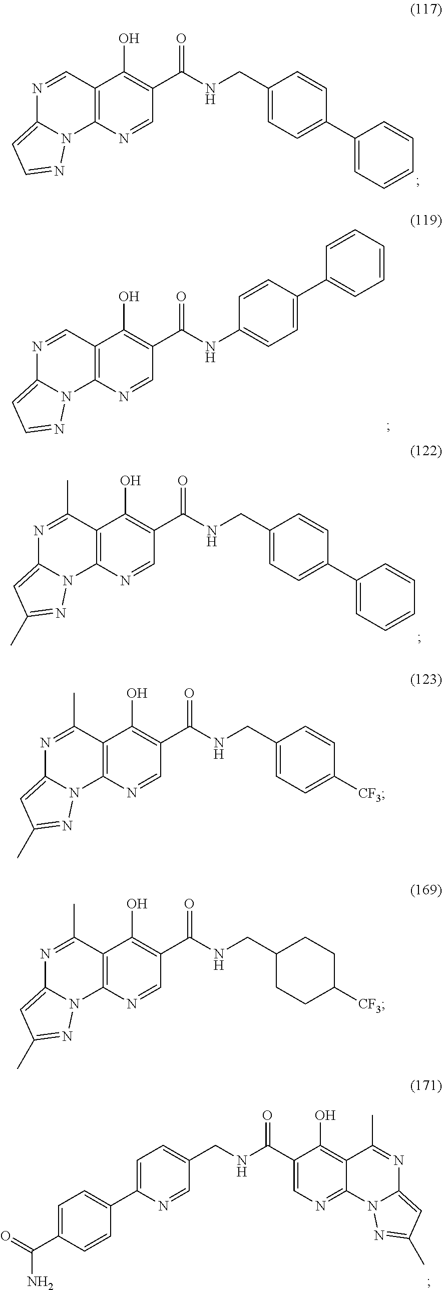 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1 ; Cc1cc2nc(C)c3c(O)c(C(=O)NCc4ccc(-c5ccc(C(N)=O)cc5)nc4)cnc3n2n1]

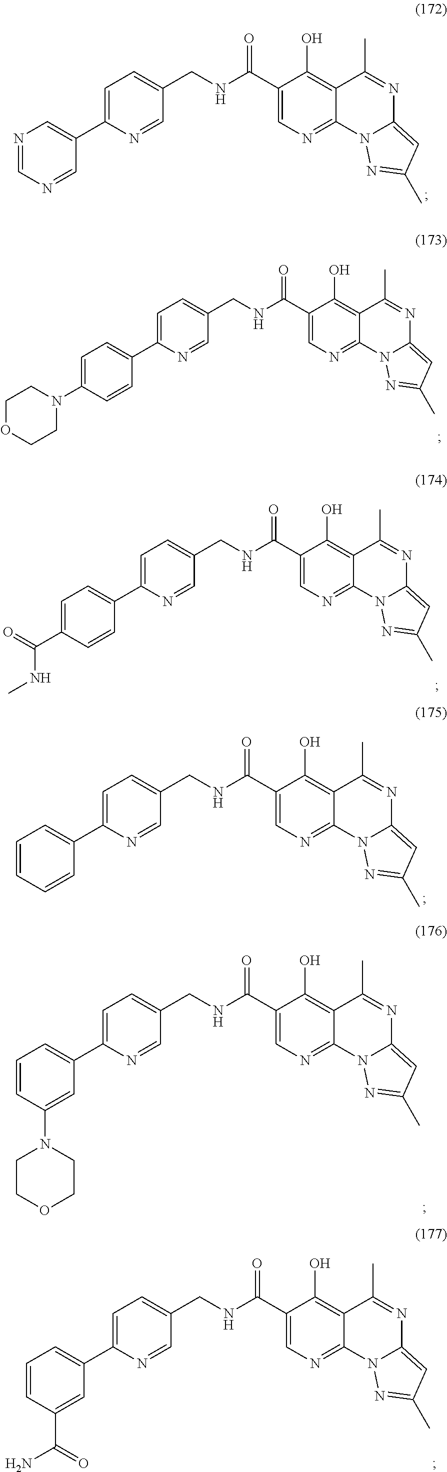

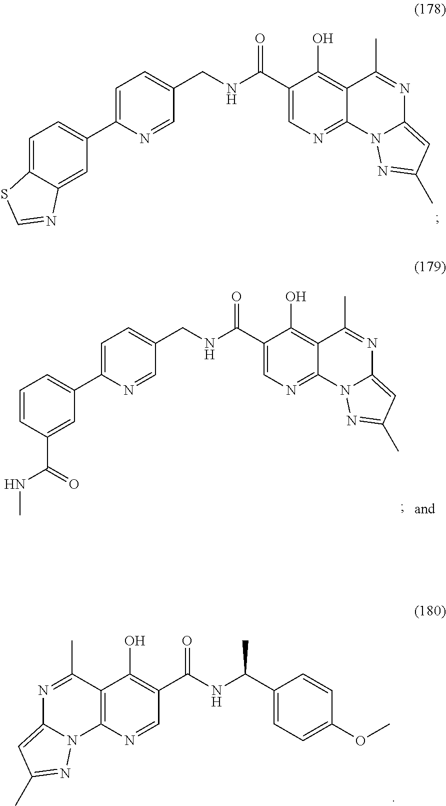

The numbers in parentheses next to the structures listed above correspond to the compound numbers given in the Examples section hereinbelow.

In another embodiment, the substituted azine of formula (I) has any one of the following structures. Accordingly, the invention provides a compound which is a substituted azine having any one of the following structures or a pharmaceutically acceptable salt thereof:

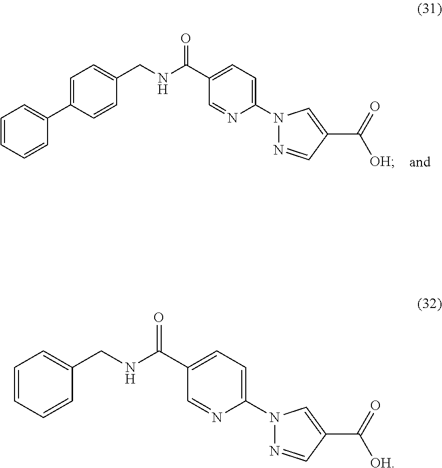 [SMILES: O=C(O)c1cnn(-c2ccc(C(=O)NCc3ccc(-c4ccccc4)cc3)cn2)c1 ; O=C(O)c1cnn(-c2ccc(C(=O)NCc3ccccc3)cn2)c1]

The numbers in parentheses next to the structures listed above correspond to the compound numbers given in the Examples section hereinbelow.

The invention also provides a compound which is a substituted azine of formula (Ia) as defined herein, or a pharmaceutically acceptable salt thereof, wherein R⁹ in said formula (Ia) is other than H (and wherein X, R⁰, R¹, R², R³, R⁷ and R^{x} are as defined herein for formula Ia). Such a compound embraces prodrugs. Typically, R⁹ in this embodiment is unsubstituted or substituted C₁₋₆ alkyl. In one aspect of this embodiment, R⁹ is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ may for instance be C₁₋₆ alkyl which is substituted with —OC(O)R⁹⁹, wherein R⁹⁹ is as defined above. In another aspect of this embodiment, R⁹ is unsubstituted C₁₋₆ alkyl. R⁹ may for instance be methyl. The substituted azine of formula (Ia) may for instance be selected from any one of the following structures:

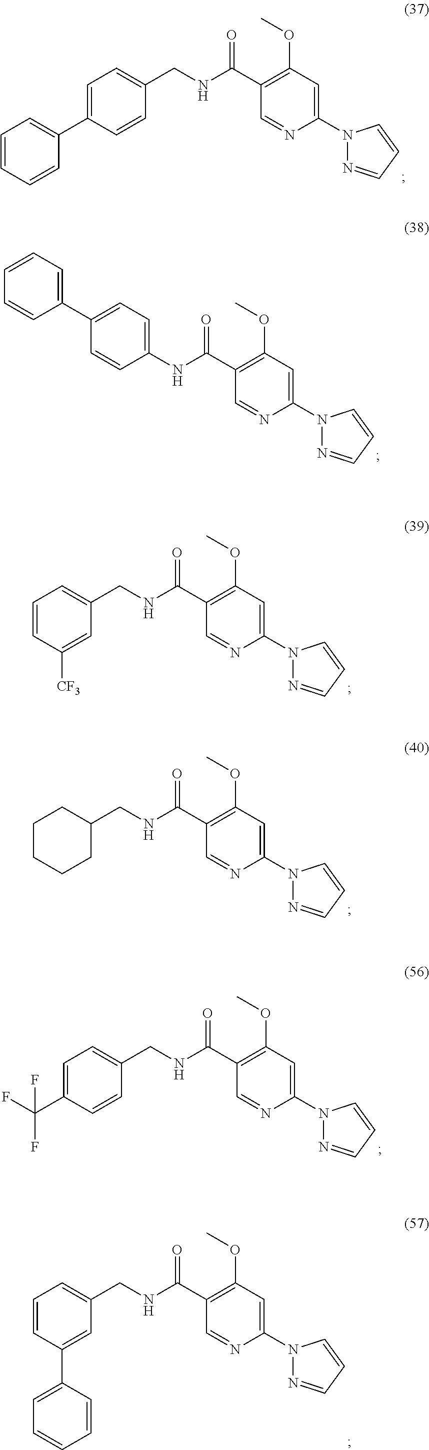 [SMILES: COc1cc(-n2cccn2)ncc1C(=O)NCc1ccc(-c2ccccc2)cc1 ; COc1cc(-n2cccn2)ncc1C(=O)Nc1ccc(-c2ccccc2)cc1 ; COc1cc(-n2cccn2)ncc1C(=O)NCc1cccc(C(F)(F)F)c1 ; COc1cc(-n2cccn2)ncc1C(=O)NCC1CCCCC1 ; COc1cc(-n2cccn2)ncc1C(=O)NCc1ccc(C(F)(F)F)cc1 ; COc1cc(-n2cccn2)ncc1C(=O)NCc1cccc(-c2ccccc2)c1]

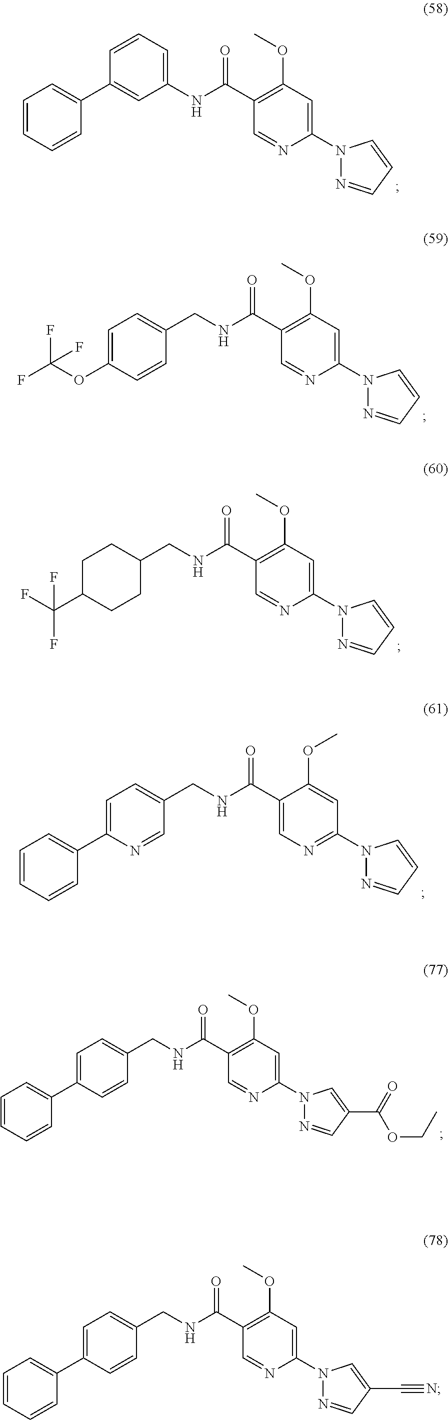 [SMILES: COc1cc(-n2cccn2)ncc1C(=O)Nc1cccc(-c2ccccc2)c1 ; COc1cc(-n2cccn2)ncc1C(=O)NCc1ccc(OC(F)(F)F)cc1 ; COc1cc(-n2cccn2)ncc1C(=O)NCC1CCC(C(F)(F)F)CC1 ; COc1cc(-n2cccn2)ncc1C(=O)NCc1ccc(-c2ccccc2)nc1 ; CCOC(=O)c1cnn(-c2cc(OC)c(C(=O)NCc3ccc(-c4ccccc4)cc3)cn2)c1 ; COc1cc(-n2cc(C#N)cn2)ncc1C(=O)NCc1ccc(-c2ccccc2)cc1]

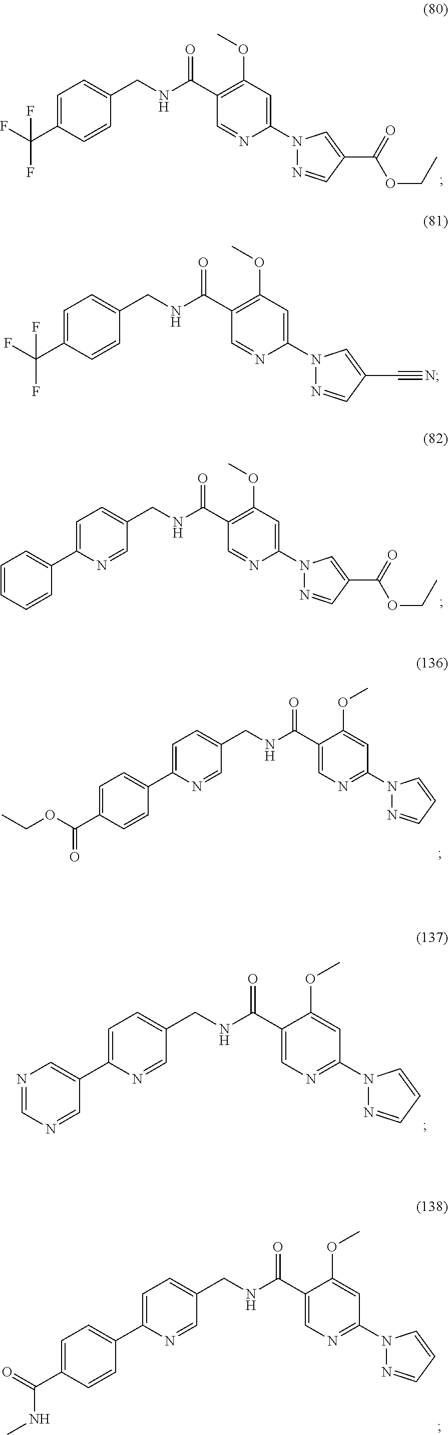

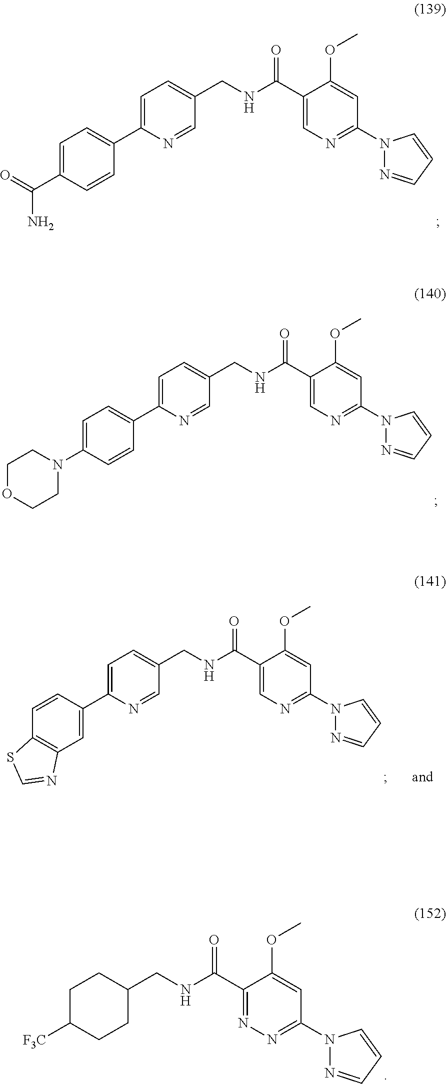 [SMILES: COc1cc(-n2cccn2)ncc1C(=O)NCc1ccc(-c2ccc(C(N)=O)cc2)nc1 ; COc1cc(-n2cccn2)ncc1C(=O)NCc1ccc(-c2ccc(N3CCOCC3)cc2)nc1 ; COc1cc(-n2cccn2)ncc1C(=O)NCc1ccc(-c2ccc3scnc3c2)nc1 ; COc1cc(-n2cccn2)nnc1C(=O)NCC1CCC(C(F)(F)F)CC1]

The numbers in parentheses next to the structures listed above correspond to the compound numbers given in the Examples section hereinbelow.

The invention also provides a compound which is a substituted azine of formula (Ib) as defined herein, or a pharmaceutically acceptable salt thereof, wherein R⁸ in said formula (Ib) is other than H (and wherein R⁰, R¹, R², R⁴, R⁶, R⁷ and R^{x} are as defined herein for formula Ib). Such a compound embraces prodrugs. Typically, R⁸ in this embodiment is unsubstituted or substituted C₁₋₆ alkyl. In one aspect of this embodiment, R⁸ is substituted C₁₋₆ alkyl. R⁸ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁸ may for instance be C₁₋₆ alkyl which is substituted with —OC(O)R⁹⁹, wherein R⁹⁹ is as defined above. In another aspect of this embodiment, R⁸ is unsubstituted C₁₋₆ alkyl. R′ may for instance be methyl. The substituted azine of formula (Ib) may for instance be selected from any one of the following structures

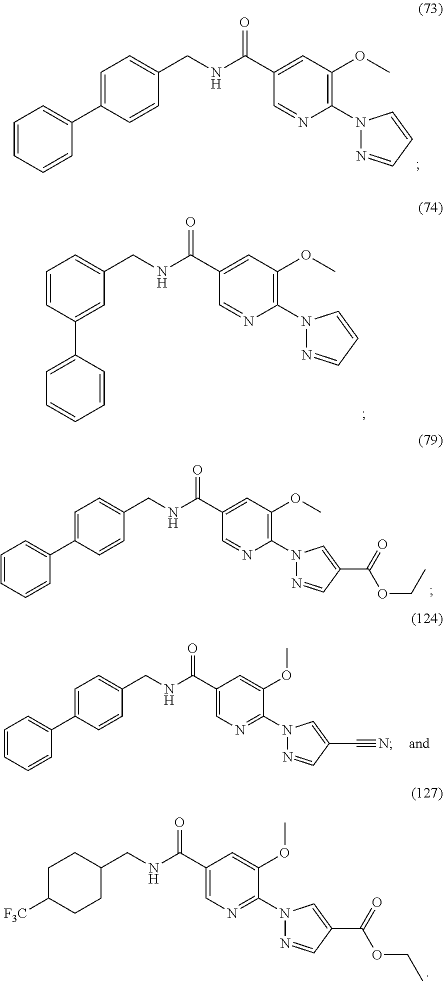 [SMILES: COc1cc(C(=O)NCc2ccc(-c3ccccc3)cc2)cnc1-n1cccn1 ; COc1cc(C(=O)NCc2cccc(-c3ccccc3)c2)cnc1-n1cccn1 ; CCOC(=O)c1cnn(-c2ncc(C(=O)NCc3ccc(-c4ccccc4)cc3)cc2OC)c1 ; COc1cc(C(=O)NCc2ccc(-c3ccccc3)cc2)cnc1-n1cc(C#N)cn1 ; CCOC(=O)c1cnn(-c2ncc(C(=O)NCC3CCC(C(F)(F)F)CC3)cc2OC)c1]

The numbers in parentheses next to the structures listed above correspond to the compound numbers given in the Examples section hereinbelow.

The invention also provides a compound which is a substituted azine of formula (Ic) as defined herein, or a pharmaceutically acceptable salt thereof, wherein R⁴ is —OR⁹ or —C(O)OR¹⁰, and/or R⁵ is —C(O)OR^{w}, in which R⁹, R¹⁰ and R^{w} are other than H (and wherein R⁰, R², R³, R⁴, R⁵, R⁶, R⁷ and R^{x} are otherwise as defined herein for formula Ic). Such a compound embraces prodrugs. Typically, in the substituted azine of formula (Ic): (a) R⁴ is —OR⁹; or (b) R⁴ is —C(O)OR¹⁰; or (c) R⁵ is —C(O)OR^{w}; or (d) R⁴ is —OR⁹ and R⁵ is —C(O)OR^{w}. R⁹, R¹⁰ and R^{w}, which are the same or different, are unsubstituted or substituted C₁₋₆ alkyl groups.

In one aspect of this embodiment, R⁹, R¹⁰ and R^{w} are substituted C₁₋₆ alkyl groups. R⁹ and R¹⁰ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ and R¹⁰ may for instance be C₁₋₆ alkyl which is substituted with —OC(O)R⁹⁹, wherein R⁹⁹ is as defined above. Similarly, R^{w}, in this aspect of this embodiment, may be a substituted C₁₋₆ alkyl group. R^{w} may for instance be C₁₋₆ alkyl which is substituted with phenyl or —OC(O)R^{ww}, wherein R^{ww} is is as defined above. Thus R^{ww} may be phenyl, unsubstituted C₁₋₆ alkyl, —N(R^{a})(R), —C(O)R^{c}, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R^{w} may for instance be C₁₋₆ alkyl which is substituted with —OC(O)R^{ww}, wherein R^{ww} is is as defined above.

In another aspect of this embodiment, R⁹, R¹⁰ and R^{w}, which may be the same or different, are unsubstituted C₁₋₆ alkyl groups. R⁹, R¹⁰ and R^{w} may for instance be selected from methyl and ethyl groups.

The substituted azine of formula (Ic) may for instance be selected from any one of the following structures

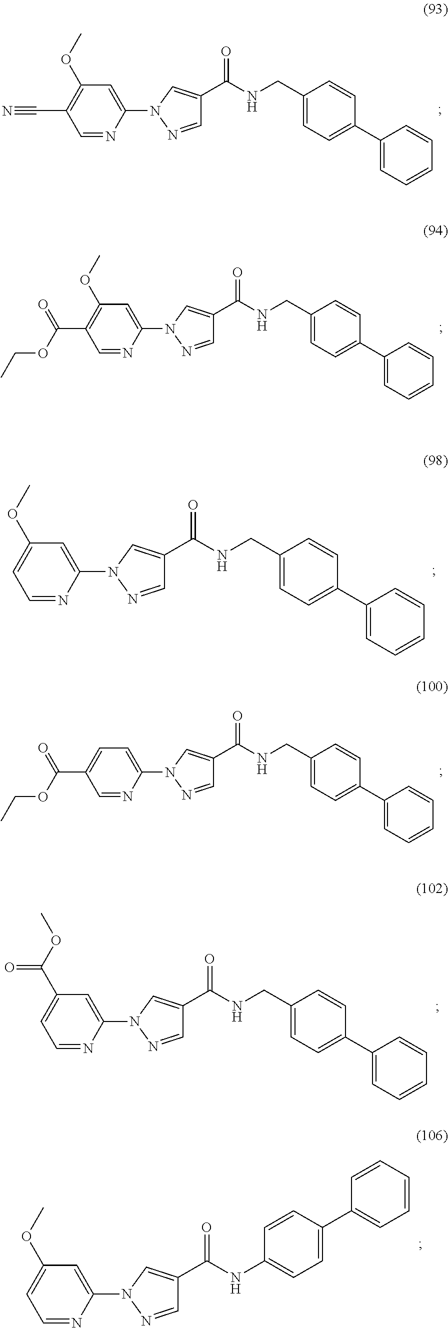 [SMILES: COc1cc(-n2cc(C(=O)NCc3ccc(-c4ccccc4)cc3)cn2)ncc1C#N ; CCOC(=O)c1cnc(-n2cc(C(=O)NCc3ccc(-c4ccccc4)cc3)cn2)cc1OC ; COc1ccnc(-n2cc(C(=O)NCc3ccc(-c4ccccc4)cc3)cn2)c1 ; CCOC(=O)c1ccc(-n2cc(C(=O)NCc3ccc(-c4ccccc4)cc3)cn2)nc1 ; COC(=O)c1ccnc(-n2cc(C(=O)NCc3ccc(-c4ccccc4)cc3)cn2)c1 ; COc1ccnc(-n2cc(C(=O)Nc3ccc(-c4ccccc4)cc3)cn2)c1]

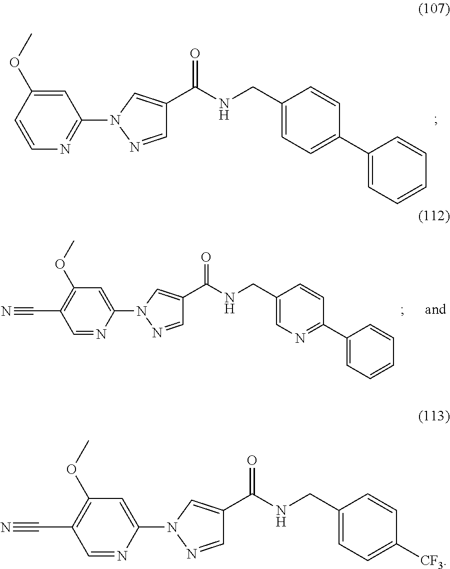 [SMILES: COc1ccnc(-n2cc(C(=O)NCc3ccc(-c4ccccc4)cc3)cn2)c1 ; COc1cc(-n2cc(C(=O)NCc3ccc(-c4ccccc4)nc3)cn2)ncc1C#N ; "and" ; COc1cc(-n2cc(C(=O)NCc3ccc(C(F)(F)F)cc3)cn2)ncc1C#N]

The numbers in parentheses next to the structures listed above correspond to the compound numbers given in the Examples section hereinbelow.

The invention also provides a compound which is a substituted azine of formula (Id) as defined herein, or a pharmaceutically acceptable salt thereof, wherein R⁹ is other than H (and wherein R⁰, R¹, R^{y}, R⁶, R⁷ and R^{x} are as defined herein for formula Id). Such a compound embraces prodrugs. Typically, R⁹ in this embodiment is unsubstituted or substituted C₁₋₆ alkyl. In one aspect of this embodiment, R⁹ is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ may for instance be C₁₋₆ alkyl which is substituted with —OC(O)R⁹⁹, wherein R⁹⁹ is as defined above. In another aspect of this embodiment, R⁹ is unsubstituted C₁₋₆ alkyl. R⁹ may for instance be methyl. The substituted azine of formula (Id) may for instance have the following structure

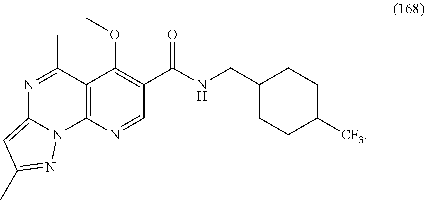 [SMILES: COc1c(C(=O)NCC2CCC(C(F)(F)F)CC2)cnc2c1c(C)nc1cc(C)nn12]

The number in parentheses next to the structure listed above corresponds to the compound numbers given in the Examples section hereinbelow.

The above compounds of formula (Ia), (Ib), (Ic) and (Id) wherein R⁸, R⁹, R¹⁰ or R^{w} are typically unsubstituted or substituted C₁₋₆ alkyl have surprising advantages as prodrug structures for compounds of formula (Ia), (Ib), (Ic) or (Id), which are effective HIF-PHD inhibitors. In particular, the compounds with the structures as described above have been surprisingly shown to improve efficacy of the inhibitors in a cellular assay, even when they themselves do not have high potency as HIF-PHD inhibitors. The reduced potency but higher activity in a cellular assay means that these compounds have the potential to provide targeting inhibition with reduced off-target effects.

The invention also provides a compound which is a substituted pyrimidine of formula (IV) or a pharmaceutically acceptable salt thereof

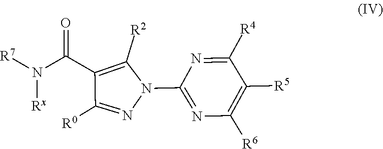

wherein
R⁰ is H or unsubstituted or substituted C₁₋₆ alkyl;
R² is H, —OR^{q} or unsubstituted or substituted C₁₋₆ alkyl;
R⁴ is —OR⁹, wherein R⁹ is selected from H and unsubstituted or substituted C₁₋₆ alkyl;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R^{x} is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

Typically, in formula (IV), R⁰ is H or unsubstituted C₁₋₆ alkyl, for instance H or methyl. Often, R⁰ is H.

Usually, in formula (IV), R² is H or unsubstituted C₁₋₆ alkyl, for instance H or methyl. Often, R² is H.

Typically, in formula (IV), R⁰ is H and R² is H.

Often, in formula (IV), R⁵ is —CN.

R⁶ in formula (IV) is often H or unsubstituted C₁₋₆ alkyl, for instance H or methyl. R⁶ in formula (IV) is typically H.

R⁴ in formula (IV) is —OR⁹, and R⁹ is selected from H and unsubstituted or substituted C₁₋₆ alkyl. However, R⁹, in formula (IV), may be as defined anywhere herein for R⁹ formula (I).

Often, in formula (IV), R⁹ is H. When R⁹ is H (i.e. when R⁴ is OH), then R⁵ is typically —CN.

In some embodiments, however, R⁹ in formula (IV) may be unsubstituted or substituted C₁₋₆ alkyl. Such embodiments embrace prodrugs. In one aspect of this embodiment, R⁹ in formula (IV) is substituted C₁₋₆ alkyl. R⁹ may for instance be C₁₋₆ alkyl which is substituted with phenyl or —OC(O)R⁹⁹, wherein R⁹⁹ is as defined above. Thus R⁹⁹ may be phenyl, unsubstituted C₁₋₆ alkyl, —N(R^{a})(R), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Typically, R^{a}, R^{b}, R^{c} and R^{d} are each independently selected from H, or unsubstituted or substituted C₁₋₆ alkyl. Usually, R^{a}, R^{b}, R^{c} and R^{d} are each independently selected from H, methyl or ethyl. R⁹ in formula (IV) may for instance be C₁₋₆ alkyl which is substituted with —OC(O)R⁹⁹, wherein R⁹⁹ is as defined above. In another aspect of this embodiment, R⁹ in formula (IV) is unsubstituted C₁₋₆ alkyl. R⁹ may for instance be methyl.

R^{x} in formula (IV) is typically H or unsubstituted C₁₋₄ alkyl, for instance H or methyl. Often, RX in formula (IV) is H.

R^{w} in formula (IV) is H, unsubstituted or substituted C₁₋₆ alkyl, or unsubstituted or substituted phenyl. R^{w} may be H, or unsubstituted or substituted C₁₋₆ alkyl. Typically, for instance, R^{w} is H, unsubstituted C₁₋₆ alkyl, or C₁₋₆ alkyl which is substituted with phenyl or —OC(O)R^{ww}, wherein R^{ww} is phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid. Often, R^{w} in formula (IV) is H.

Typically, R^{z} in formula (IV) is H or unsubstituted C₁₋₄ alkyl, for instance H or methyl. Often, R^{z} in formula (IV) is H.

R^{q} in formula (IV) is typically H or unsubstituted C₁₋₆ alkyl, or unsubstituted phenyl. It is often, for instance, H or unsubstituted C₁₋₄ alkyl, for instance H or methyl. Usually, R^{q} in formula (IV) is H.

Usually, R⁷ in formula (IV) is —CH(R¹¹)—Ar, —CH(R¹¹)-Ary-Ar, or —CH(R¹¹)-Cyc, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl. R″ is typically H or unsubstituted C₁₋₄ alkyl, for instance H or methyl. Often, R¹¹ is H.

R⁷ in formula (IV) may for instance be —CH(R¹¹)—Ar, —CH(R¹¹)-Ary-Ar, or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)OEt, —C(O)NH₂, —C(O)N(H)Me, —OMe or N-morpholino; Ary is unsubstituted phenylene or unsubstituted pyridylene; Cyc is unsubstituted cyclohexyl or cyclohexyl substituted with —CF₃ or —OCF₃; and R″ is as defined above, typically H. Often, R⁷ in formula (IV) is —CH(R¹¹)—Ar, —CH(R¹¹)-Ary-Ar, or —CH(R¹¹)-Cyc; wherein Ar is unsubstituted phenyl or phenyl substituted with —C(O)OH or —C(O)OMe; Ary is unsubstituted phenylene or unsubstituted pyridylene; Cyc is unsubstituted cyclohexyl or cyclohexyl substituted with —CF₃; and R″ is as defined above, typically H.

A compound of formula (IV) may be represented by one of the following structures or a pharmaceutically acceptable salt thereof:

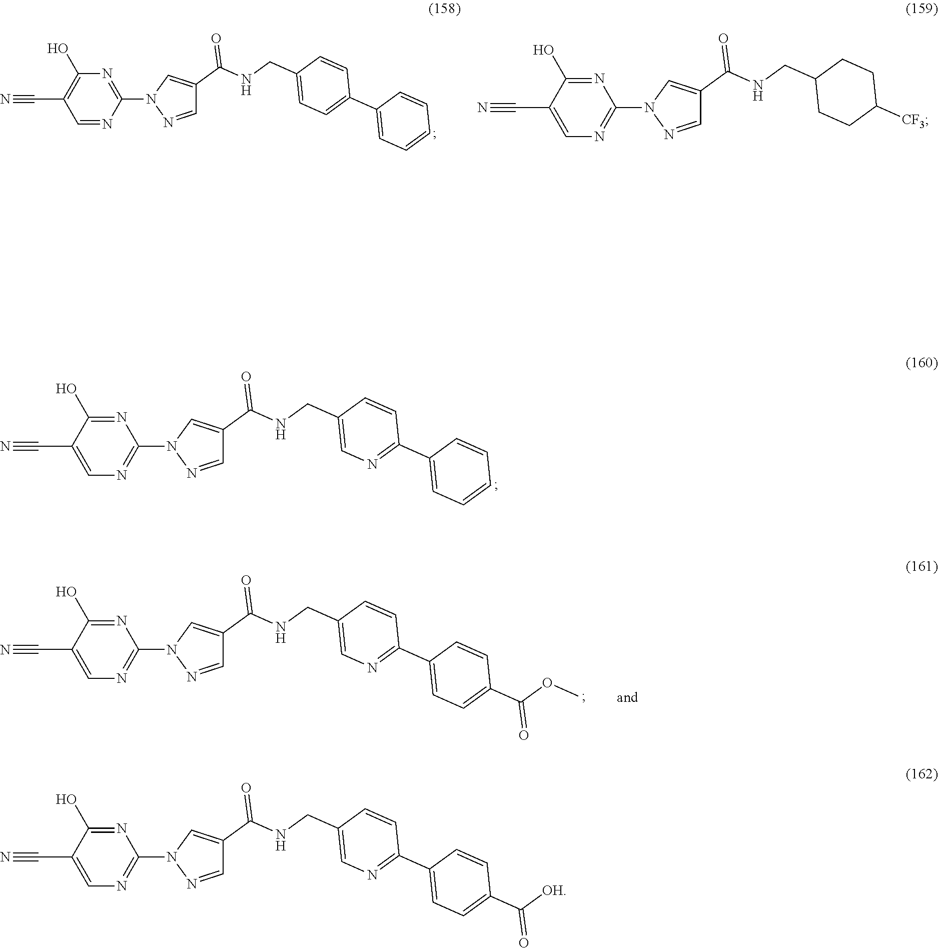 [SMILES: N#Cc1cnc(-n2cc(C(=O)NCc3ccc(-c4ccccc4)cc3)cn2)nc1O ; N#Cc1cnc(-n2cc(C(=O)NCC3CCC(C(F)(F)F)CC3)cn2)nc1O ; N#Cc1cnc(-n2cc(C(=O)NCc3ccc(-c4ccccc4)nc3)cn2)nc1O ; COC(=O)c1ccc(-c2ccc(CNC(=O)c3cnn(-c4ncc(C#N)c(O)n4)c3)cn2)cc1 ; N#Cc1cnc(-n2cc(C(=O)NCc3ccc(-c4ccc(C(=O)O)cc4)nc3)cn2)nc1O]

The numbers in parentheses next to the structures listed above correspond to the compound numbers given in the Examples section hereinbelow.

### General Synthetic Methodology

The compounds of the invention can be prepared by any suitable method. Detailed general synthetic routes for compounds of the invention are set out below and in the Examples.

The substituted azines of formula (I) and the substituted pyrimidines of formula (IV) may for instance be synthesised using the methodology set forth under the headings “General Procedure A”, “General Procedure B”, “General Procedure C” and “General Procedure D” in the Examples section hereinbelow. The application of these General Procedures to produce substituted azines of formula (I) is shown and described below with reference to schemes 2, 3, 4, 5, 6, 7, 8, 9, 10, 11 and 12, and specific synthesis examples are described in the Examples section hereinbelow. Similarly, the application of the General Procedures to produce substituted pyrimidines of formula (IV) is shown and described below with reference to scheme 11.

Substituted azines of formula (I) and substituted pyrimidines of formula (IV) may be synthesised using an amide coupling procedure (General Procedure A or B), which can be used to introduce an amide group (e.g. the amide group of formula —C(O)N(R^{x})R⁷) by coupling it with a carboxylic acid or an ester group in a precursor compound. A Pd-catalysed coupling procedure (General Procedure C) may then be employed to further modify the amide group introduced in the previous step, to arrive at the desired C(O)N(R^{x})R⁷ group in the final compound. An alkoxy-dealkylation step (General Procedure D) may then be employed as a final step to render an OH group in the final compound.

Scheme 10 below, for instance, shows how substituted azines of formula (Ia) may be produced using the General Procedures B, C and D provided in the Example section. The same General Procedures B, C and D can also be used to produce substituted azines of formula (Ib). Scheme 6 below shows how substituted azines of formula (Ic) may be produced using the General Procedures B and D described in the Example section. Schemes 7, 8 and 12 below illustrate the synthesis of the pyrazolo[1,5-a]pyrido[3,2-e]-pyrimidine-7-amido structures of formula (Id) using the general procedures A and D in the Examples. Furthermore, scheme 11 illustrates how substituted pyrimidines of formula (IV) can be produced using General Procedure B. Schemes 1 and 2 are also provided below, to illustrate general methods for preparation of some of the reference Examples described herein. As the skilled person will appreciate, alternative precursor compounds, with substituent groups that are different from those shown in the schemes below, may be employed in the same methods in order to achieve variation within the scopes of formulae (I), (Ia), (Ib), (Ic), (Id) and (IV) herein.

Thus, compounds described herein can be prepared according to the following reaction schemes:

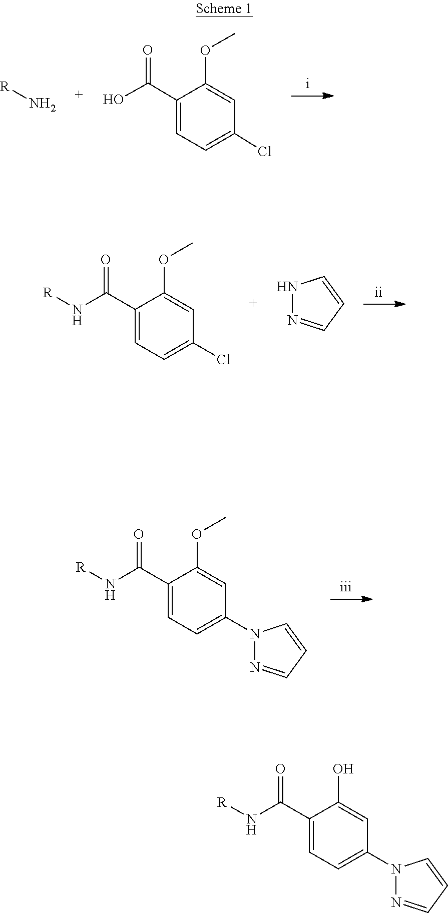

Scheme 1 step (i) may be carried out using treatment with any appropriate peptide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at standard atmospheric temperature and pressure (SATP), i.e. approximately 25° C., and 1 atmospheric pressure (around 100,000 Pa). The step may last for between 1 and 24 hours, for example about 16 hours.

Step (ii) of Scheme 1 comprises treatment in the presence of a catalyst. The catalyst may be a palladium catalyst. Typically, the catalyst may be Pd^{t}BuXPhos. The step may also take place in the presence of a base. The base may be a carbonate. Usually, the base is cesium carbonate (Cs₂CO₃). The reaction typically occurs in a solvent. The solvent may be a mixture of solvents. Typically, the solvent may be a mixture of one or more polar solvents, often polar aprotic solvents. Usually, the reaction occurs in a mixture of dimethylacetamide (DMAc) and tetrahydrofuran (THF). This mixture may be a 1:1 mixture (1:1). Step (ii) typically occurs at a temperature greater than room temperature. For example, step (ii) typically occurs between 60° C. and 100° C. Usually the step occurs at around 80° C. The step may last for between 1 and 24 hours, for example about 16 hours.

Step (iii) of Scheme 1 is a deprotection step that may comprise treatment with any suitable reagents known to the skilled person. In some instances, a silyl halide compound is used. Usually, trimethylsilyl iodide (TMS-I) is used. This step typically takes place in the presence of a solvent. The solvent is typically a polar solvent, and more typically a polar aprotic solvent. The solvent may be dichloromethane (CH₂Cl₂). Step (iii) typically occurs at a temperature greater than room temperature. For example, step (iii) typically occurs between 40° C. and 80° C. Usually the step occurs at around 60° C. The step may last for between 1 and 24 hours, for example about 8 hours.

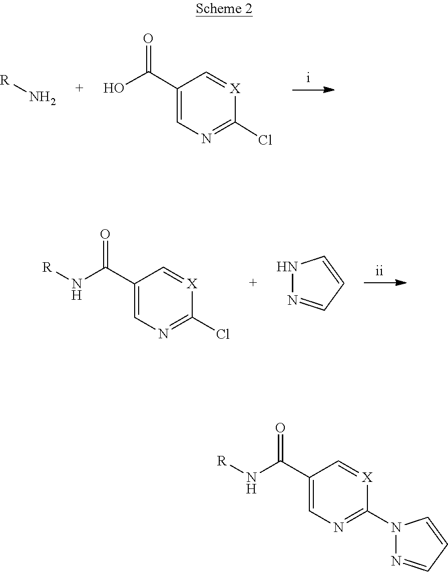

Scheme 2 step (i) may comprise treatment with any appropriate amide coupling reagents. Typically, step (i) comprises treatment with an acid anhydride, for example propanephosphonic acid anhydride (T3P) in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 2 comprises treatment in the presence of a catalyst. The catalyst may be a palladium catalyst. Typically, the catalyst is Pd^{t}BuXPhos. The step may also take place in the presence of a base. The base may be a carbonate. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in a mixture of DMAc and tetrahydrofuran (THF). This mixture may be an approximately 1:1 mixture (1:1). Step (ii) typically occurs at a temperature greater than room temperature. For example, step (ii) typically occurs between 80° C. and 120° C. Usually, the step occurs at around 100° C. The step may last for between 30 minutes and 6 hours, for example about 1 hour.

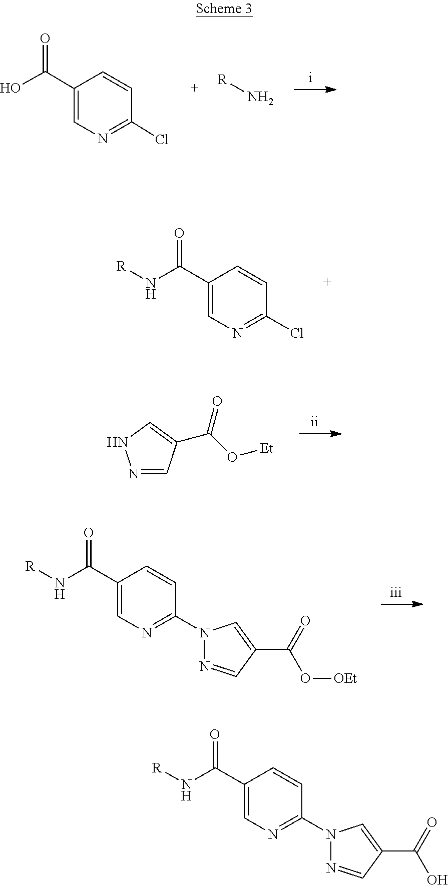

Scheme above shows ow certain substituted azines of formula (I) may be produced using the general synthesis procedures set forth in the Examples section hereinbelow (see the General Procedures A, B, C and D in the Examples).

Scheme 3 step (i) may comprise treatment with any appropriate peptide coupling reagents. Typically step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 3 comprises treatment in the presence of a catalyst. The catalyst may be a palladium catalyst. Typically, the catalyst may be Pd^{t}BuXPhos. The step may also take place in the presence of a base. The base may be a carbonate. Typically, the base is Cs₂CO₃, Na₂CO₃ or K₂CO₃. Usually, the base is Cs₂CO₃. The reaction typically occurs in a solvent. The solvent may be a mixture of solvents. Typically, the solvent may be a mixture of one or more polar solvents, often polar aprotic solvents. Usually, the reaction occurs in a mixture of DMAc and tetrahydrofuran (THF). This mixture may be a 1:1 mixture (1:1). Step (ii) typically occurs at a temperature greater than room temperature. For example, step (ii) typically occurs between 80° C. and 120° C. Usually the step occurs at around 100° C. The step may last for between 30 minutes and 6 hours, for example about 1 hour.

Step (iii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THE and H₂O. Usually, the mixture is approximately a 1:1 mixture. Typically step (iv) occurs at SATP. The step may last for between 1 and 24 hours, for example about 16 hours.

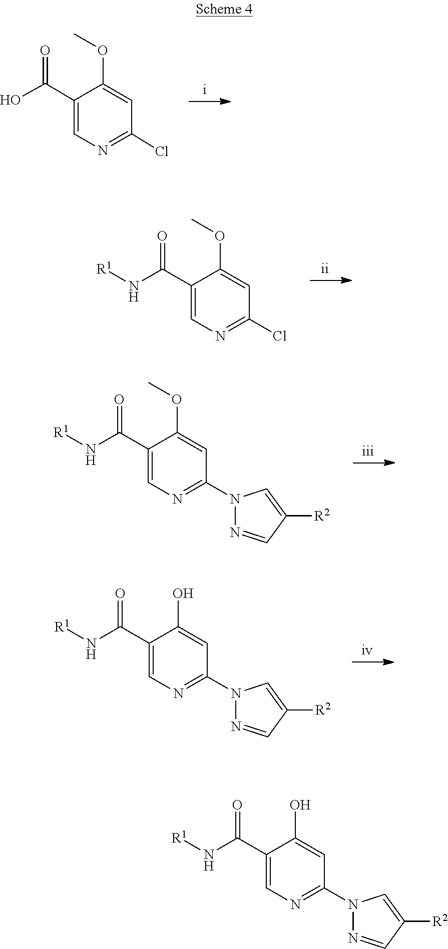

Scheme 4 above shows how the 4-hydroxypyridine/pyridinone structures of formula (Ia) may be produced using the general synthesis procedures set forth in the Examples section hereinbelow (see the General Procedures A and D in the Examples). Step (i) of Scheme 4 typically comprises treatment with R-NH₂. Scheme 4 step (i) may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent, which may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 4 may comprise treatment with further reagents such as pyrazole and substituted pyrazoles. Step (ii) of Scheme 4 comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is cesium carbonate (Cs₂CO₃). The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours.

Step (iii) of Scheme 4 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (iv) of Scheme 4, if required, may be carried out using any suitable reagents for ester hydrolysis. Typically, the reaction may be carried out in the presence of a hydroxide compound, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THE and H₂O. Usually the mixture is 1:1 mixture. Typically step (ii) occurs at SATP. The step may last for between 1 and 24 hours, for example about 16 hours.

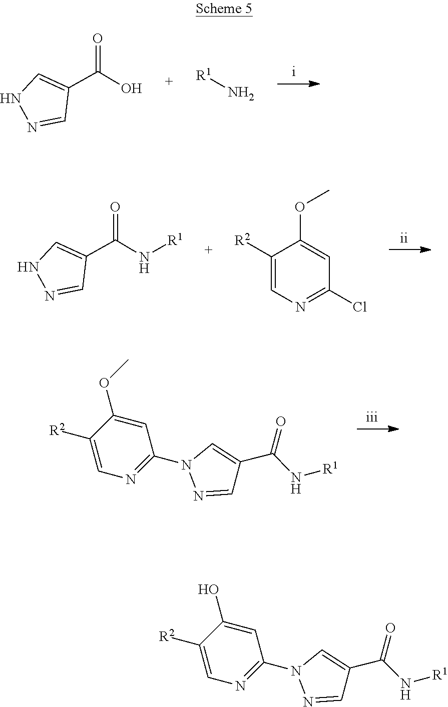

Scheme 5 above shows how the 4-hydroxypyridine/pyridinone structures of formula (Ic) may be produced using the general synthesis procedures set forth in the Examples section hereinbelow (see the General Procedures A and D in the Examples).

Step (i) of Scheme 5 may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 5 comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is cesium carbonate (Cs₂CO₃). The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours. Step (ii) may also comprise treatment with further reagents such as pyrazole and substituted pyrazoles.

Step (iii) of Scheme 5 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

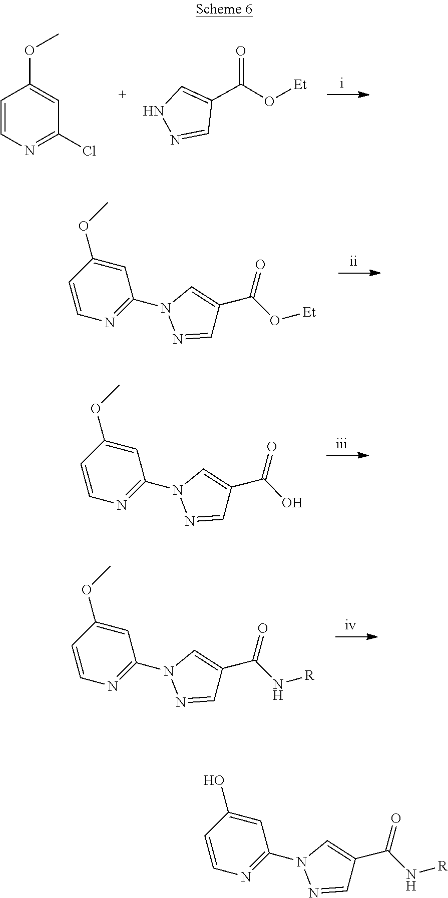

Scheme 6 above shows how substituted azines of formula (Ic) may be produced using the general synthesis procedures set forth in the Examples section hereinbelow (see the General Procedures B and D in the Examples).

Step (i) of Scheme 6 comprises treatment in the presence of a catalyst. The catalyst may be a palladium catalyst. Typically, the catalyst may be RockPhos Pd G3. The step may also take place in the presence of a base. The base may be a carbonate. Typically, the base is cesium carbonate (Cs₂CO₃). The reaction typically occurs in a solvent. The solvent may be a non-polar solvent. Typically, the solvent is a non-polar protic solvent. Usually, the solvent is tert-butanol (^{t}BuOH). Step (i) typically occurs at a temperature greater than room temperature. For example, step (i) typically occurs between 60° C. and 100° C. Usually the step occurs at around 80° C. The step may last for between 1 hour to 24 hours, for example about 16 hours.

Step (ii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THE and H₂O. Usually the mixture is 1:1 mixture. Typically step (ii) occurs at SATP. The step may last for between 1 and 24 hours, for example about 16 hours.

In Scheme 6 step (iii) the starting material is treated with a group R—NH₂. For step (iii) any appropriate peptide coupling reagents may be used. Typically, Scheme 6 step (iii) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be dimethylacetamide (DMAc). Typically step (iii) occurs at SATP. The step may last for between 1 and 24 hours, for example about 16 hours.

Step (iv) of Scheme 6 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (iv) typically occurs at a temperature greater than room temperature. For example, step (iv) typically occurs between 80° C. and 120° C. Usually the step occurs at around 100° C. The step may last for between 30 minutes and 6 hours, for example about 2 hours.

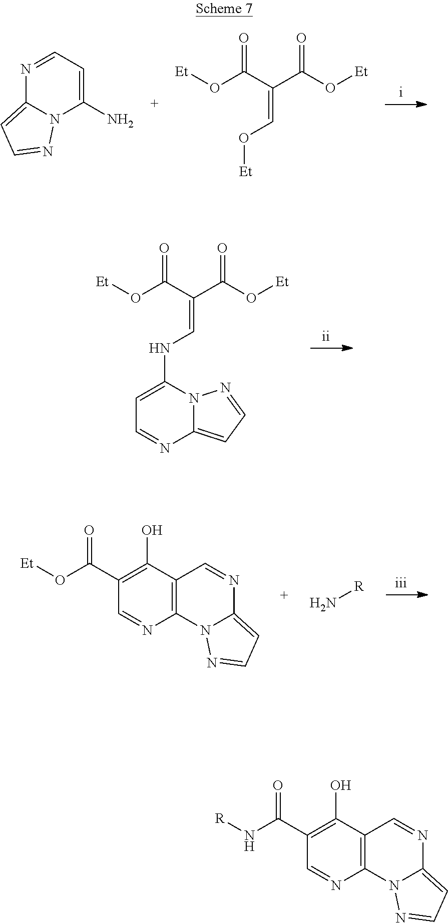

Scheme 7 above shows how the pyrazolo[1,5-a]pyrido[3,2-e]-pyrimidine-7-amido structures of formula (Id) may be produced using the general synthesis procedures set forth.

Step (i) of Scheme 7 is a Michael addition reaction that may compromise treatment with any suitable reagents known to the skilled person. In some instances, sodium ethoxide is used. This step typically takes place in the presence of a solvent. The solvent is typically a polar solvent, and more typically a polar aprotic solvent. The solvent may be EtOH. Step (i) typically occurs at a temperature greater than room temperature. Typically occurs between 80° C. and 120° C. Usually, the step occurs at around 100° C. The step may last for between 1 hour to 48 hours, for example about 2 hours.

Step (ii) of Scheme 7 is an intramolecular cyclisation that may compromise treatment with any suitable reagents known to the skilled person. This step typically takes place in the presence of a solvent. The solvent is typically one of high boiling point. The solvent may be diphenylether. Step (ii) typically occurs at a temperature greater than room temperature. Typically the temperature is between 140° C. and 250° C. Usually, the step occurs at around 250° C. The step may last for between 10 minutes to 8 hours, for example about 30 minutes.

Step (iii) of Scheme 7 is an amide coupling directly from the ethyl ester and comprises treatment in the presence of a catalyst. Typically, the catalyst may be DABCO-(AlMe₃)₂. The reaction typically occurs in a solvent. The solvent may be a mixture of solvents. Usually, the reaction occurs in tetrahydrofuran. Step (iii) typically occurs at a temperature greater than room temperature. For example, step (iii) typically occurs between 40° C. and 150° C. Usually, the step occurs at around 130° C. The step may last for between 10 minutes to 12 hours, for example about 1 hour.

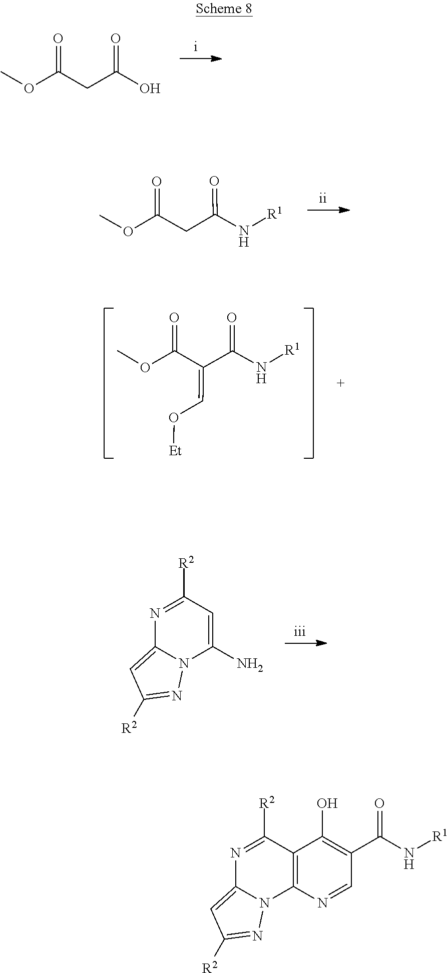

Scheme 8 above shows how the pyrazolo[1,5-a]pyrido[3,2-e]-pyrimidine-7-amido structures of formula (Id) may be produced using the general synthesis procedures set forth in the Examples section hereinbelow (see the General Procedures A and D in the Examples).

In Scheme 8 step (i) the starting material treated with a group R—NH₂. For step (i) any appropriate amide coupling reagents may be used. Typically, Scheme 4 step (i) comprises treatment with an acid anhydride, for example propanephosphonic acid anhydride in the presence of a base such as N,N-diisopropylethylamine (DIPEA). The reaction typically occurs in a solvent. The solvent may be a polar solvent. Typically, the solvent is a polar, aprotic solvent. The polar aprotic solvent may be dimethylacetamide (DMAc). Typically step (i) occurs at SATP. The step may last for between 1 and 24 hours, for example about 16 hours.

Scheme 8 step (ii) comprises treatment with ethyl orthoformate. The reaction typically occurs neat. Typically, step (ii) occurs at a temperature greater than room temperature. For example, step (ii) may occur at between 100° C. and 140° C. Usually this step occurs at around 120° C. The step may last for between 30 minutes and 6 hours, for example about 2 hours.

Step (iii) of Scheme 8 comprises heating the reagents at a temperature greater than room temperature. Typically, the reagents are heated to greater than 200° C., typically to around 240° C. This step may last for between 10 minutes and 2 hours, for example around 30 minutes. The step may also take place in the presence of further reagents, such a diphenylether.

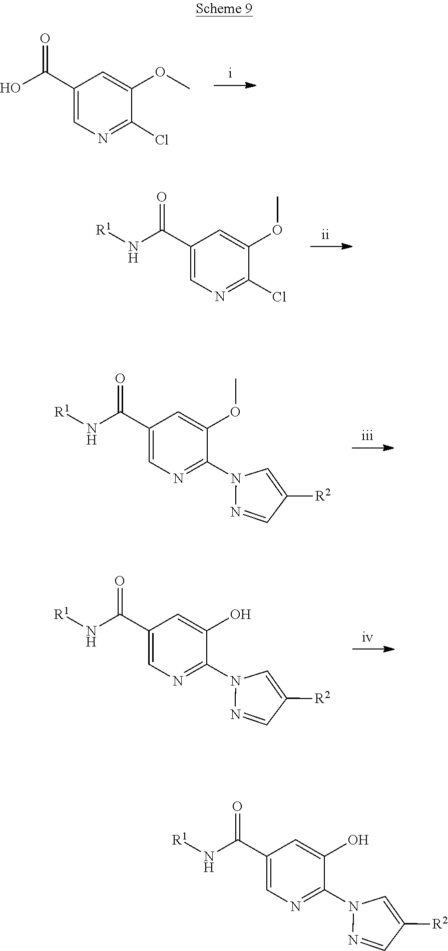

Scheme 9 above shows how the 3-hydroxypyridine structures of formula (Ib) may be produced using the general synthesis procedures set forth in the Examples section hereinbelow (see the General Procedures A and D in the Examples).
Step (i) of Scheme 9 typically comprises treatment with R-NH₂. Scheme 9 step (i) may comprise treatment with any appropriate amide coupling reagents. Typically, step (i) comprises treatment with an acid anhydride, for example propanephosphonic acid anhydride in the presence of a base such as N,N-diisopropylethylamine (DIPEA). The reaction typically occurs in a solvent, which may be a polar solvent. Typically, the solvent may be a polar, aprotic solvent. The polar aprotic solvent may be dimethylacetamide (DMAc). Typically step (i) occurs at SATP. The step may last for between 1 and 24 hours, for example about 16 hours.

Step (ii) of Scheme 9 comprise treatment with further reagents such as pyrazole and substituted pyrazoles. Step (ii) of Scheme 9 comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is cesium carbonate (Cs₂CO₃). The reaction typically occurs in a solvent. The solvent may be a mixture of solvents. Typically, the solvent may be a mixture of one or more polar solvents, often polar aprotic solvents. Usually, the reaction occurs in tert butanol or 1,4-dioxane. Step (ii) typically occurs at a temperature greater than room temperature. For example, step (ii) typically occurs between 40° C. and 80° C. Usually, the step occurs at around 60° C. The step may last for between 1 hour to 48 hours, for example about 16 hours. Step (ii) may also comprise treatment with further reagents such as pyrazole and substituted pyrazoles.

Step (iii) of Scheme 9 is a deprotection step that may comprise treatment with any suitable reagents known to the skilled person. In some instances, lithium chloride is used. This step typically takes place in the presence of a solvent, which is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (iv) of Scheme 9, if required, may be carried out using any suitable reagents for ester hydrolysis. Typically, the reaction may be carried out in the presence of a hydroxide compound, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THE and H₂O. Usually the mixture is 1:1 mixture. Typically step (ii) occurs at SATP. The step may last for between 1 and 24 hours, for example about 16 hours.

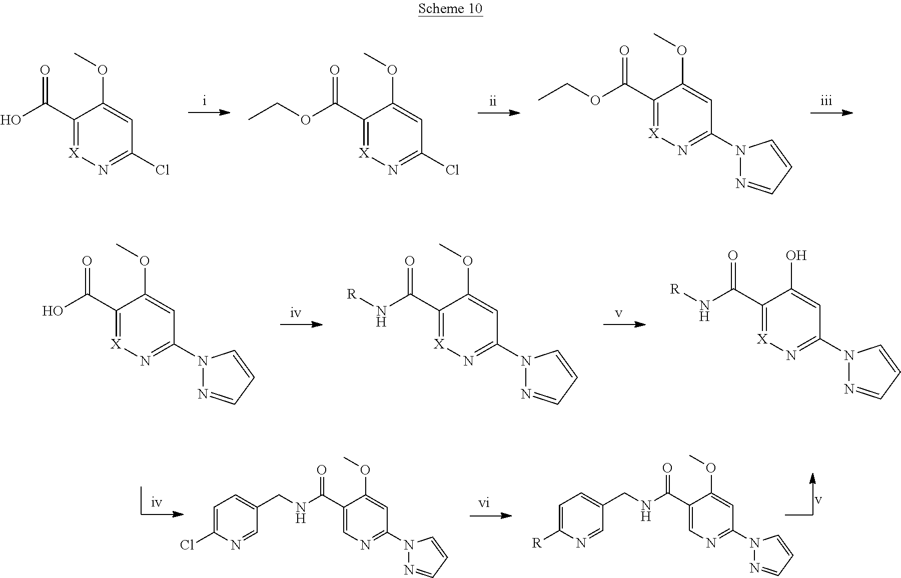

Scheme 10 above shows how the substituted azines of formula (Ia) may be produced using the general synthesis procedures set forth in the Examples section hereinbelow (see the General Procedures B, C and D in the Examples).

Step (i) of Scheme 10 may be carried out using any appropriate esterification reagents known to the skilled person. In some cases N′-ethylcarboiimide hydrochloride (EDC·HCl) is used. Typically, a catalyst is also present. Usually the catalyst present is an organocatalyst. The catalyst may be 4-dimthylaminopyridine (DMAP). A base may also be present in step (i) of Scheme 10. The base may be N,N-diisopropylethylamine (DIPEA). Typically step (i) of Scheme 10 takes place in the presence of a solvent. The solvent may be a mixture of two solvents. Typically, the solvent is a mixture of two polar solvents. Usually, the solvent is a mixture of a polar protic solvent and a polar aprotic solvent. Therefore, the solvent may be a mixture of dimethylformamide (DMF) and ethanol. Typically, step (i) occurs at SATP. This step may last for between 1 hour and 24 hours, for example around 16 hours.

In step (ii) of Scheme 10 the product of step (i) is treated with pyrazole. Step (ii) comprises treatment in the presence of a catalyst. The catalyst may be a palladium catalyst. Typically, the catalyst may be Pd^{t}BuxPhos G3. The step may also take place in the presence of a base. The base may be a carbonate. Usually, the base is cesium carbonate (Cs₂CO₃). Step (ii) typically occurs at a temperature greater than room temperature. For example, step (ii) typically occurs between 40° C. and 80° C. Usually, the step occurs at around 60° C. The step may last for between 1 hour to 24 hours, for example about 16 hours.

Step (iii) may be carried out using any suitable reagents for ester hydrolysis. Typically, the reaction may be carried out in the presence of a hydroxide, usually lithium hydroxide. The reaction is usually carried out in the presence of a solvent. The solvent may be a mixture of one or more solvents, typically one or more polar solvents. The one or more polar solvents may be protic or aprotic. For example, the solvent may be a mixture of THE and H₂O. Usually, the mixture is an approximately 1:1 mixture. Typically step (iii) occurs at SATP. The step may last for between 1 and 24 hours, for example about 16 hours.

In Scheme 10 step (iv) the product of step (iii) is treated with a group R—NH₂. For step (iv) any appropriate peptide coupling reagents may be used. Typically, Scheme 10 step (iv) comprises treatment with HATU in the presence of a base such as DIPEA. The reaction typically occurs in a solvent. The solvent may be a polar solvent. Typically, the solvent may be a polar, aprotic solvent. The polar aprotic solvent may be N,N-dimethylacetamide (DMAc). Typically step (iv) occurs at SATP. The step may last for between 1 and 24 hours, for example about 16 hours.

Step (v) of Scheme 10 is a deprotection step that may comprise treatment with any suitable reagents known to the skilled person. In some instances, lithium chloride is used. This step typically takes place in the presence of a solvent. The solvent is typically a polar solvent, and more typically a polar aprotic solvent. The solvent may be DMAc. Step (v) typically occurs at a temperature greater than room temperature. For example, step (v) typically occurs between 80° C. and 120° C. Usually, the step occurs at around 100° C. The step may last for between 30 minutes and 6 hours, for example about 2 hours.

Step (vi) of Scheme 10 comprises reaction in the presence of a catalyst. The catalyst may be a palladium catalyst. Typically, the catalyst may be PdAmPhos. The step may also take place in the presence of a base. The base may be a carbonate. Usually, the base is cesium carbonate (Cs₂CO₃). In step (vi) the product of step (iv) is usually treated with an organoborane compound comprising a group —R. Typically this compound is a compound of the formula RB(OH)₂ or R-B-pinacol ester. Step (vi) typically occurs at a temperature greater than room temperature. For example, step (vi) typically occurs between 80° C. and 120° C. Usually, the step occurs at around 100° C. The step may last for between 30 minutes to 6 hours, for example about 2 hours. This step typically takes place in the presence of a solvent. The solvent is typically a non-polar solvent, and more typically a non-polar aprotic solvent. The solvent may be 1,4-dioxane.

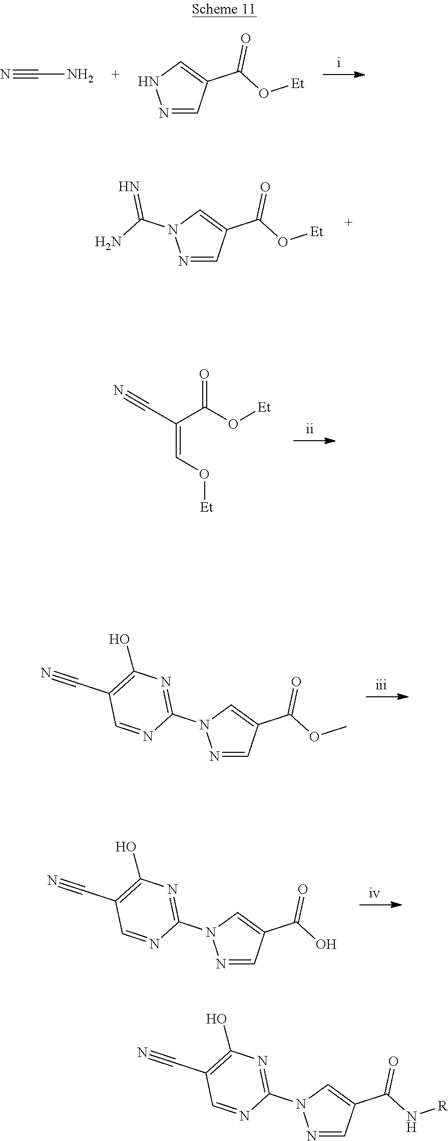

Scheme 11 above shows how the substituted pyrimidines of formula (IV) may be produced using the general synthesis procedure set forth in the Examples section hereinbelow (see the General Procedure B in the Examples).

Step (i) of Scheme 11 typically comprises treatment with an acid. Usually, the acid is a protic acid, such as HCl. For example, 4M HCl in 1,4-dioxane may be used. Step (i) typically occurs at a temperature greater than room temperature. For example, step (i) typically occurs between 80° C. and 120° C. Usually, the step occurs at around 100° C. The step may last for between 1 hour and 24 hours, for example about 16 hours.

Step (ii) of Scheme 11 typically comprises treatment in the presence of a base. Any suitable base may be used. Typically, the base is a carbonate. K₂CO₃ may be used. Step (ii) typically takes place in a solvent. The solvent may be a polar solvent, typically a polar protic solvent. Usually, the solvent is methanol. Typically step (ii) occurs at SATP. The step may last for between 1 and 24 hours, for example about 16 hours.

Step (iii) may be carried out using any suitable reagents for ester hydrolysis. Typically, the reaction may be carried out in the presence of a hydroxide base, usually lithium hydroxide. The reaction is usually carried out in the presence of a solvent. The solvent may be a mixture of one or more solvents, typically one or more polar solvents. The one or more polar solvents may both be protic. For example, the solvent may be a mixture of methanol and H₂O. Usually the mixture is 1:1 mixture. Typically step (iii) occurs at SATP. The step may last for between 1 and 24 hours, for example about 16 hours.

In Scheme 11 step (iv) the product of step (iii) is treated with a group R—NH₂. . For step (iv) any appropriate peptide coupling reagents may be used. Typically, Scheme 11 step (iv) comprises treatment with T3P in the presence of a base such as N,N-diisopropylethylamine (DIPEA). The reaction typically occurs in a solvent. The solvent may be a polar solvent. Typically, the solvent may be a polar, aprotic solvent. The polar aprotic solvent may be N,N-dimethylacetamide (DMAc). Typically, step (iv) occurs at SATP. The step may last for between 1 and 24 hours, for example about 16 hours.

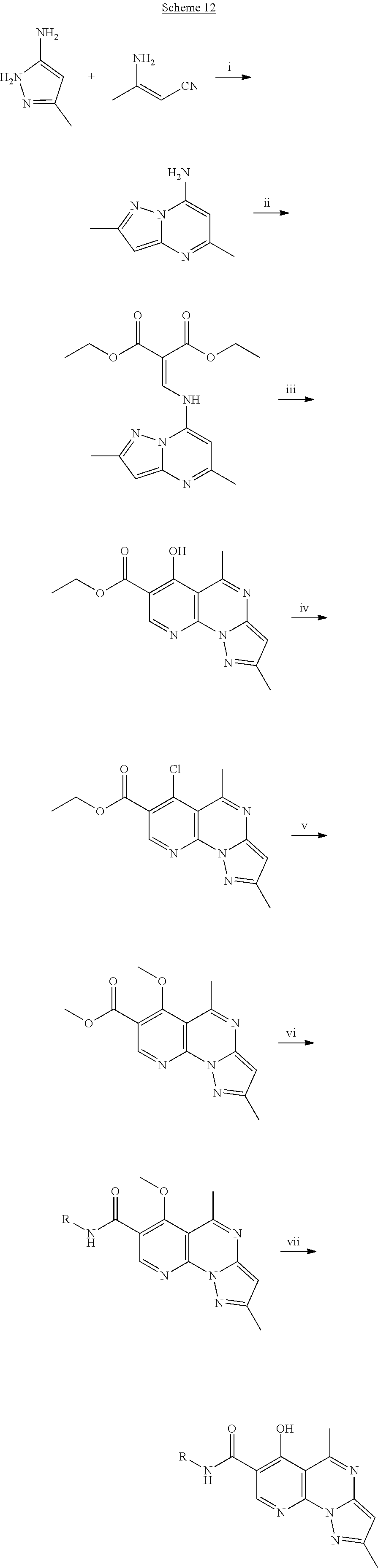

Scheme 12 above shows how the pyrazolo[1,5-a]pyrido[3,2-e]-pyrimidine-7-amido structures of formula (Id) may be produced using the general synthesis procedures set forth in the Examples section hereinbelow (see the General Procedures A and D in the Examples).

In Scheme 12, step (i) usually comprises heating together the starting materials. Typically heating occurs at a temperature of 100° C. to 200° C., more typically heating occurs at from 140° C. to 160° C. Heating may occur at about 150° C. The step may last for between 30 minutes and 6 hours, for example about 1 hour.

Step (ii) of Scheme 12 comprises heating the product of step (i) with diethyl ethoxymethylenemalonate. Step (ii) typically takes place in a solvent. The solvent may be a polar solvent or apolar protic solvent. Usually, the solvent is toluene. Typically heating occurs at a temperature of 80° C. to 160° C., more typically heating occurs at from 100° C. to 140° C. Heating may occur at about 120° C. Step (ii) typically lasts for about 24 hours to 72 hours. For example, step (ii) may last for around 48 hours.

Step (iii) of Scheme 12 typically comprises treating with a reagent to promote ring formation. Any appropriate reagent may be used. Typically, Eaton's reagent (10 wt % phosphorous pentoxide solution in methanesulfonic acid) is used. Step (iii) typically occurs at a temperature greater than room temperature. For example, step (iii) typically occurs between 50° C. and 90° C. Usually, the step occurs at around 70° C. The step may last for between 16 hours and 30 hours, for example about 24 hours.

Step (iv) of Scheme 12 typically comprises treatment with a chlorinating agent. Any appropriate chlorinating agent known to the skilled person may be used. Usually phosphoryl chloride (POCl₃) is used. Step (iv) typically occurs at a temperature greater than room temperature. For example, step (iv) typically occurs between 50° C. and 90° C. Usually, the step occurs at around 70° C. The step may last for between 1 hour and 6 hours, for example about 3 hours.

Step (v) of Scheme 12 typically comprises treatment with sodium methoxide (NaOMe). Step (v) typically takes place in a solvent. The solvent may be a polar solvent, typically a polar protic solvent. Usually, the solvent is methanol. Typically step (v) occurs at SATP. The step may last for between 1 and 10 hours, for example about 4 hours.

Step (vi) of Scheme 12 typically comprises treatment with R-NH₂ in the presence of an agent to promote amide formation. The agent may be an organoaluminium reagent, typically Bis(trimethylaluminum)-1,4-diazabicyclo[2.2.2]octane adduct (DABAL-AlMe₃). Typically step (vi) takes place in the presence of a solvent. The solvent is usually a non-polar solvent, and often a non-polar aprotic solvent such as THF. Step (vi) typically occurs at a temperature greater than room temperature. For example, step (vi) typically occurs between 100° C. and 140° C. Usually, the step occurs at around 120° C. The step may last for between 30 minutes and 6 hours, for example about 3 hours.

Scheme 12 step (vii) can comprise treatment with any agent appropriate for converting an ether to a hydroxyl. Typically, lithium chloride can be used. The reaction typically occurs in a solvent. The solvent may be a polar solvent. Typically, the solvent may be a polar, aprotic solvent. The polar aprotic solvent may be N,N-dimethylacetamide (DMAc). Step (vii) typically occurs at a temperature greater than room temperature. For example, step (vii) typically occurs between 80° C. and 120° C. Usually, the step occurs at around 100° C. The step may last for between 30 minutes and 6 hours, for example about 2 hours.

Compounds of the invention containing one or more chiral centres may be used in enantiomerically or diastereoisomerically pure form, or in the form of a mixture of isomers. For the avoidance of doubt, the compounds of the invention can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. A substituted azine of formula (I), (Ia), (Ib), (Ic) or (Id), or a substituted pyrimidine of formula (IV), may be converted into a pharmaceutically acceptable salt, and salts may be converted into the free compound, by conventional methods.

### Pharmaceutical Compositions

Also provided by the invention is a pharmaceutical composition comprising a compound of the invention as defined anywhere herein, and a pharmaceutically acceptable carrier or diluent.

Typically, the composition contains up to 85 wt % of a compound of the invention. More typically, it contains up to 50 wt % of a compound of the invention. Preferred pharmaceutical compositions are sterile and pyrogen free. Further, when the pharmaceutical compositions provided by the invention contain a compound of the invention which is optically active, the compound of the invention is typically a substantially pure optical isomer.

The composition of the invention may be provided as a kit comprising instructions to enable the kit to be used as described herein or details regarding which subjects the composition may be used for.

The composition of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The composition of the invention may be formulated for inhaled (aerosolised) administration as a solution or suspension. The compound or combination of the invention may be administered by a metered dose inhaler (MDI) or a nebulizer such as an electronic or jet nebulizer. Alternatively, the compound or combination of the invention may be formulated for inhaled administration as a powdered drug, such formulations may be administered from a dry powder inhaler (DPI). When formulated for inhaled administration, the compound or combination of the invention may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the compound or combination of the invention is delivered as a nebulized aerosol, the reference to particle diameters defines the MMAD of the droplets of the aerosol. The MMAD can be measured by any suitable technique such as laser diffraction.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

The composition of the invention may further comprise one or more additional active agents. The additional active agents may be selected from ACE inhibitors, angiotensin II receptor agonists, beta receptor blockers, calcium antagonists, PDE inhibitors, mineralocorticoid receptor antagonists, diuretics, aspirin, iron supplements, vitamin B12 and folic acid supplements, statins, digitalis (digoxin) derivatives, tumor chemotherapeutics and antibiotics.

### Therapeutic Uses

The compounds of the invention have been shown have high efficacy and specificity as hypoxia inducible factor (HIF) prolyl hydroxylase (PHD) inhibitors. For instance, some compounds of the invention have been shown to have IC₅₀ for PHD2 of less than 200 nM, which is a significant improvement compared to known inhibitors (e.g. roxadustat has an IC₅₀ of 2.7 μM the LCMS PHD2 hydroxylation assay used herein). As well as their potency, compounds of the invention have been found to be highly selective for the PHDs, with greater than 100-fold selectivity compared to other tested 2OG oxygenases.

As well as these desirable biochemical properties, compounds of the invention have been shown to have desirable physical properties including good solubility and permeability in cells. These physical properties mean that compounds of the invention of have been found to efficiently stabilise cellular HIF-1α. Additionally, low in vivo doses of the compounds have been shown to induce erythropoiesis in animal models. The compounds and pharmaceutical compositions of the invention therefore have potential utility in treating conditions for which the PHDs are a therapeutic target.

Accordingly, the invention provides a compound of the invention as defined herein, or a pharmaceutical composition of the invention as defined herein, for use in the treatment of the human or animal body by therapy. As discussed hereinbefore, the terms “treatment”, “treat” and “treating” herein refer to both therapeutic treatment and prophylactic or preventative measures.

The invention also provides a compound of the invention as defined herein, or a pharmaceutical composition of the invention as defined herein, for use as a modulator of hypoxia inducible factor prolyl hydroxylase activity. Typically, the compound or pharmaceutical composition is for use as an inhibitor of hypoxia inducible factor prolyl hydroxylase activity. Thus, the invention also provides a compound of the invention as defined herein, or a pharmaceutical composition of the invention as defined herein, for use as a PHD inhibitor.

The invention also provides a compound of the invention as defined herein, or a pharmaceutical composition of the invention as defined herein, for use in treating a PHD-related disorder.

The term, “PHD-related disorder”, as used herein, means a disorder that can be treated by modulating hypoxia inducible factor prolyl hydroxylase activity. Typically, the PHD-related disorder is one that can be treated by inhibiting hypoxia inducible factor prolyl hydroxylase activity. The skilled person can readily identify PHD-related disorders experimentally.

PHD-related disorders include, but are not limited to, anaemia, ischemia, inflammation, Parkinson's disease, sickle cell anaemia (including via upregulation of fetal haemoglobin F), Alzheimer's disease, non-fatty liver disease, irritable bowel disease, cardiovascular disease, cardiac insufficiency, chronic kidney disease, cancer and renal insufficiency. Thus, a compound or pharmaceutical composition of the invention may be for use in treating any of the aforementioned conditions.

Similarly, a compound or pharmaceutical composition of the invention may be for use in skeletal muscle injury repair, increasing red blood cell count (RBC), increasing haemoglobin (HGB) production, increasing haematocrit (HCT) production, increasing Erythropoietin (EPO) production, wound healing, angiogenesis, revascularisation, stem cell activation or cardioprotection after myocardial infarction. Thus the invention provides a compound of the invention as defined herein, or a pharmaceutical composition of the invention as defined herein, for use in treating anaemia, ischemia, inflammation, Parkinson's disease, Alzheimer's disease, non-fatty liver disease, irritable bowel disease, sickle cell anaemia, cancer, cardiovascular disease, cardiac insufficiency, chronic kidney disease, or renal insufficiency; or for use in skeletal muscle injury repair, in increasing red blood cell count (RBC), increasing haemoglobin (HGB) production, increasing haematocrit (HCT) production, increasing Erythropoietin (EPO) production, wound healing, angiogenesis, revascularisation, stem cell activation, or cardioprotection after myocardial infarction. The sickle cell anaemia may be sickle cell anaemia via upregulation of fetal haemoglobin F.

Often, the compound or pharmaceutical composition of the invention is for use in treating anaemia. The anaemia may be renal anaemia, for instance anaemia associated with chronic kidney disease, or anaemia in a dialysis patient, anaemia induced by chemotherapy, cancer-associated anaemia, age-related anaemia or anaemia occurring as a result of cancer such as leukaemia, multiple myeloma and smouldering myeloma. The anaemia may be sickle cell anaemia, for instance sickle cell anaemia via upregulation of fetal haemoglobin F.

Often, the compound or pharmaceutical composition of the invention is for use in in increasing red blood cell count (RBC), increasing haemoglobin (HGB) production, increasing haematocrit (HCT) production, or increasing Erythropoietin (EPO) production.

Also, typically, the compound or pharmaceutical composition of the invention is for use in treating ischemia. Thus, ischemia-related diseases may be treated by the compound or pharmaceutical composition of the invention. The compound or pharmaceutical composition of the invention may therefore be for use in treating ischemia in a circulatory or cardiovascular disease, myocardial infarction, ischemia during a surgical operation, organ ischemia, an ischaemic disease or diabetic limb ischemia or sickle cell anaemia. The compound or pharmaceutical composition of the invention may be for use in providing cardioprotection after myocardial infarction.
The invention also provides a method for treating a subject suffering from or susceptible to a PHD-related disorder, which method comprises administering to said subject an effective amount of a compound of the invention as defined herein, or the pharmaceutical composition of the invention. The PHD-related disorder may for instance be anaemia, ischemia, sickle cell anaemia, cancer, inflammation, Parkinson's disease, Alzheimer's disease, non-fatty liver disease, irritable bowel disease, cardiovascular disease, cardiac insufficiency, chronic kidney disease or renal insufficiency. The disorder may for instance be a HIF-related disorder, an EPO-related disorder or a VHL-related disorder, for instance Von Hippel-Lindau (VHL) syndrome.
The invention also provides a method of repairing skeletal muscle injury, increasing red blood cell count (RBC), increasing haemoglobin (HGB) production, increasing haematocrit (HCT) production, increasing Erythropoietin (EPO) production, wound healing, angiogenesis, revascularisation, stem cell activation, or cardioprotection after myocardial infarction, in a subject, which method comprises administering to said subject an effective amount of a compound of the invention as defined herein, or the pharmaceutical composition of the invention.

The subject is generally a mammal, and typically a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters.

Diseases such as anaemia, ischemia, and inflammation, which may be related to a hypoxic state, have been shown to be closely linked to deficiencies in erythropoietin (EPO). Previous studies have shown that by activating the HIF pathway the effects of EPO can be improved. As described earlier, PHDs are oxygenases which catalyse hydroxylation of specific prolyl residues within the oxygen degradation domains of hypoxia inducing factor alpha (HIF-a) sub-units. Therefore, compounds and compositions of the invention which are effective PHD inhibitors can be useful in the treatment of HIF pathway related diseases, and EPO related diseases such as anaemia, ischemia and inflammation.

Increasing EPO production can also lead to an increasing red blood cell count (RBC), and increasing haemoglobin (HGB) production. Increasing the red blood cell count can, in turn, facilitate wound healing, angiogenesis, revascularisation or stem cell activation.

Parkinson's disease and Alzheimer's disease have both been linked to neuronal hypoxia. In particular, HIF stabilisation in patients with Parkinson's disease has been suggested to increase dopamine synthesis and dopaminergic neutron growth. For patients with Alzheimer's disease, it is known that hypoxia can stimulate amyloid p peptide generation, which can disrupt membrane localisation of glucose transporters (GLUT), affecting the level of glucose in the brain. Stabilising HIF can upregulate neuronal glucose transporters such as GLUT-1 and GLUT-3, which may mitigate this effect.

The compound or pharmaceutical composition of the invention is thus typically for use in treating anaemia.

Anaemia as mentioned herein may be any form of anaemia. For example, anaemia includes anaemias related to impaired production of blood cells, such as iron deficiency anaemia, vitamin deficiency anaemia, anaemia of inflammation, aplastic anaemia (such as pure red cell aplasia, and fanconi anaemia), anaemia associated with bone marrow disease, haemolytic anaemia, sickle cell anaemia, thalassaemia, renal anaemia (such anaemia associated with chronic kidney disease, or anaemia in a dialysis patient), anaemia of endocrine disease, megaloblastic anaemia (such as pernicious anaemia, and anaemia of folate deficiency), anaemia of prematurity, congenital dyserthropoietic anaemia, and myelophthisic anaemia, myelodysplastic syndrome.

Anaemia may also be anaemia associated with increased destruction of red blood cells (i.e. haemolytic anaemia). Haemolytic anaemias may be caused by intrinsic abnormalities (such as hereditary spherocytosis, hereditary elliptocytosis, abetalipoproteinaemia, enzyme deficiencies, and sickle cell anaemia), by extrinsic abnormalities (such as antibody mediated anaemias including rhesus disease and transfusion reaction, or mechanical trauma to red blood cells including heart surgery, haemodialysis, and infection), or by parasites such as trypanosoma congolense.

Anaemia may also be anaemia associated with blood loss, such as anaemia of prematurity, trauma or surgery, gastrointestinal tract lesions, gynaecologic disturbances, menstruation, and iatrogenic anaemia.

Further causes of anaemia may include fluid overload and intestinal inflammation (caused, for example, by infection with Helicobacter pylori, gluten-related disorders such as coeliac disease, or by inflammatory bowel disease).

Typically, the anaemia is renal anaemia (for instance anaemia associated with chronic kidney disease, or anaemia in a dialysis patient), anaemia induced by chemotherapy, sickle cell anaemia, cancer associated anaemia, age-related anaemia, or anaemia occurring as a result of cancer such as leukaemia, multiple myeloma and smouldering myeloma.

Often the anaemia may be renal anaemia, for instance anaemia associated with chronic kidney disease or anaemia in a dialysis patient.

A compound or composition of the invention may also be for use in treating ischemia.

Herein ischemia may refer to any type of ischemia. Ischemia includes cardiac or circulatory ischemia (such as ischemia in a circulatory or cardiovascular disease, myocardial infarction, coronary ischemia, coronary artery disease, myocardial ischemia, and ischaemic heart disease).

Ischemia also includes organ ischemia, such as bowel ischemia (such as intestinal ischemia, including ischaemic colitis, and mesenteric ischemia); brain ischemia (including acute ischemia such as ischaemic stroke and transient ischaemic attack, and chronic ischemia which may lead to vascular dementia); and kidney ischemia.

Ischemia may also be limb ischemia (such as acute limb ischemia, chronic limb threatening ischemia and diabetic limb ischemia).

Ischemia may also be related to ischaemic disease. Ischemia may be ischemia during a surgical operation. Ischemia may also be cutaneous ischemia (such as cyanosis and gangrene).

Typically, ischemia is ischemia in a circulatory or cardiovascular disease, myocardial infarction, ischemia during a surgical operation, organ ischemia, an ischaemic disease or diabetic limb ischemia.

A therapeutically effective amount of the compound of the invention is administered to a subject, the term “therapeutically effective amount” as used herein meaning a therapeutically or prophylactically effective amount. Similarly, a composition comprising a therapeutically effective amount of the compound of the invention may be administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 1 mg to 2 g.

The invention will be further described in the Examples and Reference Examples which follow:

### EXAMPLES

### Synthetic Examples

### General Procedures

All reactions involving moisture-sensitive reagents were carried out under a nitrogen atmosphere using standard vacuum line techniques. Glassware was oven dried and cooled under nitrogen before use. Commercial anhydrous solvents used in reactions and HPLC grade solvents were employed for work-up and chromatography. Aqueous solutions were made using de-ionized water, purified using an Elix UV-10 system. Thin layer chromatography (TLC) was carried out using Merck (Darmstadt, Germany) silica gel 60 F254 TLC plates. TLC visualization was carried out under UV light and stained with one of three stains; ninhydrin, potassium permanganate, or anisaldehyde. Chromatography was carried out using a Biotage (Uppsala, Sweden) Isolera One or Biotage SP4 flash purification system, using Biotage pre-packed SNAP columns. Reactions were monitored using an Agilent (Cheshire, UK) 1200 series, 6120 quadrupole LC-MS system using a Merck Chromolith® Performance RP-18 HPLC column. Deuterated solvents were from Sigma-Aldrich, and H NMR spectra were obtained using Bruker AVANCE AVIII HD 400 nanobay (400 MHz) machine or a machine Bruker AV500 (500M Hz) with a ¹³C cryoprobe. All signals are described in 6 ppm with multiplets being denoted as singlet, doublet, triplet, quartet, and multiplet using the abbreviations s, d, t, q, and m, respectively. Chemical shifts in presented NMR spectra were referenced using residual solvent peaks with coupling constants, J, reported in hertz (Hz) to an accuracy of 0.5 Hz. For high-resolution mass spectrometry (HR-MS), a Bruker MicroTOF instrument with an ESI source and Time of Flight (TOF) analyzer was used. MS data are represented as a ratio of mass to charge (m/z) in Daltons. A Bruker Tensor 27 instrument was used to obtain Fourier transform infrared spectra (FT-IR). Spectroscopic grade solvents and a Perkin Elmer 241 Polarimeter were used to obtain optical rotations.

All chemicals, reagents, and solvents were obtained from Sigma-Aldrich (Dorset, UK) and used without further purification. HPLC grade solvents were used for reactions, chromatography, and work-ups.

### General Procedure A

Ethyl ester amide coupling: the relevant ethyl ester (1 equiv), the relevant amine (1 equiv) and DABACO-(AlMe₃)₂ (1.0 equiv) were added; the microwave vial was flushed with N₂ which was removed in vacuo (3 times) before the addition of anhydrous THF. The reaction mixture was then heated at 130° C. for 8 minutes with biotage microwave irradiation (unless stated differently). The reaction mixture was diluted with a mixture of CH₃C₁: IPA(3:1, 20 ml), followed by the addition of KNaC₄H₄O₆·4H₂O N (50 ml). The resultant mixture was stirred for 1 hr. The phases were then separated, the organic phase was washed with water, brine and dried over Na₂SO₄. The solvent was removed in vacuo. The crude compound was purified by flash column chromatography using (conditions stated per reaction) over 20 column volumes to give the desired compound.

### General Procedure B:

Amide coupling: The carboxylic acid (1 equiv) and DIPEA (2.5 equiv) were dissolved in DMF. T3P (1.5 equiv, 50% in DMF) or HATU (2 equiv) were then added. The resultant reaction mixture was stirred at room temperature for 30 mins before the addition of the amine (1.2 equiv). The resultant mixture was stirred overnight at room temperature. EtOAc (20 ml) and H₂O (100 ml) was added to the reaction mixture. The organic and aqueous fractions were separated. The aqueous layer was extracted with EtOAc (30 ml) twice more. The organic fractions were combined before washing with brine and drying with anhydrous Na₂SO₄. The crude compound was then purified using flash column chromatography over 20 column volumes to give the desired compound.

### General Procedure C:

Pd-catalysed amination: The aryl halide (1 equiv), amine (1.2 equiv), Cs₂CO₃ (2 equiv), Pd-ligand conjugate (0.1 equiv) were put under an anhydrous N₂ atmosphere before the addition of tert-butanol. The resultant mixture was heated at 80° C. for 16 hr. The reaction mixture was then allowed to cool to room temperature. EtOAc (20 ml) and H₂O (100 ml) was added to the reaction mixture. The organic and aqueous fractions were separated. The aqueous layer was extracted with EtOAc (30 ml) twice more. The organic fractions were combined, then washed with brine and dried using anhydrous Na₂SO₄. The crude compound was then purified using flash column chromatography using (0-100% in EtOAc in cyclohexane) over 20 column volumes to give the desired compound.

### General Procedure D:

C-4 Methoxy Demethylation: The methoxy starting material (1 equiv) was dissolved in DMAc; LiCl·H₂O (10 equivs) was then added. The resultant mixture was heated with microwave irradiation at 120° C. for 2 hrs (unless stated). The resultant mixture was diluted with water (100 ml) and extracted with EtOAc (3×20 ml). The organic phases were combined and washed with water, brine and dried with anhydrous Na₂SO₄. The volatiles were then evaporated in vacuo and purified by flash column chromatography using (100%-95% CH₂Cl₂, 0%-20% MeOH) over 15 column volumes (unless otherwise stated) to give the desired compound.

### Reference Example 1— Synthesis of N-([1,1′-Biphenyl]-4-ylmethyl)-4-chloro-2-methoxybenzamide (14)

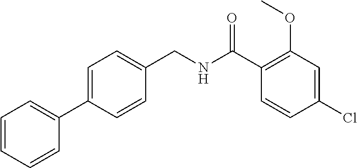 [SMILES: COc1cc(Cl)ccc1C(=O)NCc1ccc(-c2ccccc2)cc1]

Following general procedure B: 4-Chloro-2-methoxybenzoic acid (250 mg, 1.34 mmol), 4-phenylbenzylamine (294 mg, 1.6 mmol), T3P (1.06 g, 3.36 mmol), DIPEA (412 mg, 3.36 mmol) gave 14 (451 mg, 1.24 mmol, 95%).

¹H NMR (400 MHz, Chloroform-d) δ 8.21 (d, J=8.5 Hz, 1H), 8.12 (t, J=6.0 Hz, 1H), 7.60-7.56 (m, 4H), 7.46-7.41 (m, 4H), 7.37-7.30 (m, 1H), 7.08 (dd, J=8.5, 2.0 Hz, 1H), 6.97 (d, J=2.0 Hz, 1H), 4.71 (d, J=6.0 Hz, 2H), 3.93 (s, 3H).

HRMS (ESI-TOF) calcd for C₂₁H₁₉O₂N³⁵Cl [M+H]⁺: 352.1098, found: 352.1098.

### Reference Example 2 Synthesis of N-([1,1′-biphenyl]-4-ylmethyl)-2-methoxy-4-(1H-pyrazol-1-yl)benzamide (15)

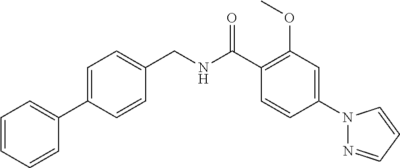 [SMILES: COc1cc(-n2cccn2)ccc1C(=O)NCc1ccc(-c2ccccc2)cc1]

Following general procedure C: 14 (100 mg, 0.284 mmol), Pd^{t}BuXPhos G3 (20 mg, 0.028 mmol), Cs₂CO₃ (185 mg, 0.568 mmol), pyrazole (23 mg, 0.34 mmol) gave 15 (28 mg, 0.073 mmol, 26%).

¹H NMR (400 MHz, DMSO-d₆) δ 8.77 (t, J=6.0 Hz, 1H), 8.66 (d, J=2.5 Hz, 1H), 7.80 (d, J=1.5 Hz, 1H), 7.69-7.32 (m, 12H), 6.60 (dd, J=2.5, 1.5 Hz, 1H), 4.56 (d, J=6.0 Hz, 2H), 4.01 (s, 3H)

HRMS (ESI-TOF) calcd for C₂₄H₂₂O₂N₃ [M+H]⁺: 384.1704, found: 384.1704.

### Reference Example 3 Synthesis of N-([1,1′-biphenyl]-4-ylmethyl)-2-hydroxy-4-(1H-pyrazol-1-yl)benzamide (16)

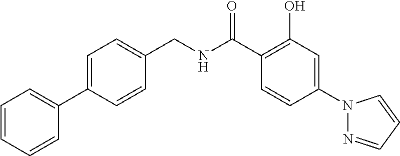 [SMILES: O=C(NCc1ccc(-c2ccccc2)cc1)c1ccc(-n2cccn2)cc1O]

TMS-I (39 mg, 0.195 mmol) was added to a solution of 15 (25 mg, 0.0652 mmol) and CH₂Cl₂ (2.5 ml). The resultant mixture was refluxed at 90° C. for 8 hrs, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 16 (7.5 mg, 0.020 mmol, 31%).

¹H NMR (400 MHz, DMSO-d₆) δ 12.96 (s, 1H), 9.43 (t, J=6.0 Hz, 1H), 8.61 (dd, J=2.5, 1.0 Hz, 1H), 8.05 (d, J=8.0 Hz, 1H), 7.79 (d, J=1.5 Hz, 1H), 7.70-7.31 (m, 11H), 6.58 (dd, J=2.5, 1.5 Hz, 1H), 4.57 (d, J=6.0 Hz, 2H).

HRMS (ESI-TOF) calcd for C₂₃H₂₀O₂N₃ [M+H]⁺: 370.1548, found: 370.1548.

### Reference Example 4 Synthesis of N-([1,1′-biphenyl]-4-ylmethyl)-2-chloropyrimidine-5-carboxamide (17)

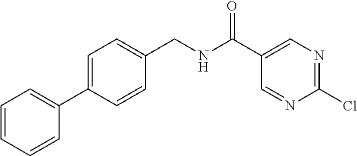 [SMILES: O=C(NCc1ccc(-c2ccccc2)cc1)c1cnc(Cl)nc1]

Following general procedure B: 2-chloro-5-carboxypyrimidine (300 mg, 1.89 mmol), 4-phenylbenzylamine (370 mg, 2.26 mmol), T3P (819 mg, 2.83 mmol) and DIPEA (365 mg, 2.83 mmol) gave 17 (285 mg, 0.88 mmol, 47%).

¹H NMR (400 MHz, DMSO-d₆) δ 9.46 (t, J=6.0 Hz, 1H), 9.18 (s, 2H), 7.87-6.86 (m, 9H), 4.56 (d, J=6.0 Hz, 2H).

HRMS (ESI-TOF) calcd for C₁₈H₁₅³⁵ClN₃O [M+H]⁺: 324.0898, found: 324.0899.

### Reference Example 5 Synthesis of 2-Chloro-N-(4-phenoxybenzyl)pyrimidine-5-carboxamide (18)

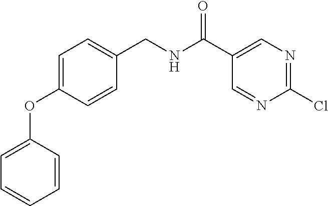 [SMILES: O=C(NCc1ccc(Oc2ccccc2)cc1)c1cnc(Cl)nc1]

Following general procedure B: 2-chloro-5-carboxypyrimidine (300 mg, 1.89 mmol), 4-phenoxybenzyamine (410 l, 2.26 mmol) and T3P (1.5 g, 4.72 mmol) gave 18 (336 mg, 0.99 mmol, 52%).

¹H NMR (400 MHz, DMSO-d₆) δ 9.41 (t, J=6.0 Hz, 1H), 9.16 (s, 2H), 7.53-7.27 (m, 4H), 7.19-6.78 (m, 5H), 4.50 (d, J=6.0 Hz, 2H).

HRMS (ESI-TOF) calcd for C₁₈H₁₄³⁵ClN₃O₂[M+H]⁺: 340.0847, found: 340.1327.

### Reference Example 6 Synthesis of N-([1,1′-Biphenyl]-4-ylmethyl)-6-chloronicotinamide (19)

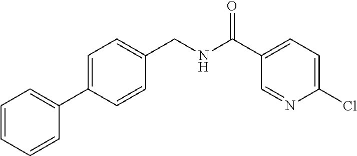 [SMILES: O=C(NCc1ccc(-c2ccccc2)cc1)c1ccc(Cl)nc1]

Following general procedure A: 2-chloroethylnicotinate (83 l, 0.53 mmol), 4-phenyl-benzylamine (97 mg, 0.53 mmol) and DABCO-(AlMe₃)₂ (108 mg, 0.424 mmol) gave 19 (154 mg, 0.48 mmol, 91%).

¹H NMR (400 MHz, DMSO-d₆) δ 9.34 (t, J=6.0 Hz, 1H), 8.90 (d, J=2.5 Hz, 1H), 8.30 (dd, J=8.5, 2.5 Hz, 1H), 7.87-7.19 (m, 10H), 4.54 (d, J=6.0 Hz, 2H).

HRMS (ESI-TOF) calcd for C₁₉H₁₅C₁N₂O [M+H]⁺: 322.0873, found: 322.0821.

### Reference Example 7 Synthesis of N-([1,1′-Biphenyl]-4-ylmethyl)-2-(1H-pyrazol-1-yl)pyrimidine-5-carboxamide (20)

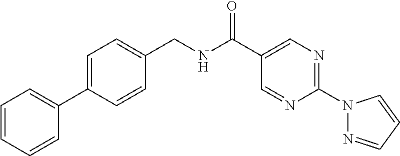 [SMILES: O=C(NCc1ccc(-c2ccccc2)cc1)c1cnc(-n2cccn2)nc1]

Following general procedure C: 17 (57 mg, 0.176 mmol), Pd^{t}BuXPhos G3 (15 mg, 0.0176 mmol), Cs₂CO₃ (201 mg, 0.619 mmol), pyrazole (42 mg, 0.619 mmol) gave 20 (10 mg, 0.0281 mmol, 16%).

¹H NMR (400 MHz, DMSO-d₆) δ 9.44 (t, J=6.0 Hz, 1H), 9.27 (s, 2H), 8.73 (d, J=3.0 Hz, 1H), 7.93 (d, J=1.5 Hz, 1H), 7.74-7.60 (m, 4H), 7.51-7.40 (m, 4H), 7.39-7.31 (m, 1H), 6.65 (dd, J=3.0, 1.5 Hz, 1H), 4.58 (d, J=5.8 Hz, 2H).

HRMS (ESI-TOF) calcd for C₂₁H₁₈ON₅ [M+H]⁺: 356.1505, found: 356.1504.

### Example 8 Synthesis of N-(4-Phenoxybenzyl)-2-(1H-pyrazol-1-yl)pyrimidine-5-carboxamide (21)

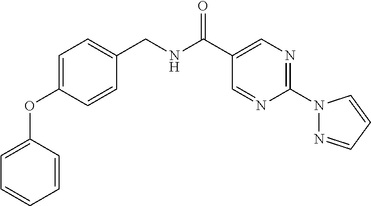 [SMILES: O=C(NCc1ccc(Oc2ccccc2)cc1)c1cnc(-n2cccn2)nc1]

Following general procedure C: 18 (100 mg, 0.294 mmol), pyrazole (40 mg, 0589 mmol), Pd^{t}BuXPhos G3 (23 mg, 0.0294 mmol), Cs₂CO₃ (238 mg, 0.735 mmol) gave 21 (64 mg, 0.172 mmol, 58%).

¹H NMR (400 MHz, DMSO-d₆) δ 8.96 (d, J=3.0 Hz, 1H), 8.68 (d, J=3.0 Hz, 1H), 8.51-8.37 (m, 1H), 8.03-7.98 (m, 1H), 7.48-6.90 (m, 10H), 6.62-6.61 (m, 1H), 4.51 (d, J=6.0 Hz, 2H).

HRMS (ESI-TOF) calcd for C₂₁H₁₇N₅O₂ [M−H]⁻: 370.1382, found: 370.1309.

### Example 9 Synthesis of N-([1,1′-Biphenyl]-4-ylmethyl)-6-(1H-pyrazol-1-yl)nicotinamide (22)

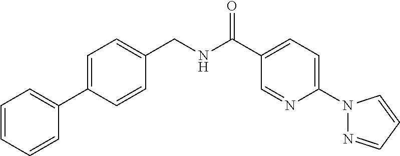 [SMILES: O=C(NCc1ccc(-c2ccccc2)cc1)c1ccc(-n2cccn2)nc1]

Following procedure C: 19 (100 mg, 0.31 mmol), pyrazole (40 mg, 0589 mmol), Pd^{t}BuXPhos G3 (25 mg, 0.031 mmol), Cs₂CO₃ (238 mg, 0.735 mmol) gave 22 (53 mg, 0.028 mmol, 48%).

¹H NMR (400 MHz, DMSO-d₆) δ 9.32 (t, J=6.0 Hz, 1H), 8.97 (d, J=2.0 Hz, 1H), 8.69 (d, J=2.5 Hz, 1H), 8.47 (dd, J=9.0, 2.0 Hz, 1H), 8.30 (dd, J=9.0, 2.0 Hz, 1H), 7.90 (d, J=1.5 Hz, 1H), 7.74-7.22 (m, 9H), 6.63 (dd, J=2.5, 1.5 Hz, 1H), 4.57 (d, J=6.0 Hz, 2H).

HRMS (ESI-TOF) calcd for C₂₂H₁₉0N₄ [M+H]⁺: 355.1553, found: 355.1551.

### Reference Example 10 Synthesis of N-Benzyl-6-chloronicotinamide (28)

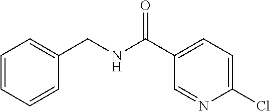 [SMILES: O=C(NCc1ccccc1)c1ccc(Cl)nc1]

Following general procedure A: 2-chloro-ethylnicotinate (200 mg, 1.081 mmol), benzylamine (116 mg, 1.08 mmol) and DABACO-AlMe₃ (221 mg, 0.864 mmol) gave 28 (220 mg, 0.897 mmol, 83%).

Solvent system used for purification: 0%-100% EtOAc in cyclohexane.

¹H NMR (400 MHz, DMSO-d₆) δ 9.32 (t, J=6.0 Hz, 1H), 8.89 (dd, J=2.5, 1.0 Hz, 1H), 8.29 (dd, J=8.5, 2.5 Hz, 1H), 7.87-7.57 (m, 1H), 7.50-6.86 (m, 5H), 4.51 (d, J=6.0 Hz, 2H).

HRMS (ESI-TOF) calcd for C₁₃H₁₀ON₂³⁵Cl [M−H]: 245.0487, found: 245.0482.

### Example 11 Synthesis of Ethyl 1-(5-(([1,1′-Biphenyl]-4-ylmethyl)carbamoyl)pyridin-2-yl)-1H-pyrazole-4-carboxylate (29)

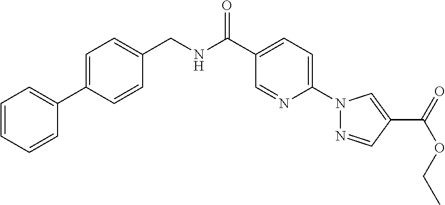 [SMILES: CCOC(=O)c1cnn(-c2ccc(C(=O)NCc3ccc(-c4ccccc4)cc3)cn2)c1]

Following general procedure C: 19 (125 mg, 0.388 mmol), pyrazole-4-carboxylate ethyl ester (65 mg, 0.465 mmol), PdtBuxPhos G3 (31 mg, 0.0388 mmol), Cs₂CO₃ (252 mg, 0.776 mmol) and ^{t}BuOH (3 ml) gave 29 (97 mg, 0.227 mmol, 59%).

¹H NMR (400 MHz, DMSO-d₆) δ 9.38 (t, J=6.0 Hz, 1H), 9.04 (s, 1H), 9.02 (d, J=2.5 Hz, 1H), 8.51 (dd, J=8.5, 2.5 Hz, 1H), 8.27 (s, 1H), 8.07 (d, J=8.5 Hz, 1H), 7.69-7.30 (m, 9H), 4.54 (d, J=6.0 Hz, 2H), 4.21 (q, J=7.0 Hz, 2H), 1.31 (t, J=7.0 Hz, 3H).

HRMS (ESI-TOF) calcd for C₂₅H₂₁O₃N₄ [M−H]: 425.1619, found: 425.1622.

### Example 12 Synthesis of Ethyl 1-(5-(Benzylcarbamoyl)pyridin-2-yl)-1H-pyrazole-4-carboxylate (30)

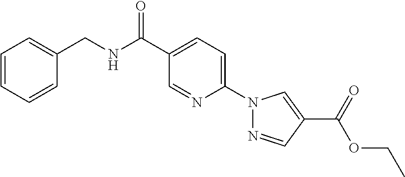 [SMILES: CCOC(=O)c1cnn(-c2ccc(C(=O)NCc3ccccc3)cn2)c1]

Following general procedure C: 28 (80 mg, 0.325 mmol), Pd^{t}BuXPhos G3 (25 mg, 0.0325 mmol), Cs₂CO₃ (317 mg, 0.97 mmol), pyrazole-4-carboxylate ethyl ester (67 mg, 0.48 mmol) and ^{t}BuOH (3 ml) gave 30 (41 mg, 0.117 mmol, 36%).

¹H NMR (400 MHz, DMSO-d₆) δ 9.33 (t, J=6.0 Hz, 1H), 9.04 (d, J=1.0 Hz, 1H), 8.99 (dd, J=2.5, 1.0 Hz, 1H), 8.49 (dd, J=8.5, 2.5 Hz, 1H), 8.27 (d, J=1.0 Hz, 1H,), 8.06 (dd, J=8.5, 1.0 Hz, 1H), 7.38-7.32 (m, 5H), 4.53 (d, J=6.0 Hz, 2H), 4.28 (q, J=7.0 Hz, 2H), 1.31 (t, J=7.0 Hz, 3H).

HRMS (ESI-TOF) calcd for C₁₉H₁₇03N₄ [M−H]: 349.1306, found: 349.1302.

### Example 13 Synthesis of 1-(5-(([1,1′-Biphenyl]-4-ylmethyl)carbamoyl)pyridine-2-yl)-1H-pyrazole-4-carboxylic acid (31)

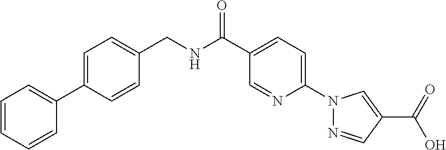 [SMILES: O=C(O)c1cnn(-c2ccc(C(=O)NCc3ccc(-c4ccccc4)cc3)cn2)c1]

29 (63 mg, 0.147 mmol) was dissolved in a mixture of THF and water (10 ml (10:1)) before the addition of LiOH-monohydrate (19 mg, 0.45 mmol). The resultant mixture was allowed to stir for 16 hr and confirmed to have gone to completion by TLC. HCl_{aq} (10 ml, 1 M) was added the reaction mixture and the resulting mixture was extracted with EtOAc (3×20 ml). The organic fractions were combined and washed with brine, dried with Na₂SO₄ and purified by flash column chromatography using (CH₂Cl₂, MeOH 0-5%, formic acid 1%) over 20 column volumes gave 31 (15 mg, 0.037 mmol, 26%).

¹H NMR (400 MHz, DMSO-d₆) δ 9.37-9.34 (m, 1H), 9.01 (d, J=2.5 Hz, 1H), 8.92 (d, J=2.5 Hz, 1H), 8.53-8.47 (m, 1H), 8.09-8.01 (m, 1H), 7.70-7.62 (m, 4H), 7.46 (t, J=7.8 Hz, 4H), 7.39-7.30 (m, 2H), 4.56 (d, J=6.0 Hz, 2H).

HRMS (ESI-TOF) calcd for C₂₃H₁₇03N₄ [M−H]: 397.1306, found: 397.1310.

### Example 14 Synthesis of 1-(5-(Benzylcarbamoyl)pyridin-2-yl)-1H-pyrazole-4-carboxylic acid (32)

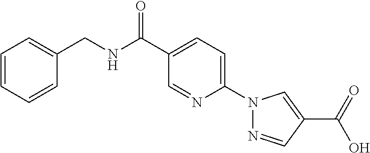 [SMILES: O=C(O)c1cnn(-c2ccc(C(=O)NCc3ccccc3)cn2)c1]

30 (20 mg, 0.043 mmol) was dissolved in THE (2 ml), MeOH (2 ml) and water (0.5 ml) was added to the reaction mixture before the addition of LiOH-monohydrate (16 mg, 0.40 mmol). The resultant mixture was stirred overnight at room temperature. The reaction mixture was acidified to pH 3 with HCl (1M) solution and extracted with EtOAc (3×25 ml), washed with brine dried with anhydrous Na₂SO₄. The crude compound was then purified using flash column chromatography using (CH₂Cl₂, MeOH 0-5%, formic acid 1%) over 20 column volumes gave 32 (13 mg, 0.040 mmol, 94%).

¹H NMR (400 MHz, DMSO-d₆) δ 9.34 (t, J=6.0 Hz, 1H), 8.99 (dd, J=2.5, 1.0 Hz, 1H), 8.96 (s, 1H), 8.48 (dd, J=8.5, 2.5 Hz, 1H), 8.19 (s, 1H), 8.08-8.02 (m, 1H), 7.48-7.21 (m, 5H), 4.54 (d, J=6.0 Hz, 2H).

HRMS (ESI-TOF) calcd for C₁₇H₁₃O₃N₄[M−H]⁻: 321.0993, found: 321.0994.

### Example 15 Synthesis of N-([1,1′-Biphenyl]-4-ylmethyl)-6-chloro-4-methoxynicotinamide (33)

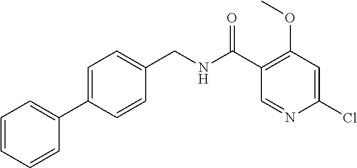 [SMILES: COc1cc(Cl)ncc1C(=O)NCc1ccc(-c2ccccc2)cc1]

Following general procedure B: 6-chloro-4-methoxy-nicotinic acid (300 mg, 1.60 mmol), 4-phenylbenzyl amine (439 mg, 2.4 mmol), T3P (1.27 g, 4 mmol) and DIPEA (825 μl, 4.8 mmol) gave 33 (432 mg, 1.22 mmol, 76%).

¹H NMR (400 MHz, DMSO-d₆) δ 8.82 (t, J=6.0 Hz, 1H), 8.51 (s, 1H), 7.68-7.60 (m, 4H), 7.57-7.27 (m, 6H), 4.53 (d, J=6.0 Hz, 2H), 3.99 (s, 3H).

HRMS (ESI-TOF) calcd for C₂₀H₁₈O₂N₂³⁵Cl [M+H]⁺: 353.1051, found: 353.1048.

### Reference Example 16 Synthesis of N-([1,1′-Biphenyl]-4-yl)-6-chloro-4-methoxynicotinamide (34)

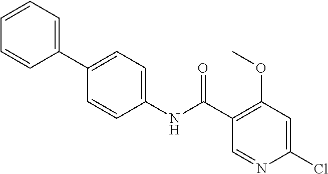 [SMILES: COc1cc(Cl)ncc1C(=O)Nc1ccc(-c2ccccc2)cc1]

Following general procedure B: 6-chloro-4-methoxy-nicotinic acid (500 mg, 2.67 mmol), 4-aminobiphenyl (540 mg, 3.2 mmol), T3P (2.12 g, 6.68 mmol) and DIPEA (846 ul, 6.68 mmol) gave 34 (311 mg, 0.92 mmol, 34%).

¹H NMR (400 MHz, DMSO-d₆) δ 10.36 (s, 1H), 8.46 (s, 1H), 7.87-7.77 (m, 2H), 7.71-7.62 (m, 4H), 7.52-7.30 (m, 4H), 3.99 (s, 3H).

HRMS (ESI-TOF) calcd for C₁₉H₁₆O₂N₂³⁵Cl [M+H]+: 339.0894, found: 339.0893.

### Reference Example 17 Synthesis of 6-Chloro-4-methoxy-N-(3-(trifluoromethyl)benzyl)nicotinamide (35)

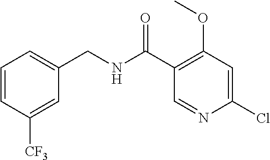 [SMILES: COc1cc(Cl)ncc1C(=O)NCc1cccc(C(F)(F)F)c1]

Following general procedure B: 6-chloro-4-methoxy-nicotinic acid (500 mg, 2.67 mmol), 3-trifluoromethylbenzyl amine (566 mg, 3.2 mmol), T3P (2.12 g, 6.68 mmol) and DIPEA (846 ul, 6.68 mmol) gave 35 (518 mg, 1.505 mmol, 56%).

¹H NMR (400 MHz, DMSO-d6) δ 8.91 (t, J=6.0 Hz, 1H), 8.48 (s, 1H), 7.75-7.53 (m, 4H), 7.35 (s, 1H), 4.57 (d, J=6.0 Hz, 2H), 3.98 (s, 3H).

HRMS (ESI-TOF) calcd for C₁₅H₁₃02N₂³⁵C₁F₃ [M+H]+: 345.0612, found: 345.0613.

### Reference Example 18—6-Chloro-N-(cyclohexylmethyl)-4-methoxynicotinamide (36)

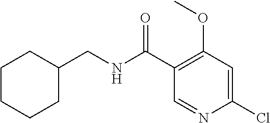 [SMILES: COc1cc(Cl)ncc1C(=O)NCC1CCCCC1]

Following general procedure B: 6-chloro-4-methoxy-nicotinic acid (500 mg, 2.67 mmol), cyclohexane-methylamine (361 mg, 3.2 mmol), T3P (2.12 g, 6.68 mmol) and DIPEA (846 μl, 6.68 mmol) gave 36 (438 mg, 1.55 mmol, 58%).

¹H NMR (400 MHz, DMSO-d₆) δ 8.40 (s, 1H), 8.17 (t, J=6.0 Hz, 1H), 7.30 (s, 1H), 3.95 (s, 3H,), 3.12-3.06 (m, 2H), 1.87-0.75 (m, 11H).

HRMS (ESI-TOF) calcd for C₁₄H₂₀O₂N₂³⁵Cl [M+H]+: 283.1207, found: 283.1208.

### Example 19—N-([1,1′-Biphenyl]-4-ylmethyl)-4-methoxy-6-(1H-pyrazol-1-yl)nicotinamide (37)

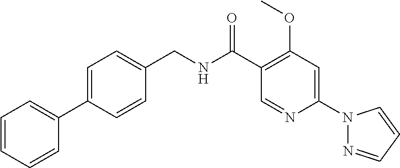 [SMILES: COc1cc(-n2cccn2)ncc1C(=O)NCc1ccc(-c2ccccc2)cc1]

Following general procedure C: 33 (50 mg, 0.142 mmol), Pd^{t}BuXPhos G3 (11.2 mg, 0.0142 mmol), Cs₂CO₃ (138 mg, 0.426 mmol), pyrazole (24 mg, 0.355 mmol) gave 37 (30 mg, 0.078 mmol, 55%).

¹H NMR (400 MHz, DMSO-d₆) δ 8.79 (t, J=6.0 Hz, 1H), 8.68-8.65 (m, 2H), 7.88 (dd, J=1.5, 1.0 Hz, 1H), 7.67-7.46 (m, 9H), 7.39-7.33 (m, 1H), 6.61 (dd, J=2.5, 1.5 Hz, 1H), 4.55 (d, J=6.0 Hz, 2H), 4.08 (s, 3H).

HRMS (ESI-TOF) calcd for C₂₃H₂₁O₂N₄[M+H]+: 385.1659, found: 385.1658.

### Example 20 Synthesis of N-([1,1′-Biphenyl]-4-yl)-4-methoxy-6-(1H-pyrazol-1-yl)nicotinamide (38)

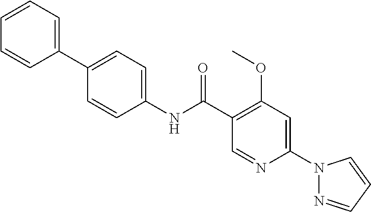 [SMILES: COc1cc(-n2cccn2)ncc1C(=O)Nc1ccc(-c2ccccc2)cc1]

Following general procedure C: 34 (150 mg, 0.43 mmol), Pd^{t}BuXPhos G3 (34 mg, 0.043 mmol), Cs₂CO₃ (354 mg, 1.09 mmol), pyrazole (74 mg, 1.09 mmol) gave 38 (67 mg, 0.181 mmol, 40%).

¹H NMR (400 MHz, DMSO-d₆) δ 8.68 (d, J=2.5 Hz, 1H), 8.59 (s, 1H), 7.90 (d, J=1.5 Hz, 1H), 7.70-7.63 (m, 6H), 7.52-7.42 (m, 4H), 6.63 (dd, J=2.5, 1.5 Hz, 1H), 4.08 (s, 3H).

HRMS (ESI-TOF) calcd for C₂₂H₁₉O₂N₄ [M+H]⁺: 371.1502, found: 371.1501.

### Example 21 Synthesis of 6-Chloro-4-methoxy-N-(3-(trifluoromethyl)benzyl)nicotinamide (39)

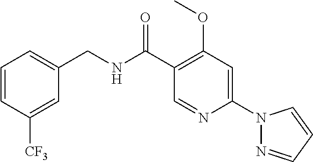 [SMILES: COc1cc(-n2cccn2)ncc1C(=O)NCc1cccc(C(F)(F)F)c1]

Following general procedure C: 35 (150 mg, 0.43 mmol), Pd^{t}BuXPhos G3 (34 mg, 0.043 mmol), Cs₂CO₃ (354 mg, 1.09 mmol), Pyrazole (74 mg, 1.09 mmol) gave 39 (72 mg, 0.191 mmol, 44%).

¹H NMR (400 MHz, DMSO-d₆) δ 9.26 (t, J=6.0 Hz, 1H), 8.66 (dd, J=2.5, 1.0 Hz, 1H), 8.62 (s, 1H), 8.33 (d, J=2.5 Hz, 1H), 7.88 (dd, J=1.5, 1.0 Hz, 1H), 7.68-7.57 (m, 4H), 6.61 (dd, J=2.5, 1.5 Hz, 1H), 4.59 (d, J=6.0 Hz, 2H), 4.07 (s, 3H).

HRMS (ESI-TOF) calcd for C₁₈H₁₆O₂N₄F₃[M+H]⁺: 377.1219, found: 377.1220.

### Example 22 Synthesis of N-(Cyclohexylmethyl)-4-methoxy-6-(1H-pyrazol-1-yl)nicotinamide (40)

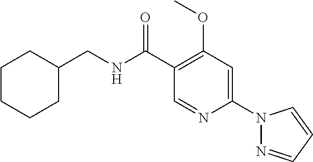 [SMILES: COc1cc(-n2cccn2)ncc1C(=O)NCC1CCCCC1]

Following general procedure C: 36 (150 mg, 0.43 mmol), Pd^{t}BuXPhos G3 (34 mg, 0.043 mmol), Cs₂CO₃ (354 mg, 1.09 mmol), pyrazole (74 mg, 1.09 mmol) gave 40 (67 mg, 0.21 mmol, 40%).

¹H NMR (400 MHz, DMSO-d₆) δ 8.68 (dd, J=2.5, 1.0 Hz, 1H), 8.56 (s, 1H), 7.90 (dd, J=1.5, 1.0 Hz, 1H), 7.57 (s, 1H), 6.64 (dd, J=2.5, 1.5 Hz, 1H), 3.95 (s, 3H), 3.06-3.03 (m, 2H), 1.79-1.58 (m, 5H), 1.28-1.09 (m, 4H), 1.00-0.83 (m, 2H).

HRMS (ESI-TOF) calcd for C₁₇H₂₃02N₄ [M+H]⁺: 315.1815, found: 315.1816.

### Example 23 Synthesis of N-([1,1′-Biphenyl]-4-ylmethyl)-4-hydroxy-6-(1H-pyrazol-1-yl)nicotinamide (41)

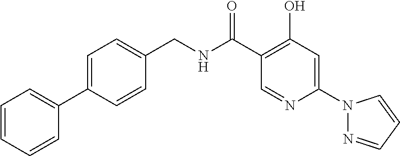 [SMILES: O=C(NCc1ccc(-c2ccccc2)cc1)c1cnc(-n2cccn2)cc1O]

TMS-I (17 mg, 0.0858 mmol) was added to the mixture of 37 (11 mg, 0.0286 mmol) and CH₂Cl₂ (1 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 41 (2 mg, 0.0054 mmol, 20%).

¹H NMR (400 MHz, DMSO-d₆) δ 8.76 (s, 1H), 8.64 (d, J=2.5 Hz, 1H), 7.86 (d, J=1.5 Hz, 1H), 7.70-7.60 (m, 5H), 7.52-7.29 (m, 6H), 6.60 (dd, J=2.5, 1.5 Hz, 1H), 4.59 (d, J=6.0 Hz, 2H).

HRMS (ESI-TOF) calcd for C₂₂H₁₉O₂N₄[M+H]⁺: 371.1502, found: 371.1502.

### Example 24 Synthesis of N-([1,1′-Biphenyl]-4-yl)-4-hydroxy-6-(1H-pyrazol-1-yl)nicotinamide (42)

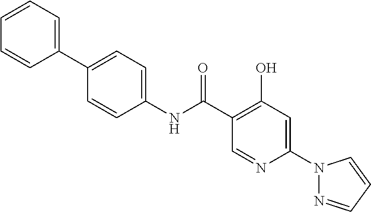 [SMILES: O=C(Nc1ccc(-c2ccccc2)cc1)c1cnc(-n2cccn2)cc1O]

TMS-I (66 mg, 0.33 mmol) was added to the reaction mixture of 38 (41 mg, 0.11 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 42 (7 mg, 0.0196 mmol, 18%).

¹H NMR (400 MHz, DMSO-d₆) δ 8.70 (d, J=2.0 Hz, 1H), 8.64 (s, 1H), 7.92 (d, J=2.0 Hz, 1H), 7.72-7.62 (m, 5H), 7.55-7.27 (m, 6H), 6.65 (dd, J=2.0 Hz, 1H).

HRMS (ESI-TOF) calcd for C₂₁H₁₇02N₄ [M+H]⁺: 357.1346, found: 357.1343.

### Example 25 Synthesis of 4-Hydroxy-6-(1H-pyrazol-1-yl)-N-(3-(trifluoromethyl)benzyl)nicotinamide (43)

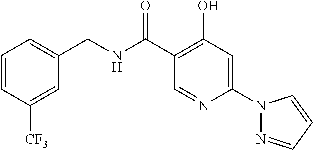 [SMILES: O=C(NCc1cccc(C(F)(F)F)c1)c1cnc(-n2cccn2)cc1O]

TMS-I (59 mg, 0.295 mmol) was added to the reaction mixture of 39 (37 mg, 0.0986 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 43 (20 mg, 0.055 mmol, 57%).

¹H NMR (400 MHz, DMSO-d₆) δ 8.66 (s, 1H), 8.59 (dd, J=2.5, 1.0 Hz, 1H), 7.82 (dd, J=1.5, 1.0 Hz, 1H), 7.71-7.49 (m, 4H), 7.26 (s, 1H), 6.55 (dd, J=2.5, 1.5 Hz, 1H), 4.59 (d, J=6.0 Hz, 2H).

HRMS (ESI-TOF) calcd for C₁₇H₁₄02N₄F₃[M+H]⁺: 363.1063, found: 363.1067.

### Example 26 Synthesis of N-(Cyclohexylmethyl)-4-hydroxy-6-(1H-pyrazol-1-yl)nicotinamide (44)

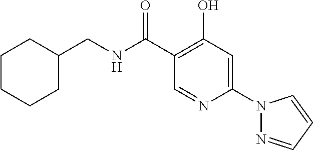 [SMILES: O=C(NCC1CCCCC1)c1cnc(-n2cccn2)cc1O]

TMS-I (128 mg, 0.64 mmol) was added to the reaction mixture of 40 (67 mg, 0.214 mmol) and CH₂Cl₂ (2.5 ml). The resultant mixture was heated at 90° C. for 90 minutes, then cooled to rt, HCl_{aq} (1.5 ml, 1M) was added before being extracted with CH₂Cl₂ (3×10 ml). The organic fractions were combined, dried with anhydrous Na₂SO₄ concentrated in vacuo and the crude compound was then purified using flash column chromatography using (0%-5% MeOH, CH₂Cl₂, 1% NH₃) over 20 column volumes gave 44 (17 mg, 0.056 mmol, 26%).

¹H NMR (400 MHz, DMSO-d₆) δ 8.63 (d, J=2.5 Hz, 1H), 8.47 (s, 1H), 8.29 (d, J=2.5 Hz, 1H), 8.21 (s, 1H), 6.65-6.63 (m, 1H), 3.05 (t, J=6.0 Hz, 2H), 1.75-1.44 (m, 5H), 1.27-1.10 (m, 3H), 1.05-0.80 (m, 2H).

HRMS (ESI-TOF) calcd for C₁₆H₂₁O₂N₄ [M+H]⁺: 301.1659, found: 301.1656.

### Reference Example 27—Synthesis of 6-Chloro-4-methoxy-N-(4-(trifluoromethyl)benzyl)nicotinamide (45)

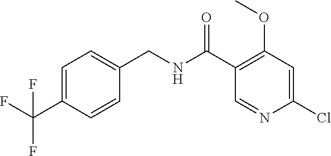 [SMILES: COc1cc(Cl)ncc1C(=O)NCc1ccc(C(F)(F)F)cc1]

Following general procedure B: 6-chloro-5-methoxy-nicotinic acid (350 mg, 1.87 mmol), 4-trifluoromethylbenzylamine (448 mg, 2.56 mmol), T3P (1.48 g, 4.67 mmol) and DIPEA (803 μl, 4.67 mmol) gave 45 (437 mg, 1.27 mmol, 66%).

¹H NMR (400 MHz, Chloroform-d) δ 9.02 (s, 1H), 7.81 (t, J=6.0 Hz, 1H), 7.58 (d, J=8.0 Hz, 2H,), 7.43 (d, J=8.0 Hz, 2H), 6.93 (s, 1H), 4.69 (d, J=6.0 Hz, 2H), 4.01 (s, 3H).

HRMS (ESI-TOF) calcd for C₁₅H₁₃02N₂³⁵C₁F₃ [M+H]+: 345.0612, found: 345.0608.

### Reference Example 28 Synthesis of N-([1,1′-Biphenyl]-3-ylmethyl)-6-chloro-4-methoxynicotinamide (46)

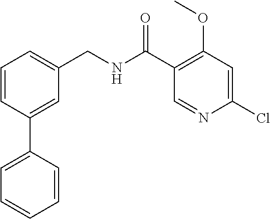 [SMILES: COc1cc(Cl)ncc1C(=O)NCc1cccc(-c2ccccc2)c1]

Following general procedure B: 6-chloro-5-methoxy-nicotinic acid (400 mg, 2.13 mmol), 3-phenylbenzyl amine (469 mg, 2.56 mmol), T3P (1.62 g, 5.12 mmol) and DIPEA (880 μl, 5.12 mmol) gave 46 (570 mg, 1.61 mmol, 76%).

¹H NMR (400 MHz, DMSO-d₆) δ 8.85 (t, J=6.0 Hz, 1H), 8.48 (s, 1H), 7.70-7.30 (m, 10H), 4.56 (d, J=6.0 Hz, 2H), 3.96 (s, 3H).

HRMS (ESI-TOF) calc'd for C₂₀H₁₈O₂N₂³⁵Cl [M+H]⁺: 353.1051, found: 353.1053.

### Reference Example 29 Synthesis of N-([1,1′-Biphenyl]-3-yl)-6-chloro-4-methoxynicotinamide (47)

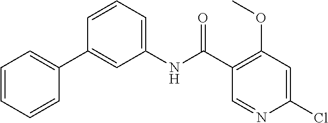 [SMILES: COc1cc(Cl)ncc1C(=O)Nc1cccc(-c2ccccc2)c1]

Following general procedure B: 6-chloro-5-methoxy-nicotinic acid (350 mg, 1.87 mmol), 3-aminobiphenyl (411 mg, 2.43 mmol), T3P (1.48 g, 4.67 mmol) and DIPEA (803 μl, 4.67 mmol) gave 47 (421 mg, 1.24 mmol, 66%).

¹H NMR (400 MHz, Chloroform-d) δ 9.24 (s, 1H), 9.05 (s, 1H), 7.90-7.87 (m, 1H), 7.66-7.30 (m, 8H), 6.91 (s, 1H), 4.06 (s, 3H).

HRMS (ESI-TOF) calcd for C₁₉H₁₆02N₂³⁵Cl [M+H]+: 339.0894, found: 339.0894.

### Reference Example 30 Synthesis of 6-Chloro-4-methoxy-N-(4-(trifluoromethoxy)benzyl)nicotinamide (48)

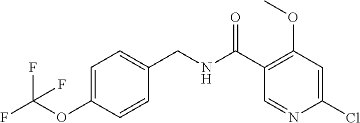 [SMILES: COc1cc(Cl)ncc1C(=O)NCc1ccc(OC(F)(F)F)cc1]

Following general procedure B: 6-chloro-5-methoxy-nicotinic acid (300 mg, 1.60 mmol), 4-trifluoromethoxybenzylamine (367 mg, 1.92 mmol), T3P (1.01 g, 3.2 mmol) and DIPEA (803 μl, 4.67 mmol) gave 48 (434 mg, 1.20 mmol, 75%).

¹H NMR (400 MHz, Chloroform-d) δ 8.97 (s, 1H), 7.78 (t, J=6.0 Hz, 1H), 7.37-7.29 (m, 2H), 7.17-7.11 (m, 2H), 6.90 (s, 1H), 4.61 (d, J=6.0 Hz, 2H), 3.98 (s, 3H).

HRMS (ESI-TOF) calcd for C₁₅H₁₃03N₂³⁵C₁F₃ [M+H]+: 361.0561, found: 361.0563.

### Reference Example 31 Synthesis of 6-Chloro-4-methoxy-N-((4-(trifluoromethyl)cyclohexyl)methyl)nicotinamide (49)

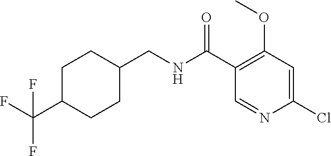 [SMILES: COc1cc(Cl)ncc1C(=O)NCC1CCC(C(F)(F)F)CC1]

Following general procedure B: 6-chloro-5-methoxy-nicotinic acid (250 mg, 1.33 mmol), C-(4-trifluoromethyl-cyclohexylamine (255 mg, 1.59 mmol), T3P (827 mg, 2.6 mmol) and DIPEA (803 μl, 4.67 mmol) gave 49 (402 mg, 1.14 mmol, 86%).

¹H NMR (400 MHz, CDCl₃) δ 8.89 (s, 1H), 7.31 (s, 1H), 6.82 (s, 1H), 3.94 (s, 3H), 3.23 (t, J=6.2 Hz, 2H), 1.95-1.74 (m, 5H), 1.57-1.42 (m, 1H), 1.21 (qd, J=12.7, 2.9 Hz, 2H), 0.93 (qd, J=12.8, 3.1 Hz, 2H).

HRMS (ESI-TOF) calc'd for C₁₅H₁₉O₂N₂³⁵C₁F₃ [M+H]+: 351.1081, found: 351.1078.

### Reference Example 32 Synthesis of tert-Butyl ((6-chloropyridin-3-yl)methyl)carbamate (50)

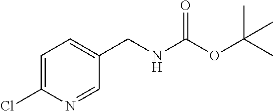 [SMILES: CC(C)(C)OC(=O)NCc1ccc(Cl)nc1]

6-Chloropyridin-3-yl)methamine (4.0 g, 0.0281 mol) was dissolved in CH₂Cl₂ (50 ml) before the addition of DIPEA (36.9 ml, 0.049 mol). Di tert-butyl dicarbonate (7.6 g, 0.035 mol) was added slowly to the reaction mixture and the resultant mixture was allowed to stir for 16 hrs at room temperature. The reaction mixture was then washed with water (3×50 ml), brine (50 ml) and dried over Na₂SO₄. The organic phase was reduced in vacuo before being purified by flash column chromatography using (Cyclohexane 100%-50%, EtOAc 0%-50%) over 20 column volumes gave 50 (6.08 g, 0.0251 mmol, 89%).

¹H NMR (400 MHz, Chloroform-d) δ 8.25 (dd, J=2.5, 1.0 Hz, 1H), 7.58 (dd, J=8.5, 2.5 Hz, 1H,), 7.24 (d, J=1.0 Hz, 1H), 5.08 (s, 1H), 4.26 (d, J=6.0 Hz, 2H), 1.41 (s, 9H).

HRMS (ESI-TOF) calcd for C₁₁H₁₆O₂N₂³⁵Cl [M+H]⁺: 243.0894, found: 243.0895.

### Reference Example 33-Synthesis of tert-Butyl ((6-phenylpyridin-3-yl)methyl)carbamate (51)

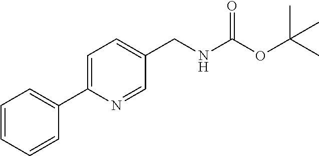 [SMILES: CC(C)(C)OC(=O)NCc1ccc(-c2ccccc2)nc1]

50 (500 mg, 2.06 mmol), phenyl boronic acid (249 mg, 2.06 mmol), Pd tetrakis (118 mg, 0.103 mmol) and Cs₂CO₃ (1.33 g, 4.12 mmol) were dissolved in anhydrous dioxane (5 ml). The resultant mixture was heated under microwave irradiation at 100° C. for 30 mins. The reaction mixture was filtered through a celite pad, water (25 ml) was added to the reaction mixture and was extracted with CH₂Cl₂ (3×25 ml). The organic fractions were combined, washed with water (3×50 ml), brine (50 ml) and dried over Na₂SO₄. The organic phase was removed in vacuo before being purified by flash column chromatography using (cyclohexane 100%-50%, EtOAc 0%-50%) over 20 column volumes gave 51 (520 mg, 1.83 mmol, 89%).

¹H NMR (400 MHz, Chloroform-d) δ 8.58 (t, J=1.5 Hz, 1H), 7.98-7.93 (m, 2H,), 7.67 (d, J=1.5 Hz, 2H), 7.48-7.38 (m, 3H), 5.08 (s, 1H), 4.34 (d, J=6.0 Hz, 2H), 1.46 (s, 9H).

HRMS (ESI-TOF) calcd for C₁₇H₂₁O₂N₂ [M+H]⁺: 285.1597, found: 285.1594.

### Reference Example 34 Synthesis of (6-Phenylpyridin-3-yl)methanamine (52)

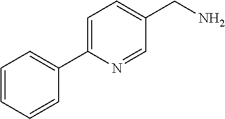 [SMILES: NCc1ccc(-c2ccccc2)nc1]

51 (500 mg, 1.76 mmol) was dissolved in CH₂Cl₂ (5 ml) and HCl ((2M) in ether (3 ml)) was added to the solution. The resultant mixture was put under vacuo and flushed with N₂; this was repeated 3 times. The resultant mixture was stirred at room temperature for 16 hrs. The volatiles were evaporated in vacuo to give 52 (312 mg, 1.69 mmol, 96%).

¹H NMR (400 MHz, DMSO-d₆) δ 8.85 (s, 2H), 8.79 (dd, J=2.5, 1.0 Hz, 1H), 8.15-8.07 (m, 3H,), 8.02 (dd, J=8.0, 1.0 Hz, 1H), 7.55-7.41 (m, 3H), 4.08 (s, 2H).

HRMS (ESI-TOF) calcd for C₁₂H₁₃N₂ [M+H]⁺: 185.1073, found: 185.1073.

### Reference Example 35 Synthesis of 6-Chloro-4-methoxy-N-((6-phenylpyridin-3-yl)methyl)nicotinamide (53)

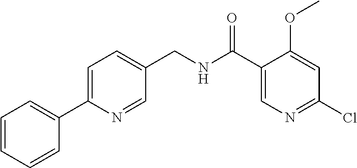 [SMILES: COc1cc(Cl)ncc1C(=O)NCc1ccc(-c2ccccc2)nc1]

Following general procedure B: 52 (100 mg, 0.54 mmol), 6-chloro-4-methoxy-nicotinic acid (85 mg, 0.45 mmol) and T3P (358 mg, 1.125 mmol) gave 53 (144 mg, 0.41 mmol, 90%).

¹H NMR (400 MHz, DMSO-d₆) δ 8.91 (t, J=6.0 Hz, 1H,), 8.51 (s, 1H), 8.13-7.79 (m, 4H), 7.63-7.37 (m, 4H), 7.32 (s, 1H), 4.54 (d, J=6.0 Hz, 2H), 3.98 (s, 3H). HRMS (ESI-TOF) calcd for C₁₉H₁₇02N₃₃⁵Cl [M+H]⁺: 354.1003, found: 354.1002.

### Example 36 Synthesis of 4-Methoxy-6-(1H-pyrazol-1-yl)-N-(4-(trifluoromethyl)benzyl)nicotinamide (56)

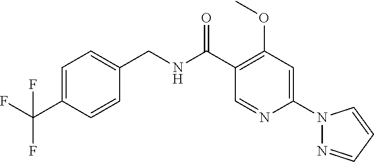 [SMILES: COc1cc(-n2cccn2)ncc1C(=O)NCc1ccc(C(F)(F)F)cc1]

Following general procedure C: 45 (200 mg, 0.58 mmol), Pd^{t}BuXPhos G3 (46 mg, 0.058 mmol), Cs₂CO₃ (377 mg, 1.16 mmol), pyrazole (42 mg, 0.63 mmol) and ^{t}BuOH (2 ml) gave 56 (69 mg, 0.185 mmol, 32%).

¹H NMR (400 MHz, DMSO-d₆) δ 8.88 (t, J=6.0 Hz, 1H), 8.66 (dd, J=2.5, 1.0 Hz, 1H), 8.64 (s, 1H), 7.89 (t, J=1.5, 1.0 Hz, 1H), 7.72 (d, J=7.0 Hz, 2H), 7.60 (s, 1H), 7.56 (d, J=7.0 Hz, 2H), 6.62 (dd, J=2.5, 1.5 Hz, 1H), 4.59 (d, J=6.0 Hz, 2H), 4.08 (s, 3H).

HRMS (ESI-TOF) calcd for C₁₈H₁₆O₂N₄F₃[M+H]⁺: 377.1221, found: 377.1221.

### Example 37 Synthesis of N-([1,1′-Biphenyl]-3-ylmethyl)-4-methoxy-6-(1H-pyrazol-1-yl)nicotinamide (57)

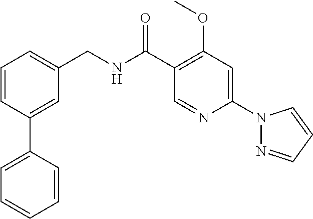 [SMILES: COc1cc(-n2cccn2)ncc1C(=O)NCc1cccc(-c2ccccc2)c1]

Following general procedure C: 46 (150 mg, 0.42 mmol), Pd^{t}BuXPhos G3 (33 mg, 0.0426 mmol), Cs₂CO₃ (341 mg, 1.05 mmol), pyrazole (42 mg, 0.63 mmol) and ^{t}BuOH (2 ml) gave 57 (78 mg, 0.203 mmol, 48%).

¹H NMR (400 MHz, Chloroform-d) δ 9.03 (d, J=2.0 Hz, 1H), 8.53 (d, J=2.5 Hz, 1H,), 7.85-7.76 (m, 1H), 7.70-7.63 (m, 1H), 7.56-7.20 (m, 9H), 7.20-7.15 (m, 1H), 6.40 (dd, J=2.5, 2.0 Hz, 1H), 4.66 (d, J=6.0 Hz, 2H), 3.97 (s, 3H).

HRMS (ESI-TOF) calcd for C₂₃H₂₁O₂N₄ [M+H]⁺: 385.1659, found: 385.1656.

### Example 38 Synthesis of N-([1,1′-Biphenyl]-3-yl)-4-methoxy-6-(1H-pyrazol-1-yl)nicotinamide (58)

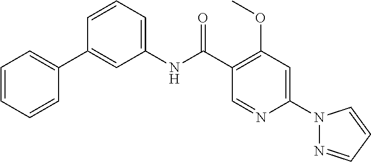 [SMILES: COc1cc(-n2cccn2)ncc1C(=O)Nc1cccc(-c2ccccc2)c1]

Following general procedure C: 47 (100 mg, 0.295 mmol), Pd^{t}BuXPhos G3 (20 mg, 0.028 mmol), Cs₂CO₃ (185 mg, 0.568 mmol), pyrazole (23 mg, 0.34 mmol) and ^{t}BuOH (2 ml) gave 58 (50 mg, 0.13 mmol, 46%).

¹H NMR (400 MHz, Chloroform-d) δ 9.42 (s, 1H), 9.18 (s, 1H), 8.64 (dd, J=2.5, 1.0 Hz, 1H), 7.91 (m, 1H), 7.78 (dd, J=1.5, 1.0 Hz, 1H,), 7.69-7.33 (m, 9H), 6.51 (dd, J=2.5, 1.5 Hz, 1H,), 4.23 (s, 3H).

HRMS (ESI-TOF) calcd for C₂₂H₁₉O₂N₄ [M+H]⁺: 371.1502, found: 371.1497.

### Example 39 Synthesis of 4-Methoxy-6-(1H-pyrazol-1-yl)-N-(4-(trifluoromethoxy)benzyl)nicotinamide (59)

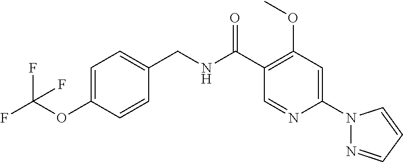 [SMILES: COc1cc(-n2cccn2)ncc1C(=O)NCc1ccc(OC(F)(F)F)cc1]

Following general procedure C: 48 (100 mg, 0.277 mmol), Pd^{t}BuXPhos G3 (19 mg, 0.027 mmol), Cs₂CO₃ (180 mg, 0.554 mmol), pyrazole (23 mg, 0.33 mmol) and ^{t}BuOH (2 ml) gave 59 (79 mg, 0.201 mmol, 73%).

¹H NMR (400 MHz, Chloroform-d) δ 9.12 (s, 1H), 8.63 (dd, J=2.5, 1.0 Hz, 1H), 7.76 (t, J=1.5, 1.0 Hz, 1H), 7.61 (s, 1H), 7.40 (d, J=7.5 Hz, 2H), 7.20 (d, J=7.5 Hz, 2H), 6.50 (dd, J=2.5, 1.5 Hz, 1H,), 4.68 (d, J=6.0 Hz, 2H), 4.10 (s, 3H).

HRMS (ESI-TOF) calcd for C₁₈H₁₆03N₄F₃[M+H]⁺: 393.1169, found: 393.1163.

### Example 40 Synthesis of 4-Methoxy-6-(1H-pyrazol-1-yl)-N-((4-(trifluoromethyl)cyclohexyl)methyl)nicotinamide (60)

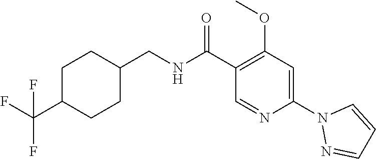 [SMILES: COc1cc(-n2cccn2)ncc1C(=O)NCC1CCC(C(F)(F)F)CC1]

Following general procedure C: 49 (100 mg, 0.277 mmol), Pd^{t}BuXPhos G3 (19 mg, 0.027 mmol), Cs₂CO₃ (180 mg, 0.554 mmol), pyrazole (23 mg, 0.33 mmol) and ^{t}BuOH (2 ml) gave 60 (49 mg, 0.127 mmol, 46%).

¹H NMR (400 MHz, CDCl₃) δ 9.07 (s, 1H), 8.62 (dd, J=2.6, 0.7 Hz, 1H), 7.75 (d, J=1.6 Hz, 1H), 7.60 (s, 1H), 7.54 (d, J=6.2 Hz, 1H), 6.49 (dd, J=2.7, 1.7 Hz, 1H), 4.13 (s, 3H), 3.35 (t, J=6.4 Hz, 2H), 2.06-1.90 (m, 5H), 1.69-1.57 (m, 1H), 1.43-1.20 (m, 2H), 1.05 (qd, J=12.8, 2.6 Hz, 2H).

HRMS (ESI-TOF) calcd for C₁₈H₂₂02N₄F₃[M+H]⁺: 383.1689, found: 383.1689.

### Example 41 Synthesis of 4-Methoxy-N-((6-phenylpyridin-3-yl)methyl)-6-(1H-pyrazol-1-yl)nicotinamide (61)

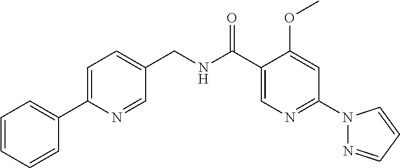 [SMILES: COc1cc(-n2cccn2)ncc1C(=O)NCc1ccc(-c2ccccc2)nc1]

Following general procedure C: 53 (80 mg, 0.226 mmol), Pd^{t}BuXPhos G1 (14 mg, 0.0226 mmol), Cs₂CO₃ (145 mg, 0.452 mmol), pyrazole (15 mg, 0.226 mmol) and ^{t}BuOH (2 ml) gave 61 (16 mg, 0.043 mmol, 19%).

¹H NMR (400 MHz, Chloroform-d) δ 9.11 (s, 1H), 8.69 (dd, J=2.5, 1.0 Hz, 1H), 8.62 (dd, J=2.5, 1.0 Hz, 1H), 8.01-7.68 (m, 7H), 7.52-7.38 (m, 3H), 6.48 (dd, J=2.5, 1.5 Hz, 1H), 4.73 (d, J=6.0 Hz, 2H), 4.10 (s, 3H).

HRMS (ESI-TOF) calcd for C₂₂H₂₀O₂N₄[M+H]⁺: 386.1611, found: 386.1604.

### Example 42 Synthesis of 4-Hydroxy-6-(1H-pyrazol-1-yl)-N-(4-(trifluoromethyl)benzyl)nicotinamide (64)

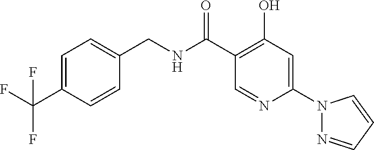 [SMILES: O=C(NCc1ccc(C(F)(F)F)cc1)c1cnc(-n2cccn2)cc1O]

Following general procedure D: 56 (12 mg, 0.0319 mmol) and LiCl (19 mg, 0.319 mmol) in DMAc (2 ml) gave 64 (6 mg, 0.016 mmol, 52%).

¹H NMR (400 MHz, DMSO-d₆) δ 10.12 (s, 1H), 8.68 (s, 1H), 8.64-8.58 (m, 1H), 7.81 (s, 1H,), 7.71 (d, J=8.0 Hz, 2H), 7.56 (d, J=8.0 Hz, 2H), 7.20 (m, 1H), 6.60-6.53 (m, 1H), 4.61 (d, J=6.0 Hz, 2H).

HRMS (ESI-TOF) calcd for C₁₇H₁₂O₂N₄F₃ [M−H]: 361.0917, found: 361.0917.

### Example 43 Synthesis of N-([1,1′-Biphenyl]-3-ylmethyl)-4-hydroxy-6-(1H-pyrazol-1-yl)nicotinamide (65)

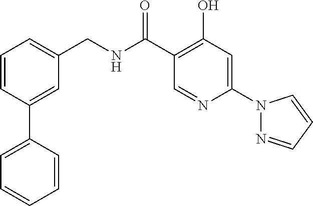 [SMILES: O=C(NCc1cccc(-c2ccccc2)c1)c1cnc(-n2cccn2)cc1O]

Following general procedure D: 57 (50 mg, 0.129 mmol) and LiCl (78 mg, 1.29 mmol) in DMAc (5 ml) gave 65 (30 mg, 0.081 mmol, 63%).

¹H NMR (400 MHz, DMSO-d₆) δ 13.21 (s, 1H), 9.26 (t, J=6.0 Hz, 1H), 8.69 (dd, J=2.5, 1.0 Hz, 1H), 8.64 (d, J=2.5 Hz, 1H), 8.28 (d, J=2.5 Hz, 1H), 7.91 (dd, J=1.5, 1.0 Hz, 1H), 7.59-7.29 (m, 9H), 6.64 (dd, J=2.5, 1.5 Hz, 1H), 4.63 (d, J=6.0 Hz, 2H,).

HRMS (ESI-TOF) calcd for C₂₂H₁₉O₂N₄[M+H]⁺: 371.1502, found: 371.1507

### Example 44 Synthesis of N-([1,1′-Biphenyl]-3-yl)-4-hydroxy-6-(1H-pyrazol-1-yl)nicotinamide (66)

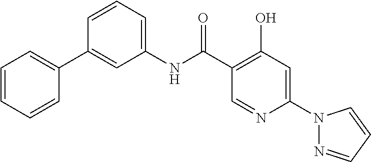 [SMILES: O=C(Nc1cccc(-c2ccccc2)c1)c1cnc(-n2cccn2)cc1O]

Following general procedure D: 58 (40 mg, 0.107 mmol) and LiCl (45 mg, 1.07 mmol) in DMAc (2 ml) gave 66 (19 mg, 0.053 mmol, 50%).

¹H NMR (400 MHz, DMSO-d₆) δ 8.74-8.64 (m, 2H), 8.04 (s, 1H), 7.93 (s, 1H), 7.76-7.63 (m, 3H), 7.56-7.29 (m, 7H), 6.68-6.63 (m, 1H).

HRMS (ESI-TOF) calcd for C₂₁H₁₇O₂N₄ [M+H]⁺: 357.1346, found: 357.1345.

### Example 45 Synthesis of 4-Hydroxy-6-(1H-pyrazol-1-yl)-N-(4-(trifluoromethyoxy)benzyl)nicotinamide (67)

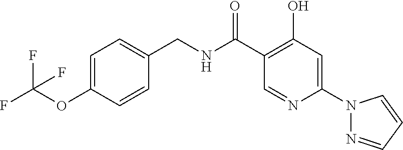 [SMILES: O=C(NCc1ccc(OC(F)(F)F)cc1)c1cnc(-n2cccn2)cc1O]

Following general procedure D: 59 (70 mg, 0.178 mmol) and LiCl (74 mg, 1.78 mmol) in DMAc (2 ml) gave 67 (43 mg, 0.114 mmol, 64%).

¹H NMR (400 MHz, DMSO-d₆) δ 13.16 (s, 1H), 9.41 (s, 1H), 8.76 (s, 1H), 8.67-8.61 (m, 1H), 7.89-7.84 (m, 1H), 7.51-7.45 (m, 2H), 7.35-7.31 (m, 3H), 6.61-6.58 (m, 1H), 4.57 (d, J=6.0 Hz, 2H).

HRMS (ESI-TOF) calcd for C₁₇H₁₂O₃N₄F₃ [M−H]: 377.0867, found: 377.865.

### Example 46 Synthesis of 4-Hydroxy-6-(1H-pyrazol-1-yl)-N-((4-(trifluoromethyl)cyclohexyl)methyl)nicotinamide (68)

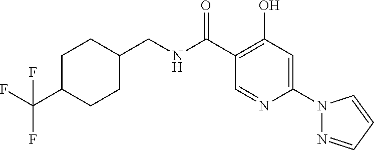 [SMILES: O=C(NCC1CCC(C(F)(F)F)CC1)c1cnc(-n2cccn2)cc1O]

Following general procedure D: 60 (30 mg, 0.078 mmol) and LiCl (33 mg, 0.78 mmol) in DMAc (2 ml) gave 68 (8.5 mg, 0.023 mmol, 30%).

¹H NMR (400 MHz, DMSO) δ 13.33 (s, 1H), 9.10 (s, 1H), 8.72 (s, 1H), 8.63 (d, J=2.6 Hz, 1H), 7.86 (d, J=1.6 Hz, 1H), 7.28 (s, 1H), 6.60 (dd, J=2.7, 1.7 Hz, 1H), 3.20 (t, J=6.3 Hz, 2H), 2.29-2.13 (m, 1H), 1.96-1.78 (m, 4H), 1.65-1.47 (m, 1H), 1.32-0.96 (m, 4H).

HRMS (ESI-TOF) calcd for C₁₇H₂₀O₂N₄F₃[M+H]⁺: 369.1532, found: 369.1533.

### Example 47 Synthesis of 4-Hydroxy-N-((6-phenylpyridin-3-yl)methyl)-6-(1H-pyrazol-1-yl)nicotinamide (69)

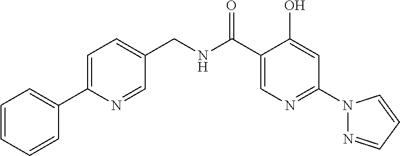 [SMILES: O=C(NCc1ccc(-c2ccccc2)nc1)c1cnc(-n2cccn2)cc1O]

Following general procedure D: 61 (16 mg, 0.041 mmol) and LiCl (17 mg, 0.41 mmol) in DMAc (2 ml) gave 69 (6 mg, 0.0161 mmol, 40%).

¹H NMR (400 MHz, DMSO-d₆) δ 8.80-8.62 (m, 3H), 8.15-7.86 (m, 6H), 7.56-7.43 (m, 4H), 6.62-6.59 (m, 1H), 4.62 (d, J=6.0 Hz, 2H).

HRMS (ESI-TOF) calcd for C₂₁H₁₈O₂N₅[M+H]⁺: 372.1455, found: 372.1447.

### Reference Example 48 Synthesis of N-([1,1′-Biphenyl]-4-ylmethyl)-6-chloro-5-methoxynicotinamide (72)

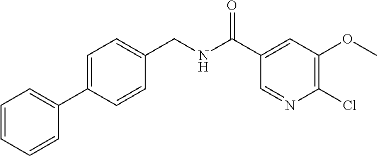 [SMILES: COc1cc(C(=O)NCc2ccc(-c3ccccc3)cc2)cnc1Cl]

Following general procedure B: 6-chloro-5-methoxy-nicotinic acid (850 mg, 4.55 mmol), 4-phenylbenzyl amine (1 g, 5.46 mmol), T3P (4.32 g, 13.6 mmol) and DIPEA (2.2 ml, 6.68 mmol) gave 72 (502 mg, 1.42 mmol, 31%).

¹H NMR (400 MHz, DMSO-d₆) δ 9.32 (t, J=6.0 Hz, 1H), 8.50 (d, J=2.0 Hz, 1H), 7.96 (d, J=2.0 Hz, 1H), 7.74-7.31 (m, 9H), 4.56 (d, J=6.0 Hz, 2H), 3.96 (s, 3H). HRMS (ESI-TOF) calcd for C₂₀H₁₈O₂N₂³⁵Cl [M+H]⁺: 353.1051, found: 353.1049.

### Example 49 Synthesis of N-([1,1′-Biphenyl]-4-ylmethyl)-5-methoxy-6-(1H-pyrazol-1-yl)nicotinamide (73)

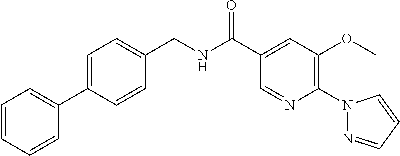 [SMILES: COc1cc(C(=O)NCc2ccc(-c3ccccc3)cc2)cnc1-n1cccn1]

Following general procedure C: 72 (100 mg, 0.284 mmol), Pd^{t}BuXPhos G3 (22 mg, 0.0284 mmol), Cs₂CO₃ (279 mg, 0.852 mmol), pyrazole (38 mg, 0.568 mmol) and ^{t}BuOH (2 ml) gave 73 (23 mg, 0.0598 mmol, 20%).

¹H NMR (400 MHz, Chloroform-d) δ 8.46 (d, J=2.0 Hz, 1H), 8.30 (dd, J=2.5, 1.0 Hz, 1H), 7.97 (d, J=2.0 Hz, 1H), 7.76 (dd, J=1.5, 1.0 Hz, 1H), 7.64-7.32 (m, 9H), 6.46 (dd, J=2.5, 1.5 Hz, 1H), 4.67 (d, J=6.0 Hz, 2H), 3.93 (s, 3H).

HRMS (ESI-TOF) calcd for C₂₃H₂₁O₂N₄ [M+H]⁺: 385.1659, found: 385.1661.

### Example 50 Synthesis of N-([1,1′-Biphenyl]-3-ylmethyl)-5-methoxy-6-(1H-pyrazol-1-yl)nicotinamide (74)

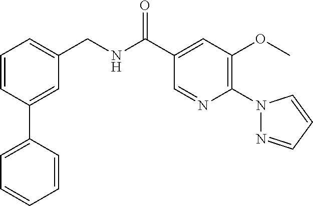 [SMILES: COc1cc(C(=O)NCc2cccc(-c3ccccc3)c2)cnc1-n1cccn1]

Following general procedure B: 6-chloro-5-methoxy-nicotinic acid (250 mg, 1.33 mmol), 3-phenylbenzyl amine (285 mg, 1.56 mmol), T3P (1.08 g, 3.4 mmol) and DIPEA (574 μl, 3.34 mmol) gave [N-([1,1′-biphenyl]-3-ylmethyl)-6-chloro-5-methoxynicotinamide. The crude material was taken onto the next step without purification and following general procedure C: N-([1,1′-biphenyl]-3-ylmethyl)-6-chloro-5-methoxynicotinamide (100 mg, 0.284 mmol), Pd^{t}BuXPhos G3 (20 mg, 0.028 mmol), Cs₂CO₃ (185 mg, 0.568 mmol), pyrazole (23 mg, 0.34 mmol) gave 74 (54 mg, 0.14 mmol, 49%).

¹H NMR (400 MHz, DMSO-d₆) δ 9.37 (t, J=6.0 Hz, 1H), 8.62 (d, J=2.0 Hz, 1H), 8.28 (dd, J=2.5, 1.0 Hz, 1H), 8.10 (d, J=2.0 Hz, 1H), 7.77 (t, J=1.5, 1.0 Hz, 1H), 7.69-7.32 (m, 9H), 6.53 (dd, J=2.5, 1.5 Hz, 1H), 4.62 (d, J=6.0 Hz, 2H), 3.94 (s, 3H).

HRMS (ESI-TOF) calcd for C₂₃H₂₁O₂N₄[M+H]⁺: 385.1659, found: 385.1661.

### Example 51 Synthesis of N-([1,1′-Biphenyl]-4-ylmethyl)-5-hydroxy-6-(1H-pyrazol-1-yl)nicotinamide (75)

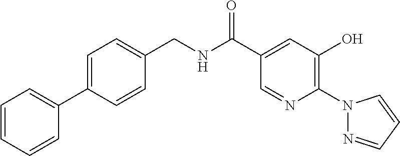 [SMILES: O=C(NCc1ccc(-c2ccccc2)cc1)c1cnc(-n2cccn2)c(O)c1]

Following general procedure D: 73 (12 mg, 0.031 mmol) and LiCl (19 mg, 0.31 mmol) in DMAc (2 ml) gave 75 (4 mg, 0.01 mmol, 35%).

¹H NMR (500 MHz, DMSO-d₆) δ 11.68 (s, 1H), 9.28 (t, J=6.0 Hz, 1H), 8.76 (d, J=2.5 Hz, 1H), 8.52 (d, J=2.0 Hz, 1H), 7.96 (d, J=2.0 Hz, 1H), 7.72-7.60 (m, 4H), 7.48-7.41 (m, 4H), 7.39-7.30 (m, 1H), 6.74-6.72 (m, 1H), 4.55 (d, J=6.0 Hz, 2H).

HRMS (ESI-TOF) calcd for C₂₂H₁₉02N₄ [M+H]⁺: 371.1502, found: 371.1503.

### Example 52 Synthesis of N-([1,1′-Biphenyl]-3-ylmethyl)-5-hydroxy-6-(1H-pyrazol-1-yl)nicotinamide (76)

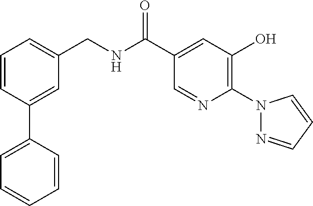 [SMILES: O=C(NCc1cccc(-c2ccccc2)c1)c1cnc(-n2cccn2)c(O)c1]

Following general procedure D: 74 (45 mg, 0.117 mmol) and LiCl (71 mg, 1.17 mmol) in DMAc (3.5 ml) gave 76 (14 mg, 0.037 mmol, 32%).

¹H NMR (400 MHz, DMSO-d₆) δ 11.68 (s, 1H), 9.28 (t, J=6.0 Hz, 1H), 8.76 (d, J=2.5 Hz, 1H), 8.52 (d, J=2.0 Hz, 1H), 7.97 (d, J=2.0 Hz, 1H), 7.71-7.32 (m, 10H), 6.75-6.71 (m, 1H), 4.59 (d, J=6.0 Hz, 2H).

HRMS (ESI-TOF) calcd for C₂₂H₁₉O₂N₄ [M+H]⁺: 371.1502, found: 371.1502.

### Example 53 Synthesis of Ethyl 1-(5-(([1,1′-biphenyl]-4-ylmethyl)carbamoyl)-4-methoxypyridin-2-yl)-1H-pyrazole-4-carboxylate (77)

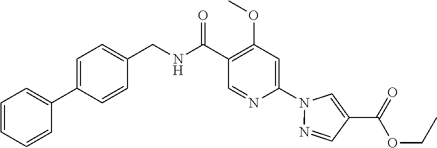 [SMILES: CCOC(=O)c1cnn(-c2cc(OC)c(C(=O)NCc3ccc(-c4ccccc4)cc3)cn2)c1]

Following general procedure C: 33 (150 mg, 0.43 mmol), Pd^{t}BuXPhos G3 (34 mg, 0.043 mmol), Cs₂CO₃ (354 mg, 1.09 mmol), pyrazole-4-carboxylate ethyl ester (89 mg, 0.649 mmol) and ^{t}BuOH (2 ml) gave 77 (93 mg, 0.20 mmol, 48%).

¹H NMR (400 MHz, DMSO-d₆) δ 9.37 (s, 1H), 9.00 (s, 1H), 8.85 (s, 1H), 8.24 (s, 1H), 7.69-7.62 (m, 4H), 7.54-7.29 (m, 6H), 4.59 (d, J=6.0 Hz, 2H), 4.28 (q, J=7.0 Hz, 2H), 4.09 (s, 3H), 1.31 (t, J=7.0 Hz, 3H).

HRMS (ESI-TOF) calcd for C₂₆H₂₅O₄N₄ [M+H]⁺: 457.1870, found: 457.1876.

### Example 54 Synthesis of N-([1,1′-Biphenyl]-4-ylmethyl)-6-(4-cyano-1H-pyrazol-1-yl)-4-methoxynicotinamide (78)

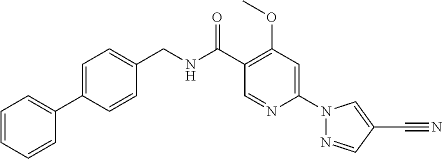 [SMILES: COc1cc(-n2cc(C#N)cn2)ncc1C(=O)NCc1ccc(-c2ccccc2)cc1]

Following general procedure C: 33 (150 mg, 0.426 mmol), Pd^{t}BuXPhos G3 (16 mg, 0.0213 mmol), Cs₂CO₃ (276 mg, 0.852 mmol), pyrazole-4-nitrile (47 mg, 0.511 mmol) and ^{t}BuOH (4 ml) gave 78 (9 mg, 0.022 mmol, 5%).

¹H NMR (400 MHz, DMSO-d₆) δ 9.47 (d, J=0.0 1 Hz, 1H), 8.87 (t, J=6.0 Hz, 1H), 8.66 (s, 1H), 8.48 (d, J=1.0 Hz, 1H), 7.70-7.31 (m, 10H), 4.55 (d, J=6.0 Hz, 2H), 4.09 (s, 3H).

HRMS (ESI-TOF) calcd for C₂₄H₁₈O₂N₅[M−H]: 408.1466, found: 408.1467.

### Example 55 Synthesis of Ethyl 1-(5-(([1,1′-biphenyl]-4-ylmethyl)carbamoyl)-3-methoxypyridin-2-yl)-1H-pyrazole-4-carboxylate (79)

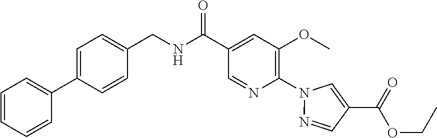 [SMILES: CCOC(=O)c1cnn(-c2ncc(C(=O)NCc3ccc(-c4ccccc4)cc3)cc2OC)c1]

Following general procedure C: 72 (150 mg, 0.43 mmol), Pd^{t}BuXPhos G3 (34 mg, 0.043 mmol), Cs₂CO₃ (354 mg, 1.09 mmol), pyrazole-4-carboxylate ethyl ester (89 mg, 0.649 mmol) and ^{t}BuOH (4 ml) gave 79 (44 mg, 0.096 mmol, 23%).

¹H NMR (400 MHz, DMSO-d₆) δ 9.41 (d, J=6.0 Hz, 1H), 8.78-8.75 (m, 1H), 8.64 (d, J=2.5 Hz, 1H), 8.15 (d, J=2.5 Hz, 1H), 7.67-7.61 (m, 5H), 7.53-7.42 (m, 4H), 7.39-7.34 (m, 1H), 4.59 (d, J=6.0 Hz, 2H), 4.27 (q, J=7.0 Hz, 2H), 4.00-3.94 (s, 3H), 1.30 (t, J=7.0 Hz, 3H).

HRMS (ESI-TOF) calcd for C₂₆H₂₅O₄N₄ [M+H]⁺: 457.1870, found: 457.1874.

### Example 56 Synthesis of Ethyl 1-(4-Methoxy-5-((4-(trifluoromethyl)benzyl)carbamoyl)pyridin-2-yl)-1H-pyrazole-4-carboxylate (80)

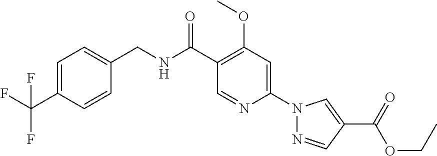 [SMILES: CCOC(=O)c1cnn(-c2cc(OC)c(C(=O)NCc3ccc(C(F)(F)F)cc3)cn2)c1]

Following general procedure C: 45 (100 mg, 0.29 mmol), RockPhos Pd G3 (20 mg, 0.029 mmol), Cs₂CO₃ (188 mg, 0.58 mmol), 4-ethyl ester pyrazole (24 mg, 0.63 mmol) and ^{t}BuOH (4 ml) gave 80 (86 mg, 0.192 mmol, 66%).

¹H NMR (400 MHz, Chloroform-d) δ 9.12 (s, 1H), 9.07 (d, J=1.0 Hz, 1H), 8.11 (d, J=1.0 Hz, 1H), 7.89 (t, J=6.0 Hz, 1H), 7.63 (s, 1H), 7.62-7.58 (m, 2H), 7.50-7.45 (m, 2H), 4.73 (d, J=6.0 Hz, 2H), 4.35 (q, J=7.0 Hz, 2H), 4.11 (s, 3H), 1.37 (t, J=7.0 Hz, 3H).

HRMS (ESI-TOF) calcd for C₂₁H₁₈O₄N₄F₃ [M−H]: 447.1285, found: 447.1281.

### Example 57 Synthesis of 6-(4-Cyano-1H-pyrazol-1-yl)-4-methoxy-N-(4-(trifluoromethyl)benzyl)nicotinamide (81)

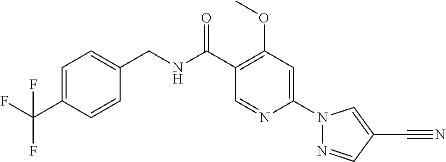 [SMILES: COc1cc(-n2cc(C#N)cn2)ncc1C(=O)NCc1ccc(C(F)(F)F)cc1]

Following general procedure C: 45 (100 mg, 0.29 mmol), RockPhos Pd G3 (24 mg, 0.029 mmol), Cs₂CO₃ (180 mg, 0.554 mmol), pyrazole-4-nitrile (24 mg, 0.35 mmol) and ^{t}BuOH (4 ml) gave 81 (23 mg, 0.057 mmol, 20%).

¹H NMR (400 MHz, Chloroform-d) δ 9.13 (s, 1H), 9.04 (d, J=1.0 Hz, 1H), 7.99 (d, J=1.0 Hz, 1H), 7.88 (t, J=6.0 Hz, 1H), 7.67-7.56 (m, 3H), 7.51-7.42 (m, 2H), 4.74 (d, J=6.0 Hz, 2H), 4.03 (s, 3H).

HRMS (ESI-TOF) calcd for C₁₈H₁₅04N₄F₃[M+H]⁺: 402.1172, found: 402.1172.

### Example 58 Synthesis of Ethyl 1-(4-methoxy-5-(((6-phenylpyridin-3-yl)methyl)carbamoyl)pyridin-2-yl)-1H-pyrazole-4-carboxylate (82)

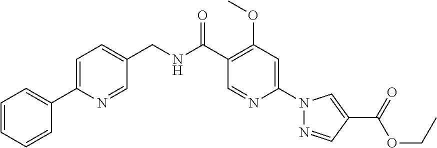 [SMILES: CCOC(=O)c1cnn(-c2cc(OC)c(C(=O)NCc3ccc(-c4ccccc4)nc3)cn2)c1]

Following general procedure C: 53 (80 mg, 0.226 mmol), RockPhosPd G3 (19 mg, 0.0226 mmol), Cs₂CO₃ (145 mg, 0.452 mmol), 4-ethyl ester pyrazole (31 mg, 0.226 mmol) and ^{t}BuOH (2.5 ml) gave 82 (11 mg, 0.024 mmol, 11%).

¹H NMR (400 MHz, Chloroform-d) δ 9.13 (s, 1H), 9.08 (s, 1H), 8.70-8.68 (m, 1H), 8.12 (s, 1H), 8.01-7.94 (m, 2H), 7.90 (t, J=6.0 Hz, 1H), 7.84-7.37 (m, 6H), 4.73 (d, J=6.0 Hz, 2H), 4.32 (q, J=7.0 Hz, 2H), 4.11 (s, 3H), 1.37 (t, J=7.0 Hz, 3H).

HRMS (ESI-TOF) calcd for C₂₅H₂₄O₄N₅[M+H]⁺: 458.1822, found: 458.1816.

### Example 59 Synthesis of Ethyl 1-(5-(([1,1′-biphenyl]-4-ylmethyl)carbamoyl)-4-hydroxypyridin-2-yl)-1H-pyrazole-4-carboxylate (83)

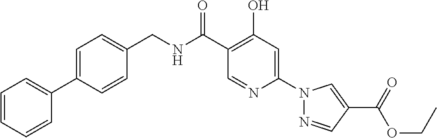 [SMILES: CCOC(=O)c1cnn(-c2cc(O)c(C(=O)NCc3ccc(-c4ccccc4)cc3)cn2)c1]

Following general procedure D: 80 (82 mg, 0.179 mmol) and LiCl (mg, 1.79 mmol) in DMAc (35 ml) gave 83 (70 mg, 0.158 mmol, 90%).

¹H NMR (400 MHz, DMSO-d₆) δ 9.42 (s, 1H), 9.00 (s, 1H), 8.85 (s, 1H), 8.24 (s, 1H), 7.72-7.58 (m, 4H), 7.54-7.39 (m, 5H), 7.38-7.29 (m, 1H), 4.59 (d, J=6.0 Hz, 2H,), 4.27 (q, J=7.0 Hz, 2H), 1.30 (t, J=7.0 Hz, 3H).

HRMS (ESI-TOF) calcd for C₂₅H₂₃O₄N₄[M+H]⁺: 443.1713, found: 443.1713.

### Example 60 Synthesis of 1-(5-(([1,1′-Biphenyl]-4-ylmethyl)carbamoyl)-4-hydroxypyridin-2-yl)-1H-pyrazole-4-carboxylic acid (84)

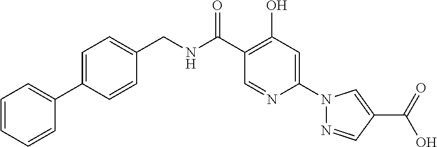 [SMILES: O=C(O)c1cnn(-c2cc(O)c(C(=O)NCc3ccc(-c4ccccc4)cc3)cn2)c1]

83 (65 mg, 0.147 mmol) was dissolved in THE (5 ml) and water (0.5 ml) was added to the reaction mixture before the addition of LiOH-monohydrate (16 mg, 0.40 mmol). The resultant mixture was stirred overnight at room temperature. The reaction mixture was acidified to pH 3 with HCL_{aq} (1M) and extracted with EtOAc (3×25 ml), washed with brine dried with anhydrous Na₂SO₄. The crude compound was then purified using flash column chromatography using (CH₂Cl₂: MeOH 0-20%: Formic Acid 1%) over 20 columns volumes gave 84 (52 mg, 0.125 mmol, 85%) as an off-white solid.

¹H NMR (400 MHz, DMSO-d₆) δ 13.34 (s, 1H), 9.42 (s, 1H), 9.00 (s, 1H), 8.96 (s, 1H), 8.19 (s, 1H), 7.71-7.31 (m, 11H), 4.59 (d, J=6.0 Hz, 2H).

HRMS (ESI-TOF) calcd for C₂₃H₁₉04N₄[M+H]⁺: 415.1400, found: 415.1401.

### Example 61 Synthesis of N-([1,1′-Biphenyl]-4-ylmethyl)-6-(4-cyano-]H-pyrazol-1-yl)-4-hydroxynicotinamide (85)

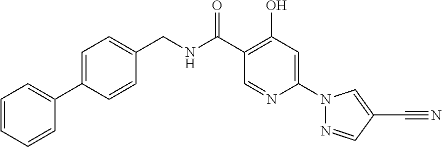 [SMILES: N#Cc1cnn(-c2cc(O)c(C(=O)NCc3ccc(-c4ccccc4)cc3)cn2)c1]

78 (6 mg, 0.0146 mmol) was dissolved in DMAc (1.5 ml) in a microwave vial, before the addition of Cs₂CO₃ (14.3 mg, 0.044 mmol). The resultant reaction mixture was heated under microwave irradiation at 130° C. for 1 hr. H₂O (50 ml) was added, and the resultant mixture was extracted with EtOAc (3×15 ml). The combined organic fractions were washed with brine, dried over Na₂SO₄ and the solvents were removed in vacuo. The crude compound was then purified using flash column chromatography using (CH₂Cl₂: MeOH 0-10%) over 20 column volumes to give 85 (2.5 mg, 0.006 mmol, 44%).

¹H NMR (400 MHz, DMSO-d₆) δ 9.45 (s, 1H), 8.87 (d, J=2.0 Hz, 1H), 8.44 (s, 1H), 7.73-7.42 (m, 11H), 4.60 (d, J=6.0 Hz, 2H).

HRMS (ESI-TOF) calcd for C₂₃H₁₆02N₅ [M−H]: 394.1309, found: 394.1309.

### Example 62 Synthesis of 1-(5-(([1,1′-Biphenyl]-4-ylmethyl)carbamoyl)-3-hydroxypyridin-2-yl)-]H-pyrazole-4-carboxylic acid (86)

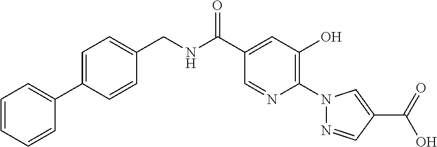 [SMILES: O=C(O)c1cnn(-c2ncc(C(=O)NCc3ccc(-c4ccccc4)cc3)cc2O)c1]

79 (20 mg, 0.043 mmol) was dissolved in THE (2 ml), MeOH (2 ml) and water (0.5 ml) was added to the reaction mixture before the addition of LiOH-monohydrate (16 mg, 0.40 mmol). The resultant mixture was stirred overnight at room temperature. The reaction mixture was acidified to PH3 with HCl_{aq} (1M) solution and extracted with EtOAc (3×25 ml), washed with brine dried with anhydrous Na₂SO₄. The crude compound was then purified using flash column chromatography using (CH₂Cl₂: MeOH 0-20%: formic acid 1%) over 20 column volumes to give 86 (8 mg, 0.019 mmol, 45%).

¹H NMR (400 MHz, DMSO-d₆) δ 9.09 (s, 1H), 8.90 (d, J=6.0 Hz, 1H), 7.92-7.09 (m, 12H), 4.47 (d, J=6.0 Hz, 2H).

HRMS (ESI-TOF) calcd for C₂₃H₁₇04N₄ [M−H]: 413.1255, found: 413.1252.

### Example 63 Synthesis of Ethyl 1-(4-hydroxy-5-((4-(trifluoromethyl)benzyl)carbamoyl)pyridin-2-yl)-]H-pyrazole-4-carboxylate (87)

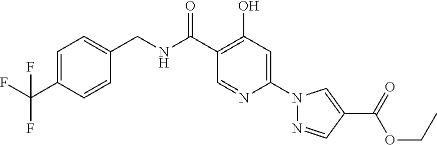 [SMILES: CCOC(=O)c1cnn(-c2cc(O)c(C(=O)NCc3ccc(C(F)(F)F)cc3)cn2)c1]

Following general procedure D: 80 (68 mg, 0.151 mmol) and LiCl (91 mg, 1.51 mmol) in DMAc (3.5 ml) gave 87 (48 mg, 0.110 mmol, 73%).

¹H NMR (400 MHz, THF-d₈) δ 13.46 (s, 1H), 8.99 (d, J=1.0 Hz, 1H), 8.93-8.85 (m, 1H), 8.74 (s, 1H), 8.07 (d, J=1.0 Hz, 1H), 7.65 (d, J=7.0 Hz, 2H), 7.58 (d, J=7.0 Hz, 2H), 7.48 (s, 1H), 4.71 (d, J=6.0 Hz, 2H), 4.29 (q, J=7.0 Hz, 2H), 1.33 (t, J=7.0 Hz, 3H).

HRMS (ESI-TOF) calcd for C₂₀H₁₆04N₄F₃[M−H]: 433.1129, found: 433.1125.

### Example 64 Synthesis of Ethyl 1-(4-hydroxy-5-(((6-phenylpyridin-3-yl)methyl)carbamoyl)pyridin-2-yl)-]H-pyrazole-4-carboxylate (88)

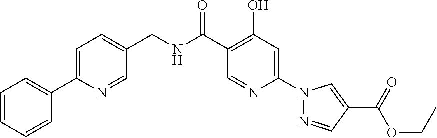 [SMILES: CCOC(=O)c1cnn(-c2cc(O)c(C(=O)NCc3ccc(-c4ccccc4)nc3)cn2)c1]

Following general procedure D: 82 (7 mg, 0.015 mmol) and LiCl (9 mg, 0.15 mmol) in DMAc (2 ml) gave 88 (3.5 mg, 0.0079 mmol, 53%).

¹H NMR (400 MHz, DMSO-d₆) δ 9.00 (s, 1H), 8.80 (s, 1H), 8.67 (d, J=2.0 Hz, 1H), 8.24 (s, 1H), 8.07 (dd, J=8.0, 2.0 Hz, 2H), 7.95 (d, J=8.0 Hz, 1H), 7.85 (dd, J=8.0, 2.0 Hz, 1H), 7.54-7.39 (m, 5H), 4.61 (d, J=6.0 Hz, 2H), 4.26 (q, J=7.0 Hz, 2H), 1.30 (t, J=7.0 Hz, 3H).

HRMS (ESI-TOF) calcd for C₂₄H₂₂04N₅[M+H]⁺: 444.1666, found: 444.1659.

### Example 65 Synthesis of 6-(4-Cyano-]H-pyrazol-1-yl)-4-hydroxy-N-(4-(trifluoromethyl)benzyl)nicotinamide (89)

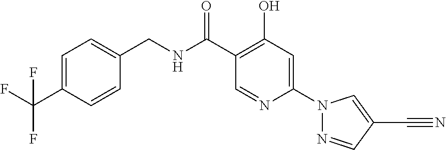 [SMILES: N#Cc1cnn(-c2cc(O)c(C(=O)NCc3ccc(C(F)(F)F)cc3)cn2)c1]

Following general procedure D: 81 (20 mg, 0.049 mmol) and LiCl (30 mg, 0.50 mmol) in DMAc (2 ml) gave 89 (5 mg, 0.012 mmol, 26%).

¹H NMR (400 MHz, DMSO-d₆) δ 9.43 (d, J=1.0 Hz, 1H), 8.81 (s, 1H), 8.43 (d, J=1.0 Hz, 1H), 7.71 (d, J=8.0 Hz, 2H), 7.57 (d, J=8.0 Hz, 2H), 7.42 (s, 1H), 4.63 (d, J=6.0 Hz, 2H).

HRMS (ESI-TOF) calcd for C₁₈H₁₁04N₄F₃[M−H]: 386.0870, found: 386.0867.

### Example 66 Synthesis of 1-(4-Hydroxy-5-((4-(trifluoromethyl)benzyl)carbamoyl)pyridin-2-yl)-]H-pyrazole-4-carboxylic acid (90)

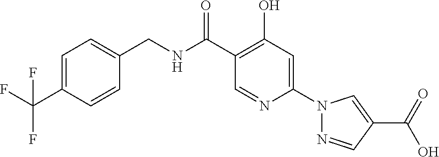 [SMILES: O=C(O)c1cnn(-c2cc(O)c(C(=O)NCc3ccc(C(F)(F)F)cc3)cn2)c1]

87 (40 mg, 0.092 mmol) was dissolved in THE (2 ml), MeOH (2 ml) and water (0.5 ml) was added to the reaction mixture before the addition of LiOH-monohydrate (19 mg, 0.46 mmol). The resultant mixture was stirred overnight at room temperature. The reaction mixture was acidified to pH 3 with HCL_{aq} (1M) and extracted with EtOAc (3×25 ml), washed with brine, dried with anhydrous Na₂SO₄. The crude compound was then purified using flash column chromatography using (CH₂Cl₂: MeOH 0-20%: Formic acid 1%) over 20 column volumes gave 90 (17 mg, 0.042 mmol, 46%).

¹H NMR (400 MHz, DMSO-d₆) δ 9.56 (s, 1H), 8.94 (s, 1H), 8.78 (s, 1H), 8.17 (s, 1H), 7.70 (d, J=8.0 Hz, 2H), 7.56 (d, J=8.0 Hz, 2H), 7.38 (s, 1H), 4.63 (d, J=6.0 Hz, 2H).

HRMS (ESI-TOF) calcd for C₁₈H₁₂O₄N₄F₃ [M−H]: 405.0816, found: 405.0815.

### Example 67 Synthesis of]-(4-Hydroxy-5-(((6-phenylpyridin-3-yl)methyl)carbamoyl)pyridin-2-yl)-1H-pyrazole-4-carboxylic acid (9])

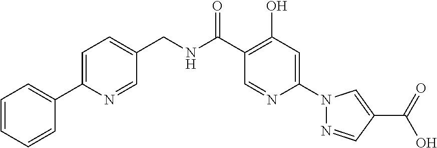 [SMILES: O=C(O)c1cnn(-c2cc(O)c(C(=O)NCc3ccc(-c4ccccc4)nc3)cn2)c1]

88 (3 mg, 0.0072 mmol) was dissolved in THE (0.8 ml), MeOH (0.15 ml) and water (0.05 ml) was added to the reaction mixture before the addition of LiOH-monohydrate (1 mg, 0.023 mmol). The resultant mixture was stirred overnight at room temperature. The reaction mixture was acidified to pH 3 with HCl_{aq} (1M) and was using preparative HPLC to give 91 (1 mg, 0.0024 mmol, 33%).

¹H NMR (500 MHz, DMSO-d₆) δ 8.94 (s, 1H), 8.68 (s, 1H), 8.19 (s, 1H), 8.13-8.05 (m, 2H), 7.96 (d, J=8.5 Hz, 1H), 7.87 (d, J=8.5 Hz, 1H), 7.55-7.39 (m, 6H), 4.62 (d, J=6.0 Hz, 2H).

HRMS (ESI-TOF) calcd for C₂₂H₁₈O₄N₅[M+H]⁺: 416.1353, found: 416.1352.

### Reference Example 68 Synthesis of N-([1,1′-Biphenyl]-4-ylmethyl)-]H-pyrazole-4-carboxamide (92)

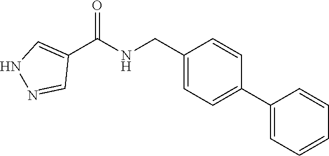 [SMILES: O=C(NCc1ccc(-c2ccccc2)cc1)c1cn[nH]c1]

Following general procedure B: 1H-pyrazole-4-carboxylic acid (1 g, 8.9 mmol), 4-phenylbenzylamine (2.44 g, 13.35 mmol), T3P (7.05 g, 22.2 mmol) and DIPEA (4.6 g, 35.7 mmol) gave 92 (462 mg, 1.66 mmol, 19%).

¹H NMR (400 MHz, DMSO-d₆) δ 8.66 (t, J=6.0 Hz, 1H), 8.09 (s, 2H), 7.70-7.58 (m, 4H), 7.50-7.30 (m, 5H,), 4.47 (d, J=6.0 Hz, 2H).

HRMS (ESI-TOF) calcd for C₁₇H₁₆ON₃ [M+H]⁺: 278.1287, found: 278.1290.

### Example 69 Synthesis of N-([1,1′-Biphenyl]-4-ylmethyl)-1-(5-cyano-4-methoxypyridin-2-yl)-]H-pyrazole-4-carboxamide (93)

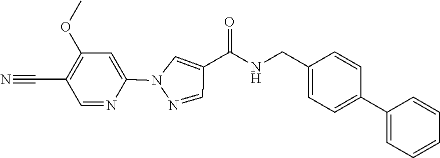 [SMILES: COc1cc(-n2cc(C(=O)NCc3ccc(-c4ccccc4)cc3)cn2)ncc1C#N]

General Procedure C: 6-chloro-4-methoxynicotinonitrile (40 mg, 0.238 mmol), 92 (65 mg, 0.238 mmol), Pd^{t}BuXPhos Pd G3 (19 mg, 0.023 mmol) and Cs₂CO₃ (232 mg, 0.714 mmol) in gave 93 (14 mg, 0.034 mmol, 12%).

¹H NMR (400 MHz, DMSO-d₆) δ 9.03 (t, J=6.0 Hz, 1H), 8.82 (s, 1H), 8.30-8.29 (m, 1H), 7.70-7.59 (m, 5H), 7.50-7.31 (m, 6H), 4.50 (d, J=6.0 Hz, 2H), 4.12 (s, 3H).

HRMS (ESI-TOF) calcd for C₂₄H₂₀O₂N₅ [M+H]⁺: 410.1622, found: 410. 1611.

### Example 70 Synthesis of Ethyl 6-(4-(([1,1′-Biphenyl]-4-ylmethyl)carbamoyl)-]H-pyrazol-1-yl)-4-methoxynicotinate (94)

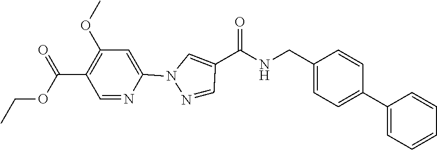 [SMILES: CCOC(=O)c1cnc(-n2cc(C(=O)NCc3ccc(-c4ccccc4)cc3)cn2)cc1OC]

Following general procedure C: Ethyl 6-chloro-4-methoxynicotinate (39 mg, 0.18 mmol), 92 (50 mg, 0.18 mmol), Pd^{t}BuXPhos Pd G3 (25 mg, 0.009 mmol) and dioxane (3 ml) gave 94 as a clear oil (23 mg, 0.05 mmol, 28%).

¹H NMR (400 MHz, THF-d₈) δ 9.08 (d, J=1.0 Hz, 1H), 8.68 (s, 1H), 8.13 (d, J=1.0 Hz, 1H), 8.05 (t, J=6.0 Hz, 1H), 7.69 (s, 1H), 7.64-7.23 (m, 9H), 4.59 (d, J=6.0 Hz, 2H), 4.30 (q, J=7.0 Hz, 2H), 4.03 (s, 3H), 1.34 (t, J=7.0 Hz, 3H).

HRMS (ESI-TOF) calcd for C₂₆H₂₅O₄N₄ [M+H]⁺: 457.1870, found: 457.1868.

### Example 71 Synthesis of N-([1,1′-Biphenyl]-4-ylmethyl)-1-(5-cyano-4-hydroxypyridin-2-yl)-]H-pyrazole-4-carboxamide (95)

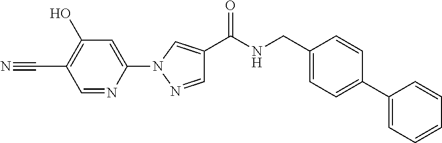 [SMILES: N#Cc1cnc(-n2cc(C(=O)NCc3ccc(-c4ccccc4)cc3)cn2)cc1O]

Following general procedure D: 93 (6 mg, 0.014 mmol) and LiCl (8.5 mg, 0.14 mmol) in DMAc (1 ml) gave 95 (1.9 mg, 0.0048 mmol, 35%).

¹H NMR (400 MHz, DMSO-d₆) δ 9.14 (s, 1H), 8.95 (t, J=6.0 Hz, 1H), 8.45 (s, 1H), 8.16 (s, 1H), 7.70-7.59 (m, 4H), 7.53-7.29 (m, 5H), 7.16 (s, 1H), 4.48 (d, J=6.0 Hz, 2H).

HRMS (ESI-TOF) calcd for C₂₃H₁₈02N₅ [M+H]⁺: 396.1466, found: 396.1456.

### Example 72 Synthesis of 6-(4-(([1,1′-Biphenyl]-4-ylmethyl)carbamoyl)-]H-pyrazol-1-yl)-4-hydroxynicotinic acid (96)

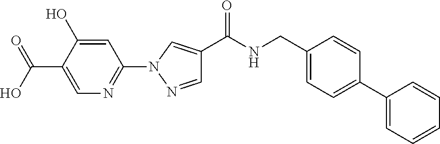 [SMILES: O=C(NCc1ccc(-c2ccccc2)cc1)c1cnn(-c2cc(O)c(C(=O)O)cn2)c1]

Following general procedure D: 94 (23 mg, 0.0504 mmol) and LiCl (30 mg, 0.504 mmol) in DMAc (3 ml) gave 96 as a yellow oil (9 mg, 0.0216 mmol, 43%).

¹H NMR (600 MHz, DMSO-d₆) δ 9.21 (s, 1H), 8.96 (t, J=6.0 Hz, 1H), 8.71 (s, 1H), 8.22 (s, 1H), 7.64 (m, 5H), 7.51-7.32 (m, 5H), 4.49 (d, J=6.0 Hz, 2H).

HRMS (ESI-TOF) calcd for C₂₃H₁₉04N₄ [M+H]⁺: 415.1400, found: 415.1401.

### Example 73 Synthesis of N-([1,1′-Biphenyl]-4-ylmethyl)-1-(5-cyanopyridin-2-yl)-]H-pyrazole-4-carboxamide (97)

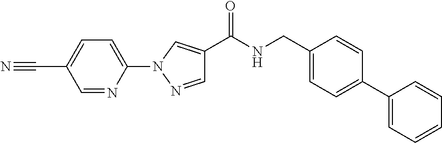 [SMILES: N#Cc1ccc(-n2cc(C(=O)NCc3ccc(-c4ccccc4)cc3)cn2)nc1]

Following general procedure C: 6-chloro-nicotinonitrile (30 mg, 0.216 mmol), 92 (30 mg, 0.108 mmol), PdtBuXPhos G3 (17 mg, 0.0216 mmol), Cs₂CO₃ (175 mg, 0.432 mmol) gave 97 (6 mg, 0.015 mmol, 16%).

¹H NMR (400 MHz, DMSO-d₆) δ 9.27 (d, J=1.0 Hz, 1H), 9.10-8.96 (m, 2H), 8.51 (dd, J=8.5, 2.0 Hz, 1H), 8.29 (d, J=1.0 Hz, 1H), 8.10 (dd, J=8.5, 1.0 Hz, 1H), 7.67-7.59 (m, 4H), 7.51-7.31 (m, 5H), 4.50 (d, J=6.0 Hz, 2H).

HRMS (ESI-TOF) calcd for C₂₃H₁₆0N₅ [M−H]: 378.1360, found: 378.1354.

### Example 74 Synthesis of N-([1,1′-Biphenyl]-4-ylmethyl)-1-(4-methoxypyridin-2-yl)-]H-pyrazole-4-carboxamide (98)

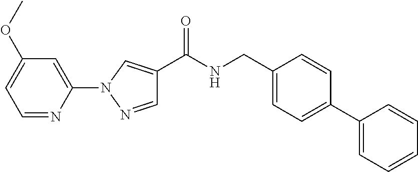 [SMILES: COc1ccnc(-n2cc(C(=O)NCc3ccc(-c4ccccc4)cc3)cn2)c1]

Following general procedure C: 2-chloro-4-methoxypyridine (16 mg, 0.114 mmol), 92 (31 mg, 0.114 mmol), RockPhos Pd G3 (9.5 mg, 0.0114 mmol) and Cs₂CO₃ (92 mg, 0.285 mmol) gave 98 (28 mg, 0.072 mmol, 63%).

¹H NMR (400 MHz, DMSO-d₆) δ 9.18 (d, J=1.0 Hz, 1H), 8.95 (t, J=6.0 Hz, 1H), 8.32 (d, J=6.0 Hz, 1H), 8.19 (d, J=1.0 Hz, 1H), 7.69-7.31 (m, 10H), 7.00 (dd, J=6.0, 2.5 Hz, 1H), 4.49 (d, J=6.0 Hz, 2H), 3.93 (s, 3H).

HRMS (ESI-TOF) calcd for C₂₃H₂₁O₂N₄ [M+H]⁺: 385.1690, found: 385.1659.

### Example 75 Synthesis of N-([1,1′-Biphenyl]-4-ylmethyl)-1-(4-hydroxypyridin-2-yl)-1H-pyrazole-4-carboxamide (99)

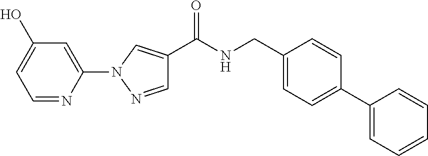 [SMILES: O=C(NCc1ccc(-c2ccccc2)cc1)c1cnn(-c2cc(O)ccn2)c1]

Following general procedure D: 92 (7 mg, 0.018 mmol) and LiCl (11 mg, 0.18 mmol) dissolved in DMAc (1 ml) gave 99 (3 mg, 0.008 mmol, 45%).

¹H NMR (400 MHz, DMSO-d₆) δ 9.14 (d, J=1.0 Hz, 1H), 8.89 (t, J=6.0 Hz, 1H), 8.73-8.66 (m, 1H), 8.33-8.29 (m, 1H), 7.91-7.89 (m, 1H), 7.50-7.28 (m, 9H), 6.77 (dd, J=6.0, 2.5 Hz, 1H), 4.48 (d, J=6.0 Hz, 2H).

HRMS (ESI-TOF) calcd for C₂₂H₁₉O₂N₄ [M+H]⁺: 371.1506, found: 371.1506.

### Example 76 Synthesis of Ethyl 6-(4-(([1,1′-biphenyl]-4-ylmethyl)carbamoyl)-]H-pyrazol-1-yl)nicotinate (100)

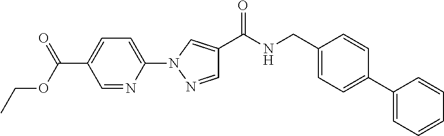 [SMILES: CCOC(=O)c1ccc(-n2cc(C(=O)NCc3ccc(-c4ccccc4)cc3)cn2)nc1]

Following general procedure C: Ethyl 6-chloronicotinate (42 mg, 0.23 mmol), 92 (30 mg, 0.108 mmol), PdtBuXPhos G3 (18 mg, 0.023 mmol), Cs₂CO₃ (149 mg, 0.46 mmol) in dioxane (5 ml) gave 100 as a (10 mg, 0.023 mmol, 21%).

¹H NMR (400 MHz, THF-d₈) δ 9.09 (d, J=1.0 Hz, 1H), 9.00 (dd, J=2.0, 1.0 Hz, 1H), 8.70-8.60 (m, 1H), 8.14-8.10 (m, 1H), 7.96 (t, J=6.0 Hz, 1H), 7.67-7.35 (m, 10H,), 4.59 (d, J=6.0 Hz, 2H), 4.40 (q, J=7.0 Hz, 2H), 1.38 (t, J=7.0 Hz, 3H).

HRMS (ESI-TOF) calcd for C₂₅H₂₃03N₄ [M+H]⁺: 427.1766, found: 427.1766.

### Example 77 Synthesis of 6-(4-(([1,1′-Biphenyl]-4-ylmethyl)carbamoyl)-]H-pyrazol-1-yl)nicotinic acid (101)

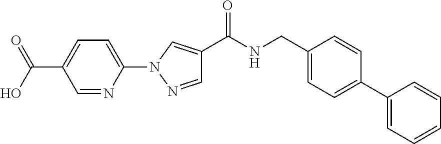 [SMILES: O=C(O)c1ccc(-n2cc(C(=O)NCc3ccc(-c4ccccc4)cc3)cn2)nc1]

100 (8 mg, 0.0187 mmol) was dissolved in a mixture of THE and water (3 ml (10:1). Lithium hydroxide monohydrate (1.5 mg, 0.0374 mmol) was added to the reaction mixture and the resulting mixture was allowed to stir at room temperature for 16 hrs. The reaction was confirmed to have gone to completion by TLC and HCl_{aq} (5 ml, 1M) was added to the reaction mixture. The resulting mixture was extracted with EtOAc (3×10 ml), then washed with brine, then dried with anhydrous Na₂SO₄ and purified using flash column chromatography using (CH₂Cl₂, MeOH 0-5%, 1% formic acid) gave 101 (2 mg, 0.005 mmol, 27%).

¹H NMR (400 MHz, DMSO-d₆) δ 9.26 (d, J=1.0 Hz, 1H), 8.97 (t, J=6.0 Hz, 1H), 8.87 (s, 1H), 8.35 (d, J=2.0 Hz, 1H), 8.19 (d, J=7.0 Hz, 1H), 7.89 (d, J=7.0 Hz, 1H), 7.68-7.55 (m, 4H), 7.52-7.29 (m, 5H), 4.49 (d, J=6.0 Hz, 2H).

HRMS (ESI-TOF) calcd for C₂₃H₁₇03N₄ [M−H]: 397.1304, found: 397.1304.

### Example 78 Synthesis of Methyl 2-(4-(([1,1′-Biphenyl]-4-ylmethyl)carbamoyl)-]H-pyrazol-1-yl)isonicotinate (102)

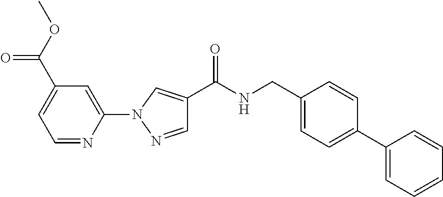 [SMILES: COC(=O)c1ccnc(-n2cc(C(=O)NCc3ccc(-c4ccccc4)cc3)cn2)c1]

Following general procedure C: Methyl 2-chloroisonictinate (37 mg, 0.216 mmol), 92 (30 mg, 0.108 mmol), ^{t}BuXPhos Pd G3 (17 mg, 0.0216 mmol) and Cs₂CO₃ (175 mg, 0.54 mmol) in dioxane (3 ml) gave 102 (6 mg, 0.014 mmol, 13%).

¹H NMR (400 MHz, DMSO-d₆) δ 9.24 (d, J=1.0 Hz, 1H), 8.99 (t, J=6.0 Hz, 1H), 8.73 (dd, J=5.0, 1.0 Hz, 1H), 8.34 (dd, J=1.5, 1.0 Hz, 1H), 8.26 (d, J=1.0 Hz, 1H), 7.85 (dd, J=5.0, 1.5 Hz, 1H), 7.66-7.61 (m, 4H), 7.51-7.29 (m, 5H), 4.50 (d, J=6.0 Hz, 2H), 3.95 (s, 3H).

HRMS (ESI-TOF) calcd for C₂₄H₂₁O₃N₄ [M+H]⁺: 413.1608, found: 413.1608.

### Example 79 Synthesis of 2-(4-(([1,1′-Biphenyl]-4-ylmethyl)carbamoyl)-]H-pyrazol-1-yl)isonicotinic acid (103)

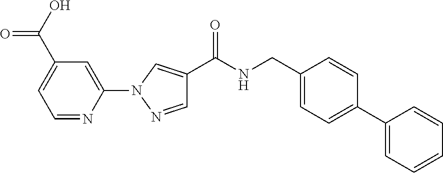 [SMILES: O=C(O)c1ccnc(-n2cc(C(=O)NCc3ccc(-c4ccccc4)cc3)cn2)c1]

102 (4 mg, 0.0097 mmol) was dissolved in a mixture of THE and water (3 ml (10:1). Lithium hydroxide monohydrate (1 mg, 0.0194 mmol) was added to the reaction mixture and the resulting mixture was allowed to stir at room temperature for 16 hrs. The reaction was confirmed to have gone to completion by LCMS and HCla_{q}(5 ml, 1M) was added to the reaction mixture. The resultant mixture was extracted with EtOAc (3×10 ml), then washed with brine, dried with anhydrous Na₂SO₄ and purified using flash column chromatography using (CH₂Cl₂, MeOH 0-5%, formic acid 1%) over 20 column volumes gave 103 (3 mg, 0.0075 mmol, 78%).

¹H NMR (400 MHz, DMSO-d₆) δ 9.24 (s, 1H), 8.98 (t, J=6.0 Hz, 1H), 8.68 (d, J=5.0 Hz, 1H), 8.33 (t, J=1.5 Hz, 1H), 8.24 (s, 1H), 7.81 (dd, J=5.0, 1.5 Hz, 1H), 7.69-7.61 (m, 4H), 7.50-7.37 (m, 5H), 4.50 (d, J=6.0 Hz, 2H).

HRMS (ESI-TOF) calcd for C₂₃H₁₇03N₄ [M−H]: 397.1304, found: 397.1304.

### Reference Example 80 Synthesis of Ethyl 1-(4-methoxypyridin-2-yl)-1H-pyrazole-4-carboxylate (104)

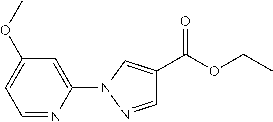 [SMILES: CCOC(=O)c1cnn(-c2cc(OC)ccn2)c1]

Following general procedure C: 4-methoxy-2-chloropyridine (500 mg, 3.49 mmol), 4-ethyl ester pyrazole (725 mg, 5.2 mmol), RockPhos Pd G3 (244 mg, 0.349 mmol), Cs₂CO₃ (2.8 g, 8.7 mmol) and ^{t}BuOH (10 ml) gave 104 (190 mg, 0.769 mmol, 22%).

¹H NMR (400 MHz, THF-d₈) δ 8.23 (d, J=5.5 Hz, 1H), 8.13 (d, J=5.5 Hz, 1H), 7.57 (d, J=2.0 Hz, 1H), 6.94 (d, J=2.0 Hz, 2H), 4.29 (q, J=7.0 Hz, 2H), 3.94 (s, 3H), 1.33 (t, J=7.0 Hz, 3H).

HRMS (ESI-TOF) calcd for C₁₂H₁₄03N₃ [M+H]⁺: 248.1029, found: 248.1033.

### Reference Example 81 Synthesis of 1-(4-Methoxypyridin-2-yl)-]H-pyrazole-4-carboxylic acid (105)

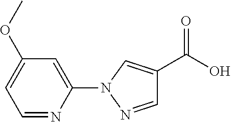 [SMILES: COc1ccnc(-n2cc(C(=O)O)cn2)c1]

104 (190 mg, 0.769 mmol) was dissolved in a mixture of THE and water (11 ml (10:1)). Lithium hydroxide monohydrate (78 mg, 2.19 mmol) was added to the reaction mixture and the resultant mixture was allowed to stir at room temperature for 16 hrs. The reaction was confirmed to have gone to completion by LCMS and HCl (1M, 5 ml) was added to the reaction mixture. The resultant mixture was extracted with EtOAc (3×10 ml), then washed with brine, then dried over Na₂SO₄. The solvents were removed in vacuo to give 105 (73 mg, 0.33 mmol, 44%).

¹H NMR (400 MHz, DMSO-d₆) δ 8.90 (s, 1H), 8.34 (d, J=6.0 Hz, 1H), 8.15 (s, 1H), 7.45 (d, J=2.5 Hz, 1H), 7.03 (dd, J=6.0, 2.5 Hz, 1H), 3.93 (s, 3H).

HRMS (ESI-TOF) calcd for C₁₀H₈O₃N₃ [M−H]: 218.0569, found: 218.0571.

### Example 82 Synthesis of N-([1,1′-Biphenyl]-4-yl)-1-(4-methoxypyridin-2-yl)-]H-pyrazole-4-carboxamide (106)

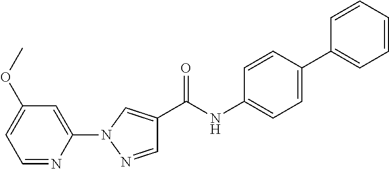 [SMILES: COc1ccnc(-n2cc(C(=O)Nc3ccc(-c4ccccc4)cc3)cn2)c1]

Following general procedure B: 105 (30 mg, 0.136 mmol), 4-aminobiphenyl (35 mg, 0.204 mmol), T3P (108 mg, 0.34 mmol) and DIPEA (116 μl, 0.68 mmol) gave 106 (27 mg, 0.072 mmol, 54%).

¹H NMR (400 MHz, THF-d₈) δ 9.29 (s, 1H), 9.18 (d, J=1.0 Hz, 1H), 8.24 (d, J=6.0 Hz, 1H), 8.17 (d, J=1.0 Hz, 1H), 7.91-7.84 (m, 2H), 7.68-7.21 (m, 8H), 6.87 (d, J=6.0 Hz, 1H), 3.95 (s, 3H).

HRMS (ESI-TOF) calcd for C₂₂H₁₉O₂N₄ [M+H]⁺: 371.1502, found: 371.1396.

### Example 83 Synthesis of N-([1,1′-Biphenyl]-3-ylmethyl)-1-(4-methoxypyridin-2-yl)-1H-pyrazole-4-carboxamide (107)

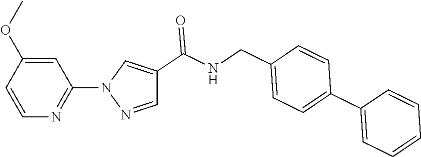 [SMILES: COc1ccnc(-n2cc(C(=O)NCc3ccc(-c4ccccc4)cc3)cn2)c1]

Following general procedure B: 105 (30 mg, 0.136 mmol), 3-aminomethylbiphenyl (36 mg, 0.204 mmol), T3P (108 mg, 0.34 mmol) and DIPEA (116 μl, 0.68 mmol) gave 107 (32 mg, 0.083 mmol, 61%).

¹H NMR (400 MHz, Chloroform-d) δ 8.97 (d, J=1.0 Hz, 1H), 8.15 (d, J=6.0 Hz, 1H), 8.07 (d, J=1.0 Hz, 1H), 7.58-7.28 (m, 10H), 6.73 (d, J=6.0 Hz, 1H), 6.59 (t, J=6.0 Hz, 1H), 4.64 (d, J=6.0 Hz, 2H), 3.90 (s, 3H).

HRMS (ESI-TOF) calcd for C₂₃H₂₁O₂N₄ [M+H]⁺: 385.1659, found: 385.1656.

### Example 84 Synthesis of N-([1,1′-Biphenyl]-4-yl)-1-(4-hydroxypyridin-2-yl)-]H-pyrazole-4-carboxamide (108)

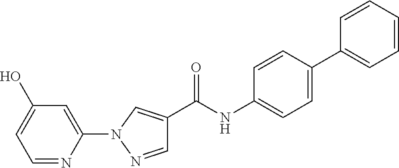 [SMILES: O=C(Nc1ccc(-c2ccccc2)cc1)c1cnn(-c2cc(O)ccn2)c1]

Following general procedure D: 106 (27 mg, 0.073 mmol) and LiCl (29 mg, 0.73 mmol) in DMAc (2 ml) for 8 hrs gave 108 (15 mg, 0.0421 mmol, 57%).

¹H NMR (400 MHz, DMSO-d₆) δ 10.18 (s, 1H), 9.39 (s, 1H), 8.32-8.18 (m, 2H), 7.91-7.82 (m, 2H), 7.70-7.63 (m, 4H), 7.50-7.31 (m, 4H), 6.82 (dd, J=5.5, 2.5 Hz, 1H).

HRMS (ESI-TOF) calcd for C₂₁H₁₇02N₄ [M+H]⁺: 357.1346, found: 357.1348.

### Example 85 Synthesis of N-([1,1′-Biphenyl]-3-ylmethyl)-1-(4-hydroxypyridin-2-yl)-1H-pyrazole-4-carboxamide (109)

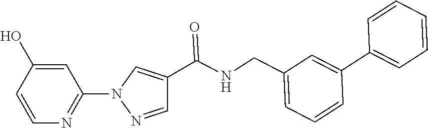 [SMILES: O=C(NCc1cccc(-c2ccccc2)c1)c1cnn(-c2cc(O)ccn2)c1]

Following general procedure D: 107 (32 mg, 0.083 mmol) and LiCl (35 mg, 0.83 mmol) in DMAc (2 ml) gave 109 (11 mg, 0.029 mmol, 36%).

¹H NMR (400 MHz, DMSO-d₆) δ 11.23 (s, 1H), 9.14 (d, J=1.0 Hz, 1H), 8.93 (t, J=6.0 Hz, 1H), 8.19 (d, J=5.5 Hz, 1H), 8.15 (d, J=1.0 Hz, 1H), 7.70-7.27 (m, 10H), 6.78 (dd, J=5.5, 2.5 Hz, 1H), 4.52 (d, J=6.0 Hz, 2H).

HRMS (ESI-TOF) calcd for C₂₂H₁₉O₂N₄ [M+H]⁺: 371.1502, found: 371.1497.

### Reference Example 86 Synthesis of Ethyl 1-(5-Cyano-4-methoxypyridin-2-yl)-]H-pyrazole-4-carboxylate (110)

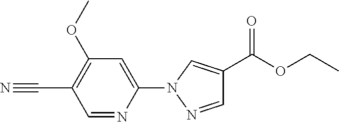 [SMILES: CCOC(=O)c1cnn(-c2cc(OC)c(C#N)cn2)c1]

Following general procedure C: 6-chloro-4-methoxynicotinonitrile (400 mg, 2.38 mmol), pyrazole-4-carboxylate ethyl ester (500 mg, 3.57 mmol), ^{t}BuxPhos Pd G3 (188 mg, 0.238 mmol) and Cs₂CO₃ (2.3 g, 7.14 mmol) in dioxane (10 ml) gave 110 (110 mg, 0.40 mmol, 17%).

¹H NMR (400 MHz, DMSO-d₆) δ 9.02 (s, 1H), 8.83 (s, 1H), 8.32 (s, 1H), 7.69 (s, 1H), 4.28 (q, J=7.0 Hz, 2H), 4.13 (s, 3H), 1.30 (t, J=7.0 Hz, 3H).

HRMS (ESI-TOF) calcd for C₁₃H₁₃03N₄ [M+H]⁺: 273.0978, found: 273.0978.

### Reference Example 87 Synthesis of 1-(5-Cyano-4-methoxypyridin-2-yl)-]H-pyrazole-4-carboxylic acid (111)

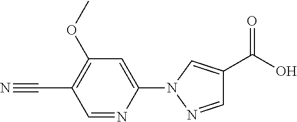 [SMILES: COc1cc(-n2cc(C(=O)O)cn2)ncc1C#N]

110 (95 mg, 0.399 mmol) was dissolved in a mixture of THF and water (11 ml (10:1)). Lithium hydroxide monohydrate (14 mg, 0.399 mmol) was added to the reaction mixture and the resulting mixture was allowed to stir at room temperature for 16 hrs. The reaction was confirmed to have gone to completion by TLC and HCl (10 ml, 1M) was added to the reaction mixture. The resultant mixture was extracted with EtOAc (3×10 ml), then washed with brine, then dried with anhydrous Na₂SO₄and concentrated under vacuo gave 111 (90 mg, 0.368 mmol, 93%).

¹H NMR (400 MHz, DMSO-d₆) δ 8.89 (d, J=1.0 Hz, 1H), 8.76 (s, 1H), 8.20 (d, J=1.0 Hz, 1H), 7.63 (s, 1H), 4.08 (s, 3H).

HRMS (ESI-TOF) calcd for C₁₁H₇03N₄ [M−H]: 243.0523, found: 243.0520.

### Example 88 Synthesis of 1-(5-Cyano-4-methoxypyridin-2-yl)-N-((6-phenylpyridin-3-yl)methyl)-]H-pyrazole-4-carboxamide (112)

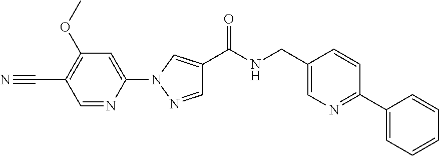 [SMILES: COc1cc(-n2cc(C(=O)NCc3ccc(-c4ccccc4)nc3)cn2)ncc1C#N]

Following general procedure B: 111 (40 mg, 0.164 mmol), 52 (56 mg, 0.306 mmol), T3P (130 mg, 0.409 mmol) and DIPEA (116 μl, 0.68 mmol) gave 112 (28 mg, 0.068 mmol, 35%).

¹H NMR (400 MHz, THF-d₈) δ 9.06 (d, J=1.0 Hz, 1H), 8.57 (s, 1H), 8.34 (dd, J=8.5, 1.5 Hz, 1H), 8.18 (t, J=6.0 Hz, 1H), 8.12-8.04 (m, 2H), 7.81 (d, J=1.5 Hz, 2H), 7.73 (s, 1H), 7.44-7.31 (m, 4H), 4.59 (d, J=6.0 Hz, 2H), 4.12 (s, 3H).

HRMS (ESI-TOF) calcd for C₂₃H₁₉O₂N₆ [M+H]⁺: 411.1564, found: 411.1555.

### Example 89 Synthesis of 1-(5-Cyano-4-methoxypyridin-2-yl)-N-(4-(trifluoromethyl)benzyl)-]H-pyrazole-4-carboxamide 113

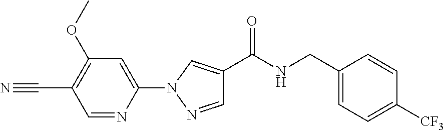 [SMILES: COc1cc(-n2cc(C(=O)NCc3ccc(C(F)(F)F)cc3)cn2)ncc1C#N]

Following general procedure B: 111 (40 mg, 0.164 mmol), 4-trifluoromethyl benzylamine (54 mg, 0.306 mmol), T3P (130 mg, 0.409 mmol) and DIPEA (116 μl, 0.68 mmol) gave 113 (38 mg, 0.0947 mmol, 47%).

¹H NMR (400 MHz, THF-d₈) δ 8.82 (s, 1H), 8.41 (dd, J=4.5, 1.5 Hz, 1H), 8.11 (dd, J=8.5, 1.5 Hz, 1H), 7.50 (s, 1H), 7.39-7.29 (m, 4H), 7.23 (t, J=6.0 Hz, 1H), 4.39 (d, J=6.0 Hz, 2H), 3.89 (s, 3H).

HRMS (ESI-TOF) calcd for C₁₉H₁₃02N₅F3[M−H]: 400.1026, found: 400.1022.

### Example 90 Synthesis of 1-(5-Cyano-4-hydroxypyridin-2-yl)-N-((6-phenylpyridin-3-yl)methyl)-]H-pyrazole-4-carboxamide (114)

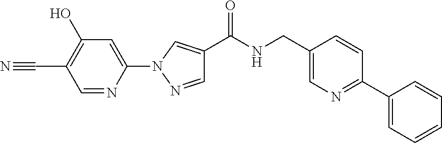 [SMILES: N#Cc1cnc(-n2cc(C(=O)NCc3ccc(-c4ccccc4)nc3)cn2)cc1O]

Following general procedure D:112 (20 mg, 0.0487 mmol) and LiCl (20.5 mg, 0.487 mmol) in DMAc (2 ml) gave 114 (8 mg, 0.020 mmol, 42%).

¹H NMR (400 MHz, DMSO-d₆) δ 9.11 (d, J=1.0 Hz, 1H), 9.10-9.04 (m, 1H), 8.64-8.61 (m, 1H), 8.37 (s, 1H), 8.16 (s, 1H), 8.09-8.03 (m, 2H), 7.93 (dd, J=8.0, 1.0 Hz, 1H), 7.81 (dd, J=8.0, 2.5 Hz, 1H), 7.53-7.38 (m, 3H), 7.15 (s, 1H), 4.49 (d, J=6.0 Hz, 2H).

HRMS (ESI-TOF) calcd for C₂₂H₁₇02N₆ [M+H]⁺: 397.1407, found: 397.1407.

### Example 91 Synthesis of 1-(5-Cyano-4-hydroxypyridin-2-yl)-N-(4-(trifluoromethyl)benzyl)-1H-pyrazole-4-carboxamide (115)

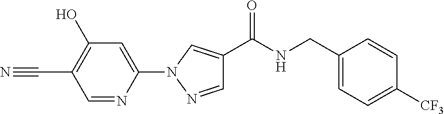 [SMILES: N#Cc1cnc(-n2cc(C(=O)NCc3ccc(C(F)(F)F)cc3)cn2)cc1O]

Following general procedure D: 113 (25 mg, 0.062 mmol) and LiCl (26 mg, 0.62 mmol) in DMAc (2 ml) gave 115 (5 mg, 0.0129 mmol, 21%).

¹H NMR (400 MHz, DMSO-d₆) δ 9.06 (s, 1H), 8.96 (t, J=6.0 Hz, 1H), 8.13 (s, 1H), 8.08 (s, 1H), 7.70 (d, J=8.0 Hz, 2H), 7.53 (d, J=8.0 Hz, 2H), 6.78 (s, 1H), 4.51 (d, J=6.0 Hz, 2H).

HRMS (ESI-TOF) calcd for C₁₈H₁₁02N₅F₃[M−H]: 386.0864, found: 386.0867.

### Reference Example 92 Synthesis of Ethyl 6-hydroxypyrazolo[1,5-a]pyrido[3,2-e]pyrimidine-7-carboxylate (116)

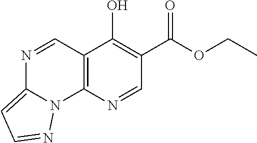 [SMILES: CCOC(=O)c1cnc2c(cnc3ccnn32)c1O]

Pyrazolo[1,5-a]pyrimidin-7-amine (250 mg, 1.86 mmol), diethyl ethoxymethylenemalonate (402 mg, 1.86 mmol) were added to a microwave vial and dissolved in anhydrous EtOH (5 ml) before the addition of NaOEt (189 mg, 2.79 mmol). The reaction mixture was heated under microwave irradiation at 100° C. for 2 hrs before allowing to cool back to room temperature. The reaction mixture was then filtered, dried and before the addition of diphenyl ether (2 ml). The resultant mixture was then heated to 240° C. for 30 minutes and then allowed to cool to room temperature. The reaction mixture was then filtered, washed with Et₂O (4×50 ml) and dried gave 116 (210 mg, 0.81 mmol, 43%).

¹H NMR (400 MHz, DMSO-d₆) δ 8.97 (s, 1H), 8.62 (s, 1H), 8.15 (d, J=2.0 Hz, 1H), 6.68 (d, J=2.0 Hz, 1H), 4.20 (q, J=7.0 Hz, 2H), 1.28 (t, J=7.0 Hz, 3H).

HRMS (ESI-TOF) calcd for C₁₂H₁₁O₃N₄ [M+H]⁺: 259.0825, found: 259.0826.

### Example 93 Synthesis of N-([1,1′-Biphenyl]-4-ylmethyl)-6-hydroxypyrazolo[1,5-a]pyrido[3,2-e]pyrimidine-7-carboxamide (117)

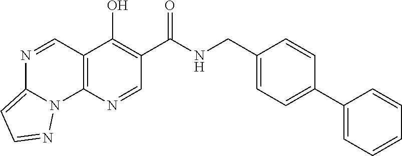 [SMILES: O=C(NCc1ccc(-c2ccccc2)cc1)c1cnc2c(cnc3ccnn32)c1O]

General Procedure A: 116 (100 mg, 0.38 mmol), 4-phenylbenzylamine (70 mg, 0.38 mmol), DABCO-(AlMe₃)₂ (100 mg, 0.38 mmol) gave 117 (4 mg, 0.01 mmol, 3%). Solvent system used for purification: 0%-5% MeOH in CH₃Cl.

¹H NMR (400 MHz, DMSO-d₆) δ 9.04 (s, 1H), 8.72 (s, 1H), 8.26 (d, J=2.0 Hz, 1H), 7.74-7.59 (m, 4H), 7.54-7.25 (m, 5H), 6.78 (d, J=2.0 Hz, 1H), 4.60 (d, J=6.0 Hz, 2H).

HRMS (ESI-TOF) calcd for C₂₃H₁₆02N₅ [M−H]: 394.1309, found: 394.1303.

### Reference Example 94 Synthesis of Methyl 3-([1,1′-biphenyl]-4-ylamino)-3-oxopropanoate (118)

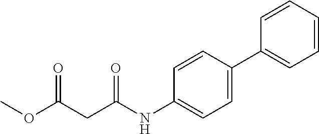 [SMILES: COC(=O)CC(=O)Nc1ccc(-c2ccccc2)cc1]

Following General Procedure B: Methyl hydrogen malonate (585 mg, 4.97 mmol), 4-aminobiphenyl (600 mg, 3.55 mmol), T3P (3.16 g, 9.94 mmol) and DIPEA (1.8 g, 14.2 mmol) gave 118 (230 mg, 0.855 mmol, 24%).

¹H NMR (400 MHz, Chloroform-d) δ 9.27 (s, 1H), 7.68-7.30 (m, 9H), 3.82 (s, 3H), 3.52 (s, 2H).

HRMS (ESI-TOF) calcd for C₁₆H₁₆03N [M+H]⁺: 270.1124, found: 270.1122.

### Example 95 Synthesis of N-([1,1′-Biphenyl]-4-yl)-6-hydroxypyrazolo[1,5-a]pyrido[3,2-e]pyrimidine-7-carboxamide (119)

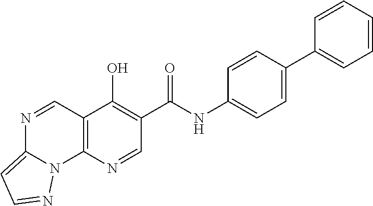 [SMILES: O=C(Nc1ccc(-c2ccccc2)cc1)c1cnc2c(cnc3ccnn32)c1O]

118 (100 mg, 0.37 mmol) were added to a microwave vial and flushed with N₂ and removed in vacuo (3 times) before the addition of anhydrous ethyl orthoformate (3 ml). The reaction mixture was heated at 120° C. for 3 hrs. The excess ethyl orthoformate was removed in vacuo and the resultant mixture was taken up in diphenyl ether (5 ml). Pyrazolo[1,5-a]pyrimidin-7-amine (75 mg, 0.55 mmol) was added, and the resultant mixture was heated to 240° C. for 30 minutes, allowed to cool to room temperature and directly purified by flash column chromatography using (CH₂Cl₂, MeOH 0-10%, 1% HCOOH) 20 column volumes gave 119 (5.5 mg, 0.015 mmol, 4%).

¹H NMR (400 MHz, DMSO-d₆) δ 13.31 (s, 1H), 9.11 (s, 1H), 8.95 (s, 1H), 8.11 (d, J=2.0 Hz, 1H), 7.85-7.27 (m, 9H), 6.68 (d, J=2.0 Hz, 1H).

HRMS (ESI-TOF) calcd for C₂₂H₁₄02N₅ [M−H]: 380.1153, found: 380.1150.

### Reference Example 96 Synthesis of Methyl 3-(([1,1′-biphenyl]-4-ylmethyl)amino)-3-oxopropanoate (120)

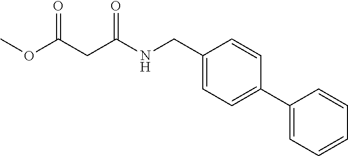 [SMILES: COC(=O)CC(=O)NCc1ccc(-c2ccccc2)cc1]

Following General Procedure B: Methyl hydrogen malonate (1.00 g, 8.9 mmol), 4-aminomethyl biphenyl (1.83 g, 10.0 mmol), T3P (6.7 g, 21.1 mmol) and DIPEA (2.7 g, 21.1 mmol) gave 120 (1.5 g, 5.3 mmol, 60.0%).

¹H NMR (400 MHz, THF-d₈) δ 7.71 (s, 1H), 7.41-7.03 (m, 9H), 4.23 (d, J=6.0 Hz, 2H), 3.46 (s, 3H), 3.10 (s, 2H).

HRMS (ESI-TOF) calcd for C₁₇H₁₈O₃N [M+H]⁺: 284.1281, found: 284.1281.

### Reference Example 97 Synthesis of Methyl 3-oxo-3-((4-(trifluoromethyl)benzyl)amino)propanoate (121)

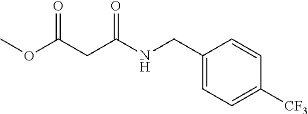 [SMILES: COC(=O)CC(=O)NCc1ccc(C(F)(F)F)cc1]

Following General Procedure B: Methyl hydrogen malonate (1.00 g, 8.9 mmol), 4-trifluorobenzyl amine (1.75 g, 10.0 mmol), T3P (6.7 g, 21.1 mmol) and DIPEA (2.7 g, 21.1 mmol) gave 121 (736 mg, 2.67 mmol, 30%).

¹H NMR (400 MHz, Chloroform-d) δ 7.86 (s, 1H), 7.52 (d, J=8.0 Hz, 2H), 7.35 (d, J=8.0 Hz, 2H), 4.46 (d, J=6.0 Hz, 2H), 3.68 (s, 3H), 3.33 (s, 2H).

HRMS (ESI-TOF) calcd for C₁₂H₁₃03NF₃ [M+H]⁺: 276.0842, found: 276.0843.

### Example 98 Synthesis of N-([1,1′-Biphenyl]-4-ylmethyl)-6-hydroxy-2,5-dimethylpyrazolo[1,5-a]pyrido[3,2-e]pyrimidine-7-carboxamide (122)

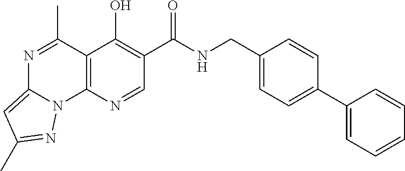 [SMILES: Cc1cc2nc(C)c3c(O)c(C(=O)NCc4ccc(-c5ccccc5)cc4)cnc3n2n1]

120 (200 mg, 0.70 mmol) were added to a microwave vial and flushed with N₂ and removed in vacuo (3 times) before the addition of anhydrous ethyl orthoformate (5 ml). The reaction mixture was heated under microwave irradiation at 120° C. for 3 hrs. The excess ethyl orthoformate was removed in vacuo and the resultant mixture was taken up in diphenyl ether (5 ml). 2,5-dimethylpyrazolo[1,5-a]pyrimidin-7-amine (114 mg, 0.70 mmol) was added and the resultant mixture was heated to 240° C. for 30 minutes, allowed to cool to room temperature and directly purified by by flash column chromatography using (CH₂Cl₂:MeOH 0-10%, 1% formic acid) over 20 column volumes to give 122 (10 mg, 0.024 mmol, 3%).

¹H NMR (400 MHz, DMSO-d₆) δ 10.54 (s, 1H), 8.59 (s, 1H), 7.69-7.30 (m, 9H), 6.44 (s, 1H), 4.59 (d, J=6.0 Hz, 2H), 2.88 (s, 3H), 2.45 (s, 3H).

HRMS (ESI-TOF) calcd for C₂₅H₂₂02N₅ [M+H]⁺: 424.1768, found: 424.1768.

### Example 99 Synthesis of 6-Hydroxy-2,5-Dimethyl-N-(4-(trifluoromethyl)benzyl)pyrazolo[1,5-a]pyrido[3,2-e]pyrimidine-7-carboxamide (123)

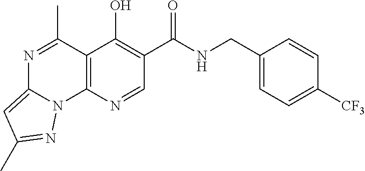 [SMILES: Cc1cc2nc(C)c3c(O)c(C(=O)NCc4ccc(C(F)(F)F)cc4)cnc3n2n1]

121 (200 mg, 0.70 mmol) were added to a microwave vial and flushed with N₂ and removed in vacuo 3 times before the addition of anhydrous ethyl orthoformate (5 ml). The reaction mixture was heated under microwave irradiation at 120° C. for 3 hrs. The excess ethyl orthformate was removed in vacuo and the resultant mixture was taken up in diphenyl ether (5 ml). 2,5-dimethylpyrazolo[1,5-a]pyrimidin-7-amine (114 mg, 0.70 mmol) was added and the resultant mixture was heated to 240° C. for 30 minutes, allowed to cool to room temperature and directly purified by flash column chromatography using (CH₂Cl₂:MeOH 0-10%, 1% formic acid) over 20 column volumes gave 123 (8 mg, 0.020 mmol, 3%).

¹H NMR (400 MHz, DMSO-d₆) δ 8.52 (s, 1H), 7.71 (d, J=8.1 Hz, 2H), 7.55 (d, J=8.0 Hz, 2H), 6.50 (s, 1H), 4.64 (d, J=6.0 Hz, 2H), 2.89 (s, 3H), 2.47 (s, 3H).

HRMS (ESI-TOF) calcd for C₂₀H₁₅O₂N₅F₃[M−H]: 414.1183, found: 414.1182.

### Example 100 Synthesis of N-([1,1′-biphenyl]-4-ylmethyl)-6-(4-cyano-]H-pyrazol-1-yl)-5-methoxynicotinamide (124)

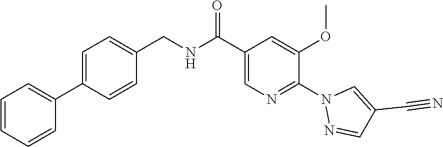 [SMILES: COc1cc(C(=O)NCc2ccc(-c3ccccc3)cc2)cnc1-n1cc(C#N)cn1]

Following general procedure C: 72 (147 mg, 0.42 mmol) PdtBuXPhos G3 (33 mg, 0.04 mmol), Cesium Carbonate (273 mg, 0.84 mmol) and 1H-pyrazole-4-carbonitrile (70 mg, 0.5 mmol) gave the titled compound (56 mg, 0.137 mmol, 33%).

¹H NMR (400 MHz, DMSO) δ 9.41 (t, J=6.0 Hz, 1H), 9.11 (d, J=1.0 Hz, 1H), 8.65 (d, J=2.0 Hz, 1H), 8.36 (d, J=1.0 Hz, 1H), 8.18 (d, J=2.0 Hz, 1H), 7.67-7.62 (m, 4H), 7.49-7.42 (m, 4H), 7.40-7.32 (m, 1H), 4.59 (d, J=6.0 Hz, 2H), 3.97 (s, 3H).

### Example 101 Synthesis of N-([1,1′-biphenyl]-4-ylmethyl)-6-(4-cyano-1H-pyrazol-1-yl)-5-hydroxynicotinamide (125)

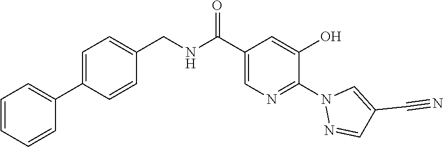 [SMILES: N#Cc1cnn(-c2ncc(C(=O)NCc3ccc(-c4ccccc4)cc3)cc2O)c1]

Following general procedure D: 125 (56 mg, 0.14 mmol) and lithium chloride (58 mg, 1.37 mmol) were combined before the addition of NN-Dimethylacetamide (3 ml). The resultant mixture was microwaved at 150° C. for 8 hrs. The crude material was then purified using flash reverse phase chromatography (H₂O 0.1% formic acid (0-100% ACN, 0.1% formic acid)) to give the titled compound (3 mg, 0.007 mmol, 5%).

¹H NMR (400 MHz, DMSO) δ 9.35 (t, J=5.9 Hz, 1H), 9.24 (s, 1H), 8.53 (d, J=1.9 Hz, 1H), 8.42 (s, 1H), 7.95 (d, J=1.9 Hz, 1H), 7.67-7.62 (m, 4H), 7.45 (qd, J=7.1, 1.9 Hz, 4H), 7.38-7.32 (m, 1H), 4.55 (d, J=6.0 Hz, 2H).

### Reference Example 102-Synthesis of 6-chloro-5-methoxy-N-((4-(trifluoromethyl)cyclohexyl)methyl)nicotinamide (126)

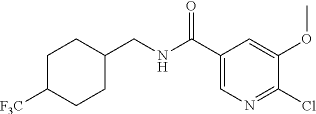 [SMILES: COc1cc(C(=O)NCC2CCC(C(F)(F)F)CC2)cnc1Cl]

Following general procedure B: 6-chloro-5-methoxynicotinic acid (1000 mg, 5.35 mmol), HATU (4064 mg, 10.70 mmol), C-4 trifluorocyclohexyl amine (1209 μL, 8.02 mmol) & DIPEA (2759 μL, 16.04 mmol) gave the title compound (1625 mg, 4.64 mol, 86%).

LRMS m/z calcd. For C₁₅H₁₉ClF₃N₂O₂[M+H]⁺: 351.1, found: 351.1

### Example 103 Synthesis of Ethyl 1-(3-methoxy-5-(((4-(trifluoromethyl)cyclohexyl)methyl)carbamoyl)pyridin-2-yl)-1H-pyrazole-4-carboxylate

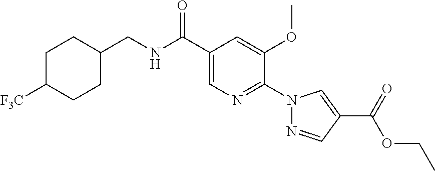 [SMILES: CCOC(=O)c1cnn(-c2ncc(C(=O)NCC3CCC(C(F)(F)F)CC3)cc2OC)c1]

Following general procedure C: 127 (300 mg, 0.86 mmol), PdtBuXPhos G3 (68 mg, 0.09 mmol), ethyl 1H-pyrazole-4-carboxylate (180 mg, 1.29 mmol) and Cs₂CO₃ (835 mg, 2.57 mmol) gave the targeted compound (92 mg, 0.20 mmol, 23%).

¹H NMR (400 MHz, DMSO) δ 8.80 (t, J=6.0 Hz, 1H), 8.74 (d, J=0.7 Hz, 1H), 8.57 (d, J=1.8 Hz, 1H), 8.14 (d, J=0.6 Hz, 1H), 8.07 (d, J=1.8 Hz, 1H), 4.27 (q, J=7.0 Hz, 2H), 3.96 (s, 3H), 3.19 (t, J=6.0 Hz, 2H), 2.35-2.16 (m, 1H), 1.94-1.82 (m, 4H), 1.63-1.50 (m, 1H), 1.30 (t, J=7.0 Hz, 3H), 1.27-0.98 (m, 4H).

LRMS m/z calcd. For C₂₁H₂₆F3N₄04[M+H]⁺: 455.19, found: 455.30

### Example 104 Synthesis of Ethyl 1-(3-hydroxy-5-(((4-(trifluoromethyl)cyclohexyl)methyl)carbamoyl)pyridin-2-yl)-1H-pyrazole-4-carboxylate (128)

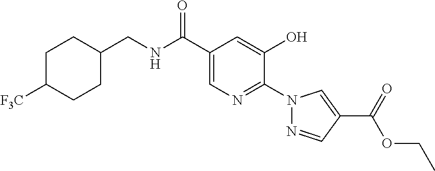 [SMILES: CCOC(=O)c1cnn(-c2ncc(C(=O)NCC3CCC(C(F)(F)F)CC3)cc2O)c1]

Follow general procedure D: 128 (92 mg, 0.20 mmol), LiCl (85 mg, 2.03 mmol) and DMSO (2 ml) gave the title compound (20 mg, 0.04 mmol, 20%).

¹H NMR (400 MHz, DMSO) δ 8.95 (s, 1H), 8.69 (t, J=6.0 Hz, 1H), 8.42 (d, J=2.0 Hz, 1H), 8.24 (s, 1H), 7.87 (d, J=2.0 Hz, 1H), 4.28 (q, J=7.0 Hz, 2H), 3.15 (t, J=6.3 Hz, 2H), 1.92-1.79 (m, 5H), 1.55 (dp, J=18.8, 7.4, 5.7 Hz, 1H), 1.29 (t, J=7.0 Hz, 3H), 1.25-1.16 (m, 2H), 1.02 (qd, J=13.8, 13.1, 3.9 Hz, 2H).

### Example 105 Synthesis of]-(3-hydroxy-5-(((4-(trifluoromethyl)cyclohexyl)methyl)carbamoyl)pyridin-2-yl)-]H-pyrazole-4-carboxylic acid (129)

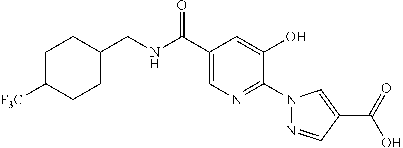 [SMILES: O=C(O)c1cnn(-c2ncc(C(=O)NCC3CCC(C(F)(F)F)CC3)cc2O)c1]

129 (20 mg, 0.04 mmol) was dissolved in THE (10 ml) before the addition of LiGH aq solution (500 μL, 2 M). The resultant mixture was stirred at room temperature of 16 hr. The reaction mixture was neutralized with formic acid before the addition of celite. The solvent was then removed under vacuum and the crude mixture was purified using flash reverse column chromatography (0%-100% ACN (0.1% formic acid) in H₂O (0.1% formic acid)) gave the title compound (6 mg, 0.014 mmol, 36%).

¹H NMR (400 MHz, DMSO) δ 8.82 (s, 1H), 8.65 (t, J=6.0 Hz, 1H), 8.40 (d, J=2.0 Hz, 1H), 8.09 (s, 1H), 7.88 (d, J=2.0 Hz, 1H), 3.14 (t, J=6.0 Hz, 2H), 2.28-2.15 (m, 1H), 1.92-1.80 (m, 5H), 1.21 (qt, J=13.9, 6.7 Hz, 3H), 1.02 (qd, J=13.7, 12.9, 4.0 Hz, 2H).

LRMS m/z calcd. For C₁₈H₁₈F3N₄04[M−H]: 411.13, found: 411.0

### Reference Example 106 Synthesis of Ethyl 6-chloro-4-methoxynicotinate (130)

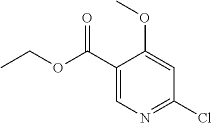

6-chloro-4-methoxynicotinic acid (5 g, 0.03 mol), EDC·HCl (6.22 g, 0.04 mol), DMAP (0.05 g, 1.2 mmol) were dissolved in DMF (50 ml). DIPEA (4.60 ml, 0.03 mmol) and EtOH (5 ml) were then added to the reaction mixture. The resultant mixture was then stirred at room temperature for 16 hr. EtOAc (50 ml), 1M HCl (50 ml) and H2O (100 ml) were added to the reaction mixture. The organic and aqueous layers were separated. This process was repeat 2 more times. The organic layers were combined before washed with brine and then dried with anhydrous Na₂SO₄. The crude compound was then purified using flash column chromatography using (cyclohexane 100%-50%, EtOAc 0%-50%) over 15 column volumes to give the desired compound (2.02 g, 9.39 mmol, 31%).

LRMS m/z calcd. For C₉H₁₁ClNO₃[M+H]⁺: 216.0, found: 216.2

### Reference Example 107 Synthesis of Ethyl 4-methoxy-6-(1H-pyrazol-1-yl) nicotinate (131)

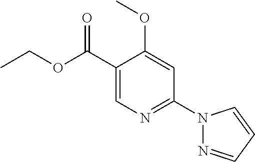 [SMILES: CCOC(=O)c1cnc(-n2cccn2)cc1OC]

Following the general procedure C: 130 (1.90 g, 0.088 mol), PdtBuXPhos G3 (0.35 g, 0.00044 mol), Cesium carbonate (4.30 g, 0.0132 mol) and pyrazole (0.90 g, 0.0132 mol) and anhydrous 1,4-dioxane (50 ml) gave the titled compound (1.9 g, 7.65 mmol, 87%).

LRMS m/z calcd. For C₁₂H₁₄N₃O₃[M+H]⁺: 248.10, found: 248.10

### Reference Example 108 Synthesis of 4-methoxy-6-(1H-pyrazol-1-yl)nicotinic acid (132)

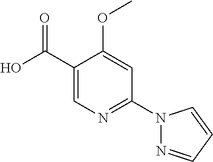 [SMILES: COc1cc(-n2cccn2)ncc1C(=O)O]

131 (1.9 g, 7.65 mmol) was dissolved in a mixture of THF (65 mL), MeOH (35 mL), H₂O (10 mL) before the addition of lithium hydroxide monohydrate (3.7 g, 90.5 mmol). The resultant mixture was stirred at room temperature for 16 hr. The reaction was confirmed to go to completion via TLC. The reaction mixture was acidified, cooled to 5° C. and filtered to collect the precipitate. The precipitate was washed with diethyl ether to give the tilted compound (1.4 g, 6.39 mmol, 84%).

LRMS m/z calcd. For C₁₀H₁₀N₃O₄[M+H]⁺: 220.07, found: 220.2.

### Example 109 Synthesis of N-(but-2-yn-1-yl)-4-methoxy-6-(1H-pyrazol-1-yl) nicotinamide (133)

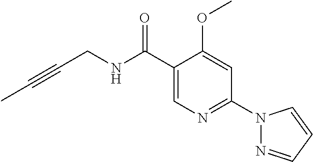 [SMILES: CC#CCNC(=O)c1cnc(-n2cccn2)cc1OC]

Following general procedure B: 132 (187 mg, 0.85 mmol), HATU (646 mg, 1.7 mmol), but-2-yn-1-amine hydrogen chloride (135 mg, 1.27 mmol) and DIPEA (438 μL, 2.55 mmol) gave the titled compound (148 mg, 0.54 mmol, 63%).

LRMS m/z calcd. For C₁₄H₁₅N₄O₂[M+H]⁺: 271.1, found: 271.2

### Example 110 Synthesis of N-(but-2-yn-1-yl)-4-hydroxy-6-(1H-pyrazol-1-yl)nicotinamide (134)

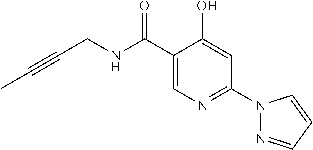 [SMILES: CC#CCNC(=O)c1cnc(-n2cccn2)cc1O]

Following general procedure D: 133 (143 mg, 0.53 mmol) and lithium chloride (111 mg, 2.64 mmol) gave the titled compound (44 mg, 0.171 mmol, 33%).

¹H NMR (400 MHz, DMSO) δ 11.38 (t, J=5.4 Hz, 1H), 8.48-8.39 (m, 2H), 7.73-7.62 (m, 1H), 6.73 (d, J=3.1 Hz, 1H), 6.42 (q, J=2.1 Hz, 1H), 4.01 (dq, J=5.2, 2.5 Hz, 2H), 1.78 (t, J=2.7 Hz, 3H).

LRMS m/z calcd. For C₁₃H₁₃N₄O₂[M+H]⁺: 257.10, found: 257.30

### Example 111 Synthesis of N-((6-chloropyridin-3-yl)methyl)-4-methoxy-6-(1H-pyrazol-1-yl)nicotinamide (135)

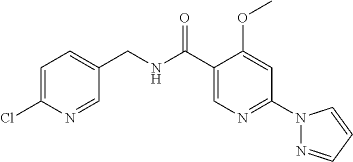 [SMILES: COc1cc(-n2cccn2)ncc1C(=O)NCc1ccc(Cl)nc1]

Following general procedure B: 132 (900 mg, 4.11 mmol), HATU (3123 mg, 8.22 mmol), (6-chloropyridin-3-yl) methanamine (700 mg, 4.93 mmol) and DIPEA (2.1 mL, 12.33 mmol) gave the titled compound (1.315 g, 3.83 mmol, 93%).

LRMS m/z calcd. For C₁₆H₁₅ClN₅O₂[M+H]⁺: 344.1, found: 344.2

### Example 112 Synthesis of Ethyl 4-(5-((4-methoxy-6-(1H-pyrazol-1-yl)nicotinamido)methyl)pyridin-2-yl)benzoate (136)

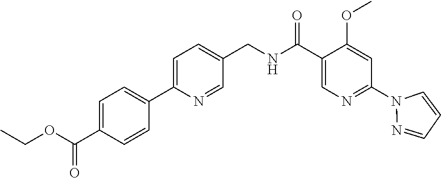 [SMILES: CCOC(=O)c1ccc(-c2ccc(CNC(=O)c3cnc(-n4cccn4)cc3OC)cn2)cc1]

Following general procedure C: 135 (44 mg, 0.128 mmol), (4-ethoxycarbonyl)phenyl)boronic acid (42 mg, 0.22 mmol), Pd Amphos (5.16 mg, 0.01 mmol) and cesium carbonate (142.13 mg, 0.44 mmol) gave the titled compound (50 mg, 0.109 mmol, 54%).

LRMS m/z calcd. For C₂₅H₂₄N₅O₄[M+H]⁺: 458.18, found: 458.3, [M+2H]/2 was observed 229.7.

### Example 113 Synthesis of 4-methoxy-6-(1H-pyrazol-1-yl)-N-((6-(pyrimidin-5-yl)pyridin-3-yl)methyl)-nicotinamide (137)

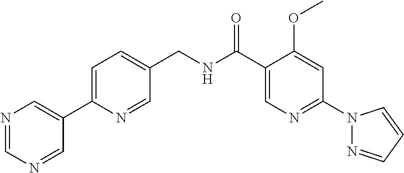 [SMILES: COc1cc(-n2cccn2)ncc1C(=O)NCc1ccc(-c2cncnc2)nc1]

Following general procedure C: 135 (27 mg, 0.078 mmol), pyrimidin-5-ylboronic acid (27 mg, 0.22 mmol), Pd Amphos (5.16 mg, 0.01 mmol) and cesium carbonate (142.13 mg, 0.44 mmol) gave the titled compound (9 mg, 0.012 mmol, 15%).

LRMS m/z calcd. For C₂₀H₁₈N₇O₂[M+H]⁺: 388.15, found: 388.00. [M+2H]/2 was observed 194.8

### Example 114 Synthesis of 4-methoxy-N-((6-(4-(methylcarbamoyl)phenyl)pyridin-3-yl)methyl)-6-(1H-pyrazol-1-yl)nicotinamide (138)

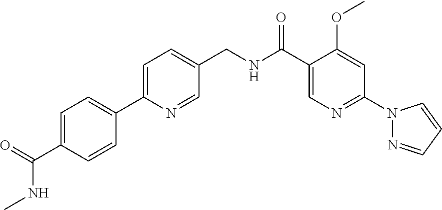 [SMILES: CNC(=O)c1ccc(-c2ccc(CNC(=O)c3cnc(-n4cccn4)cc3OC)cn2)cc1]

Following general procedure C: 135 (50 mg, 0.15 mmol), (4-(methylcarbamoyl)phenyl)boronic acid (39 mg, 0.22 mmol), Pd Amphos (5.16 mg, 0.01 mmol) and cesium carbonate (142.13 mg, 0.44 mmol) gave the titled compound (39 mg, 0.088 mmol, 59%).

LRMS m/z calcd. For C₂₄H₂₃N₆O₃[M+H]⁺: 443.18, found: 443.1, [M+2H]/2 was observed 222.20.

### Example 115 Synthesis of N-((6-(4-carbamoylphenyl)pyridin-3-yl)methyl)-4-methoxy-6-(1H-pyrazol-1-yl)nicotinamide (139)

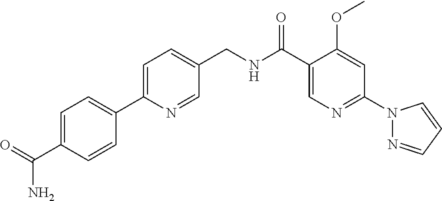 [SMILES: COc1cc(-n2cccn2)ncc1C(=O)NCc1ccc(-c2ccc(C(N)=O)cc2)nc1]

Following general procedure C: 135 (50 mg, 0.15 mmol), (4-(carbamoyl)phenyl)boronic acid (36 mg, 0.22 mmol), Pd Amphos (5.16 mg, 0.01 mmol) and cesium carbonate (142.13 mg, 0.44 mmol) gave the titled compound (22 mg, 0.051 mmol, 34%).

LRMS m/z calcd. For C₂₃H₂₁N₆O₃[M+H]⁺: 429.17, found: 429.2

### Example 116 Synthesis of 4-methoxy-N-((6-(4-morpholinophenyl)pyridin-3-yl)methyl)-6-(1H-pyrazol-1-yl)nicotinamide (140)

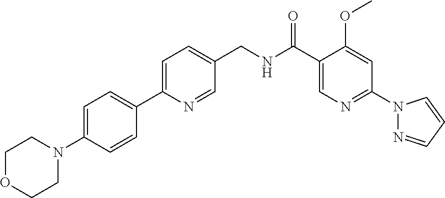 [SMILES: COc1cc(-n2cccn2)ncc1C(=O)NCc1ccc(-c2ccc(N3CCOCC3)cc2)nc1]

Following general procedure C: 135 (50 mg, 0.15 mmol), (4-morpholinophenyl)boronic acid (59 mg, 0.22 mmol), Pd Amphos (5.16 mg, 0.01 mmol) and cesium carbonate (142.13 mg, 0.44 mmol) gave the titled compound (50 mg, 0.106 mmol, 71%).

LRMS m/z calcd. For C₂₆H₂₇N₆O₃[M+H]⁺: 471.21, found: 471.20, [M+2H]/2 was observed 236.20.

### Example 117 Synthesis of N-((6-(benzo[d]thiazol-5-yl)pyridin-3-yl)methyl)-4-methoxy-6-(1H-pyrazol-1-yl)nicotinamide (141)

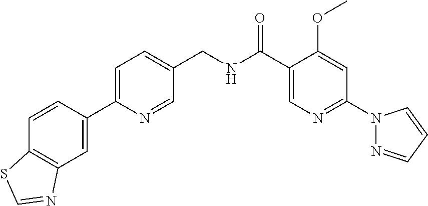 [SMILES: COc1cc(-n2cccn2)ncc1C(=O)NCc1ccc(-c2ccc3scnc3c2)nc1]

Following general procedure C: 135 (50 mg, 0.15 mmol), (5-(4,4,5,5-tetramethyl-1,3,2-dioxaborolan-2-yl)benzo[d]thiazole (57.07 mg, 0.22 mmol), Pd Amphos (5.16 mg, 0.01 mmol) and cesium carbonate (142.13 mg, 0.44 mmol) gave the titled compound (22 mg, 0.0497 mmol, 33%).

LRMS m/z calcd. For C₂₃H₁₉N₆O₂S[M+H]⁺: 443.13, found [M+2H]²⁺/2: 222.0

### Example 118 Synthesis of Ethyl 4-(5-((4-hydroxy-6-(1H-pyrazol-1-yl)nicotinamido)methyl)pyridin-2-yl)benzoate (142)

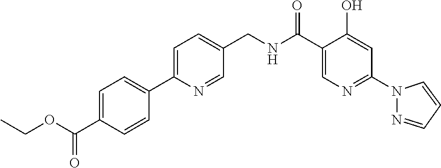 [SMILES: CCOC(=O)c1ccc(-c2ccc(CNC(=O)c3cnc(-n4cccn4)cc3O)cn2)cc1]

Following general procedure D: 140 (50 mg, 0.11 mmol) and lithium chloride (46 mg, 1.09 mmol) gave the titled compound (10 mg, 0.022 mmol, 22%) and was purified via reverse flash chromatography (0-100% ACN (0.1% formic acid) in H₂O (0.1% formic acid)) gave the titled compound (10 mg, 0.0225 mmol, 22%).

LRMS m/z calcd. For C₂₄H₂₂N₅O₄[M+H]⁺: 444.10, found: 444.2, [M+2H]²⁺/2 was observed 222.7.

### Example 119 Synthesis of 4-(5-((4-hydroxy-6-(1H-pyrazol-1-yl)nicotinamido)methylpyridin-2-yl)benzoic acid (143)

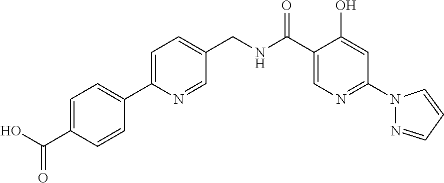 [SMILES: O=C(O)c1ccc(-c2ccc(CNC(=O)c3cnc(-n4cccn4)cc3O)cn2)cc1]

143 (6 mg, 0.0135 mmol) was dissolved in a mixture of MeOH (5 mL) and H₂O (1 mL) before the addition of lithium hydroxide (2.84 mg, 0.07 mmol). The resultant mixture was stirred at 16 hr at room temperature. 1M HCl (1 mL) was added to the reaction mixture and the resultant precipitate was filtered, washed with Et₂O, and allowed to dry to give the title compound (2 mg, 0.0048 mmol, 35%).

¹H NMR (400 MHz, DMSO) δ 11.88 (t, J=5.8 Hz, 1H), 8.65 (d, J=2.3 Hz, 1H), 8.48 (s, 1H), 8.44 (s, 1H), 8.15 (d, J=8.2 Hz, 2H), 8.01 (dd, J=8.3, 6.3 Hz, 3H), 7.82 (dd, J=8.0, 2.5 Hz, 1H), 7.65 (d, J=1.5 Hz, 1H), 6.68 (s, 1H), 6.41 (dd, J=2.1, 2.1 Hz, 1H), 4.55 (d, J=5.8 Hz, 2H).

LRMS m/z calcd. For C₂₂H₁₈N₅O₄[M+H]⁺: 416.14, found: 416.20, [M+2H]⁺/2 was observed 208.6

### Example 120 Synthesis of 4-hydroxy-6-(1H-pyrazol-1-yl)-N-((6-(pyrimidin-5-yl)pyridin-3-yl)methyl)nicotinamide (144)

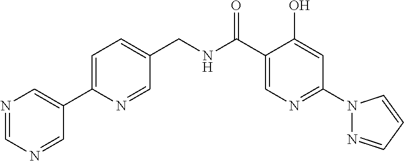 [SMILES: O=C(NCc1ccc(-c2cncnc2)nc1)c1cnc(-n2cccn2)cc1O]

Following general procedure D: 137 (9 mg, 0.02 mmol) and lithium chloride (9.77 mg, 0.23 mmol) gave the titled compound (6 mg, 0.0160 mmol, 81%).

¹H NMR (400 MHz, DMSO) δ 9.43 (d, J=1.9 Hz, 2H), 9.24 (s, 1H), 8.75 (d, J=2.2 Hz, 1H), 8.64 (d, J=2.7 Hz, 1H), 8.15-8.10 (m, 1H), 7.97-7.85 (m, 2H), 7.35 (s, 1H), 6.61 (q, J=2.1 Hz, 1H), 4.63 (d, J=5.9 Hz, 2H).

LRMS m/z calcd. For C₁₉H₁₆N₇O₂[M+H]⁺: 374.14, found: 374.00, [M+2H]⁺/2=187.5

### Example 121 Synthesis of 4-hydroxy-N-((6-(4-(methylcarbamoyl)phenyl)pyridin-3-yl)methyl)-6-(1H-pyrazol-1-yl)nicotinamide (145)

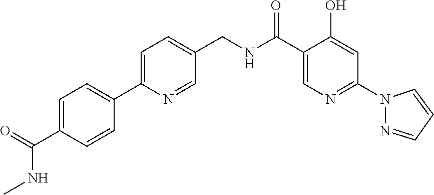 [SMILES: CNC(=O)c1ccc(-c2ccc(CNC(=O)c3cnc(-n4cccn4)cc3O)cn2)cc1]

Following general procedure D: 138 (35 mg, 0.09 mmol) and lithium chloride (37 mg, 0.88 mmol) gave the titled compound (6 mg, 0.0160 mmol, 81%).

¹H NMR (400 MHz, DMSO) δ 9.57 (s, 1H), 8.74 (s, 1H), 8.70 (d, J=2.2 Hz, 1H), 8.64 (d, J=2.6 Hz, 1H), 8.50 (q, J=4.5 Hz, 1H), 8.21-8.09 (m, 2H), 8.02 (d, J=8.2 Hz, 1H), 7.98-7.90 (m, 2H), 7.90-7.79 (m, 2H), 7.33 (s, 1H), 6.60 (t, J=2.7 Hz, 1H), 4.62 (d, J=5.9 Hz, 2H), 2.81 (d, J=4.5 Hz, 3H).

LRMS m/z calcd. For C₂₃H₂₁N₆O₃[M+H]⁺: 429.17, found: 429.10, [M+2H]²⁺/2=215.1

### Example 122 Synthesis of N-((6-(4-carbamoylphenyl)pyridin-3-yl)methyl)-4-hydroxy-6-(1H-pyrazol-1-yl)nicotinamide (146)

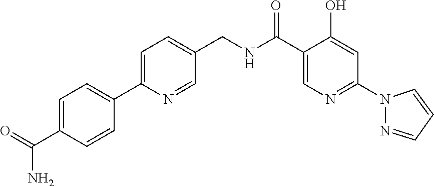 [SMILES: NC(=O)c1ccc(-c2ccc(CNC(=O)c3cnc(-n4cccn4)cc3O)cn2)cc1]

Following general procedure D: 139 (22 mg, 0.05 mmol) and lithium chloride (21.59 mg, 0.51 mmol) gave the titled compound (8 mg, 0.019 mmol, 38%).

¹H NMR (400 MHz, DMSO) δ 9.72 (s, 1H), 8.75-8.66 (m, 2H), 8.62 (d, J=2.6 Hz, 1H), 8.18-8.10 (m, 2H), 8.06-7.94 (m, 4H), 7.92-7.78 (m, 2H), 7.40 (s, 1H), 7.30 (s, 1H), 6.58 (dd, J=2.7, 1.7 Hz, 1H), 4.61 (d, J=5.9 Hz, 2H).

LRMS m/z calcd. For C₂₂H₁₉N₆O₃[M+H]⁺: 415.15, found: 415.20, [M+2H]²⁺/2=208.40

### Example 123 Synthesis of 4-hydroxy-N-((6-(4-morpholinophenyl)pyridin-3-yl)methyl)-6-(1H-pyrazol-1-yl)nicotinamide (147)

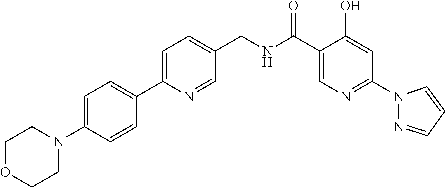 [SMILES: O=C(NCc1ccc(-c2ccc(N3CCOCC3)cc2)nc1)c1cnc(-n2cccn2)cc1O]

Following general procedure D: 140 (35 mg, 0.11 mmol) and lithium chloride (44.68 mg, 1.06 mmol) gave the titled compound (6 mg, 0.0160 mmol, 81%).

¹H NMR (400 MHz, DMSO) δ 9.65 (s, 1H), 8.72 (s, 1H), 8.63 (dd, J=2.6, 0.7 Hz, 1H), 8.58 (dd, J=2.3, 0.9 Hz, 1H), 8.00-7.92 (m, 2H), 7.88-7.80 (m, 2H), 7.77 (dd, J=8.2, 2.3 Hz, 1H), 7.29 (s, 1H), 7.06-6.98 (m, 2H), 6.59 (dd, J=2.6, 1.7 Hz, 1H), 4.56 (d, J=5.9 Hz, 2H), 3.75 (dd, J=5.8, 3.9 Hz, 4H), 3.22-3.15 (m, 4H).

LRMS m/z calcd. For C₂₅H₂₅N₆O₃[M+H]⁺: 457.20, found: 457.40, [M+2H]²⁺/2=229.2

### Example 123a Synthesis of N-((6-(benzo[d]thiazol-5-yl)pyridin-3-yl)methyl)-4-hydroxy-6-(1H-pyrazol-1-yl)nicotinamide (148)

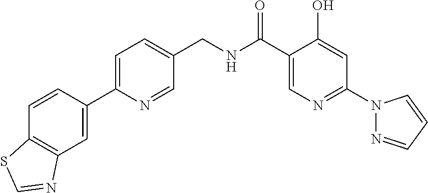 [SMILES: O=C(NCc1ccc(-c2ccc3scnc3c2)nc1)c1cnc(-n2cccn2)cc1O]

Following general procedure D: 141 (22 mg, 0.05 mmol) and lithium chloride (21 mg, 0.5 mmol) gave the titled compound (14 mg, 0.032 mmol, 65%).

¹H NMR (400 MHz, DMSO) δ 9.45 (s, 1H), 8.84-8.59 (m, 3H), 8.35-8.19 (m, 2H), 8.17-8.07 (m, 2H), 7.95-7.82 (m, 2H), 7.34 (s, 1H), 6.60 (dd, J=3.8, 3.0 Hz, 1H), 4.62 (d, J=6.2 Hz, 2H).

LRMS m/z calcd. For C₂₂H₁₇N₆O₂S[M+H]⁺: 429.11, found: 429.10, [M+2H]²⁺/2=215.2

### Reference Example 124 Synthesis of methyl 6-chloro-4-methoxypyridazine-3-carboxylate (149)

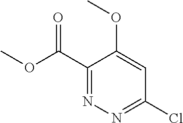

Methyl 4,6-dichloropyridazine-3-carboxylate (1000 mg, 4.93 mmol) was dissolved in THE (10 mL). The resultant mixture was cooled to 0° C. before the slow addition of sodium methoxide (319 mg, 5.91 mmol). The resultant mixture was allowed to warm up to room temperature and stirred for 6 hours. The solvent was then removed under vacuum and the resultant residue was taken up in a mixture of EtOAc (50 mL) and H₂O (50 mL). The organic and aqueous layers were separated, and the aqueous layer was washed with EtOAc (50 mL) twice more. The organic layers were combined, dried (Na₂SO₄) and purified using flash column chromatography (0%-100% EtOAc, cyclohexane 100%-0%) to give the titled compound (103 mg, 0.509 mmol, 10%).

LRMS m/z calcd. For C₇H₈ClN₂O₃[M+H]⁺: 203.02, found: 203.1

### Reference Example 125 Synthesis of methyl 4-methoxy-6-(1H-pyrazol-1-yl)pyridazine-3-carboxylate (150)

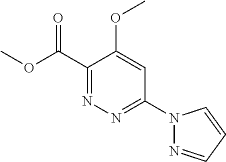 [SMILES: COC(=O)c1nnc(-n2cccn2)cc1OC]

Following general procedure C: 149 (103 mg, 0.29 mmol), pyrazole (30 mg, 0.44 mmol), Pd ^{t}BuXPhos G3 (23 mg, 0.03 mmol) and cesium carbonate (284 mg, 0.88 mmol) gave the titled compound (37 mg, 0.16 mmol, 55%).

LRMS m/z calcd. For C₁₀H₁₁N₄O₃[M+H]⁺: 235.08, found: 235.2

### Reference Example 126 Synthesis of 4-methoxy-6-(1H-pyrazol-1-yl)pyridazine-3-carboxylic acid (151)

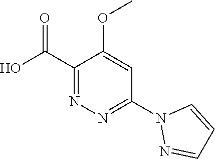 [SMILES: COc1cc(-n2cccn2)nnc1C(=O)O]

150 (37 mg, 0.16 mmol) was taken up in a mixture of methanol (5 mL) and H2O (0.5 mL) before the addition of lithium hydroxide (33.21 mg, 0.79 mmol). The resultant mixture was stirred at room temperature for 16 hrs. 1M HCl (5 mL) was added to the reaction mixture. The precipitate was then collected and washed with Et₂O to give the titled compound (22 mg, 0.099 mmol, 62%).

LRMS m/z calcd. For C₉H₉N₄04[M+H]⁺: 221.07, found: 221.1

### Example 127 Synthesis of 4-methoxy-6-(1H-pyrazol-1-yl)-N-((4-(trifluoromethyl)cyclohexyl)methyl)pyridazine-3-carboxamide (152)

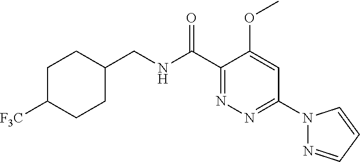 [SMILES: COc1cc(-n2cccn2)nnc1C(=O)NCC1CCC(C(F)(F)F)CC1]

Following general procedure B: 151 (22 mg, 0.10 mmol), HATU (76 mg, 0.20 mmol), (4-trifluoromethyl)cyclohexyl)methanamine (15 μL, 0.1 mmol) and DIPEA (51 μL, 0.3 mmol) gave the titled compound (34 mg, 0.088 mmol, 89%).

LRMS m/z calcd. For C₁₇H₂₁F₃N₅O₂[M+H]⁺: 384.16, found: 384.2

### Example 128 Synthesis of 4-hydroxy-6-(1H-pyrazol-1-yl)-N-((4-(trifluoromethyl)cyclohexyl)methyl)pyridazine-3-carboxamide (153)

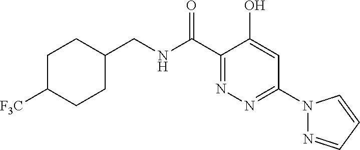 [SMILES: O=C(NCC1CCC(C(F)(F)F)CC1)c1nnc(-n2cccn2)cc1O]

Following general procedure D: 152 (34 mg, 0.08 mmol) and lithium chloride (78 mg, 1.86 mmol) gave the titled compound (8 mg, 0.0216 mmol, 11%).

¹H NMR (400 MHz, DMSO) δ 9.92 (s, 1H), 8.79-8.74 (m, 1H), 7.94 (d, J=1.7 Hz, 1H), 7.46 (s, 1H), 6.68 (dd, J=2.7, 1.7 Hz, 1H), 3.22 (t, J=6.5 Hz, 2H), 2.32-2.14 (m, 1H), 1.93-1.79 (m, 4H), 1.63 (ddt, J=11.7, 8.3, 4.4 Hz, 1H), 1.32-0.99 (m, 4H).

LRMS m/z calcd. For C₁₆H₁₉N₅O₂[M+H]⁺: 370.15, found: 370.2

### Example 129 Synthesis of Methyl]-(3-hydroxy-5-(((4-(trifluoromethyl)cyclohexyl)methyl)carbamoyl)pyridin-2-yl)-]H-pyrazole-4-carboxylate (154)

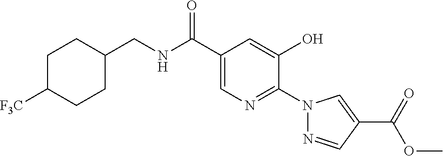 [SMILES: COC(=O)c1cnn(-c2ncc(C(=O)NCC3CCC(C(F)(F)F)CC3)cc2O)c1]

128 (10 mg, 0.02 mmol) was added to a 10 mL round bottom flask and flushed with N₂ before the addition of anhydrous MeOH (3 mL). 10% sodium methoxide in methanol solution (500 L) was added to the reaction mixture and the resultant mixture was stirred at room temperature of 16 hr. Formic acid was added to neutralise the reaction mixture before the addition of celite. The solvent was removed under vacuum and the crude mixture was purified using flash column chromatography (0-10% MeOH in CH₂Cl₂) to give the titled compound (4.5 mg, 0.010 mmol, 52%).

¹H NMR (400 MHz, DMSO) δ 8.93 (d, J=0.7 Hz, 1H), 8.71 (t, J=6.0 Hz, 1H), 8.47 (d, J=1.9 Hz, 1H), 8.28 (d, J=0.7 Hz, 1H), 7.90 (d, J=1.9 Hz, 1H), 3.82 (s, 3H), 3.15 (t, J=6.5 Hz, 2H), 1.95-1.78 (m, 5H), 1.30-0.96 (m, 5H).

LRMS m/z calcd. For C₁₉H₂₂F3N₄04M+H]⁺: 427.16, found: 427.2

### Reference Example 130 Synthesis of Ethyl 1-carbamimidoyl-]H-pyrazole-4-carboxylate (155)

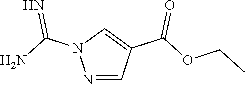

To a solution of 1H-pyrazole-4-carboxylic acid ethyl ester (6.66 g, 47.62 mmol), cyanamide (2.0 g, 47.62 mmol) and dioxane (30 mL) was added a solution of 4M HCl in dioxane (20 mL). The reaction mixture was heated to 100° C. for 3 hrs. The reaction was cooled to room temperature and Et₂O (30 mL was added. The precipitate was collected to yield the title compound (3 g, 16.4 mmol, 35%).

LRMS m/z calcd. For C₇H₁₁N₄02[M+H]⁺: 183.09, found: 183.1

### Reference Example 131 Synthesis of Methyl 1-(5-cyano-4-hydroxypyrimidin-2-yl)-]H-pyrazole-4-carboxylate (156)

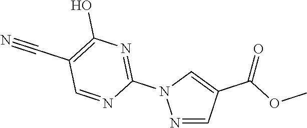 [SMILES: COC(=O)c1cnn(-c2ncc(C#N)c(O)n2)c1]

Ethyl (E)-2-cyano-3-ethoxyacrylate (3.0 g, 17.75 mmol) was added to a solution of 155 (3.23 g, 17.75 mmol), K₂CO₃ (4.9 g, 35.50 mmol) and methanol (100 mL). The resultant mixture was stirred at room temperature for 1 hr. H₂O (50 mL) was added, and the white precipitate was filtered to give the titled compound (3.52 g, 13.59 mmol, 76%).

LRMS m/z calcd. For C₁₀H₈N₅O₃[M+H]⁺: 246.06, found: 246.1

### Reference Example 132 Synthesis of 1-(5-cyano-4-hydroxypyrimidin-2-yl)-1H-pyrazole-4-carboxylic acid (157)

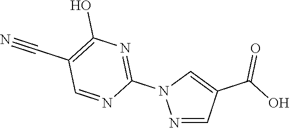 [SMILES: N#Cc1cnc(-n2cc(C(=O)O)cn2)nc1O]

A solution of 156 (3.0 g, 13.1 mmol) in MeOH (15 mL) and H₂O (10 mL) was prepared, before the addition of lithium hydroxide monohydrate (904 mg, 39.3 mmol). The reaction mixture was stirred for 4 hrs. The reaction mixture was neutralised with 1N HCl and the MeOH was removed under vacuum. The precipitate was collected to give the titled compound (2.49 g, 10.8 mmol, 83%).

LRMS m/z calcd. For C₉H₄N₅O₃[M−H]: 230.03, found: 230.4

### Example 133 Synthesis of N-([1,1′-biphenyl]-4-ylmethyl)-1-(5-cyano-4-hydroxypyrimidin-2-yl)-]H-pyrazole-4-carboxamide (158)

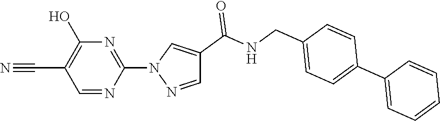 [SMILES: N#Cc1cnc(-n2cc(C(=O)NCc3ccc(-c4ccccc4)cc3)cn2)nc1O]

Following general procedure B: 157 (10 mg, 0.04 mmol), HATU (33 mg, 0.09 mmol), 4-phenylbenzylamine (11.88 mg, 0.06 mmol) and DIPEA (22 μL, 0.13 mmol) gave the titled compound (8 mg, 0.020 mmol, 57%).

¹H NMR (400 MHz, DMSO) δ 9.15 (s, 1H), 8.99 (t, J=6.0 Hz, 1H), 8.59 (s, 1H), 8.23 (s, 1H), 7.64 (dd, J=7.9, 4.7 Hz, 4H), 7.51-7.31 (m, 5H), 4.48 (d, J=6.0 Hz, 2H).

LRMS m/z calcd. For C₂₂H₁₇N₆O₂[M+H]⁺: 397.14, found: 397.2

### Example 134 Synthesis of 1-(5-cyano-4-hydroxypyrimidin-2-yl)-N-((4-(trifluoromethyl)cyclohexyl)methyl)-]H-pyrazole-4-carboxamide (159)

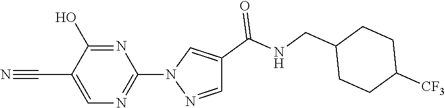 [SMILES: N#Cc1cnc(-n2cc(C(=O)NCC3CCC(C(F)(F)F)CC3)cn2)nc1O]

Following general procedure B: 157 (37 mg, 0.16 mmol), C-4 trifluoromethyl-cyclohexyl-methamine (28 μL, 0.19 mmol), T3P (190 μL, 0.32 mmol) DIPEA (83 μL, 0.48 mmol) gave the title compound (9 mg, 0.022 mmol, 14%).

¹H NMR (400 MHz, DMSO) δ 9.05 (s, 1H), 8.27 (d, J=6.7 Hz, 2H), 7.98 (s, 1H), 3.07 (t, J=6.0 Hz, 2H), 2.29-2.12 (m, 1H), 1.85 (t, J=16.4 Hz, 4H), 1.64-1.42 (m, 1H), 1.27-1.13 (m, 2H), 1.05-0.91 (m, 2H).

LRMS m/z calcd. For C₁₇H₁₈F3N₆02[M+H]⁺: 395.14, found: 395.2

### Example 135 Synthesis of 1-(5-cyano-4-hydroxypyrimidin-2-yl)-N-((6-phenylpyridin-3-yl)methyl)-1H-pyrazole-4-carboxamide (160)

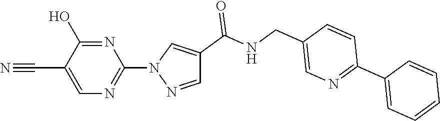 [SMILES: N#Cc1cnc(-n2cc(C(=O)NCc3ccc(-c4ccccc4)nc3)cn2)nc1O]

Following general procedure B: 157 (100 mg, 0.43 mmol), (6-phenylpyridin-3-yl)methanamine (119 mg, 0.65 mmol), T3P (515 μL, 0.87 mmol) DIPEA (223 μL, 0.130 mmol) gave the title compound (65 mg, 0.163 mmol, 38%).

¹H NMR (400 MHz, DMSO) δ 9.28-8.99 (m, 2H), 8.87-8.60 (m, 2H), 8.43-8.19 (m, 1H), 8.09-8.03 (m, 2H), 7.95 (d, J=8.1 Hz, 1H), 7.82 (ddd, J=16.8, 8.3, 2.4 Hz, 1H), 7.54-7.39 (m, 3H), 4.46 (d, J=5.5 Hz, 2H).

LRMS m/z calcd. For C₂₁H₁₆N₇O₂[M+H]⁺: 398.14, found: 398.1

### Example 136 Synthesis of Methyl4-(5-((1-(5-cyano-4-hydroxypyrimidin-2-yl)-]H-pyrazole-4-carboxamido)-methyl)-pyridin-2-yl)-benzoate (161)

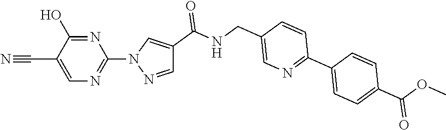 [SMILES: COC(=O)c1ccc(-c2ccc(CNC(=O)c3cnn(-c4ncc(C#N)c(O)n4)c3)cn2)cc1]

Following general procedure B: 157 (100 mg, 0.43 mmol), methyl 4-(5-(aminomethyl)pyridin-2-yl)benzoate (157 mg, 0.65 mmol), T3P (515 μL, 0.87 mmol) DIPEA (223 μL, 0.130 mmol) gave the title compound (11 mg, 0.0241 mmol, 6%).

¹H NMR (400 MHz, DMSO) δ 9.05 (s, 1H), 8.27 (d, J=6.7 Hz, 2H), 7.98 (s, 1H), 3.07 (t, J=6.0 Hz, 2H), 2.29-2.12 (m, 1H), 1.85 (t, J=16.4 Hz, 4H), 1.64-1.42 (m, 1H), 1.27-1.13 (m, 2H), 1.05-0.91 (m, 2H).

LRMS m/z calcd. For C₂₃H₁₈N₇O₄[M+H]⁺: 456.14, found: 456.2.

### Example 137 Synthesis of 4-(5-((1-(5-cyano-4-hydroxypyrimidin-2-yl)-1H-pyrazole-4-carboxamido) methyl)pyridin-2-yl)benzoic acid (162)

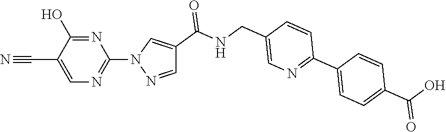 [SMILES: N#Cc1cnc(-n2cc(C(=O)NCc3ccc(-c4ccc(C(=O)O)cc4)nc3)cn2)nc1O]

Following general procedure D: 161 (5 mg, 0.01 mmol), LiCl (4.51 mg, 0.11 mmol) in DMF (1 mL) gave the titled compound (2 mg, 0.0045 mmol, 45%).

¹H NMR (400 MHz, DMSO) δ 9.13 (s, 1H), 9.04 (t, J=5.5 Hz, 1H), 8.68 (d, J=2.2 Hz, 1H), 8.57 (s, 1H), 8.23-8.16 (m, 3H), 8.04 (dd, J=8.3, 4.0 Hz, 3H), 7.86 (dd, J=8.3, 2.3 Hz, 1H), 4.52 (d, J=5.5 Hz, 2H).

LRMS m/z calcd. For C₂₂H₁₄N₇O₄[M−H]⁻: 440.11, found: 439.3, [M-2H]²⁻/2=220.0

### Example 138 Synthesis of Ethyl 6-(4-(([1,1′-biphenyl]-4-ylmethyl)carbamoyl)-]H-pyrazol-1-yl)-5-methoxynicotinate (163a)

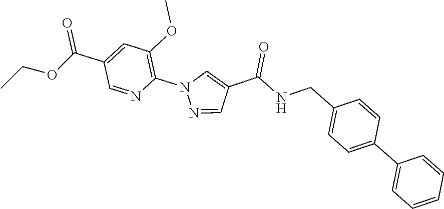 [SMILES: CCOC(=O)c1cnc(-n2cc(C(=O)NCc3ccc(-c4ccccc4)cc3)cn2)c(OC)c1]

Ethyl 6-chloro-5-methoxynicotinate (30 mg, 0.14 mmol), 92 (30 mg, 0.108 mmol), RockPhos G3 (10 mg, 0.014 mmol), Cs₂CO₃ (77 mg, 0.238 mmol) in dioxane (5 ml) for 7 days gave 163a (5 mg, 0.01 mmol, 9%) as a clear oil.

1H NMR (400 MHz, THF-d8) δ 8.71 (s, 1H,), 8.65 (d, J=1.0 Hz, 1H), 8.07-8.05 (m, 1H), 7.91 (t, J=6.0 Hz, 1H), 7.75 (d, J=1.0 Hz, 1H), 7.46-7.24 (m, 9H), 4.58 (d, J=6.0 Hz, 2H), 4.40 (q, J=7.0 Hz, 2H), 3.98 (s, 3H), 1.42 (t, J=7.0 Hz, 3H).

HRMS (ESI-TOF) calcd for C₂₆H₂₅O₄N₄ [M+H]⁺: 457.1870, found: 457.1867.

### Example 139 Synthesis of 6-(4-(([1,1′-biphenyl]-4-ylmethyl)carbamoyl)-]H-pyrazol-1-yl)-5-methoxynicotinic acid (164a)

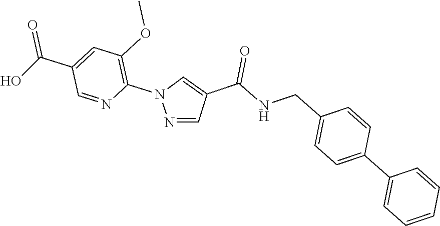 [SMILES: COc1cc(C(=O)O)cnc1-n1cc(C(=O)NCc2ccc(-c3ccccc3)cc2)cn1]

163a (5 mg, 0.0109 mmol) was dissolved in a mixture of THF and water (1.5 ml (10:1). Lithium hydroxide monohydrate (1.0 mg, 0.021 mmol) was added to the reaction mixture and the resulting mixture was allowed to stir at room temperature for 16 hrs. The reaction was confirmed to have gone to completion by TLC and HCLaq (5 ml, 1M) was added to the reaction mixture. The resultant mixture was extracted with EtOAc (3×10 ml), then washed with brine, then dried with anhydrous Na₂SO₄ and purified using flash column chromatography using (CH₂Cl₂, MeOH 0-5%, 1% formic acid) over 20 column volumes gave 164a (3.5 mg, 0.0082 mmol, 76%) as a clear oil.

1H NMR (400 MHz, DMSO-d6) δ 8.69 (s, 1H), 8.47 (s, 1H), 8.12 (s, 1H), 7.99 (s, 1H), 7.70-7.61 (m, 4H), 7.50-7.32 (m, 5H), 4.50 (d, J=6.0 Hz, 2H), 3.89 (s, 3H).

HRMS (ESI-TOF) calcd for C₂₄H₂₁O₄N₄ [M+H]⁺: 429.1557, found: 429.1557.

### Reference Example 140 Synthesis of 2,5-dimethylpyrazolo[1,5-a]pyrimidin-7-amine (163)

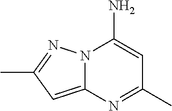

3-aminocrotononitrile (10 g, 0.12 mol) and 3-methyl-1H-pyrazol-5-amine (11.83 g, 0.12 mol) were combined and heated to 140° C. for 2 hrs. The resultant mixture was allowed to cool. The crude mixture was then recrystallised with EtOH to give the titled compound (6.68 g, 0.041 mol, 35%).

LRMS m/z calcd. For C₈H₁₁N₄ [M+H]⁺: 163.1, found: 163.10

### Reference Example 141 Synthesis of Diethyl 2-(((2,5-dimethylpyrazolo[1,5-a]pyrimidin-7-yl)amino)methylene)malonate (164)

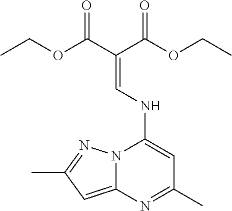 [SMILES: CCOC(=O)C(=CNc1cc(C)nc2cc(C)nn12)C(=O)OCC]

163 (1.50 g, 0.01 mmol) was taken up in toluene (10 mL) before the addition of diethyl 2-(ethoxymethylene)malonate (2.24 ml, 0.01 mmol). The resultant mixture was heated to 120° C. for 48 hr. The reaction mixture was cooled to room temperature. Celite was added to the reaction mixture before the solvent was removed under vacuum. The crude compound was purified using flash chromatography (0-10% MeOH) to give the titled compound (2.26 g, 0.0068 mmol, 74%).

LRMS m/z calcd. For C₁₆H₂₁N₄O₄ [M+H]⁺: 333.16, found: 333.2

### Reference Example 142 Synthesis of Ethyl 6-hydroxy-2,5-dimethylpyrazolo[1,5-a]pyrido[3,2-e]pyrimidine-7-carboxylate (165)

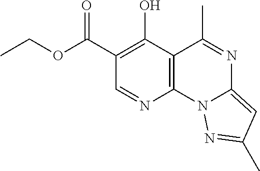 [SMILES: CCOC(=O)c1cnc2c(c(C)nc3cc(C)nn32)c1O]

164 (6.15 g, 18.52 mmol) was dissolved in Eaton's reagent (10 mL). The resultant mixture was heated to 70° C. for 4 hrs, and the progress was followed by LCMS. Once the starting material had be consumed, the reaction mixture was cooled to 0° C. and slowly poured into a saturated NaHCO₃ solution to quench the Eaton's reagent. The resultant mixture was then extracted with a CHCl₃ and IPA mixture (3×100 mL (3:1)). The organic fractions were combined, dried (Na₂SO₄), the solvent was removed under vacuum. The crude compound was purified using flash column chromatography (CHCl₃: MeOH (0-20%) to give the titled compound (4.67 g, 16.32 mmol, 88%).

LRMS m/z calcd. For C₁₄H₁₅N₄O₃[M+H]⁺: 287.11, found: 287.20

### Reference Example 143 Synthesis of Ethyl 6-chloro-2,5-dimethylpyrazolo[1,5-a]pyrido[3,2-e]pyrimidine-7-carboxylate (166)

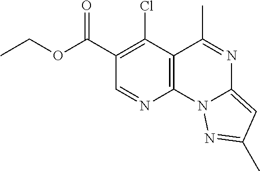 [SMILES: CCOC(=O)c1cnc2c(c(C)nc3cc(C)nn32)c1Cl]

165 (4.67 g, 16.32 mmol) was dissolved in phosphorus oxychloride (25 mL) and refluxed at 100° C. for 3 hrs. The reaction mixture was cooled to 0° C. and slowly quenched with saturated NaHCO₃ solution. The resultant mixture was extracted with a CHCl₃: IPA mixture (3:1 (3×100 mL)). The organic fractions were combined, dried (Na₂SO₄) and the solvents removed under vacuum. The crude mixture was then taken onto the next step without further purification (4.54 g, 14.93 mmol, 91%).

LRMS m/z calcd. For C₁₄H₁₄C₁N₄04[M+H]⁺: 305.08, found: 305.2

### Reference Example 144 Synthesis of Methyl 6-methoxy-2,5-dimethylpyrazolo[1,5-a]pyrido[3,2-e]pyrimidine-7-carboxylate (167)

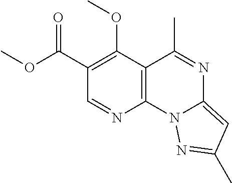

166 (156 mg, 0.51 mmol) was dissolved in MeOH (10 mL) before the addition of sodium methoxide solution (5 mL, 0.5 M). The resultant mixture was stirred at room temperature for 4 hrs. Celite was added to the reaction mixture before the solvent was removed under vacuum. The crude mixture was then purified using flash column chromatography (MeOH in CHCl₃ (0-10%) to give the titled compound (38 mg, 0.13 mmol, 26%).

LRMS m/z calcd. For C₁₄H₁₅N₄O₃[M+H]⁺: 287.11, found: 287.2

### Example 145 Synthesis of 6-methoxy-2,5-dimethyl-N-((4-(trifluoromethyl)cyclohexyl)methyl)pyrazolo[1,5-a]pyrido[3,2-e]pyrimidine-7-carboxamide (168)

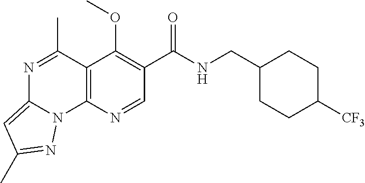 [SMILES: COc1c(C(=O)NCC2CCC(C(F)(F)F)CC2)cnc2c1c(C)nc1cc(C)nn12]

Following general procedure A: 167 (30 mg, 0.1 mmol), 4-trifluoromethyl-cyclohexylamine (19 μL, 0.16 mmol) and DABAL (26.85 mg, 0.1 mmol) gave the titled compound (9 mg, 0.02 mmol, 20%).
Solvent system used for purification: 0%-15% MeOH in CH₃Cl.

LRMS m/z calcd. For C₂₁H₂₅F3N₅02[M+H]:436.20, found: 436.2.

### Example 146 Synthesis of N-([1,1′-Biphenyl]-4-ylmethyl)-6-hydroxypyrazolo[1,5-a]pyrido[3,2-e]pyrimidine-7-carboxamide (169)

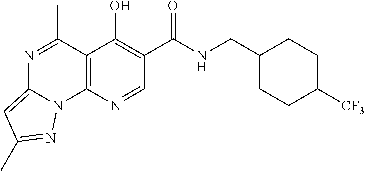 [SMILES: Cc1cc2nc(C)c3c(O)c(C(=O)NCC4CCC(C(F)(F)F)CC4)cnc3n2n1]

Following general procedure D: 168 (9 mg, 0.02 mmol), LiCl (8.6 mg, 0.21 mmol) in DMSO (1 mL) gave the titled compound (1.5 mg, 0.0035 mmol, 18%).
Solvent system used for purification: Reverse phase (0%-100% ACN (0.1% formic acid)) in H₂O (0.1% formic acid).

¹H NMR (400 MHz, DMSO) δ 8.91 (s, 1H), 6.42 (s, 1H), 3.23 (d, J=5.1 Hz, 2H), 2.66 (s, 3H), 2.41 (s, 3H), 2.07 (s, 1H), 1.83-1.75 (m, 2H), 1.65 (d, J=12.7 Hz, 2H), 1.48 (d, J=9.6 Hz, 1H), 1.25-1.08 (m, 2H), 0.95-0.81 (m, 2H).

LRMS m/z calcd. For C₂₀H₂₃F3N₅02[M+H]⁺: 422.18, found⁺: 422.2

### Example 147 Synthesis of N-((6-chloropyridin-3-yl)methyl)-6-methoxy-2,5-dimethylpyrazolo[1,5-a]pyrido[3,2-e]pyrimidine-7-carboxamide (170)

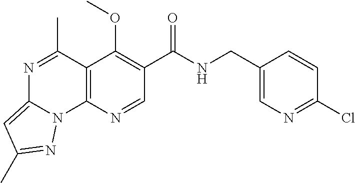 [SMILES: COc1c(C(=O)NCc2ccc(Cl)nc2)cnc2c1c(C)nc1cc(C)nn12]

Following general procedure A: 167 (900 mg, 3.15 mmol), (6-chloropyridin-3-yl)methanamine (532 mg, 3.78 mmol) and DABAL (805 mg, 3.15 mmol) gave the titled compound (364 mg, 0.91 mmol, 29%).
Solvent system used for purification: 0%-15% MeOH in CH₃Cl.

LRMS m/z calcd. For C₁₉H₁₈ClN₆O₂[M+H]⁺: 397.12, found: 397.10

### Example 148 Synthesis of N-((6-(4-carbamoylphenyl)pyridin-3-yl)methyl)-6-hydroxy-2,5-dimethylpyrazolo[1,5-a]pyrido[3,2-e]pyrimidine-7-carboxamide (171)

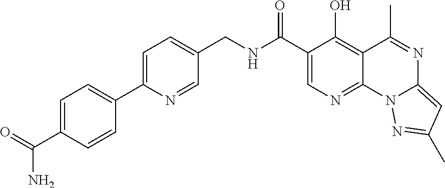 [SMILES: Cc1cc2nc(C)c3c(O)c(C(=O)NCc4ccc(-c5ccc(C(N)=O)cc5)nc4)cnc3n2n1]

Following general procedure C: 170 (15 mg, 0.04 mmol), (4-(carbamoyl)phenyl)boronic acid (9.75 mg, 0.06 mmol), Pd Amphos (2.77 mg, 0.0039 mmol) and cesium carbonate (38.19 mg, 0.12 mmol) gave the titled compound (2 mg, 0.0042 mmol, 11%).

¹H NMR (400 MHz, DMSO) δ 8.77 (s, 1H), 8.69-8.66 (m, 1H), 8.31 (s, 1H), 8.14 (t, J=4.2 Hz, 3H), 7.99 (dd, J=17.9, 8.4 Hz, 3H), 7.86 (dd, J=8.1, 2.5 Hz, 1H), 6.53 (s, 2H), 6.20 (s, 1H), 4.59 (d, J=6.0 Hz, 2H), 2.87 (s, 3H), 2.36 (s, 3H).

LRMS m/z calcd. For C₂₅H₂₂N₇O₃[M+H]⁺: 468.18, found: 468.1, [M+2H]²⁺/2=234.70

### Example 149 Synthesis of 6-hydroxy-2,5-dimethyl-N-((6-(pyrimidin-5-yl)pyridin-3-yl)methyl)pyrazolo[1,5-a]pyrido[3,2-e]pyrimidine-7-carboxamide (172)

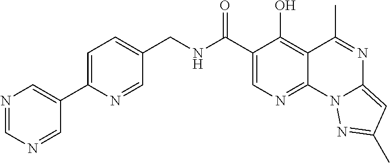 [SMILES: Cc1cc2nc(C)c3c(O)c(C(=O)NCc4ccc(-c5cncnc5)nc4)cnc3n2n1]

Following general procedure C: 170 (15 mg, 0.04 mmol), 5-(4,4,5,5-tetramethyl-1,3,2-dioxaborolan-2-yl) pyrimidine (12.1 mg, 0.06 mmol), Pd Amphos (1.39 mg, 0.0019 mmol) and cesium carbonate (38.19 mg, 0.12 mmol) gave the titled compound (2.5 mg, 0.0058 mmol, 15%).

¹H NMR (400 MHz, DMSO) δ 10.69 (s, 1H), 9.42 (s, 2H), 9.24 (s, 1H), 8.74 (d, J=2.2 Hz, 1H), 8.59 (s, 1H), 8.14-8.09 (m, 1H), 7.93 (dd, J=8.2, 2.3 Hz, 1H), 6.52 (s, 1H), 6.42 (s, 1H), 4.63 (d, J=6.0 Hz, 2H), 2.89 (s, 3H), 2.45 (s, 3H).

LRMS m/z calcd. For C₂₂H₁₉N₈O₂[M+H]⁺: 427.16, found: 427.1, [M+2H]²⁺/2=214.20

### Example 150 Synthesis of 6-hydroxy-2,5-dimethyl-N-((6-(4-morpholinophenyl)pyridin-3-yl)methyl)pyrazolo[1,5-a]pyrido[3,2-e]pyrimidine-7-carboxamide (173)

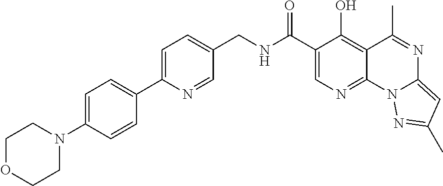 [SMILES: Cc1cc2nc(C)c3c(O)c(C(=O)NCc4ccc(-c5ccc(N6CCOCC6)cc5)nc4)cnc3n2n1]

Following general procedure C: 170 (15 mg, 0.04 mmol), 4-morpholine-phenylboronic acid (12.1 mg, 0.06 mmol), Pd Amphos (1.39 mg, 0.0019 mmol) and cesium carbonate (38.19 mg, 0.12 mmol) gave the titled compound (5 mg, 0.009 mmol, 25%).

¹H NMR (400 MHz, DMSO) δ 10.50 (s, 1H), 8.59-8.48 (m, 2H), 7.98-7.93 (m, 2H), 7.83 (dd, J=8.3, 0.9 Hz, 1H), 7.76 (dd, J=8.2, 2.3 Hz, 1H), 7.05-6.99 (m, 2H), 6.53 (s, 1H), 6.45 (s, 1H), 4.57 (d, J=5.9 Hz, 2H), 3.77-3.71 (m, 4H), 3.23-3.14 (m, 4H), 2.88 (s, 3H), 2.46 (s, 3H).

LRMS m/z calcd. For C₂₈H₂₈N₇₀₃[M+H]⁺: 510.22, found [M+2H]²⁺/2=255.70

### Example 151 Synthesis of 6-hydroxy-2,5-dimethyl-N-((6-(4-(methylcarbamoyl)phenyl)pyridin-3-yl)methyl)pyrazolo[1,5-a]pyrido[3,2-e]pyrimidine-7-carboxamide (174)

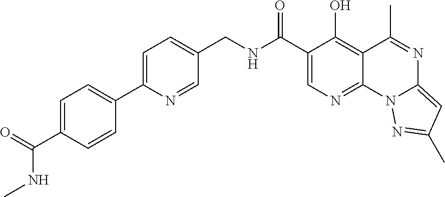

Following general procedure C: 170 (15 mg, 0.04 mmol), 4-N methylcarbonyl-phenylboronic acid acid (10.52 mg, 0.06 mmol), Pd Amphos (2.77 mg, 0.0039 mmol) and cesium carbonate (38.19 mg, 0.12 mmol) gave the titled compound (1 mg, 0.0021 mmol, 5%).

¹H NMR (400 MHz, DMSO) δ 8.67 (d, J=6.2 Hz, 2H), 8.51 (d, J=5.0 Hz, 1H), 8.18-8.10 (m, 2H), 8.02 (d, J=8.2 Hz, 1H), 7.93 (d, J=8.4 Hz, 2H), 7.88-7.78 (m, 1H), 6.52 (s, 1H), 6.34 (s, 1H), 4.61 (d, J=5.8 Hz, 2H), 2.88 (s, 3H), 2.80 (d, J=2.3 Hz, 3H), 2.41 (s, 3H).

LRMS m/z calcd. For C₂₆H₂₄N₇₀₃[M+H]⁺: 482.19, found: [M+2H]²⁺/2=241.50

### Example 152 Synthesis of 6-hydroxy-2,5-dimethyl-N-((6-phenylpyridin-3-yl)methyl)pyrazolo[1,5-a]pyrido[3,2-e]pyrimidine-7-carboxamide (175)

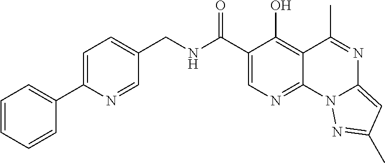 [SMILES: Cc1cc2nc(C)c3c(O)c(C(=O)NCc4ccc(-c5ccccc5)nc4)cnc3n2n1]

Following general procedure C: 170 (50 mg, 0.13 mmol), phenylboronic acid (46.21 mg, 0.38 mmol), Pd Amphos (8.94 mg, 0.01 mmol) and cesium carbonate (123 mg, 0.38 mmol) gave the titled compound (9 mg, 0.0212 mmol, 22%).

¹H NMR (400 MHz, DMSO) δ 9.42 (s, 1H), 8.95 (s, 1H), 8.49 (s, 1H), 8.09-8.00 (m, 2H), 7.89 (d, J=8.2 Hz, 1H), 7.69-7.62 (m, 1H), 7.51-7.36 (m, 3H), 6.47 (s, 1H), 4.64 (s, 2H), 2.84 (s, 3H), 2.43 (s, 3H).

LRMS m/z calcd. For C₂₄H₂₁N₆O₂[M+H]⁺: 425.17, found: 425.2, [M+2H]²⁺/2=213.2

### Example 153 Synthesis of 6-hydroxy-2,5-dimethyl-N-((6-(3-morpholinophenyl)pyridin-3-yl)methyl)pyrazolo[1,5-a]pyrido[3,2-e]pyrimidine-7-carboxamide (176)

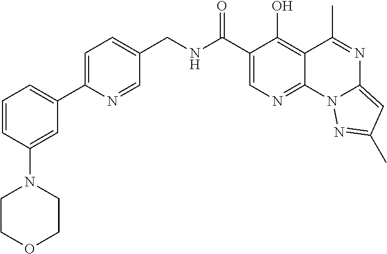 [SMILES: Cc1cc2nc(C)c3c(O)c(C(=O)NCc4ccc(-c5cccc(N6CCOCC6)c5)nc4)cnc3n2n1]

Following general procedure C: 170 (40 mg, 0.10 mmol), 3-morpholo-phenylboronic acid (63 mg, 0.30 mmol), Pd Amphos (7.15 mg, 0.01 mmol) and cesium carbonate (99 mg, 0.30 mmol) gave the titled compound (14 mg, 0.275 mmol, 28%).

¹H NMR (400 MHz, DMSO) δ 9.52 (s, 1H), 8.95 (s, 1H), 8.46 (d, J=2.2 Hz, 1H), 7.88 (d, J=8.2 Hz, 1H), 7.68-7.56 (m, 2H), 7.46 (d, J=7.6 Hz, 1H), 7.31 (t, J=7.9 Hz, 1H), 7.00 (dd, J=8.3, 2.5 Hz, 1H), 6.46 (s, 1H), 4.67-4.60 (m, 2H), 3.75 (t, J=4.7 Hz, 4H), 3.16 (dd, J=6.6, 3.2 Hz, 4H), 2.84 (s, 3H), 2.43 (s, 3H).

LRMS m/z calcd. For C₂₈H₂₈N₇O₃[M+H]⁺: 510.22, found: 510.30, [M+2H]²⁺/2=255.80

### Example 154 Synthesis of N-((6-(3-carbamoylphenyl)pyridin-3-yl)methyl)-6-hydroxy-2,5-dimethylpyrazolo[1,5-a]pyrido[3,2-e]pyrimidine-7-carboxamide (177)

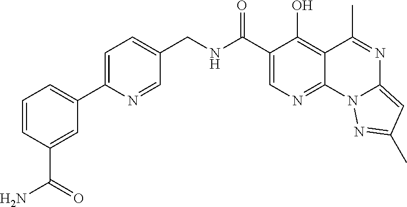 [SMILES: Cc1cc2nc(C)c3c(O)c(C(=O)NCc4ccc(-c5cccc(C(N)=O)c5)nc4)cnc3n2n1]

Following general procedure C: 170 (40 mg, 0.10 mmol), 3-amido-phenylboronic acid (50 mg, 0.30 mmol), Pd Amphos (7.15 mg, 0.01 mmol) and cesium carbonate (99 mg, 0.30 mmol) gave the titled compound (11 mg, 0.0235 mmol, 24%).

¹H NMR (400 MHz, DMSO) δ 9.41 (s, 1H), 8.95 (s, 1H), 8.56-8.49 (m, 2H), 8.21-8.06 (m, 2H), 7.97 (d, J=8.2 Hz, 1H), 7.91 (d, J=7.7 Hz, 1H), 7.71 (dd, J=8.3, 2.3 Hz, 1H), 7.55 (t, J=7.7 Hz, 1H), 7.43 (s, 1H), 6.47 (s, 1H), 4.66 (d, J=4.1 Hz, 2H), 2.85 (s, 3H), 2.43 (s, 3H).

LRMS m/z calcd. For C₂₅H₂₂N₇O₃[M+H]⁺: 468.18, found: 468.2, [M+2H]²⁺/2=234.80 Example 155 Synthesis of N-((6-(benzo[d]thiazol-5-yl)pyridin-3-yl)methyl)-6-hydroxy-2,5-dimethylpyrazolo[1,5-a]pyrido[3,2-e]pyrimidine-7-carboxamide (178)

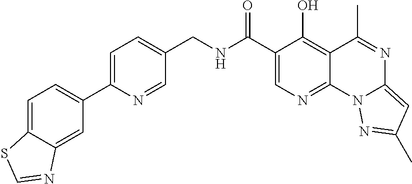 [SMILES: Cc1cc2nc(C)c3c(O)c(C(=O)NCc4ccc(-c5ccc6scnc6c5)nc4)cnc3n2n1]

Following general procedure C: 170 (50 mg, 0.13 mmol), 5-(4,4,5,5-tetramethyl-1,3,2-dioxaborolan-2-yl)benzo[d]thiazole (98.86 mg, 0.38 mmol), Pd Amphos (8.94 mg, 0.01 mmol) and cesium carbonate (123 mg, 0.38 mmol) gave the titled compound (5 mg, 0.0103 mmol, 8%).

¹H NMR (400 MHz, DMSO) δ 9.44 (s, 1H), 8.96 (s, 1H), 8.73 (d, J=1.6 Hz, 1H), 8.54 (d, J=2.3 Hz, 1H), 8.27-8.19 (m, 2H), 8.08 (d, J=8.2 Hz, 1H), 7.71 (dd, J=8.3, 2.3 Hz, 1H), 6.46 (s, 1H), 4.65 (d, J=4.6 Hz, 2H), 2.85 (s, 3H), 2.43 (s, 3H).

LRMS m/z calcd. For C₂₅H₂₀N₇O₂S[M+H]⁺: 482.14, found 482.20, [M+2H]²⁺/2=241.70

### Example 156 Synthesis of 6-hydroxy-2,5-dimethyl-N-((6-(3-(methylcarbamoyl)phenyl)pyridin-3-yl)methyl)pyrazolo[1,5-a]pyrido[3,2-e]pyrimidine-7-carboxamide (179)

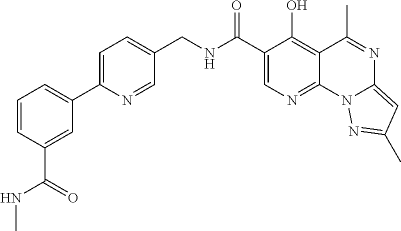 [SMILES: CNC(=O)c1cccc(-c2ccc(CNC(=O)c3cnc4c(c(C)nc5cc(C)nn54)c3O)cn2)c1]

Following general procedure C: 170 (29 mg, 0.07 mmol), 3-(methylcarbamoyl)phenyl)boronic acid (39.33 mg, 0.22 mmol), Pd Amphos (5.18 mg, 0.01 mmol) and cesium carbonate (71 mg, 0.22 mmol) gave the titled compound (11 mg, 0.0228 mmol, 32%).

¹H NMR (400 MHz, DMSO) δ 9.96 (s, 1H), 8.96 (s, 1H), 8.60-8.44 (m, 3H), 8.16 (d, J=7.9 Hz, 1H), 7.95 (d, J=8.2 Hz, 1H), 7.86 (d, J=7.7 Hz, 1H), 7.70 (dd, J=8.2, 2.3 Hz, 1H), 7.54 (t, J=7.7 Hz, 1H), 6.45 (s, 1H), 4.64 (s, 2H), 2.84 (s, 3H), 2.80 (d, J=4.4 Hz, 3H), 2.42 (s, 3H).

LRMS m/z calcd. for C₂₆H₂₃N₇O₃ [M+H]⁺: 482.19, found: 482.2, [M+2H]²⁺/2=241.60

### Example 157 Synthesis of (S)-6-hydroxy-N-(1-(4-methoxyphenyl)ethyl)-2,5-dimethylpyrazolo[1,5-a]pyrido[3,2-e]pyrimidine-7-carboxamide (180)

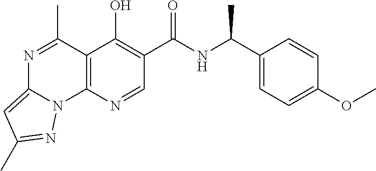

Following general procedure A: 167 (50 mg, 0.17 mmol), (S)-1-(4-methoxyphenyl)ethan-1-amine (31 μL, 0.21 mmol) and DABAL-Me₃ (44.76 mg, 0.17 mmol) gave the titled compound (7 mg, 0.0179 mmol, 11%).
Solvent system used for purification: 0%-15% MeOH in CH₃Cl.

¹H NMR (400 MHz, DMSO) δ 10.40 (d, J=7.9 Hz, 1H), 8.51 (s, 1H), 7.35-7.28 (m, 2H), 6.91 (dd, J=8.5, 1.9 Hz, 2H), 6.47 (s, 1H), 5.16-5.05 (m, 1H), 3.74 (s, 3H), 2.89 (s, 3H), 2.46 (s, 3H), 1.47 (d, J=6.9 Hz, 3H).

LRMS m/z calcd. For C₂₁H₂₂N₅O₃[M+H]⁺: 392.17, found: 392.20

### Activity Examples

### General Experimental Methods

### Preparation of tPHD2 (residues 181-426)

In brief, cDNA encoding for the catalytic domain of tPHD2 (181-426 residues) was cloned into the pET28a(+)/pET24a(+) vectors (Novagen), to enable production of tPHD2 (residues 181-426) protein with/without an N-terminal His6-tag. The tPHD2 (residues 181-426) encoding construct was transformed into the Escherichia coli BL21 DE3 cell line; protein production was induced with 0.5 mM isopropyl-b-D-thiogalactosidase (3-5 hr at 28° C.). Cells were harvested and lysed by sonication in 20 mM Tris-HCl (pH 7.0) and 0.3 M NaCl; soluble protein (about 5% total soluble extract) was purified by immobilized Ni ion affinity chromatography using pentadentate tris-carboxymethyl ethylene diamine resin followed by cleavage of the His6-tag by thrombin (or alternately by cation exchange chromatography) with a final purification by gel filtration chromatography. The protein was exchanged into 50 mM Tris-HCl buffer (pH 7.5) and concentrated to % 40 mg/ml. The protein was of >95% purity, as determined by SDS-PAGE analysis and electrospray ionization mass spectrometric analysis.

### PHD2 Hydroxylation Assays

The PHD2 RF-MS RapidFire chromatography mass spectrometry (RF-MS) assay monitors turnover of a C-terminal oxygenase dependent domain (CODD) peptide substrate DLDLEMLAPYIPMDDDFQL-CONH₂ and appearance of the hydroxylated peptide product (Pro564) in an endpoint type assay format (typical enzyme incubation time of 15 minutes). Tris(hydroxymethyl)aminomethane was from Fisher. Ferrous ammonium sulfate (FAS), 2-oxoglutarate (2OG) and L-ascorbic acid were from Sigma Aldrich; solutions of these were prepared freshly each day. All inhibition assays were carried out in 384-well polypropylene plates (Greiner Bio-One). PHD2 assays were performed in assay buffer (50 mM Tris·Cl pH 7.5, 50 mM NaCl). Titrations of compounds for IC₅₀ determinations (3-fold and 11-point IC₅₀) were prepared using an ECHO 550 acoustic dispenser (Labcyte) and dry dispensed into 384-well polypropylene assay plates. The final assay concentration of DMSO was kept constant at 0.5%. PHD2 protein was prepared at a concentration of 300 nM in assay buffer and 25 μl dispensed across each 384-well assay plate. The PHD2 solution was allowed to equilibrate with the inhibitors for 15 minutes at room temperature; the enzyme reaction then initiated by dispense of 25 μl of substrate (20 M FAS, 200 M L-ascorbic acid, 10 M CODD peptide and 20 M 2OG in the assay buffer. Enzyme reactions were allowed to proceed for 20 minutes at room temperature and the reaction terminated by addition of 10% formic acid (5 l). Assay plates were then transferred to a RapidFire RF360 sampling robot (Agilent) connected to an Agilent 6530 accurate mass quadrupole-time-of-flight (Q-TOF) mass spectrometer. Assay samples were aspirated under vacuum and loaded onto a C4 solid phase extraction (SPE) cartridge. After loading the C4 SPE was washed with 0.1% formic acid in water to remove non-volatile buffer salts and then peptide was eluted from the SPE with 85% acetonitrile, 15% water containing 0.1% formic acid onto the mass spectrometer. Peptide charge states were monitored in positive mode. Ion chromatogram data were extracted for the +2 charge state and peak area data integrated using RapidFire Integrator software (Agilent). % conversion of the CODD peptide substrate to the +16 hydroxylated peptide was calculated using the equation: % conversion=100×hydroxylated/(hydroxylated+non-hydroxylated peptide). IC₅₀ values were determined from non-linear regression plots using GraphPad prism.

Cell Culture and Immunoblotting with Hep3B Cells
These were carried out as reported in T. L. Yeh et al, Chem Sci, 2017, 8, 7651-7668
Cell Culture with HEK293T Cells:
HEK293T cells were grown in Dulbecco's Modified Eagle's Medium DMIEM (high glucose, pyruvate, no glutamine, Gibco) supplemented with 1 0% of FBS (Sigma Aldrich F7524-500ML) and 1% GlutaMAX (Gibco) in a 37° C. incubator at 5% CO₂. Cells were grown to 90% confluency.

Inhibitor addition and incubation:

Cells were plated at a density of 1.2×10⁶ in T75 flasks. Cells were exposed to the inhibitor at a final concentration at 1% DMSO and were incubated at 37° C. for 3-18 hours.
Protein extraction and analysis with HEK293T cells:
The cells were washed twice with ice-cold PBS (Sigma, D8537). 1× RIPA buffer [Sigma, R0278] buffer, and a protease inhibitor [Complete™, Mini, EDTA-free Protease Inhibitor Cocktail, Roche]) were used for protein extraction. Adherent cells were scraped using a scraper and transferred to an ice-cooled microcentrifuge tube. The cell suspension was either frozen at −20° C. or incubated for 45 min on ice and sonicated for 3 cycles of 10 seconds pulse with 5 seconds intervals. After centrifuging (16,000 g, 15 min, 4° C.), the cell supernatant was collected in a new microcentrifuge tube. The protein concentrations were determined using a BCA protein assay kit (Thermo Scientific™ Pierce™ BCA Protein Assay Kit).
SDS-PAGE Analyses with HEK293T Cells
To perform the Polyacrylamide gel electrophoresis all the samples were loaded into a pre-cast NuPAGE 4-12% Bis-Tris Protein Gels (Life Tech). The gels were run in 1× Tris/glycine/SDS running buffer (20× NuPAGE MES SDS Running Buffer, Life tech) at 180 V for 45 min (Mini Gel Tank, Life Technologies). 5 μl of protein ladder marker (Page Ruler Prestained Protein Ladder, Thermo Scientific) was used to compare protein sizes.
Western Blot Analyses with HEK293T Cells
A gel with the resolved proteins was then transferred onto a nitrocellulose membrane (Amersham Protran Premium 0.2 NC 300 mm, GE Healthcare) by using a 1× transfer buffer (20× NuPAGE Transfer Buffer, Invitrogen). Mini Protean Tetra Cell, Bio-Rad used for the transfer. The transfer was performed at 100 volts for 1 hour. Membranes were blocked with 5% milk powder in 1× PBS-T for 30 mins then incubated overnight at 4° C. with primary antibodies (in 1:1000 dilutions) which were prepared in 1% milk powder in 1× PBS-T buffer. The membranes were washed three times for 10-minutes with 1× PBS-T, then incubated for the second time for 1 hour at RT in a horseradish peroxidase (HRP) conjugated secondary antibody (in 1:5000 dilutions), prepared with 1% milk powder in 1× PBS-T. Later the blots were washed three times 10-min with 1× PBS-T and followed by GE Healthcare Amersham™ ECL™ Prime Western Blotting Detection Reagent (RPN2236) and protein levels were measured by densitometric analysis using Bio-Rad Universal Hood iii. Values were normalized to glyceraldehyde 3-phosphate dehydrogenase (GAPDH) and the corresponding controls such as 1% (v/v) dimethyl sulfoxide (DMSO), 20 μM of Roxadustat. The primary antibodies used were Purified Mouse Anti-Human HIF-1α [Catalog Number 610959]. Secondary antibodies used were Rabbit Anti-Mouse IgG (D3V2A) mAb (HRP Conjugate).
In vivo Blood Counts—Reticulocytes and Hemoglobin
Groups of seven C57BL/6 mice were administered with a twice daily dose (IP) with either vehicle (1% methylcellulose), compound 68 (Example 46) at 15 mg/kg or 30 mg/kg or Daprodustat (2) at 30 mg/kg. Blood samples were taken either pre-treatment, on the 4^{th} day into treatment or on the 8th day of treatment. Blood samples were analysed using Celltac Alpha MEK-6500K. Statically significant increases in red blood cell count (RBC), haemoglobin (HGB) and haematocrit (HCT) were observed in compound treated mice.

### Example 158 Structure Activity Relationship (SAR) Studies for PHD Inhibition

SAR studies for PHD2 inhibition were carried out on the compounds of the invention. Compounds were screened against PHD2 using the RF-MS hydroxylation assay. The results are displayed in Table 1 and show the compounds of the invention have high potency as PHD2 inhibitors.

TABLE 1

| Cpd | | IC₅₀ |
|---|---|---|
| No | Example | μM |
| 16 | 3 | + |
| 20 | 7 | + |
| 22 | 9 | + |
| 31 | 13 | +++ |
| 32 | 14 | ++ |
| 41 | 23 | +++ |
| 42 | 24 | +++ |
| 43 | 25 | ++ |
| 44 | 26 | + |
| 64 | 42 | +++ |
| 65 | 43 | +++ |
| 66 | 44 | +++ |
| 67 | 45 | +++ |
| 68 | 46 | +++ |
| 69 | 47 | +++ |
| 75 | 51 | ++ |
| 76 | 52 | ++ |
| 84 | 60 | +++ |
| 85 | 61 | +++ |
| 86 | 62 | +++ |
| 89 | 65 | +++ |
| 90 | 66 | +++ |
| 91 | 67 | +++ |
| 93 | 69 | + |
| 95 | 71 | +++ |
| 96 | 72 | ++ |
| 97 | 73 | + |
| 99 | 75 | ++ |
| 101 | 77 | ++ |
| 103 | 79 | + |
| 108 | 84 | + |
| 109 | 85 | ++ |
| 117 | 93 | +++ |
| 119 | 95 | +++ |
| 122 | 98 | +++ |
| 123 | 99 | +++ |
| 124 | 100 | +++ |
| 125 | 101 | +++ |
| 129 | 105 | +++ |
| 158 | 133 | +++ |
| 162 | 137 | ++ |
| 176 | 153 | ++ |
| 180 | 157 | +++ |

Key: +++ = 1 nM-1 μM, ++ = 1 μM-10 μM, +=> 10 μM

### Example 159—2OG oxygenase selectivity 68 (Example 46)

Thee selectivity of 68 (example 46) against the following purified human 2OG dependent oxygenases was examined using reported assay procedures (T. L. Yeh et al, Chem Sci, 2017, 8, 7651-7668): FIH (factor inhibiting HIF) (IC₅₀ for 68=>100 μM), KDM4A: lysine specific demethylase 4A (IC₅₀ for 68=>100 M), KDM5B: lysine specific demethylase 5B (IC₅₀ for 68=>100 μM) and KDM6B: lysine specific demethylase 6B (IC₅₀ for 68=>100 μM). These results show 68 is selective for the PHDs.

### Example 160 Immunoblots of Hep3B cells treated with compounds of the invention

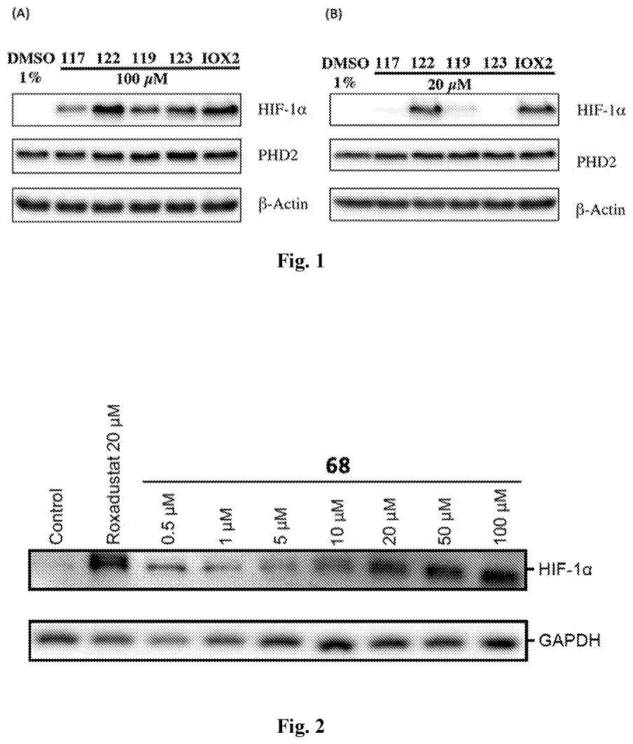

Hep3B cells were treated with compounds 117, 119, 122 and 123 of the invention (Examples 93, 95, 98 and 99) at 100 M (A) and 20 M (B) PHD inhibitors for 3 hours. The blots show protein levels of HIF1-α and β-actin at the 3^{rd} hour. Protocols for cell culture and immunoblotting were as performed in T. L. Yeh et al, Chem Sci, 2017, 8, 7651-7668. The results of this example show, as can be seen in FIG. 1, that the compounds of the invention stabilise HIF-1α.

### Example 161 Immunoblots of HEK293 Cells Treated with a Compound of the Invention at Lower Concentrations

HEK293 T cells were treated with the 68 of the invention (Example 46). Cells were treated at 0.5, 1, 5, 10, 20, 50 and 100 M for 18 hours. The blots show protein levels of HIF1-α and GAPDH at the identified hour following treatment. Protocols for cell culture and immunoblotting were performed using established methods. The results of this example show, as can be seen in FIG. 2, that the compounds of the invention stabilise cellular HIF-1α.

### Example 162 In Vivo Efficacy Studies

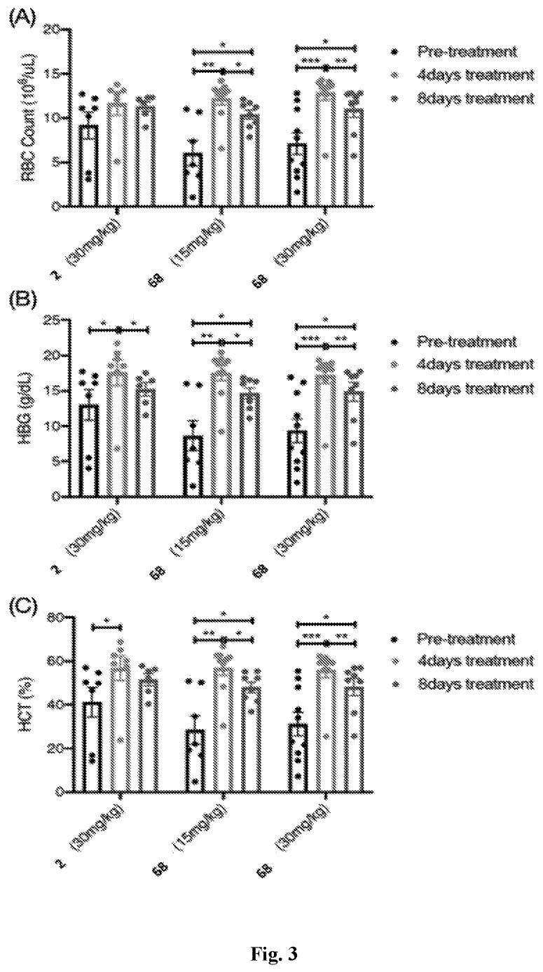

Groups of seven C57BL/6 mice were administered with a twice daily dose (IP) with either vehicle (1% methylcellulose), compound 68 (Example 46) at 15 mg/kg or 30 mg/kg or Daprodustat (2) at 30 mg/kg (FIG. 3). Samples were taken either pre-treatment, on the 4th day into treatment or on the 8^{th} day of treatment. Blood samples were analysed using Celltac Alpha MEK-6500K. Statically significant increases in red blood cell count (RBC), haemoglobin (HGB) and haematocrit (HCT) were observed in compound treated mice. 68 showed similar levels compared to 2. 15 mg/kg of 68 produced the same levels of RBC, HCT and HGB compared to 2 at 30 mg/kg. Indeed, the similarities between the different doses of 68 suggest the dose can be lowered. The mice showed no adverse effects to the dosed compounds.

### Example 163 Selectivity for PHD

Lack of selectivity of an enzyme inhibitor can lead to unpredictable and undesirable off-target effects. Currently available PHD inhibitors, including roxadustat, daprodustat, molidustat, desidustat and vadadustat, display only limited target selectivity for the PHDs, with, for example, inhibition by one or more of them being observed with collagen prolyl hydroxylases (CPHs); 2-oxoglutarate and iron dependent oxygenase domain containing 1 (OFGOD1); and jumonji domain containing 6 (JMJD6).

Compound 68 (Example 46) was assessed to observe its inhibitory activity of sites which are commonly inhibited by existing PHD inhibitors.

### Recombinant PHD2 Production, Purification and SPE-MS IC₅₀ Determination

Recombinant PHD2 was produced, purified and IC50 determination as reported by Yeh et al¹.

### Recombinant FIH Production, Purification and SPE-MS IC₅₀ Determination

Recombinant FIH was produced, purified and IC50 determination as reported by Yeh et al¹.

### Recombinant JMJD6 Production and Purification

Recombinant JMJD6 was produced as the full-length protein in E. coli and purified as reported by Cockman et al and Islam et al^{2,3}.

### JMJD6 IC₅₀ Determination

All reagents were from Sigma Aldrich and of the highest grade available. Ferrous ammonium sulphate (FAS) was prepared freshly by dissolving to 400 mM in 20 mM HCl and subsequently diluted to 1 mM in deionized water. 2-Oxoglutarate (2OG, 10 mM) and L-ascorbic acid (LAA, 50 mM) were prepared fresh by dissolving in deionized water. Inhibition of the catalytic activity of recombinant human JMJD6 was assessed using an N-terminal peptide (RSKKRKKSKSRS) of RNA Binding Motif Protein 39 (RBM39 residues 31-42) and monitoring the appearance of the hydroxylated peptide product in 50 mM Tris·Cl pH 7.5. Titrations of 68 for IC₅₀ determinations (3-fold and 11-point IC₅₀ curves) were performed using an ECHO 550 acoustic dispenser (Labcyte) and dry dispensed into 384-well polypropylene assay plates. The final assay concentration of DMSO was kept constant at 0.5% (v/v). Full length JMJD6 was prepared at a concentration of 1.0 mM in 50 mM Tris·Cl pH 7.5 and 25 μl dispensed across the 384-well plates, JMJD6 was preincubated with compound dilutions for 15 minutes. The reaction was initiated by dispensing 25 μl of substrate (20 M ferrous iron sulfate, 200 μM L-ascorbic acid, 10 μM RBM39₃₁₋₄₂ and 20 M 2-oxoglutarate) across each 384-well assay plate. The reaction was allowed to progress for 30 minutes, then quenched by dispensing 10% formic acid (5 μl). Peptide analysis was performed by Liquid Chromatography Mass Spectrometry (LCMS) using an Agilent 1290 infinity II LC system equipped with an Agilent 1290 multisampler and an Agilent 1290 high speed pump and connected to an Agilent 6550 accurate mass iFunnel quadrupole time of flight (QTOF) mass spectrometer. 10 ml of the assay mixture was injected onto a ZORBAX RRHD Eclipse Plus C₁₈ column (Agilent). Solvent A consisted of LCMS grade water containing 0.1% (v/v) formic acid and solvent B consisted of acetonitrile containing 0.1% (v/v) formic acid. Peptides were separated using a step wise gradient (0 min—95% solvent A, 1.0 min—80% solvent A, 3.0 min—45% solvent A, 4.0 min—45% solvent A, 5.0 min—0% solvent A, 6.0 min—0% solvent A, 7.0 min—95% solvent A). This was followed by a 1 min post run with 95% (v/v/) solvent A to re-equilibrate the column, all flow rates were 0.2 ml/min. The mass spectrometer was operated in the positive ion mode with a drying gas temperature (280° C.), drying gas flow rate (13 L/min), nebulizer pressure (40 psig), sheath gas temperature (350° C.), sheath gas flow rate (12 L/min), capillary voltage (4000 V), nozzle voltage (1000 V). All acquired data were analysed using Agilent MassHunter Qualitative Analysis (Version B.07.00) software.

### JMJD6 Solid Phase Extraction Mass Spectrometry (SPE-MS) Assays

The JMJD6 inhibitory activities of Daprodustat, Roxadustat and Molidustat were assessed using a 40-mer peptide substrate of bromodomain-containing protein 4 (BRD4₅₁₁₋₅₅₀)² by monitoring the hydroxylation of the peptide product in 50 mM Tris·HCl pH 7.5 buffer. Titrations of compounds were prepared using an ECHO 550 acoustic dispenser (Labcyte). An 11-point and 3-fold dilution for each compound was prepared and dry dispensed into 384-well polypropylene plates. A solution of full length JMJD6 was prepared at a concentration of 1.0 mM and 25 μl dispensed across the plate using a multidrop dispenser equipped with a low volume dispensing cassette (Thermo). Compound dilutions were pre-incubated with JMJD6 for 15 minutes. The enzyme reaction was initiated by 25 μl dispensing of the substrate mixture in 50 mM Tris·HCl pH 7.5 (200 mM L-ascorbate, 20 mM ferrous ammonium sulfate, 20 mM 2-oxoglutarate and 10 mM of the JMJD6 substrate BRD4₅₁₁₋₅₅₀ 2. Reactions were allowed to progressed for 15 minutes at room temperature and stopped by dispensing 10% (v/v) formic acid (5 l). The final concentration of DMSO was 0.5% (v/v). Assay plates were transferred to a RapidFire RF365 high throughput sampling robot (Agilent) connected to an Agilent 6550 quadrupole-time-of-flight (Q-TOF) mass spectrometer. Samples were aspirated under vacuum and loaded onto a C₄ solid phase extraction (SPE) cartridge. The C₄ SPE cartridge was washed with 0.1% (v/v) formic acid in water for 5.5 s at a flow rate of 1.5 ml/min to remove non-volatile buffer salts. The peptide was then eluted from the SPE with 80% (v/v) acetonitrile, 20% (v/v) water containing 0.1% (v/v) formic acid for 5.5 s at a flow rate of 1.6 ml/min into the mass spectrometer. The mass spectrometer was operated in the positive ion mode with a drying gas temperature of 280° C., drying gas flow rate of 13 L/min, nebulizer pressure of 40 psig, sheath gas temperature of 350° C., sheath gas flow rate of 12 L/min, capillary voltage of 4000 V, and nozzle voltage of 1000 V. Peak area data for the +8 charge state was integrated using RapidFire Integrator software (Agilent). The % conversion of the BRD4₅₁₁₋₅₅₀ to the hydroxylated product was calculated using:

% conversion=100×hydroxylated/(hydroxylated+non-hydroxylated peptide): IC₅₀ data were determined from non-linear regression plots using GraphPad prism 6.0.

### KDM4A, KDM5B and KDM6B Liquid Chromatography Mass Spectrometry (LCMS) Assay

The inhibitory activity of 68 was assessed by monitoring demethylation of the respective peptide substrates for KDM4A, KDM5B and KDM6B. The peptide substrate for KDM4A was a 15-mer histone-H₃ derivative (ARTAQTARK(me3)STGGI) as reported by Hutchinson et al 4 and synthesized by GL Biochem (Shanghai) Ltd (Shanghai, China). The peptide substrate for KDM5B was a 21-mer histone-H₃ peptide (ARTK(me3)QTARKSTGGKAPRKQLA), as synthesized by Peptide Protein Research (Hampshire, UK). The KDM6B peptide substrate was a 17-mer histone-H₃ peptide (LATKAARK(me3)SAPATGGVK), as synthesized by GL Biochem (Shanghai) Ltd (Shanghai, China). Recombinant KDM4A, residues M1-L359, was produced in E. coli and purified as reported by Ng et al 5. Recombinant KDM5B, residues, M1-R⁸²², was expressed in a baculoviral expression system and purified as previously described by Johansson et al⁶. KDM6B, residues D1141-E1590, was expressed in E. coli and purified as previously described by Rose et al⁷.
KDM4A reactions were performed under optimized buffer conditions (50 mM MES pH 7.0. KDM4A (0.15 mM) was pre-incubated for 15 minutes in the presence of 68 (100 mM) and the enzyme reaction initiated by addition of substrate (100 mM L-ascorbate, 10 mM ferrous ammonium sulphate, 10 mM 2-oxoglutarate and 10 mM peptide substrate). The enzyme reaction was progressed for 50 minutes, and the reaction stopped by addition of formic acid to a final concentration of 1% (v/v). Control reactions in the presence of 0.5% (v/v) DMSO and a control in the presence of a known inhibitor of KDM4A (50 mM 2, 4-pyridine dicarboxylic acid, 8 were also set up.
KDM5B enzyme reactions were performed under optimized buffer conditions (50 mM MES pH 7.0, 50 mM NaCl, 1 mM TCEP). KDM5A (0.15 mM) was pre-incubated for 15 minutes in the presence of 68 (100 mM) and the enzyme reaction was initiated by addition of substrate (100 mM L-ascorbate, 10 mM ferrous ammonium sulphate, 10 mM 2-oxoglutarate and 5 mM peptide). The enzyme reaction was progressed for 30 minutes, and the reaction stopped by addition of formic acid to a final concentration of 1% (v/v). Control reactions included a 0.5% DMSO control and a reaction with a known inhibitor of KDM5B (10 mM KDOAM25, ⁹).
KDM6B reactions were performed under optimized buffer conditions (50 mM MES pH 7.0). KDM6B (0.15 mM) was pre-incubated for 15 minutes in the presence of 68 (100 mM) and the enzyme reaction initiated by addition of substrate (100 mM L-ascorbate, 10 mM ferrous ammonium sulphate, 10 mM 2-oxoglutarate and 5 mM peptide). The enzyme reaction was progressed for 30 minutes and the reaction stopped by addition of formic acid to a final concentration of 1% (v/v). Control reactions included a 0.5% DMSO control and a reaction with a known inhibitor of KDM6B (10 mM GSKJ1, ¹⁰)
Enzyme reactions were transferred to a 96-well polypropylene plate and peptide analysis was performed by LCMS using an Agilent 1290 infinity II LC system equipped with an Agilent 1290 multisampler and an Agilent 1290 high speed pump and connected to an Agilent 6550 accurate mass iFunnel quadrupole time of flight (QTOF) mass spectrometer. 4 ml of enzyme reaction were injected and loaded onto a ZORBAX RRHD Eclipse Plus C₁₈ column (Agilent Technologies, CA, US). Solvent A consisted of LCMS grade water containing 0.1% (v/v) formic acid and solvent B consisted of acetonitrile containing 0.1% (v/v) formic acid. Peptides were separated using a step wise gradient (0 min—95% solvent A, 1.0 min—80% solvent A, 3.0 min—45% solvent A, 4.0 min—45% solvent A, 5.0 min-0% solvent A, 6.0 min—0% solvent A, 7.0 min—95% solvent A). This was followed by a 3 min post run with 95% solvent A to re-equilibrate the column, all flow rates were 0.2 ml/min. The mass spectrometer was operated in the positive ion mode with a drying gas temperature of 280° C., drying gas flow rate of 13 L/min, nebulizer pressure of 40 psig, sheath gas temperature of 350° C., sheath gas flow rate of 12 L/min, capillary voltage of 4000 V, nozzle voltage of 1000 V. All acquired data were analyzed using Agilent MassHunter Qualitative Analysis (Version B.07.00) software.

### OGFOD1 Solid Phase Extraction Mass Spectrometry (SPE-MS) Assay

OGFOD1 gene was cloned into the pET-28a vector and expressed and purified as the full-length enzyme (Met1-Glu542) with an N-terminal 6-His tag in E. coli strain BL21 (DE3). The synthetic peptide substrate RPS23 (Ala47-Lys76 AKGIVLEKVGVEAKQPNSAIRKAVRVQLIK-NH2) was synthesized by GL Biochem (Shanghai, China) to >95% purity. Ferrous ammonium sulphate (FAS), 2-oxoglutarate (2-OG) and L-ascorbic acid (LAA) were from Sigma Aldrich. Ferrous ammonium sulphate was prepared fresh by dissolving 50-100 mg in 20 mM HCl to 400 mM concentration which was further diluted to 1 mM in deionized water. 2-OG (10 mM) and L-AA (50 mM) were both prepared fresh in deionized water.

IC₅₀determinations were performed in 384-well plate format using polypropylene plates (Greiner Bio One, Cat Number 781096). Compounds were prepared as 20 mM DMSO stock solutions and all compound dispenses were performed using an ECHO 550 acoustic dispenser (Labcyte, Sunnyvale, CA). A positive control compound (2, 4-PDCA, 100 mM) was dispensed into column 1 (250 nl) and DMSO was dispensed into column 13 (250 nl). All test compounds were serially diluted (an approximately 3-fold dilution series across an 11-point IC₅₀) and 250 nl of each dilution dispensed in duplicate into the polypropylene plate. OGFOD1 was diluted to 0.3 mM in assay buffer (50 mM Tris·Cl pH 7.5) and was dispensed (25 ml) into the 384-well compound plates using a multidrop combi reagent dispenser (Thermo Scientific, Code 5840300) with a small tube plastic tip dispensing cassette (Thermo Scientific, Code 24073290). The compounds were pre-incubated with OGFOD1 for 15 minutes and the enzyme reaction initiated by dispense of 25 ml of substrate solution (200 mM LAA, 20 mM FAS, 20 mM 2-OG, 10 mM RPS23 (47-76) peptide) in assay buffer. The final concentration of DMSO in the assay was 0.5%. The reactions were allowed to progress for 20 min, then stopped by addition of 10% (v/v) formic acid (5 ml) and the assay plate transferred to a RapidFire RF 365 coupled to a 6550 Accurate-Mass Quadrupole Time-of-Flight (QTOF) mass spectrometer (Agilent). Samples were aspirated under vacuum and loaded onto a C₄ solid phase extraction (SPE) cartridge and the SPE cartridge washed with 0.1% (v/v) formic acid in LCMS grade water for 5.5 seconds at a flow rate of 1.5 ml/min to remove non-volatile buffer components. After the aqueous wash, peptides were eluted from the C₄ SPE cartridge in an organic elution step (80% (v/v) acetonitrile, 20% (v/v) LCMS grade water containing 0.1% formic acid) at a flow rate of 1.6 ml/min for 5.5 seconds. The mass spectrometer was operated in positive ion mode with a drying gas temperature (280° C.), drying gas flow rate (13 L/min), nebulizer pressure (40 psig), sheath gas temperature (350° C.), sheath gas flow rate (12 L/min), capillary voltage (4000 V), nozzle voltage (1000 V). Ion data for the substrate and hydroxylated (+16) peptide product was extracted and peak area data integrated using RapidFire integrator software (version 4.3.017235, Agilent). The % conversion of peptide substrate to hydroxylated product was calculated in excel and IC50 curves generated using graphpad prism version 7.0.

### CONCLUSION

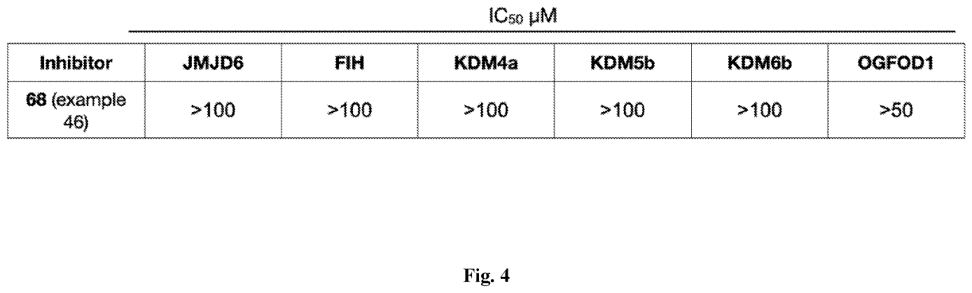

As shown in FIG. 4 of the present application, compound 68 (Example 46), had a very high IC₅₀ for each of the off-target sites tested. This demonstrates that the compound has good selectivity and inhibition of PHD specifically. Therefore compounds of the invention would be expected to have reduced side-effects compared to existing PHD inhibitors.

### REFERENCES

1 Yeh, T. L. et al. Molecular and cellular mechanisms of HIF prolyl hydroxylase inhibitors in clinical trials. Chem Sci 8, 7651-7668 (2017). https://doi.org/10.1039/c7sc02103h
2 Cockman, M. E. et al. Widespread hydroxylation of unstructured lysine-rich protein domains by JMJD6. Proc Natl Acad Sci USA 119, e2201483119 (2022). https://doi.org/10.1073/pnas.2201483119
3 Islam, M. S. et al. Biochemical and structural investigations clarify the substrate selectivity of the 2-oxoglutarate oxygenase JMJD6. J Biol Chem 294, 11637-11652 (2019). https://doi.org/10.1074/jbc.RA119.008693
4 Hutchinson, S. E. et al. Enabling Lead Discovery for Histone Lysine Demethylases by High-Throughput RapidFire Mass Spectrometry. Journal of Biomolecular Screening 17, 39-48 (2011). https://doi.org/10.1177/1087057111416660
5 Ng, S. S. et al. Crystal structures of histone demethylase JMJD2A reveal basis for substrate specificity. Nature 448, 87-91 (2007). https://doi.org/10.1038/nature05971
6 Johansson, C. et al. Structural analysis of human KDM5B guides histone demethylase inhibitor development. Nature Chemical Biology 12, 539-5-[5 (2016). https://doi.org/10.1038/nchembio.2087
7 Rose, N. R. et al. Plant Growth Regulator Daminozide Is a Selective Inhibitor of Human KDM2/7 Histone Demethylases. Journal of Medicinal Chemistry 55, 6639-6643 (2012). https://doi.org/10.1021/im300677i
8 Rose, N. R. et al. Inhibitor scaffolds for 2-oxoglutarate-dependent histone lysine demethylases. J Med Chem 51, 7053-7056 (2008). https://doi.org/10.1021/jm800936s
9 Tumber, A. et al. Potent and Selective KDM5 Inhibitor Stops Cellular Demethylation of H3K4me3 at Transcription Start Sites and Proliferation of MM1S Myeloma Cells. Cell Chemical Biology 24, 371-380 (2017). https://doi.org/https://doi.org/10.1016/j.chembiol.2017.02.006
10 Kruidenier, L. et al. A selective jumonji H₃K27 demethylase inhibitor modulates the proinflammatory macrophage response. Nature 488, 404-408 (2012). https://doi.org/10.1038/nature11262
   Further aspects and embodiments of the invention are defined in the following numbered clauses.
   1. A compound which is a substituted azine of formula (I) or a pharmaceutically acceptable salt thereof

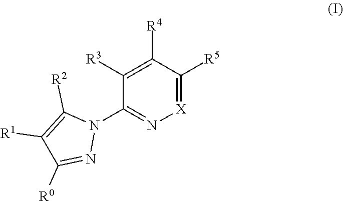

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w} or —C(O)N(R^{x})R⁷;
R² is H, —OR^{q} or unsubstituted or substituted C₁₋₆ alkyl; and R³ is H, —OR⁸ or unsubstituted or substituted C₁₋₆ alkyl; or R² is —N═ and R³ is =C(R^{y})— and R² and R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN, —N(R^{t})C(O)N(R^{u})R, —C(O)OR^{w}, or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸, R⁹ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl;
R^{q} is H, unsubstituted or substituted C₁₋₆ alkyl, or unsubstituted or substituted phenyl;
provided that one of R¹ and R⁵ is —C(O)N(R)R⁷ and the other of R¹ and R⁵ is H, unsubstituted or substituted C₁₋₆ alkyl, unsubstituted or substituted aryl, unsubstituted or substituted heteroaryl, —CN, —N(R^{t})C(O)N(R^{u})R^{v} or —C(O)OR^{w}.
2. A compound according to clause 1 wherein:
   R⁰ is H or unsubstituted C₁₋₆ alkyl;
   R¹ is H, —CN, —C(O)OR^{w} or —C(O)N(R^{x})R⁷;
   R² is H or unsubstituted C₁₋₆ alkyl; and R³ is H or —OR⁸; or R² is —N═ and R³ is =C(R^{y})— and R² and R³ together form a group of formula —N═C(R^{y})—;
   R⁴ is H, —OR⁹ or —C(O)OR¹⁰;
   R⁵ is H, —CN, —C(O)OR^{w}, or —C(O)N(R^{x})R⁷;
   R⁶ is H;
   R⁷ is —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR^{z} or unsubstituted C₁₋₄ alkyl;
   R⁸, R⁹ and R¹⁰ are each independently selected from H and unsubstituted or substituted C₁₋₆ alkyl; and
   R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R″ wherein R^{ww} is phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid;
   provided that one of R¹ and R⁵ is —C(O)N(R)R⁷ and the other of R¹ and R⁵ is H, —CN or —C(O)OR^{w}.
3. A compound according to clause 1 or clause 2 wherein:
   R⁰ is H or methyl;
   R¹ is H, —CN, —C(O)OR^{w}, or —C(O)N(R′)R⁷;
   R² is H or methyl; and R³ is H or —OR⁸; or R² is —N═ and R³ is =C(R^{y})— and R² and R³ together form a group of formula —N═C(R^{y})—;
   R⁴ is H, —OR⁹ or —C(O)OR¹⁰;
   R⁵ is H, —CN, —C(O)OR^{w}, or —C(O)N(R′)R⁷;
   R⁶ is H;
   R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)OMe, —C(O)Oet, —C(O)NH₂, —C(O)N(H)Me, -Ome or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl;
   R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R), —C(O)R⁰, —OR^{d} or an amino acid, wherein R^{a}, R^{b}, R⁰ and R^{d} are each independently selected from H, unsubstituted or substituted C₁₋₆ alkyl and an amino acid; and
   R^{x} is H;
   R^{z} is H;
   R^{w} is H, unsubstituted C₁₋₆ alkyl, or C₁₋₆ alkyl which is substituted with phenyl or —OC(O)R^{ww} wherein R″ is phenyl or unsubstituted C₁₋₆ alkyl; and
   R is H or methyl;
   provided that one of R¹ and R⁵ is —C(O)N(R^{x})R⁷ and the other of R¹ and R⁵ is H, —CN or —C(O)OR^{w}.
4. A compound according to any one of clauses 1 to 3 wherein either:
(1) (a) R⁵ is —C(O)N(R^{x})R⁷ and (b) R³ is —OR⁸ or R⁴ is —OR⁹; or
(2) (a) R¹ is —C(O)N(R^{x})R⁷ and (b) R⁴ is —OR⁹ or —C(O)OR¹⁰, or R⁵ is —C(O)OR^{w}.
5. A compound according to any one of the preceding clauses wherein the substituted azine has the formula (Ia)

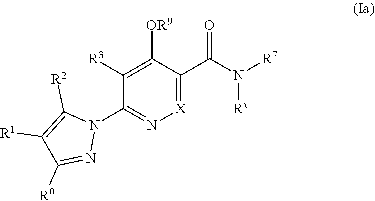

wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R³ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is H or unsubstituted or substituted C₁₋₆ alkyl; and
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.
6. A compound according to clause 5 wherein R⁹ is H.
7. A compound according to any one of the preceding clauses wherein the substituted azine has any one of the following structures:

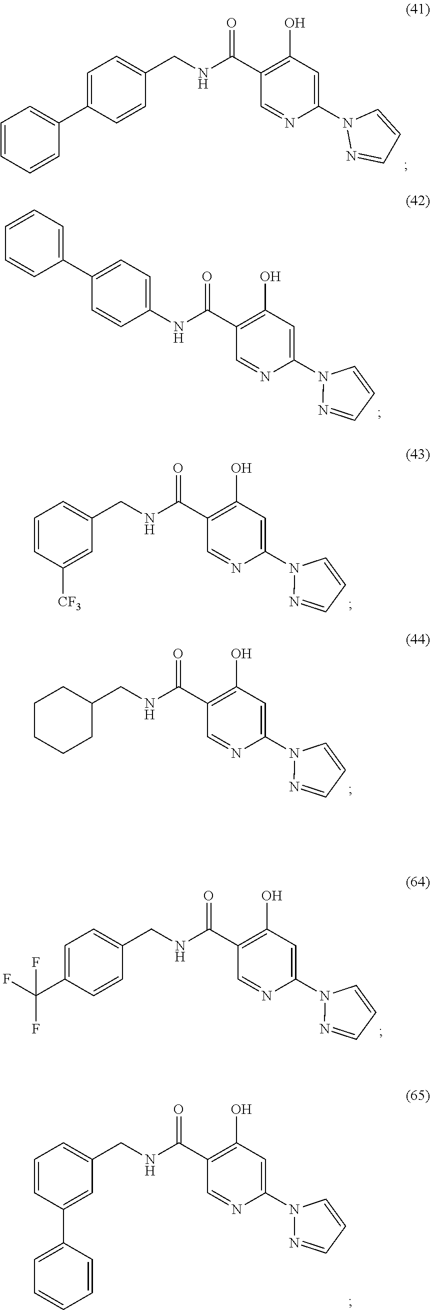

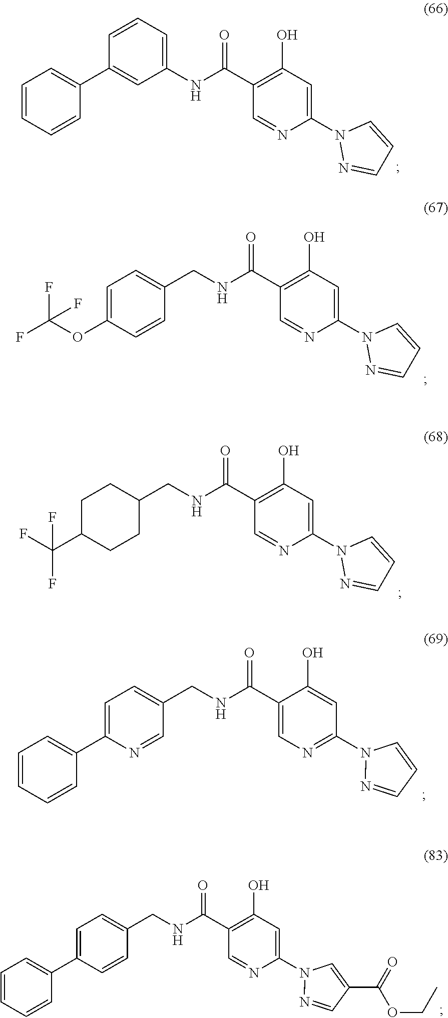 [SMILES: O=C(Nc1cccc(-c2ccccc2)c1)c1cnc(-n2cccn2)cc1O ; O=C(NCc1ccc(OC(F)(F)F)cc1)c1cnc(-n2cccn2)cc1O ; O=C(NCC1CCC(C(F)(F)F)CC1)c1cnc(-n2cccn2)cc1O ; O=C(NCc1ccc(-c2ccccc2)nc1)c1cnc(-n2cccn2)cc1O ; CCOC(=O)c1cnn(-c2cc(O)c(C(=O)NCc3ccc(-c4ccccc4)cc3)cn2)c1]

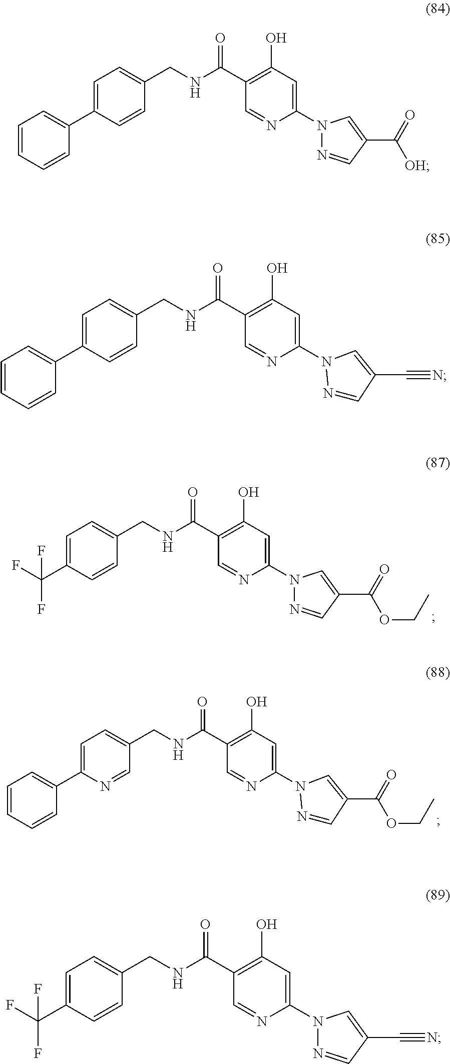 [SMILES: O=C(O)c1cnn(-c2cc(O)c(C(=O)NCc3ccc(-c4ccccc4)cc3)cn2)c1 ; N#Cc1cnn(-c2cc(O)c(C(=O)NCc3ccc(-c4ccccc4)cc3)cn2)c1 ; CCOC(=O)c1cnn(-c2cc(O)c(C(=O)NCc3ccc(C(F)(F)F)cc3)cn2)c1 ; CCOC(=O)c1cnn(-c2cc(O)c(C(=O)NCc3ccc(-c4ccccc4)nc3)cn2)c1 ; N#Cc1cnn(-c2cc(O)c(C(=O)NCc3ccc(C(F)(F)F)cc3)cn2)c1]

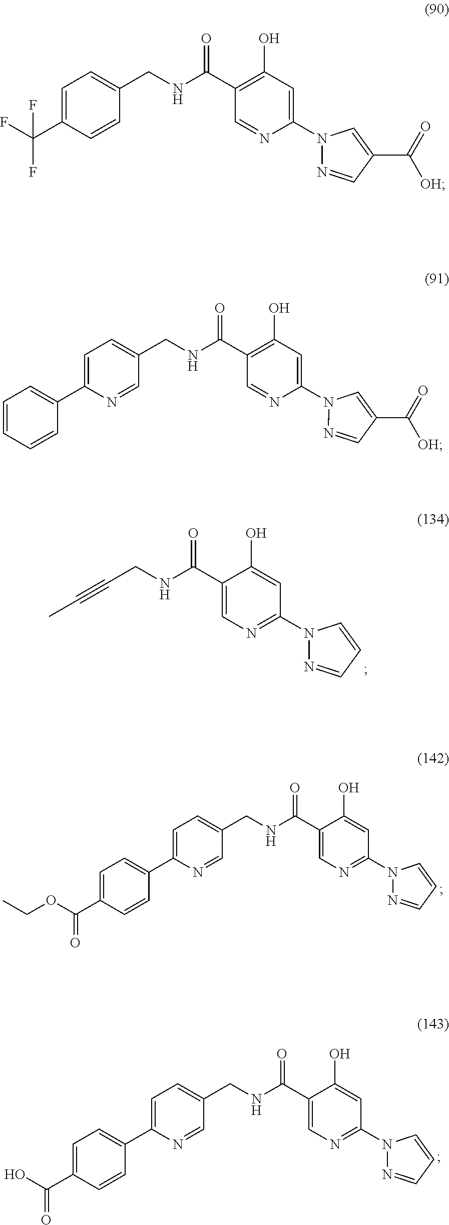 [SMILES: O=C(O)c1cnn(-c2cc(O)c(C(=O)NCc3ccc(C(F)(F)F)cc3)cn2)c1 ; O=C(O)c1cnn(-c2cc(O)c(C(=O)NCc3ccc(-c4ccccc4)nc3)cn2)c1 ; CC#CCNC(=O)c1cnc(-n2cccn2)cc1O ; CCOC(=O)c1ccc(-c2ccc(CNC(=O)c3cnc(-n4cccn4)cc3O)cn2)cc1 ; O=C(O)c1ccc(-c2ccc(CNC(=O)c3cnc(-n4cccn4)cc3O)cn2)cc1]

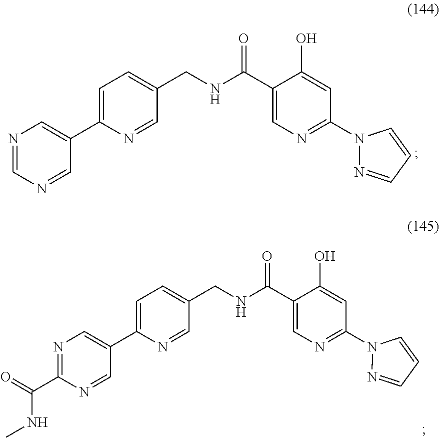 [SMILES: O=C(NCc1ccc(-c2cncnc2)nc1)c1cnc(-n2cccn2)cc1O ; CNC(=O)c1ncc(-c2ccc(CNC(=O)c3cnc(-n4cccn4)cc3O)cn2)cn1]

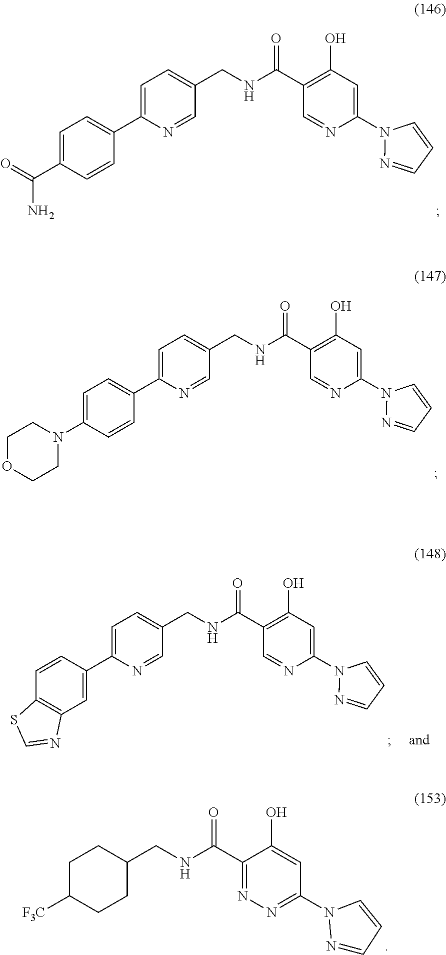 [SMILES: NC(=O)c1ccc(-c2ccc(CNC(=O)c3cnc(-n4cccn4)cc3O)cn2)cc1 ; O=C(NCc1ccc(-c2ccc(N3CCOCC3)cc2)nc1)c1cnc(-n2cccn2)cc1O ; O=C(NCc1ccc(-c2ccc3scnc3c2)nc1)c1cnc(-n2cccn2)cc1O ; O=C(NCC1CCC(C(F)(F)F)CC1)c1nnc(-n2cccn2)cc1O]

8. A compound according to any one of clauses 1 to 4 wherein the substituted azine has the formula (Ib)

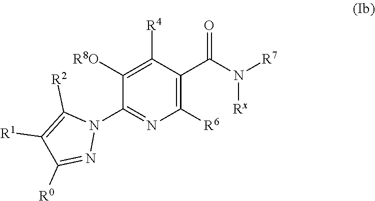

wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R⁴ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)—Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸ is H or unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.
9. A compound according to clause 8 wherein R⁸ is H.
10. A compound according to clause 8 or clause 9 wherein the substituted azine has any one of the following structures

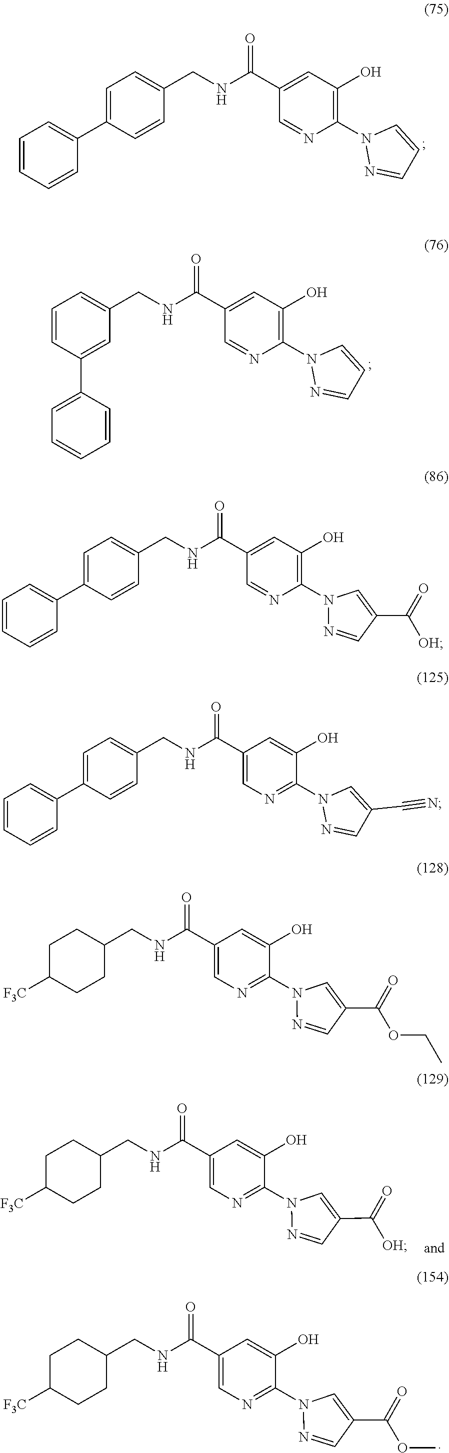

11. A compound according to any one of clauses 1 to 4 wherein the substituted azine has the formula (Ic)

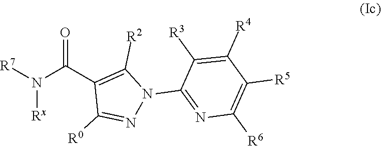

wherein
R⁰ is H or unsubstituted or substituted C₁₋₆ alkyl;
R² is H, —OR^{q} or unsubstituted or substituted C₁₋₆ alkyl;
R³ is H, —OR⁸ or unsubstituted or substituted C₁₋₆ alkyl;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)—Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸, R⁹ and R¹⁰ are each independently selected from H and unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.
12. A compound according to clause 11 wherein (i) R⁴ is OH or C(O)OH, and/or (ii) R⁵ is C(O)OH.
13. A compound according to clause 11 or clause 12 wherein the substituted azine has any one of the following structures

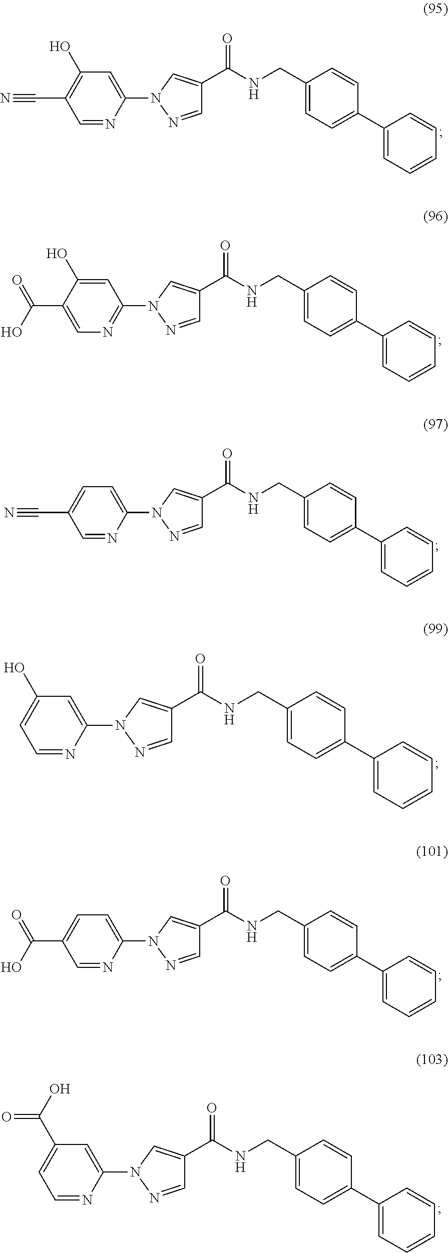 [SMILES: N#Cc1cnc(-n2cc(C(=O)NCc3ccc(-c4ccccc4)cc3)cn2)cc1O ; O=C(NCc1ccc(-c2ccccc2)cc1)c1cnn(-c2cc(O)c(C(=O)O)cn2)c1 ; N#Cc1ccc(-n2cc(C(=O)NCc3ccc(-c4ccccc4)cc3)cn2)nc1 ; O=C(NCc1ccc(-c2ccccc2)cc1)c1cnn(-c2cc(O)ccn2)c1 ; O=C(O)c1ccc(-n2cc(C(=O)NCc3ccc(-c4ccccc4)cc3)cn2)nc1 ; O=C(O)c1ccnc(-n2cc(C(=O)NCc3ccc(-c4ccccc4)cc3)cn2)c1]

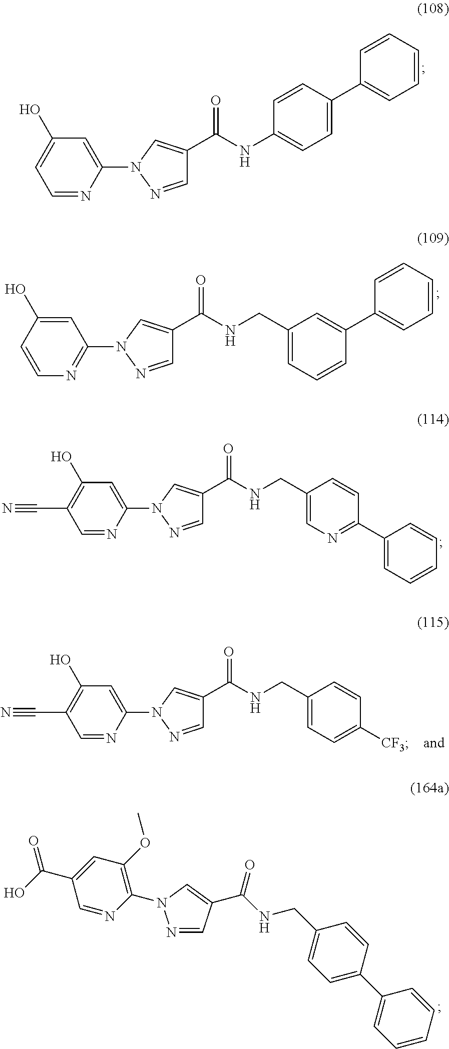 [SMILES: O=C(Nc1ccc(-c2ccccc2)cc1)c1cnn(-c2cc(O)ccn2)c1 ; O=C(NCc1cccc(-c2ccccc2)c1)c1cnn(-c2cc(O)ccn2)c1 ; N#Cc1cnc(-n2cc(C(=O)NCc3ccc(-c4ccccc4)nc3)cn2)cc1O ; N#Cc1cnc(-n2cc(C(=O)NCc3ccc(C(F)(F)F)cc3)cn2)cc1O ; COc1cc(C(=O)O)cnc1-n1cc(C(=O)NCc2ccc(-c3ccccc3)cc2)cn1]

14. A compound according to any one of clauses 1 to 4 wherein the substituted azine has the formula (Id)

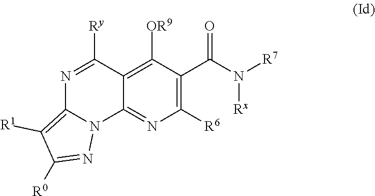

wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is H or unsubstituted or substituted C₁₋₆ alkyl; and
R^{w}, R^{x}, R^{y}, and R^{z} are each independently selected from H, unsubstituted or substituted C₁₋₄ alkyl, and unsubstituted or substituted phenyl.
15. A compound according to clause 14 wherein R⁹ is H.
16. A compound according to clause 14 or clause 15 wherein the substituted azine has any one of the following structures

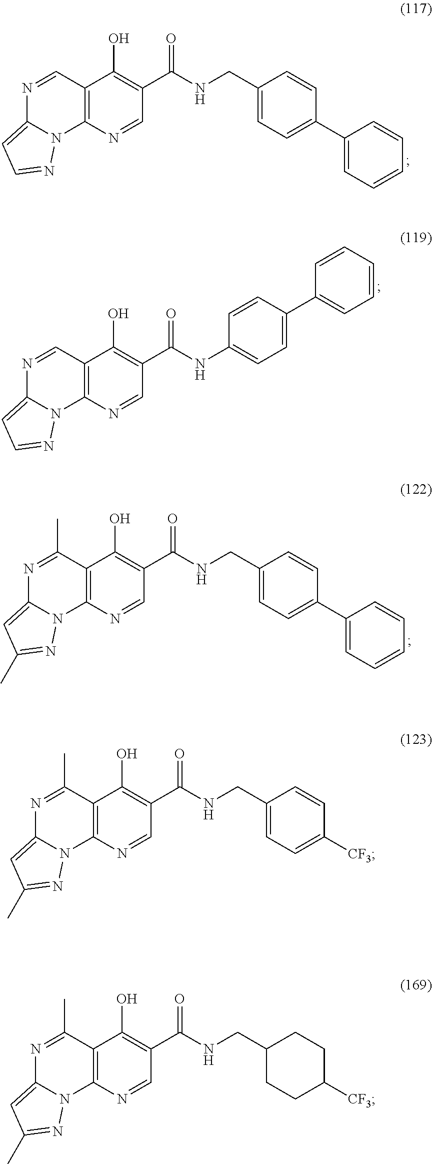 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1]

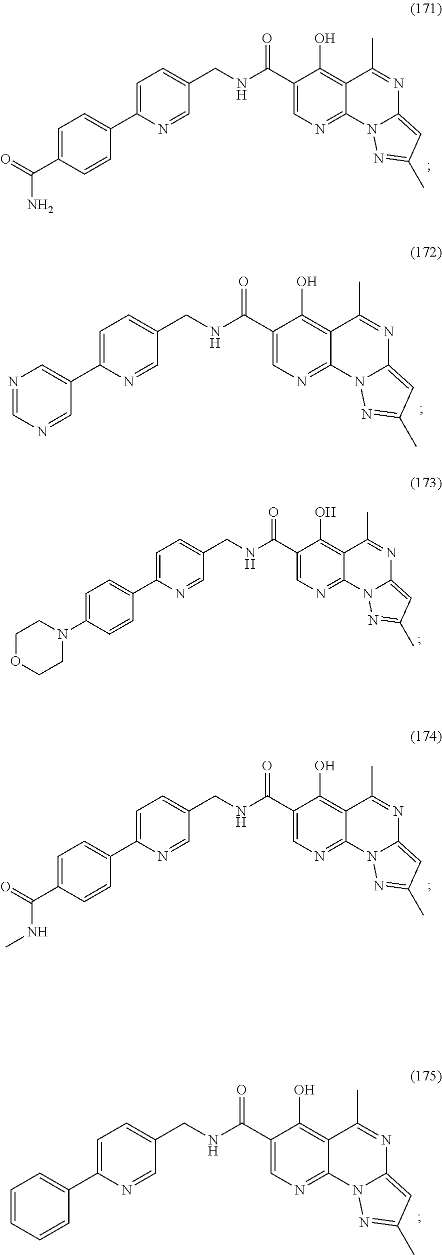 [SMILES: Cc1cc2nc(C)c3c(O)c(C(=O)NCc4ccc(-c5ccc(C(N)=O)cc5)nc4)cnc3n2n1 ; Cc1cc2nc(C)c3c(O)c(C(=O)NCc4ccc(-c5cncnc5)nc4)cnc3n2n1 ; Cc1cc2nc(C)c3c(O)c(C(=O)NCc4ccc(-c5ccc(N6CCOCC6)cc5)nc4)cnc3n2n1 ; CNC(=O)c1ccc(-c2ccc(CNC(=O)c3cnc4c(c(C)nc5cc(C)nn54)c3O)cn2)cc1 ; Cc1cc2nc(C)c3c(O)c(C(=O)NCc4ccc(-c5ccccc5)nc4)cnc3n2n1]

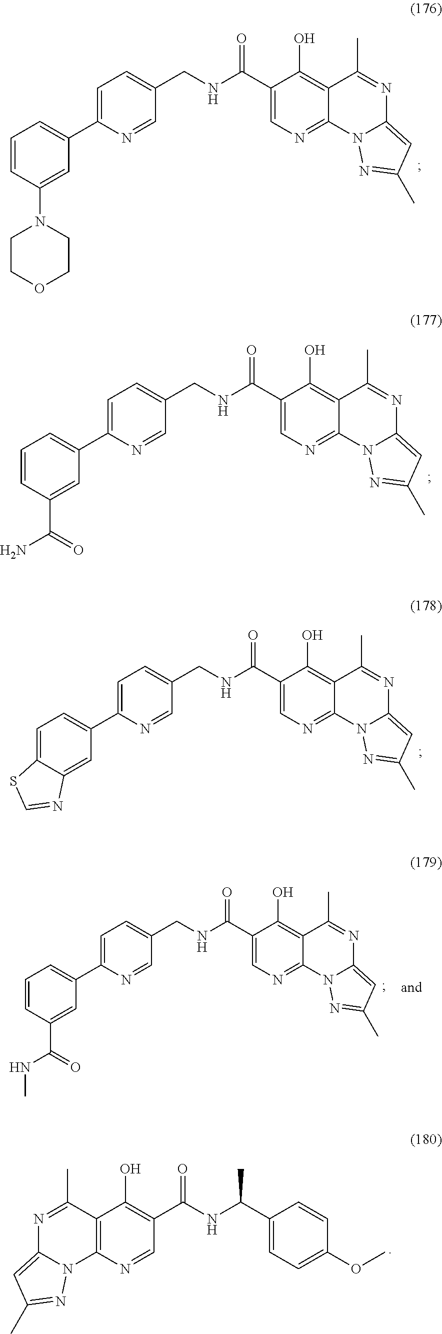

17. A compound according to any one of clauses 1 to 3 wherein the substituted azine has any one of the following structures

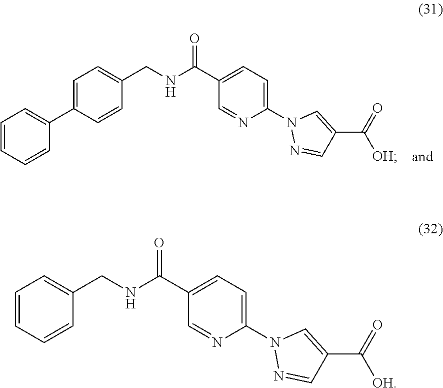 [SMILES: O=C(O)c1cnn(-c2ccc(C(=O)NCc3ccc(-c4ccccc4)cc3)cn2)c1 ; "and" ; O=C(O)c1cnn(-c2ccc(C(=O)NCc3ccccc3)cn2)c1]

18. A compound according to clause 5 wherein R⁹ is unsubstituted or substituted C₁₋₆ alkyl, optionally wherein the substituted azine of formula (Ia) has any one of the following structures

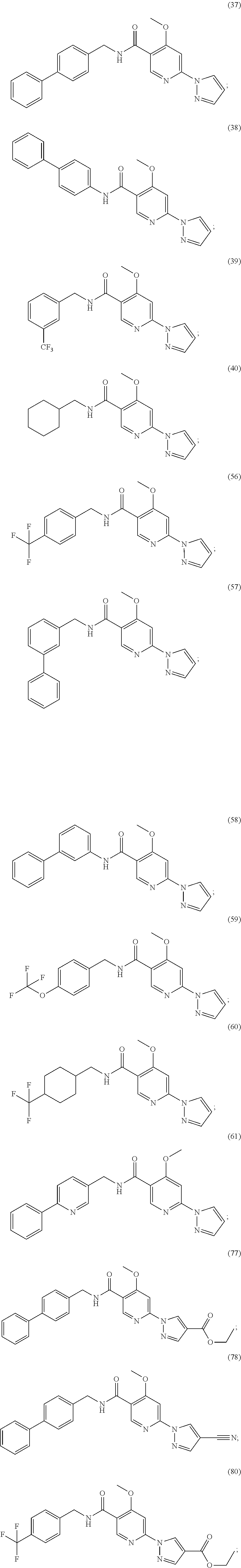

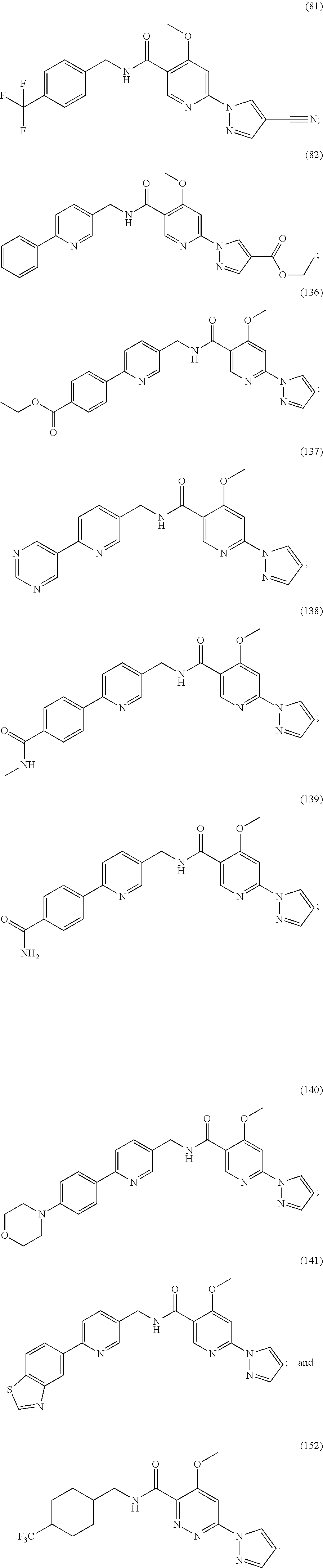

19. A compound according to clause 8 wherein R⁸ is unsubstituted or substituted C₁₋₆ alkyl, optionally wherein the substituted azine of formula (Ib) has any one of the following structures

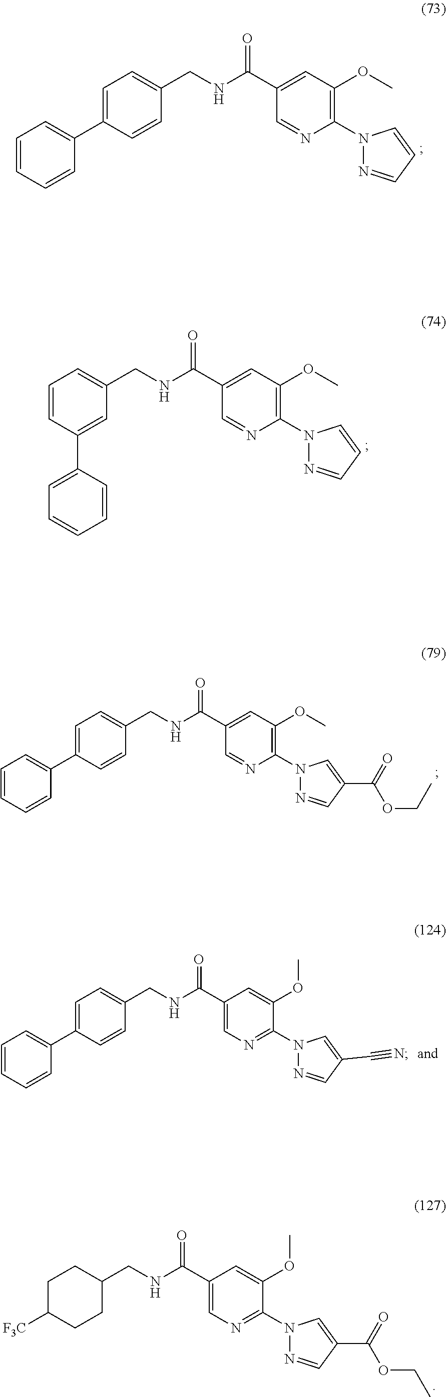 [SMILES: COc1cc(C(=O)NCc2ccc(-c3ccccc3)cc2)cnc1-n1cccn1 ; COc1cc(C(=O)NCc2cccc(-c3ccccc3)c2)cnc1-n1cccn1 ; CCOC(=O)c1cnn(-c2ncc(C(=O)NCc3ccc(-c4ccccc4)cc3)cc2OC)c1 ; COc1cc(C(=O)NCc2ccc(-c3ccccc3)cc2)cnc1-n1cc(C#N)cn1 ; CCOC(=O)c1cnn(-c2ncc(C(=O)NCC3CCC(C(F)(F)F)CC3)cc2OC)c1]

20. A compound according to clause 11 wherein R⁴ is —OR⁹ or —C(O)OR¹⁰, and/or R⁵ is —C(O)OR^{w}, wherein each of R⁹, R¹⁰ and R^{w} is independently unsubstituted or substituted C₁₋₆ alkyl; optionally wherein the substituted azine of formula (Ic) has any one of the following structures

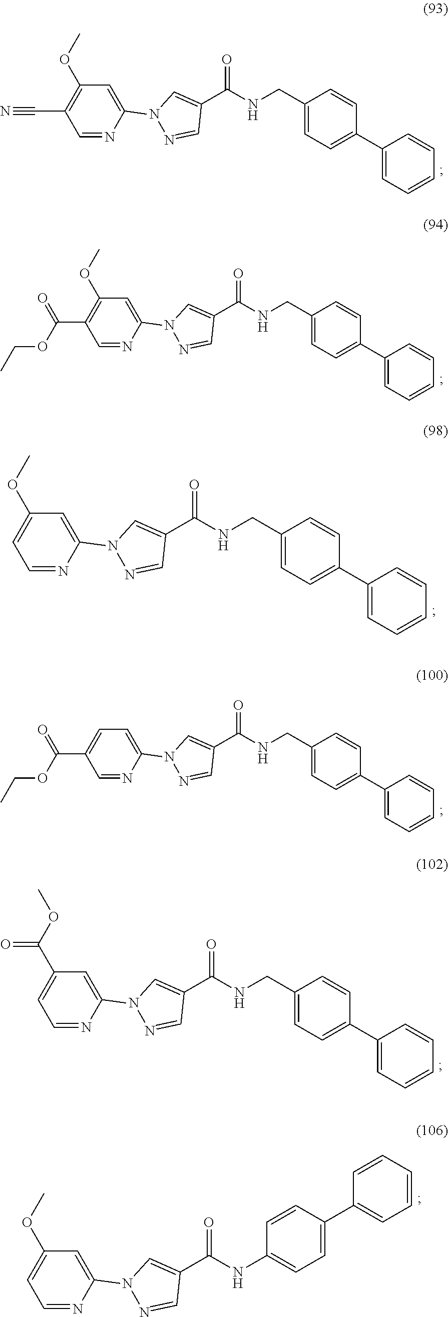 [SMILES: COc1cc(-n2cc(C(=O)NCc3ccc(-c4ccccc4)cc3)cn2)ncc1C#N ; CCOC(=O)c1cnc(-n2cc(C(=O)NCc3ccc(-c4ccccc4)cc3)cn2)cc1OC ; COc1ccnc(-n2cc(C(=O)NCc3ccc(-c4ccccc4)cc3)cn2)c1 ; CCOC(=O)c1ccc(-n2cc(C(=O)NCc3ccc(-c4ccccc4)cc3)cn2)nc1 ; COC(=O)c1ccnc(-n2cc(C(=O)NCc3ccc(-c4ccccc4)cc3)cn2)c1 ; COc1ccnc(-n2cc(C(=O)Nc3ccc(-c4ccccc4)cc3)cn2)c1]

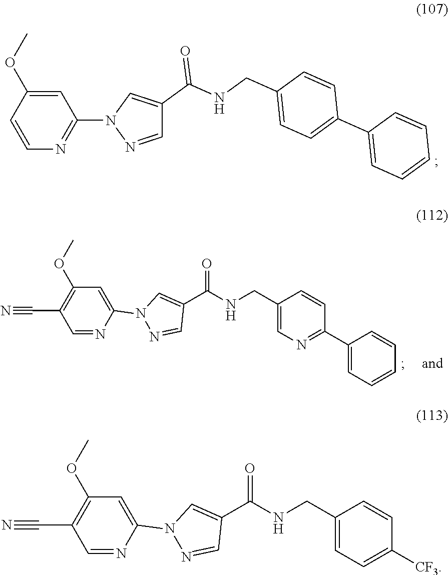 [SMILES: COc1ccnc(-n2cc(C(=O)NCc3ccc(-c4ccccc4)cc3)cn2)c1 ; COc1cc(-n2cc(C(=O)NCc3ccc(-c4ccccc4)nc3)cn2)ncc1C#N ; COc1cc(-n2cc(C(=O)NCc3ccc(C(F)(F)F)cc3)cn2)ncc1C#N]

21. A compound according to clause 14 wherein R⁹ is unsubstituted or substituted C₁₋₆ alkyl; optionally wherein the substituted azine of formula (Id) has the following structure

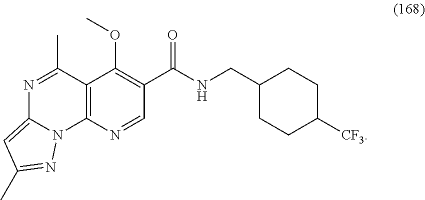 [SMILES: COc1c(C(=O)NCC2CCC(C(F)(F)F)CC2)cnc2c1c(C)nc1cc(C)nn12]

22. A compound which is a substituted pyrimidine of formula (IV) or a pharmaceutically acceptable salt thereof

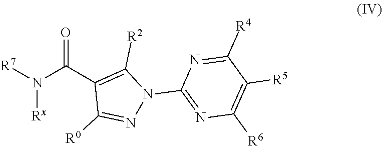

wherein
R⁰ is H or unsubstituted or substituted C₁₋₆ alkyl;
R² is H, —OR^{q} or unsubstituted or substituted C₁₋₆ alkyl;
R⁴ is —OR⁹, wherein R⁹ is selected from H and unsubstituted or substituted C₁₋₆ alkyl;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R″ ¹)—Ar, —CH(R″ ¹)-Ary-Ar, -Ary-Ar, —CH(R″ ¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R^{x} is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.
23. A compound according to clause 22 wherein R⁴ is OH, and preferably wherein R⁴ is OH and R⁵ is CN.
24. A compound according to clause 22 or clause 23 wherein the substituted pyrimidine has any one of the following structures

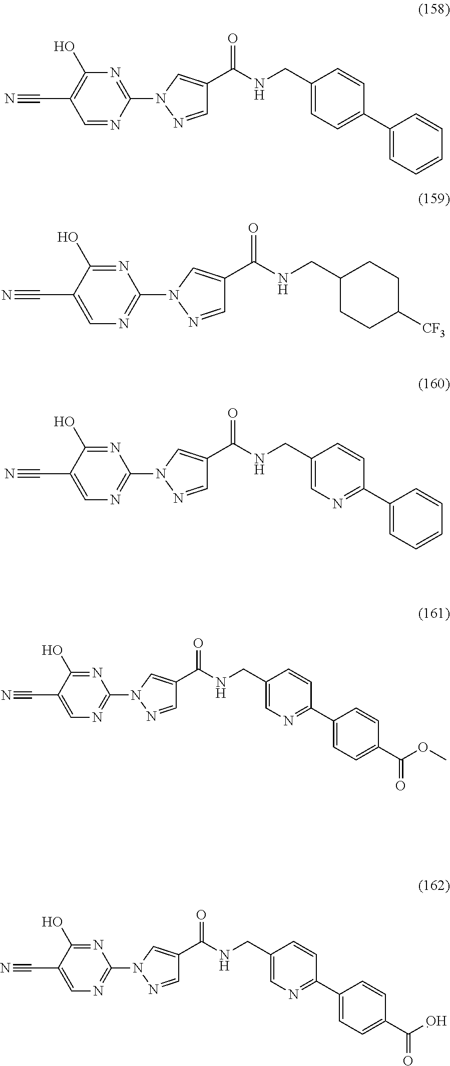 [SMILES: N#Cc1cnc(-n2cc(C(=O)NCc3ccc(-c4ccccc4)cc3)cn2)nc1O ; N#Cc1cnc(-n2cc(C(=O)NCC3CCC(C(F)(F)F)CC3)cn2)nc1O ; N#Cc1cnc(-n2cc(C(=O)NCc3ccc(-c4ccccc4)nc3)cn2)nc1O ; COC(=O)c1ccc(-c2ccc(CNC(=O)c3cnn(-c4ncc(C#N)c(O)n4)c3)cn2)cc1 ; N#Cc1cnc(-n2cc(C(=O)NCc3ccc(-c4ccc(C(=O)O)cc4)nc3)cn2)nc1O]

25. A pharmaceutical composition comprising a compound as defined in any one of the preceding clauses and a pharmaceutically acceptable carrier or diluent;
   optionally wherein the pharmaceutical composition further comprises one or more additional active agents selected from: ACE inhibitors, angiotensin II receptor agonists, beta receptor blockers, calcium antagonists, PDE inhibitors, mineralocorticoid receptor antagonists, diuretics, aspirin, iron supplements, vitamin B12 and folic acid supplements, statins, digitalis (digoxin) derivatives, tumor chemotherapeutics and antibiotics.
26. A compound as defined in any one of clauses 1 to 24, or a pharmaceutical composition as defined in clause 25, for use in treating the human or animal body by therapy.
27. A compound as defined in any one of clauses 1 to 24, or a pharmaceutical composition as defined in clause 25, for use in treating anaemia, ischemia, inflammation, Parkinson's disease, Alzheimer's disease, cancer, non-fatty liver disease, irritable bowel disease, cardiovascular disease, cardiac insufficiency, chronic kidney disease, renal insufficiency or sickle cell anaemia, or for use in skeletal muscle injury repair, increasing red blood cell count (RBC), increasing haemoglobin (HGB) production, increasing haematocrit (HCT) production, increasing Erythropoietin (EPO) production, wound healing, angiogenesis, revascularisation, stem cell activation or cardioprotection after myocardial infarction;
   optionally wherein the anaemia is renal anaemia, for instance anaemia associated with chronic kidney disease or anaemia in a dialysis patient; anaemia induced by chemotherapy; age-related anaemia; or anaemia occurring as a result of cancer such as leukaemia, multiple myeloma and smouldering myeloma;
   optionally wherein the ischemia is ischemia in a circulatory or cardiovascular disease, myocardial infarction, ischemia during a surgical operation, organ ischemia, an ischaemic disease, diabetic limb ischemia or sickle cell anaemia.

1. A compound which is a substituted azine of formula (I) or a pharmaceutically acceptable salt thereof 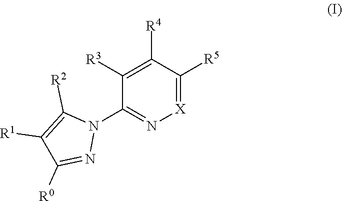
wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w} or —C(O)N(R^{x})R⁷;
R² is H, —OR^{q1} or unsubstituted or substituted C₁₋₆ alkyl; and R³ is H, —OR⁸ or unsubstituted or substituted C₁₋₆ alkyl; or R² is —N═ and R³ is =C(R^{y})— and R² and R³ together form a group of formula —N═C(R^{y})—;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN, —N(R^{t})C(O)N(R^{u})R^{v}, —C(O)OR^{w}, or —C(O)N(R^{x})R⁷;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸, R⁹ and R¹⁰ are each independently selected from H and unsubstituted or substituted C₁₋₆ alkyl; and
R^{t}, R^{u}, R^{v}, R^{w}, R^{x}, R^{y}, and R^{z} are each independently selected from H, unsubstituted or substituted C₁₋₆ alkyl, and unsubstituted or substituted phenyl;
R^{q} is H, unsubstituted or substituted C₁₋₆ alkyl, or unsubstituted or substituted phenyl;
provided that one of R¹ and R⁵ is —C(O)N(R)R⁷ and the other of R¹ and R⁵ is H, unsubstituted or substituted C₁₋₆ alkyl, unsubstituted or substituted aryl, unsubstituted or substituted heteroaryl, —CN, —N(R^{t})C(O)N(R^{u})RW or —C(O)OR^{w}.

2. A compound according to claim 1 wherein:
R⁰ is H or unsubstituted C₁₋₆ alkyl;
R¹ is H, —CN, —C(O)OR^{w} or —C(O)N(R^{x})R⁷;
R² is H or unsubstituted C₁₋₆ alkyl; and R³ is H or —OR⁸; or R² is —N═ and R³ is =C(R^{y})— and R² and R³ together form a group of formula —N═C(R^{y})—;
R⁴ is H, —OR⁹ or —C(O)OR¹⁰;
R⁵ is H, —CN, —C(O)OR^{w}, or —C(O)N(R^{x})R⁷;
R⁶ is H;
R⁷ is —CH(R¹¹)—Ar, —CH(R¹¹)-Ary-Ar, -Ary-Ar or —CH(R¹¹)-Cyc, wherein Ar is unsubstituted or substituted aryl or unsubstituted or substituted heteroaryl, Ary is unsubstituted arylene or unsubstituted heteroarylene, Cyc is unsubstituted or substituted C₃₋₁₀ cycloalkyl, and R¹¹ is H, —C(O)OR^{z} or unsubstituted C₁₋₄ alkyl;
R⁸, R⁹ and R¹⁰ are each independently selected from H and unsubstituted or substituted C₁₋₆ alkyl; and
R^{x} is H, R^{z} is H, R^{y} is H or unsubstituted C₁₋₆ alkyl, and R^{w} is H, unsubstituted C₁₋₆ alkyl, or C₁₋₆ alkyl which is substituted with phenyl or —OC(O)R″ wherein R^{ww} is phenyl, unsubstituted C₁₋₆ alkyl, —N(R^{a})(R^{b}), —C(O)R⁰, —OR^{d} or an amino acid, wherein R^{a}, R^{b}, R^{c} and R^{d} are each independently selected from H, unsubstituted or substituted C₁₋₆ alkyl and an amino acid;
provided that one of R¹ and R⁵ is —C(O)N(R)R⁷ and the other of R¹ and R⁵ is H, —CN or —C(O)OR^{w}.

3. A compound according to claim 1 or claim 2 wherein:
R⁰ is H or methyl;
R¹ is H, —CN, —C(O)OR^{w}, or —C(O)N(R′)R⁷;
R² is H or methyl; and R³ is H or —OR⁸; or R² is —N═ and R³ is =C(R^{y})— and R² and R³ together form a group of formula —N═C(R^{y})—;
R⁴ is H, —OR⁹ or —C(O)OR¹⁰;
R⁵ is H, —CN, —C(O)OR^{w}, or —C(O)N(R′)R⁷;
R⁶ is H;
R⁷ is —CH(R¹¹)—Ar, —CH(R¹¹)-Ary-Ar, -Ary-Ar or —CH(R¹¹)-Cyc; wherein Ar is unsubstituted phenyl, unsubstituted pyrimidyl, unsubstituted benzothiazole or phenyl substituted with —C(O)OH, —C(O)Ome, —C(O)Oet, —C(O)NH₂, —C(O)N(H)Me, -Ome or N-morpholino; Ary is unsubstituted phenylene or unsubstituted pyridylene; Cyc is unsubstituted cyclohexyl or cyclohexyl substituted with —CF₃ or —OCF₃; and R¹¹ is H, —C(O)OR^{z} or methyl;
R⁸, R⁹ and R¹⁰ are each independently selected from H, unsubstituted C₁₋₆ alkyl, and C₁₋₆ alkyl which is substituted with phenyl or —OC(O)R⁹⁹ wherein R⁹⁹ is phenyl, unsubstituted C₁₋₆ alkyl, —N(R^{a})(R), —C(O)R⁰, —OR^{d} or an amino acid, wherein R^{a}, R^{b}, R⁰ and R^{d} are each independently selected from H, unsubstituted or substituted C₁₋₆ alkyl and an amino acid; and
R^{x} is H;
R^{z} is H;
R^{w} is H, unsubstituted C₁₋₆ alkyl, or C₁₋₆ alkyl which is substituted with phenyl or —OC(O)R^{ww} wherein R″ is phenyl or unsubstituted C₁₋₆ alkyl; and
R^{y} is H or methyl;
provided that one of R¹ and R⁵ is —C(O)N(R^{x})R⁷ and the other of R¹ and R⁵ is H, —CN or —C(O)OR^{w}.

4. A compound according to any one of claims 1 to 3 wherein either:
(1) (a) R⁵ is —C(O)N(R^{x})R⁷ and (b) R³ is —OR⁸ or R⁴ is —OR⁹; or
(2) (a) R¹ is —C(O)N(R^{x})R⁷ and (b) R⁴ is —OR⁹ or —C(O)OR¹⁰, or R⁵ is —C(O)OR^{w}.

5. A compound according to any one of the preceding claims wherein the substituted azine has the formula (Ia) 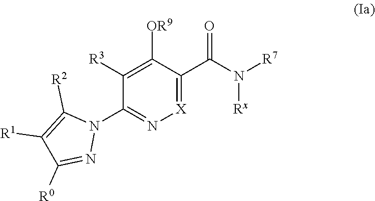
wherein
X is CR⁶ or N;
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R³ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, —CH₂C≡CCH₃, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is H or unsubstituted or substituted C₁₋₆ alkyl; and
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

6. A compound according to claim 5 wherein R⁹ is H.

7. A compound according to any one of the preceding claims wherein the substituted azine has any one of the following structures:

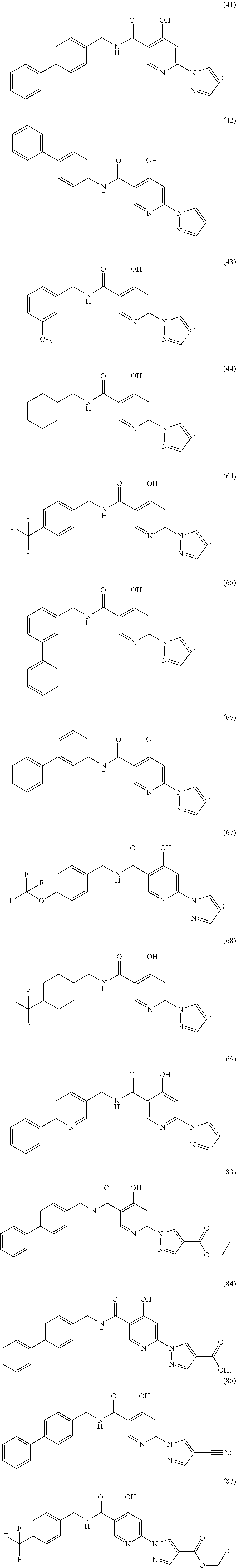

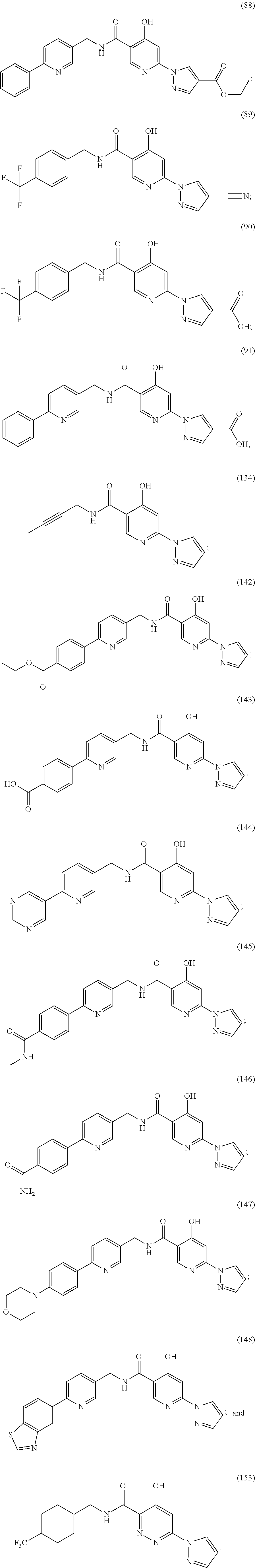

8. A compound according to any one of claims 1 to 4 wherein the substituted azine has the formula (Ib) 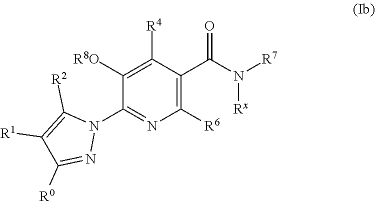
wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R² is H, —OR^{q} or unsubstituted or substituted C₁₋₆ alkyl;
R⁴ is H or unsubstituted or substituted C₁₋₆ alkyl;
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸ is H or unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

9. A compound according to claim 8 wherein R⁸ is H.

10. A compound according to claim 8 or claim 9 wherein the substituted azine has any one of the following structures

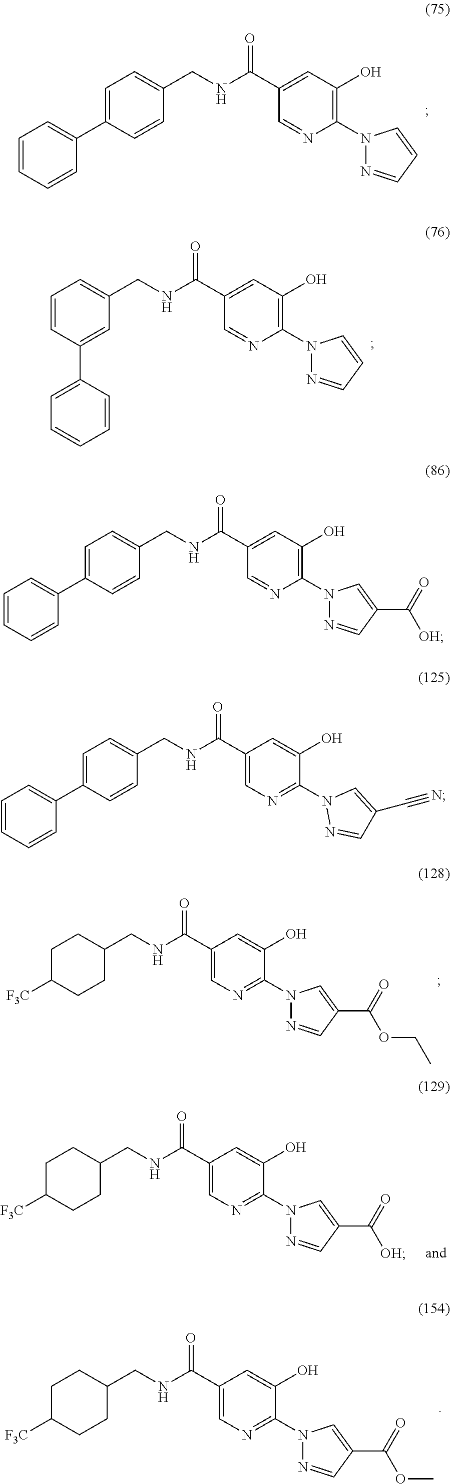

11. A compound according to any one of claims 1 to 4 wherein the substituted azine has the formula (Ic) 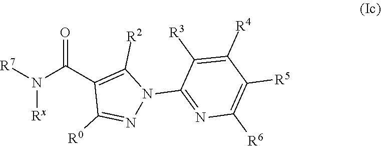
wherein
R⁰ is H or unsubstituted or substituted C₁₋₆ alkyl;
R² is H, —OR^{q} or unsubstituted or substituted C₁₋₆ alkyl;
R³ is H, —OR⁸ or unsubstituted or substituted C₁₋₆ alkyl;
R⁴ is H, unsubstituted or substituted C₁₋₆ alkyl, —OR⁹ or —C(O)OR¹⁰;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁸, R⁹ and R¹⁰ are each independently selected from H and unsubstituted or substituted C₁₋₆ alkyl;
R^{w}, R^{x} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

12. A compound according to claim 11 wherein the substituted azine has any one of the following structures:

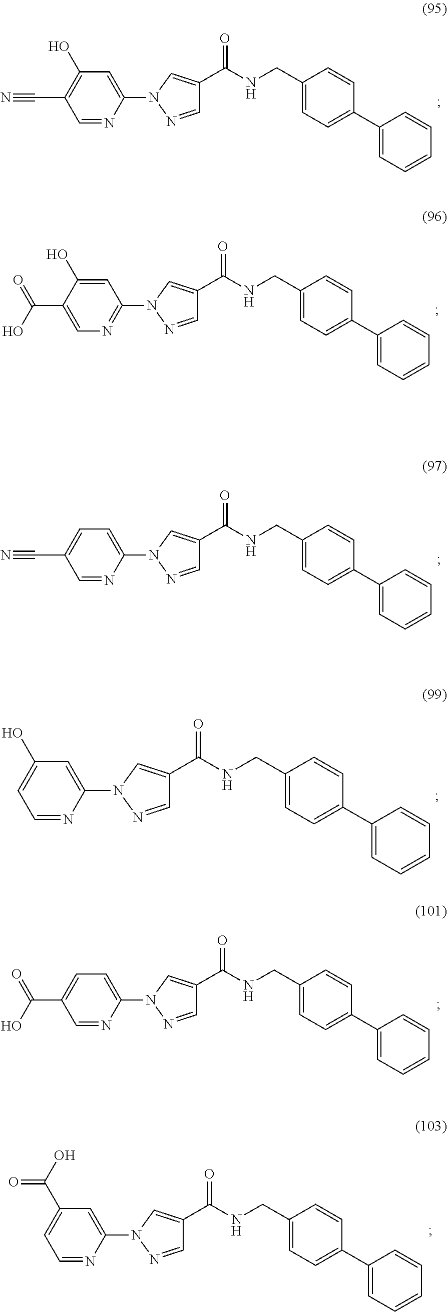 [SMILES: N#Cc1cnc(-n2cc(C(=O)NCc3ccc(-c4ccccc4)cc3)cn2)cc1O ; O=C(NCc1ccc(-c2ccccc2)cc1)c1cnn(-c2cc(O)c(C(=O)O)cn2)c1 ; N#Cc1ccc(-n2cc(C(=O)NCc3ccc(-c4ccccc4)cc3)cn2)nc1 ; O=C(NCc1ccc(-c2ccccc2)cc1)c1cnn(-c2cc(O)ccn2)c1 ; O=C(O)c1ccc(-n2cc(C(=O)NCc3ccc(-c4ccccc4)cc3)cn2)nc1 ; O=C(O)c1ccnc(-n2cc(C(=O)NCc3ccc(-c4ccccc4)cc3)cn2)c1]

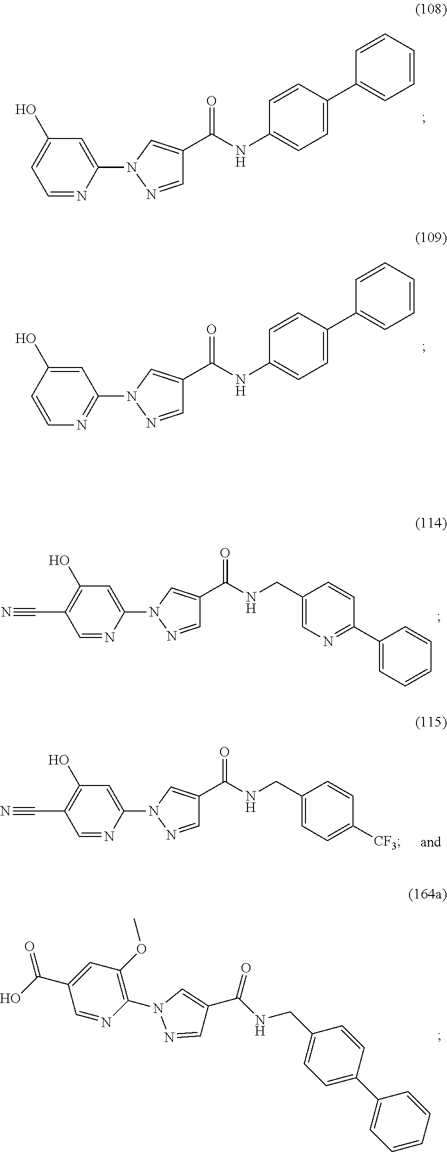 [SMILES: O=C(Nc1ccc(-c2ccccc2)cc1)c1cnn(-c2cc(O)ccn2)c1 ; O=C(Nc1ccc(-c2ccccc2)cc1)c1cnn(-c2cc(O)ccn2)c1 ; N#Cc1cnc(-n2cc(C(=O)NCc3ccc(-c4ccccc4)nc3)cn2)cc1O ; N#Cc1cnc(-n2cc(C(=O)NCc3ccc(C(F)(F)F)cc3)cn2)cc1O ; COc1cc(C(=O)O)cnc1-n1cc(C(=O)NCc2ccc(-c3ccccc3)cc2)cn1]

13. A compound according to any one of claims 1 to 4 wherein the substituted azine has the formula (Id) 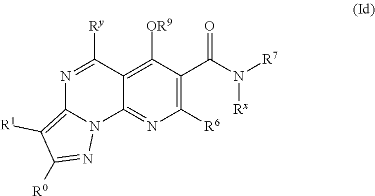
wherein
R⁰ is H or unsubstituted or substituted C₁₋₆ alkyl;
R¹ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R⁹ is H or unsubstituted or substituted C₁₋₆ alkyl; and
R^{w}, R^{x}, R^{y}, and R^{z} are each independently selected from H, unsubstituted or substituted C₁₋₄ alkyl, and unsubstituted or substituted phenyl.

14. A compound according to claim 13 wherein the substituted azine has any one of the following structures:

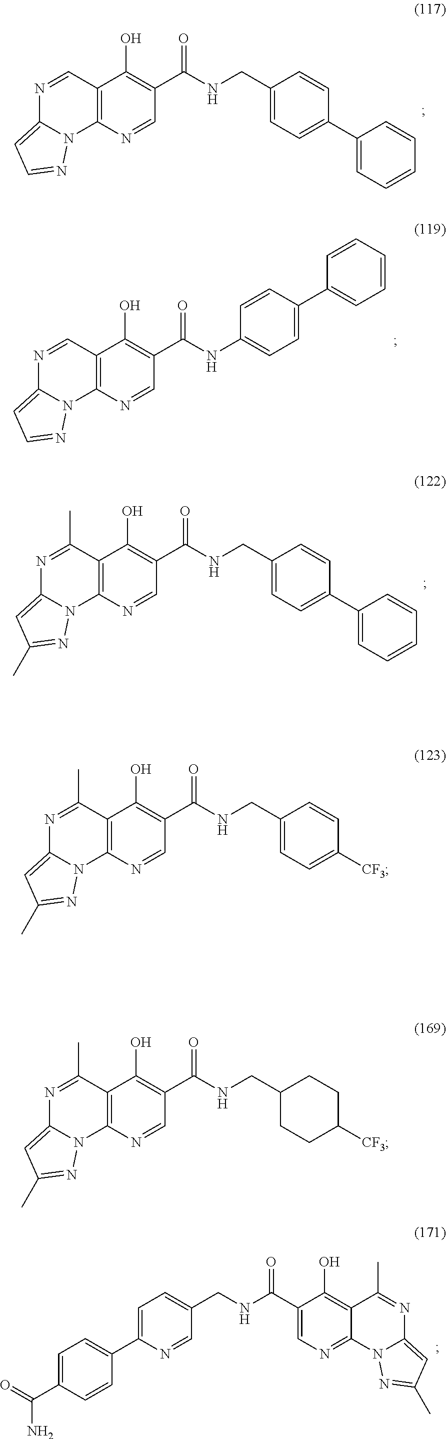 [SMILES: O=C(NCc1ccc(-c2ccccc2)cc1)c1cnc2c(cnc3ccnn32)c1O ; O=C(Nc1ccc(-c2ccccc2)cc1)c1cnc2c(cnc3ccnn32)c1O ; Cc1cc2nc(C)c3c(O)c(C(=O)NCc4ccc(-c5ccccc5)cc4)cnc3n2n1 ; Cc1cc2nc(C)c3c(O)c(C(=O)NCc4ccc(C(F)(F)F)cc4)cnc3n2n1 ; Cc1cc2nc(C)c3c(O)c(C(=O)NCC4CCC(C(F)(F)F)CC4)cnc3n2n1 ; Cc1cc2nc(C)c3c(O)c(C(=O)NCc4ccc(-c5ccc(C(N)=O)cc5)nc4)cnc3n2n1]

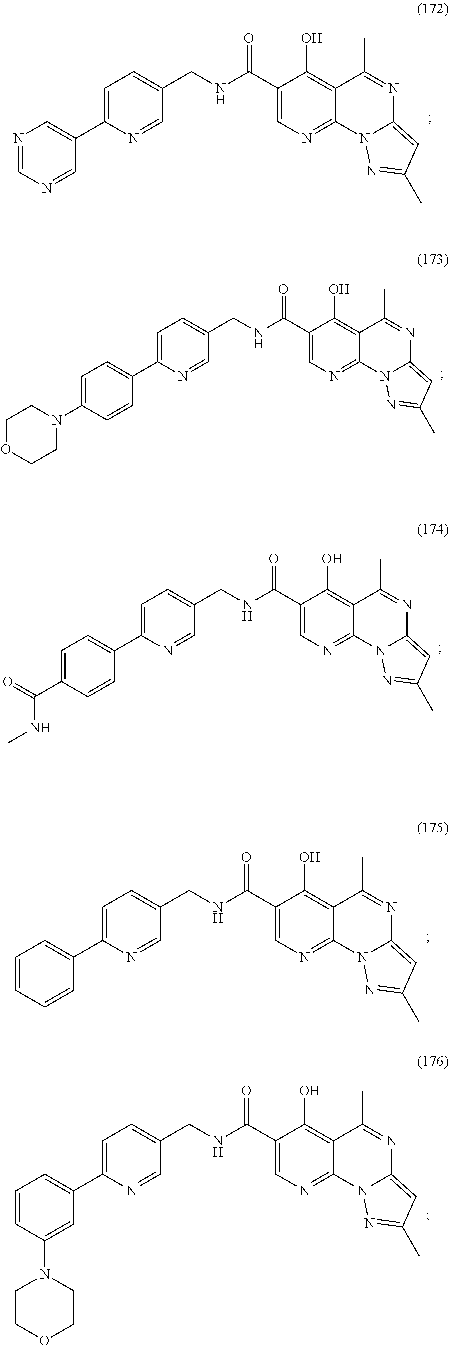 [SMILES: Cc1cc2nc(C)c3c(O)c(C(=O)NCc4ccc(-c5cncnc5)nc4)cnc3n2n1 ; Cc1cc2nc(C)c3c(O)c(C(=O)NCc4ccc(-c5ccc(N6CCOCC6)cc5)nc4)cnc3n2n1 ; CNC(=O)c1ccc(-c2ccc(CNC(=O)c3cnc4c(c(C)nc5cc(C)nn54)c3O)cn2)cc1 ; Cc1cc2nc(C)c3c(O)c(C(=O)NCc4ccc(-c5ccccc5)nc4)cnc3n2n1 ; Cc1cc2nc(C)c3c(O)c(C(=O)NCc4ccc(-c5cccc(N6CCOCC6)c5)nc4)cnc3n2n1]

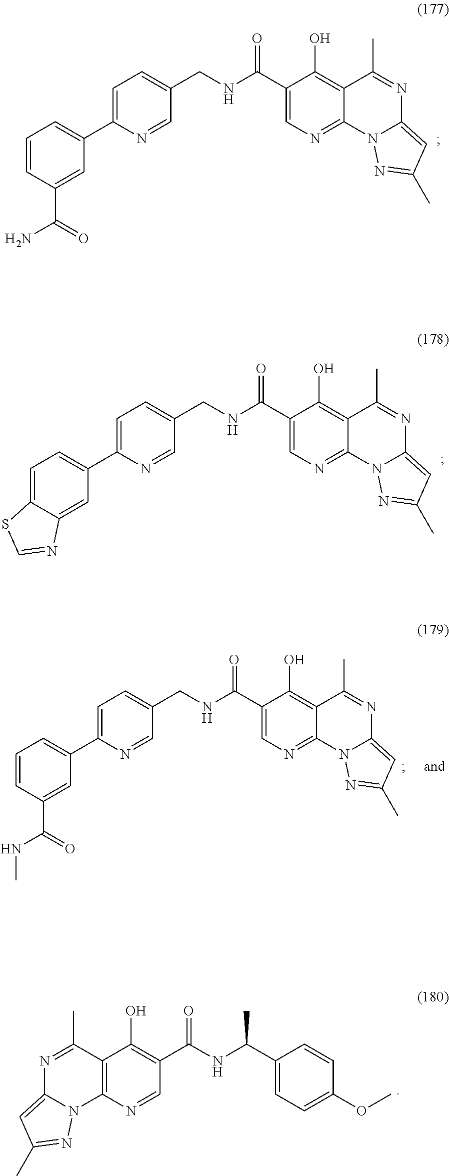

15. A compound according to any one of claims 1 to 3 wherein the substituted azine has any one of the following structures

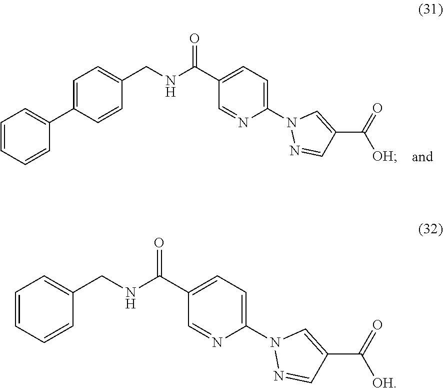 [SMILES: O=C(O)c1cnn(-c2ccc(C(=O)NCc3ccc(-c4ccccc4)cc3)cn2)c1 ; "and" ; O=C(O)c1cnn(-c2ccc(C(=O)NCc3ccccc3)cn2)c1]

16. A compound according to claim 5 wherein R⁹ is unsubstituted or substituted C₁₋₆ alkyl, optionally wherein the substituted azine of formula (Ia) has any one of the following structures

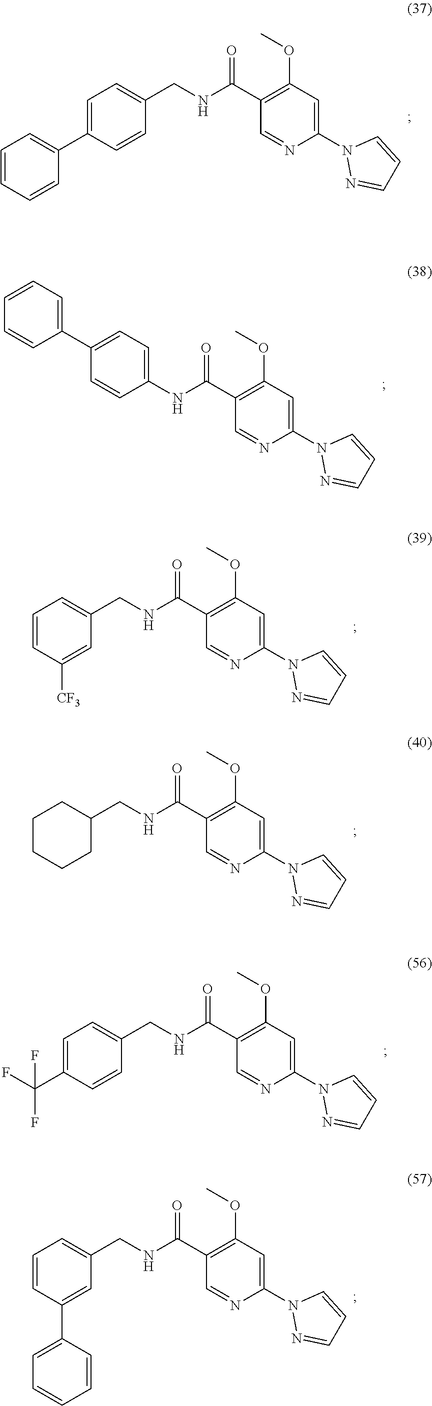 [SMILES: COc1cc(-n2cccn2)ncc1C(=O)NCc1ccc(-c2ccccc2)cc1 ; COc1cc(-n2cccn2)ncc1C(=O)Nc1ccc(-c2ccccc2)cc1 ; COc1cc(-n2cccn2)ncc1C(=O)NCc1cccc(C(F)(F)F)c1 ; COc1cc(-n2cccn2)ncc1C(=O)NCC1CCCCC1 ; COc1cc(-n2cccn2)ncc1C(=O)NCc1ccc(C(F)(F)F)cc1 ; COc1cc(-n2cccn2)ncc1C(=O)NCc1cccc(-c2ccccc2)c1]

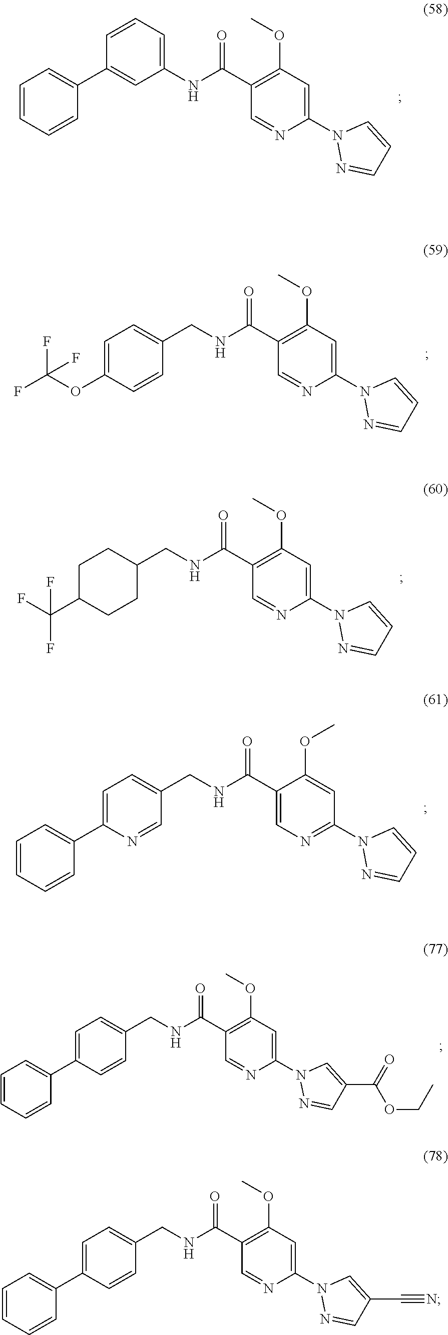 [SMILES: COc1cc(-n2cccn2)ncc1C(=O)Nc1cccc(-c2ccccc2)c1 ; COc1cc(-n2cccn2)ncc1C(=O)NCc1ccc(OC(F)(F)F)cc1 ; COc1cc(-n2cccn2)ncc1C(=O)NCC1CCC(C(F)(F)F)CC1 ; COc1cc(-n2cccn2)ncc1C(=O)NCc1ccc(-c2ccccc2)nc1 ; CCOC(=O)c1cnn(-c2cc(OC)c(C(=O)NCc3ccc(-c4ccccc4)cc3)cn2)c1 ; COc1cc(-n2cc(C#N)cn2)ncc1C(=O)NCc1ccc(-c2ccccc2)cc1]

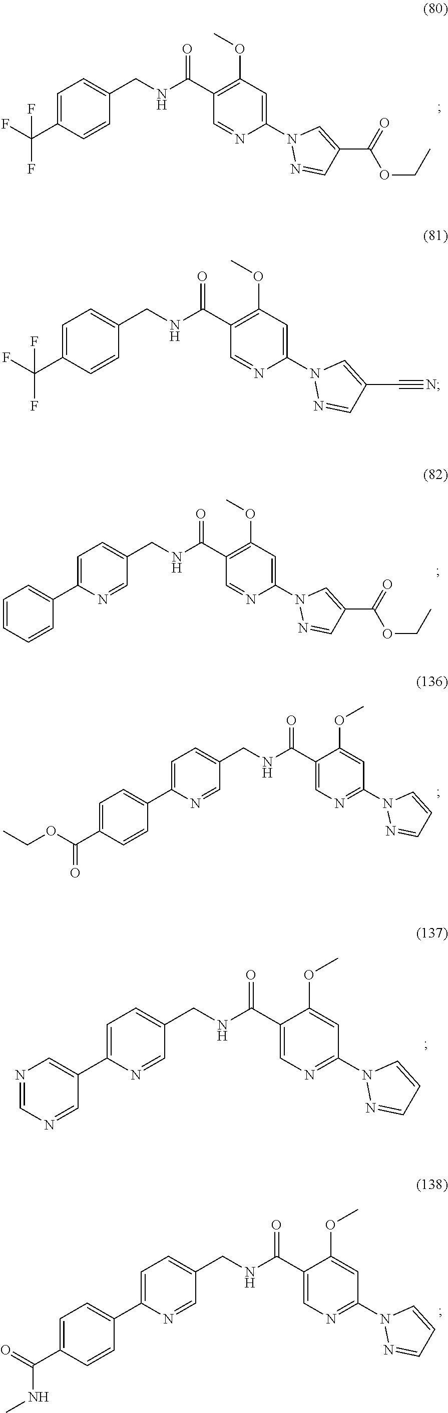

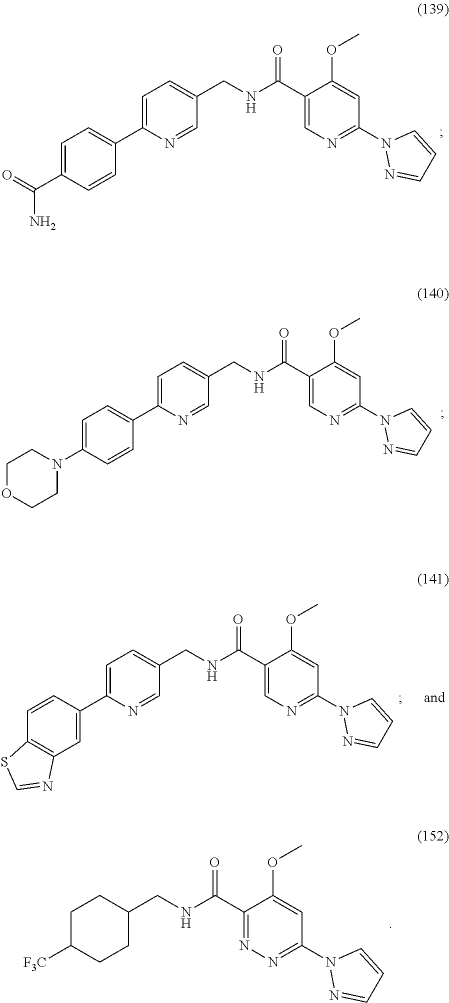 [SMILES: COc1cc(-n2cccn2)ncc1C(=O)NCc1ccc(-c2ccc(C(N)=O)cc2)nc1 ; COc1cc(-n2cccn2)ncc1C(=O)NCc1ccc(-c2ccc(N3CCOCC3)cc2)nc1 ; COc1cc(-n2cccn2)ncc1C(=O)NCc1ccc(-c2ccc3scnc3c2)nc1 ; COc1cc(-n2cccn2)nnc1C(=O)NCC1CCC(C(F)(F)F)CC1]

17. A compound according to claim 8 wherein R⁸ is unsubstituted or substituted C₁₋₆ alkyl, optionally wherein the substituted azine of formula (Ib) has any one of the following structures

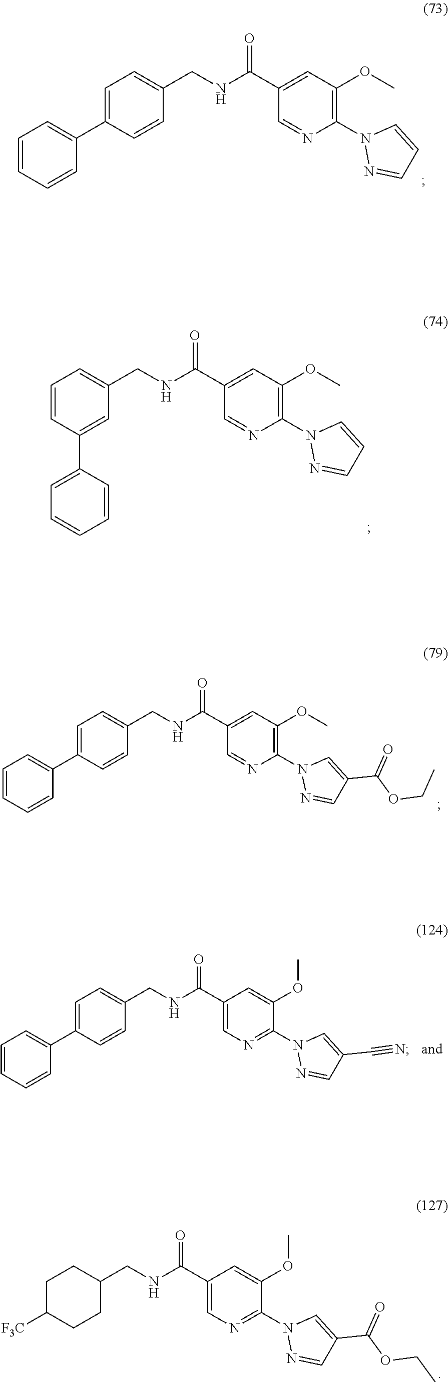 [SMILES: COc1cc(C(=O)NCc2ccc(-c3ccccc3)cc2)cnc1-n1cccn1 ; COc1cc(C(=O)NCc2cccc(-c3ccccc3)c2)cnc1-n1cccn1 ; CCOC(=O)c1cnn(-c2ncc(C(=O)NCc3ccc(-c4ccccc4)cc3)cc2OC)c1 ; COc1cc(C(=O)NCc2ccc(-c3ccccc3)cc2)cnc1-n1cc(C#N)cn1 ; CCOC(=O)c1cnn(-c2ncc(C(=O)NCC3CCC(C(F)(F)F)CC3)cc2OC)c1]

18. A compound according to claim 11 wherein R⁴ is —OR⁹ or —C(O)OR¹⁰, and/or R⁵ is —C(O)OR^{w}, wherein each of R⁹, R¹⁰ and R^{w} is independently unsubstituted or substituted C₁₋₆ alkyl; optionally wherein the substituted azine of formula (Ic) has any one of the following structures

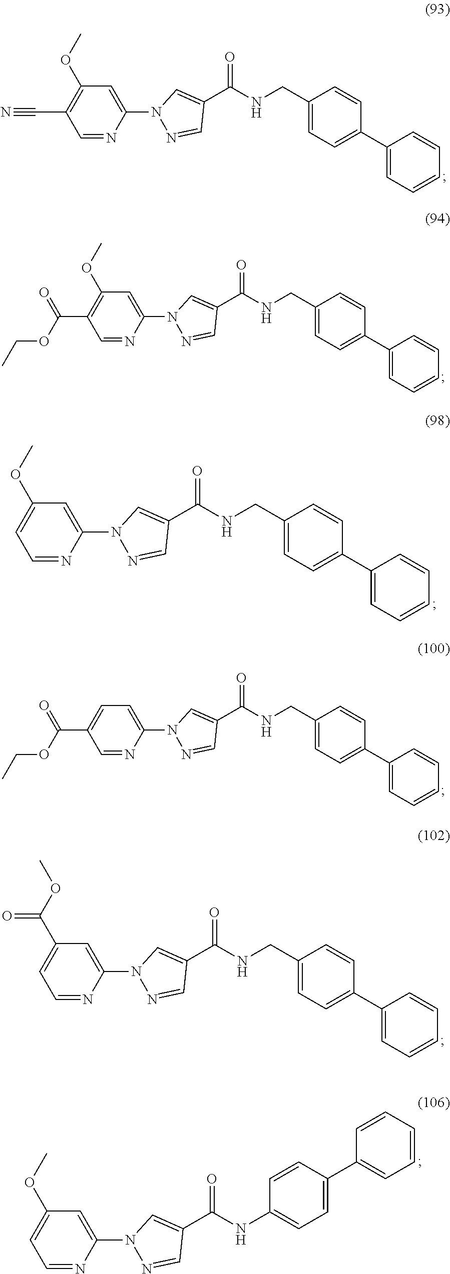 [SMILES: COc1cc(-n2cc(C(=O)NCc3ccc(-c4ccccc4)cc3)cn2)ncc1C#N ; CCOC(=O)c1cnc(-n2cc(C(=O)NCc3ccc(-c4ccccc4)cc3)cn2)cc1OC ; COc1ccnc(-n2cc(C(=O)NCc3ccc(-c4ccccc4)cc3)cn2)c1 ; CCOC(=O)c1ccc(-n2cc(C(=O)NCc3ccc(-c4ccccc4)cc3)cn2)nc1 ; COC(=O)c1ccnc(-n2cc(C(=O)NCc3ccc(-c4ccccc4)cc3)cn2)c1 ; COc1ccnc(-n2cc(C(=O)Nc3ccc(-c4ccccc4)cc3)cn2)c1]

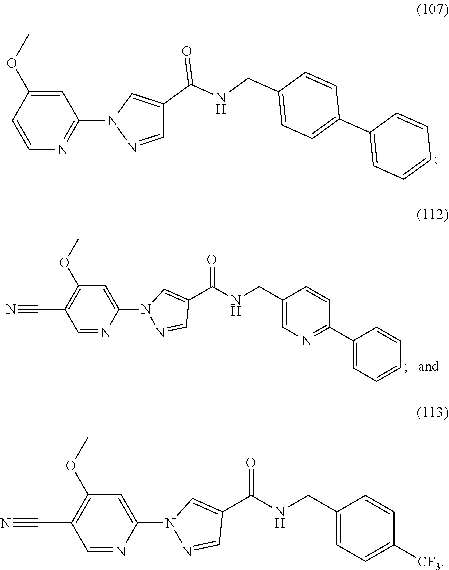 [SMILES: COc1ccnc(-n2cc(C(=O)NCc3ccc(-c4ccccc4)cc3)cn2)c1 ; COc1cc(-n2cc(C(=O)NCc3ccc(-c4ccccc4)nc3)cn2)ncc1C#N ; COc1cc(-n2cc(C(=O)NCc3ccc(C(F)(F)F)cc3)cn2)ncc1C#N]

19. A compound according to claim 13 wherein R⁹ is unsubstituted or substituted C₁₋₆ alkyl; optionally wherein the substituted azine of formula (Id) has the following structure

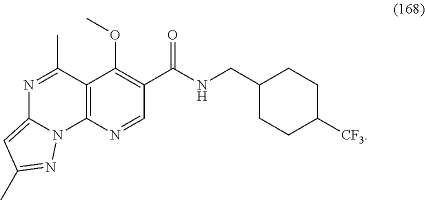 [SMILES: COc1c(C(=O)NCC2CCC(C(F)(F)F)CC2)cnc2c1c(C)nc1cc(C)nn12]

20. A compound which is a substituted pyrimidine of formula (IV) or a pharmaceutically acceptable salt thereof 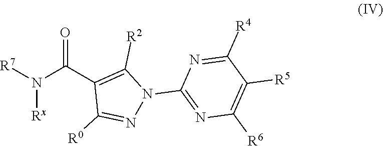
wherein
R⁰ is H or unsubstituted or substituted C₁₋₆ alkyl;
R² is H, —OR^{q} or unsubstituted or substituted C₁₋₆ alkyl;
R⁴ is —OR⁹, wherein R⁹ is selected from H and unsubstituted or substituted C₁₋₆ alkyl;
R⁵ is H, unsubstituted or substituted C₁₋₆ alkyl, unsubstituted or substituted aryl, unsubstituted or substituted heteroaryl, —CN or —C(O)OR^{w};
R⁶ is H or unsubstituted or substituted C₁₋₆ alkyl;
R⁷ is —CH(R¹¹)—Ar, —CH(R¹¹)-Ary-Ar, -Ary-Ar, —CH(R¹¹)-Cyc, -Cyc or —Ar, wherein Ar is unsubstituted or substituted aryl or unsubstituted or substituted heteroaryl, Ary is unsubstituted or substituted arylene or unsubstituted or substituted heteroarylene, Cyc is unsubstituted or substituted C₃₋₁₀ cycloalkyl, and R¹¹ is H, —C(O)OR^{z} or unsubstituted or substituted C₁₋₄ alkyl;
R^{x} is H, unsubstituted or substituted C₁₋₄ alkyl, or unsubstituted or substituted phenyl;
R^{w} and R^{z} are each independently selected from H, unsubstituted or substituted C₁₋₄ alkyl, and unsubstituted or substituted phenyl; and
R^{q} is H, unsubstituted or substituted C₁₋₆ alkyl, or unsubstituted or substituted phenyl.

21. A compound according to claim 20 wherein R⁴ is OH, and preferably wherein R⁴ is OH and R⁵ is CN.

22. A compound according to claim 20 or claim 21 wherein the substituted pyrimidine has any one of the following structures:

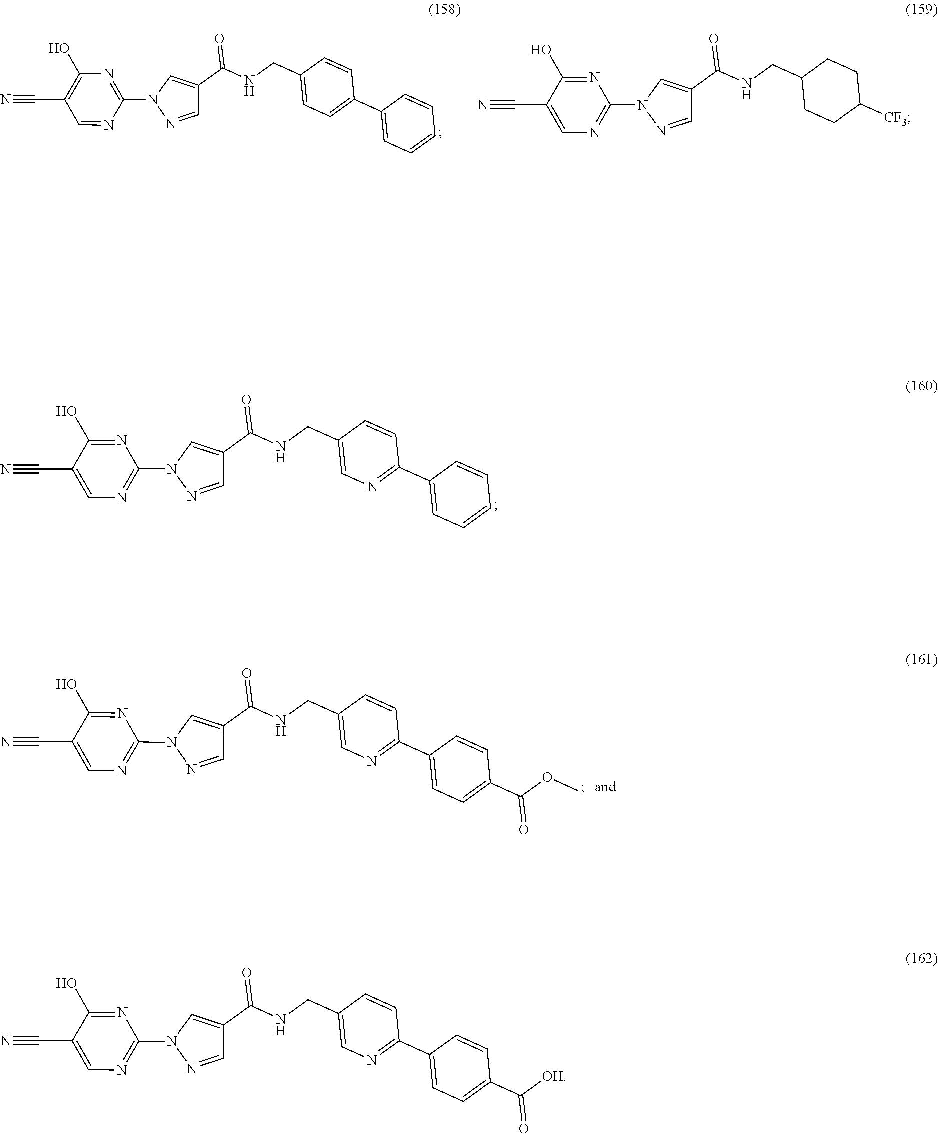

23. A pharmaceutical composition comprising a compound as defined in any one of the preceding claims and a pharmaceutically acceptable carrier or diluent;
optionally wherein the pharmaceutical composition further comprises one or more additional active agents selected from: ACE inhibitors, angiotensin II receptor agonists, beta receptor blockers, calcium antagonists, PDE inhibitors, mineralocorticoid receptor antagonists, diuretics, aspirin, iron supplements, vitamin B12 and folic acid supplements, statins, digitalis (digoxin) derivatives, tumor chemotherapeutics and antibiotics.

24. A compound as defined in any one of claims 1 to 22, or a pharmaceutical composition as defined in claim 23, for use in treating the human or animal body by therapy.

25. A compound as defined in any one of claims 1 to 22, or a pharmaceutical composition as defined in claim 23, for use in treating anaemia, ischemia, inflammation, Parkinson's disease, Alzheimer's disease, non-fatty liver disease, irritable bowel disease, cardiovascular disease, cardiac insufficiency, chronic kidney disease, renal insufficiency or sickle cell anaemia, or for use in skeletal muscle injury repair, increasing red blood cell count (RBC), increasing haemoglobin (HGB) production, increasing haematocrit (HCT) production, increasing Erythropoietin (EPO) production, wound healing, angiogenesis, revascularisation, stem cell activation or cardioprotection after myocardial infarction;
optionally wherein the anaemia is renal anaemia, for instance anaemia associated with chronic kidney disease or anaemia in a dialysis patient; anaemia induced by chemotherapy; age-related anaemia; or anaemia occurring as a result of cancer such as leukaemia, multiple myeloma and smouldering myeloma;
optionally wherein the ischemia is ischemia in a circulatory or cardiovascular disease, myocardial infarction, ischemia during a surgical operation, organ ischemia, an ischaemic disease, diabetic limb ischemia or sickle cell anaemia.
